SEQUENCE LISTING (159 sequences)

SEQ ID NO: 1 - protein, 122 aa, synthetic construct
EVILVESGGGLVKPGGSLKLSCAASGFTFSSFAMSWVRQTPEKRLEWVATISSGSIYIYYTDGVKGRFTISRDNAKNTVHLQMSSLRSEDTAMYYCARRGIYYGYDGYAMDYWGQGTSVTVS

SEQ ID NO: 2 - protein, 112 aa, synthetic construct
AVVMTQTPLSLPVSLGDQASISCRSSQSLVHSNGNTYLHWYMQKPGQSPKVLIYKVSNRFSGVPDRFSGSGSGTDFTLKISRVEADDLGIYFCSQSTHIPLAFGAGTKLELK

SEQ ID NO: 3 - protein, 123 aa, synthetic construct
QIQLVQSGPELKKPGETVKISCKASGYSFRDYGMNWVKQAPGRTFKWMGWINTYTGAPVYAADFKGRFAFSLDTSASAAFLQINNLKNEDTATYFCARWVSYGNNRNWFFDFWGAGTTVTVSS

SEQ ID NO: 4 - DNA, 369 bp, synthetic construct
  source 1..369: mol_type = other DNA
cagatccagttggtgcagtctggacctgagctgaagaagcctggagagacagtcaagatctcctgcaaggcttctgggtattccttcagagactatggaatgaactgggtgaagcaggctccaggaaggacttttaagtggatgggctggataaacacctacactggagcgccagtatatgctgctgacttcaagggacggtttgccttctctttggacacctctgccagcgctgcctttttgcagatcaacaacctcaaaaatgaagacacggctacatatttctgtgcaagatgggtctcctacggtaataaccgcaactggttcttcgatttttggggcgcagggaccacggtcaccgtctcctca

SEQ ID NO: 5 - DNA, 369 bp, synthetic construct
  source 1..369: mol_type = other DNA
caaattcagttggttcaatccggccctgagctcaagaagcctggagagacagtgaagataagttgtaaggctagtggctattcatttcgagattatgggatgaattgggtcaagcaggccccagggcggaccttcaaatggatggggtggatcaatacttacactggcgcaccagtatatgcagctgattttaagggtcgctttgcattttcacttgatacttcagccagtgccgcttttttgcaaatcaacaatctcaaaaatgaagacactgctacatatttctgcgccaggtgggtgagctatggcaataacagaaattggttctttgacttttggggcgcaggcaccaccgtcactgtctcatca

SEQ ID NO: 6 - protein, 10 aa, synthetic construct
GYSFRDYGMN

SEQ ID NO: 7 - protein, 17 aa, synthetic construct
WINTYTGAPVYAADFKG

SEQ ID NO: 8 - protein, 14 aa, synthetic construct
WVSYGNNRNWFFDF

SEQ ID NO: 9 - protein, 112 aa, synthetic construct
DVLMTQTPLSLPVRLGDQASISCRSSQTLVHSNGNTYLEWYLQKPGQSPKLLIYKVSNRLSGVPDRFSGSGSGTDFTLKISRVETEDLGVYYCFQGSHGPWTFGGGTKLEIK

SEQ ID NO: 10 - DNA, 336 bp, synthetic construct
  source 1..336: mol_type = other DNA
gatgttttgatgacccaaactccactctccctgcctgtccgtcttggagatcaggcctccatctcttgtagatctagtcagacccttgtacatagtaatggaaacacctatttagaatggtacctgcagaaaccaggccagtctccaaaactcttgatctacaaagtttccaatcgactttctggggtcccagacaggttcagtggcagtggatcagggacagatttcacactcaagatcagcagagtggagactgaggatctgggagtttattactgctttcaaggttcacatggtccgtggacgttcggtggaggcaccaagctggaaatcaaa

SEQ ID NO: 11 - DNA, 336 bp, synthetic construct
  source 1..336: mol_type = other DNA
gacgtacttatgacacaaactcccttgagcttgccagtacggcttggcgatcaagcttcaatttcatgtcgttcttctcaaacacttgtccactcaaatgggaatacatatttggaatggtatctccaaaagcccggccaatccccaaaattgttgatttacaaggtgtctaatcgactctcaggcgtccccgaccgattctccgggagcgggtccggtacagacttcaccttgaaaatctccagggtagaaactgaagacctcggagtctactattgtttccaggggtcacacggcccctggacatttggaggaggaactaagctcgagatcaaa

SEQ ID NO: 12 - protein, 16 aa, synthetic construct
RSSQTLVHSNGNTYLE

SEQ ID NO: 13 - protein, 7 aa, synthetic construct
KVSNRLS

SEQ ID NO: 14 - protein, 9 aa, synthetic construct
FQGSHGPWT

SEQ ID NO: 15 - protein, 118 aa, synthetic construct
EVQLQQSGPELVKPGASVKISCKTSGYTFTDYTMHWVRQSHGKSLEWIGSFNPNNGGLTNYNQKFKGKATLTVDKSSSTVYMDLRSLTSEDSAVYYCTRIRATGFDSWGQGTTLTVSS

SEQ ID NO: 16 - DNA, 354 bp, synthetic construct
  source 1..354: mol_type = other DNA
gaggtccagctgcaacagtctggacctgagctggtgaagcctggggcttcagtgaagatatcctgcaagacttctggatacacattcactgattacaccatgcactgggtgaggcagagccatggaaagagccttgagtggattggaagttttaatcctaacaatggtggtcttactaactacaaccagaagttcaagggcaaggccacattgactgtggacaagtcttccagcacagtgtacatggacctccgcagcctgacatctgaggattctgcagtctattactgtacaagaatccgggctacgggctttgactcctggggccagggcaccactctcacagtctcctca

SEQ ID NO: 17 - DNA, 354 bp, synthetic construct
  source 1..354: mol_type = other DNA
gaggtacaactgcaacagagtggacctgaacttgtcaaacctggagcaagtgtgaagattagctgtaaaaccagtggctacacatttaccgattatactatgcactgggtaagacagagccacggaaaatcactggagtggattggtagtttcaatcctaacaacggaggattgacaaattacaaccagaagttcaaagggaaagccaccttgacagttgataagtcctcaagtaccgtgtatatggatctgcgttctctcacaagtgaagatagcgcagtttactactgtacccgcatccgagccaccgggttcgattcatggggtcaggggacaacactgactgtttcttct

SEQ ID NO: 18 - protein, 10 aa, synthetic construct
GYTFTDYTMH

SEQ ID NO: 19 - protein, 18 aa, synthetic construct
SFNPNNGGLTNYNQKFKG

SEQ ID NO: 20 - protein, 8 aa, synthetic construct
IRATGFDS

SEQ ID NO: 21 - protein, 113 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISNVQAEDLTVYYCKQSYNPPWTFGGGTKLEIK

SEQ ID NO: 22 - DNA, 339 bp, synthetic construct
  source 1..339: mol_type = other DNA
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcaatgtgcaggctgaagacctgacagtttattactgcaagcaatcttataatcctccgtggacgttcggtggaggcaccaagctggaaatcaaa

SEQ ID NO: 23 - DNA, 339 bp, synthetic construct
  source 1..339: mol_type = other DNA
gacatagttatgtcccagtctccatccagcttggctgtcagcgccggagagaaagtgactatgagttgtaaatcttcccagtccctgcttaactcacgtactcggaagaattatcttgcctggtatcaacaaaagccaggtcaaagtcctaagctccttatttactgggcctcaacacgggagtcaggtgtccccgatcgcttcacaggtagtgggagtggtactgacttcactctcaccatttcaaatgtccaagcagaagacttgactgtgtattactgtaagcagagttacaaccctccttggacctttggtggggggaccaaactggagatcaag

SEQ ID NO: 24 - protein, 17 aa, synthetic construct
KSSQSLLNSRTRKNYLA

SEQ ID NO: 25 - protein, 7 aa, synthetic construct
WASTRES

SEQ ID NO: 26 - protein, 9 aa, synthetic construct
KQSYNPPWT

SEQ ID NO: 27 - protein, 116 aa, synthetic construct
EVQVQQSGPELVKPGASVKMSCKASGYTFTSYVMHWVKQKPGQGLEWIGYINPNNDNINYNEKFKGKASLTSDKSSNTVYMELSSLTSEDSAVYYCAGYGNSGANWGQGTLVTVSA

SEQ ID NO: 28 - DNA, 348 bp, synthetic construct
  source 1..348: mol_type = other DNA
gaggtccaggtacagcagtctggacctgaactggtaaagcctggggcttcagtgaagatgtcctgtaaggcttctggatacacattcactagctatgtcatgcactgggtgaagcagaagcctgggcagggccttgagtggattggatatattaatcctaacaatgataatattaactacaatgagaagttcaaaggcaaggcctcactgacttcagacaaatcctccaacacagtctacatggagctcagcagcctgacctctgaggactctgcggtctattactgtgcaggctatggtaactccggagctaactggggccaagggactctggtcactgtctctgca

SEQ ID NO: 29 - DNA, 348 bp, synthetic construct
  source 1..348: mol_type = other DNA
gaagttcaagttcagcaaagcgggcctgagcttgtcaagccaggcgcatcagtcaaaatgagctgtaaggcttccgggtacaccttcaccagttatgtcatgcattgggtaaaacaaaagccaggacagggactcgagtggataggatacattaacccaaataacgacaacattaactacaacgagaaattcaagggcaaagcatcattgacttccgataaatcctctaacaccgtgtacatggagctgagttcattgaccagcgaggattctgccgtgtactactgtgcaggttatggcaactctggtgctaactgggggcaggggactctggtcacagtcagcgca

SEQ ID NO: 30 - protein, 10 aa, synthetic construct
GYTFTSYVMH

SEQ ID NO: 31 - protein, 17 aa, synthetic construct
YINPNNDNINYNEKFKG

SEQ ID NO: 32 - protein, 7 aa, synthetic construct
YGNSGAN

SEQ ID NO: 33 - protein, 107 aa, synthetic construct
DIQMTQSPASLSASVGETVTITCRTSKNIFNFLAWYHQKQGRSPRLLVSHTKTLAAGVPSRFSGSGSGTQFSLKINSLQPEDFGIYYCQHHYGTPWTFGGGTKLEIK

SEQ ID NO: 34 - DNA, 321 bp, synthetic construct
  source 1..321: mol_type = other DNA
gacatccagatgactcagtctccagcctccctatctgcatctgtgggagaaactgtcaccatcacatgtcgaacaagtaaaaatattttcaattttttagcatggtatcaccagaaacagggaagatctcctcgactcctggtctctcatacaaaaaccttagcagcaggtgtgccatcaaggttcagtggcagtggctcaggcacacagttttctctgaagatcaacagcctgcagcctgaagattttgggatttattactgtcaacatcattatggtactccgtggacgttcggtggaggcaccaaactggaaatcaaa

SEQ ID NO: 35 - DNA, 321 bp, synthetic construct
  source 1..321: mol_type = other DNA
gacattcagatgacccagtcaccagcatctttgagcgcatccgttggggagactgtgacaatcacatgccgaaccagtaagaacatcttcaacttcctcgcatggtaccatcaaaagcagggcaggtctcccagactgcttgtctctcacaccaagacactggcagcaggcgtccccagccggtttagtggtagtggatctggcacacagtttagtttgaaaatcaattccctgcaacccgaagacttcggcatatactattgccagcaccactatgggacaccttggactttcggaggtggtactaaacttgagattaaa

SEQ ID NO: 36 - protein, 11 aa, synthetic construct
RTSKNIFNFLA

SEQ ID NO: 37 - protein, 7 aa, synthetic construct
HTKTLAA

SEQ ID NO: 38 - protein, 9 aa, synthetic construct
QHHYGTPWT

SEQ ID NO: 39 - protein, 120 aa, synthetic construct
QIQLVQSGPELKKPGETVKISCKASGYTFTNYGVKWVKQAPGKDLKWMGWINTYTGNPIYAADFKGRFAFSLETSASTAFLQINNLKNEDTATYFCVRFQYGDYRYFDVWGAGTTVTVSS

SEQ ID NO: 40 - DNA, 360 bp, synthetic construct
  source 1..360: mol_type = other DNA
cagatccagttggtgcagtctggacctgagctgaagaagcctggagagacagtcaagatctcctgcaaggcttctgggtataccttcacaaactatggagtgaagtgggtgaagcaggctccaggaaaggatttaaagtggatgggctggataaacacctacactggaaatccaatttatgctgctgacttcaagggacggtttgccttctctttggagacctctgccagcactgcctttttgcagatcaacaacctcaaaaatgaggacacggctacatatttctgtgtaagattccaatatggcgattaccggtacttcgatgtctggggcgcagggaccacggtcaccgtctcctca

SEQ ID NO: 41 - DNA, 360 bp, synthetic construct
  source 1..360: mol_type = other DNA
caaatccaacttgtccagagcggtcccgagttgaagaagcctggcgaaaccgtgaaaatctcatgcaaggccagtggatatacatttacaaactatggcgtcaagtgggtgaaacaagccccaggtaaagacttgaaatggatgggatggatcaacacatacacagggaatcctatctatgcagccgactttaaaggcagatttgccttcagtttggagacatctgcctccaccgctttcctgcaaataaataacctgaaaaatgaagataccgctacatacttctgtgtacggttccagtacggagattaccgctatttcgatgtgtggggcgcaggtaccacagtaaccgtctcctca

SEQ ID NO: 42 - protein, 10 aa, synthetic construct
GYTFTNYGVK

SEQ ID NO: 43 - protein, 17 aa, synthetic construct
WINTYTGNPIYAADFKG

SEQ ID NO: 44 - protein, 11 aa, synthetic construct
FQYGDYRYFDV

SEQ ID NO: 45 - protein, 106 aa, synthetic construct
QIILSQSPAILSASPGEKVTMTCRANSGISFINWYQQKPGSSPKPWIYGTANLASGVPARFGGSGSGTSYSLTISRVEAEDAATYYCQQWSSNPLTFGAGTKLELR

SEQ ID NO: 46 - DNA, 318 bp, synthetic construct
  source 1..318: mol_type = other DNA
caaattattctctcccagtctccagcaatcctgtctgcatctccaggggagaaggtcacgatgacttgcagggccaactcaggtattagtttcatcaactggtaccagcagaagccaggatcctcccccaaaccctggatttatggcacagccaacctggcttctggagtccctgctcgcttcggtggcagtgggtctgggacttcttactctctcacaatcagcagagtggaggctgaagacgctgccacttattactgccagcagtggagtagtaacccgctcacgttcggtgctgggaccaagctggagttgaga

SEQ ID NO: 47 - DNA, 318 bp, synthetic construct
  source 1..318: mol_type = other DNA
caaataattctgtcacagtcccccgctatacttagtgcttcaccaggagaaaaagtgaccatgacttgtagagctaattctggcatatcattcatcaactggtatcaacaaaagccaggttcctcccccaagccatggatttacgggaccgccaaccttgcttctggggtacccgctcgtttcggcggatcaggttcaggaacttcctatagcctcactatcagtcgggttgaagctgaggatgccgctacatattactgccagcaatggtctagtaatccacttacctttggagctggcaccaaattggaacttcgt

SEQ ID NO: 48 - protein, 10 aa, synthetic construct
RANSGISFIN

SEQ ID NO: 49 - protein, 7 aa, synthetic construct
GTANLAS

SEQ ID NO: 50 - protein, 9 aa, synthetic construct
QQWSSNPLT

SEQ ID NO: 51 - protein, 116 aa, synthetic construct
EVQLQQSGPELVKPGASVKLSCKASGYTVTSYVMHWVKQKPGQGLEWIGYINPYSDVTNCNEKFKGKATLTSDKTSSTAYMELSSLTSEDSAVYYCSSYGGGFAYWGQGTLVTVSA

SEQ ID NO: 52 - DNA, 348 bp, synthetic construct
  source 1..348: mol_type = other DNA
gaggtccagctgcagcagtctggacctgagctggtaaagcctggggcttcagtgaagctgtcctgcaaggcttctggatacacagtcactagctatgttatgcactgggtgaagcagaagcctgggcagggccttgagtggattggatatattaatccttacagtgatgttactaactgcaatgagaagttcaaaggcaaggccacactgacttcagacaaaacctccagcacagcctacatggagctcagcagcctgacctctgaggactctgcggtctattactgttcctcctacggtggggggtttgcttactggggccaagggactctggtcactgtctctgca

SEQ ID NO: 53 - DNA, 348 bp, synthetic construct
  source 1..348: mol_type = other DNA
gaagtccagcttcagcaatccggcccagaactggtaaaaccaggcgcaagtgttaagttgagttgcaaagccagtggttataccgttacttcatacgtcatgcattgggtaaaacaaaagcccggccaagggcttgaatggatcggctacatcaacccttactctgacgtcaccaactgcaacgagaaattcaaagggaaagccacattgacctctgacaagacaagcagtaccgcctacatggagctttctagtttgacttctgaagactctgctgtctactactgtagcagctacggcggcggctttgcttactggggccagggtacattggtgactgtgagtgca

SEQ ID NO: 54 - protein, 10 aa, synthetic construct
GYTVTSYVMH

SEQ ID NO: 55 - protein, 17 aa, synthetic construct
YINPYSDVTNCNEKFKG

SEQ ID NO: 56 - protein, 7 aa, synthetic construct
YGGGFAY

SEQ ID NO: 57 - protein, 107 aa, synthetic construct
DIQMTQSPASLSASVGEPVTITCRASKNIYTYLAWYHQKQGKSPQFLVYNARTLAGGVPSRLSGSGSVTQFSLNINTLHREDLGTYFCQHHYDTPYTFGGGTNLEIK

SEQ ID NO: 58 - DNA, 321 bp, synthetic construct
  source 1..321: mol_type = other DNA
gacatccagatgactcagtctccagcctccctatctgcatctgtgggagaacctgtcaccatcacatgtcgagcaagtaagaatatttacacatatttagcatggtatcaccagaaacagggaaaatctcctcagttcctggtctataatgcaagaaccttagcaggaggtgtgccatcaaggctcagtggcagtggatcagtcacgcagttttctctaaacatcaacaccttgcatcgagaagatttagggacttacttctgtcaacatcattatgatactccgtacacgttcggaggggggaccaacctggaaataaaa

SEQ ID NO: 59 - DNA, 321 bp, synthetic construct
  source 1..321: mol_type = other DNA
gacatccagatgacacagtcaccagcatccctgtccgcctcagttggggagcctgttaccataacttgtcgggcaagcaaaaacatatacacctatttggcttggtatcaccaaaagcaaggtaagtcacctcagtttcttgtatataatgcccgcacacttgctggcggagtaccctctcgattgtctggatctggcagcgttacccaattcagcctgaacatcaacaccctccatcgggaagatttgggtacctatttctgtcaacatcactacgacaccccatacaccttcggaggcggcacaaatttggaaattaaa

SEQ ID NO: 60 - protein, 11 aa, synthetic construct
RASKNIYTYLA

SEQ ID NO: 61 - protein, 7 aa, synthetic construct
NARTLAG

SEQ ID NO: 62 - protein, 9 aa, synthetic construct
QHHYDTPYT

SEQ ID NO: 63 - protein, 113 aa, synthetic construct
EVQLQQSGPELVKPGASVKMSCKASGYTFSSYVMHWVKQKPGQGLEWIGYINPYSDVTNYNEKFKGKATLTSDRSSNTAYMELSSLTSEDSAVYYCARNYFDWGRGTLVTVSA

SEQ ID NO: 64 - DNA, 339 bp, synthetic construct
  source 1..339: mol_type = other DNA
gaggtccagctgcagcagtctggacctgagctggtaaagcctggggcttcagtgaagatgtcctgcaaggcttctggatacacattctctagctatgttatgcactgggtgaagcagaagcctgggcagggccttgagtggattggatatattaatccttacagtgatgtcactaactacaatgagaagttcaaaggcaaggccacactgacttcagacagatcctccaacacagcctacatggaactcagcagcctgacctctgaggactctgcggtctattactgtgcaagaaattacttcgactggggccgagggactctggtcacagtctctgca

SEQ ID NO: 65 - DNA, 339 bp, synthetic construct
  source 1..339: mol_type = other DNA
gaggtacagcttcagcagagtggtccagaactcgtcaagcctggggcaagcgttaagatgagttgtaaagcatccggttacacattcagtagctatgttatgcactgggtcaaacagaagcctgggcaggggttggagtggatcggatatataaatccctattcagacgtaactaattataatgaaaagttcaaggggaaagcaaccttgacaagtgaccggtcatctaataccgcatacatggagctgagctcattgacaagtgaggactctgctgtgtattactgtgcccggaactacttcgactggggtaggggcacactggtaactgttagtgca

SEQ ID NO: 66 - protein, 10 aa, synthetic construct
GYTFSSYVMH

SEQ ID NO: 67 - protein, 17 aa, synthetic construct
YINPYSDVTNYNEKFKG

SEQ ID NO: 68 - protein, 4 aa, synthetic construct
NYFD

SEQ ID NO: 69 - protein, 107 aa, synthetic construct
DIQMTQSPASLSASVGETVTITCRASKNVYSYLAWFQQKQGKSPQLLVYNAKTLAEGVPSRFSGGGSGTQFSLKINSLQPADFGSYYCQHHYNIPFTFGSGTKLEIK

SEQ ID NO: 70 - DNA, 321 bp, synthetic construct
  source 1..321: mol_type = other DNA
gacatccagatgactcagtctccagcctccctatctgcatctgtgggagaaactgtcaccatcacatgtcgagcaagtaaaaatgtttacagttatttagcatggtttcaacagaaacaggggaaatctcctcagctcctggtctataatgctaaaaccttagcagaaggtgtgccatcaaggttcagtggcgggggatcaggcacacagttttctctgaagatcaacagcctgcagcctgcagattttgggagttattactgtcaacatcattataatattccattcacgttcggctcggggacaaagttggaaataaaa

SEQ ID NO: 71 - DNA, 321 bp, synthetic construct
  source 1..321: mol_type = other DNA
gacatacaaatgacacaaagtcccgctagtctttcagccagtgttggtgagactgtgacaataacctgtagagctagcaaaaatgtctactcctatctggcttggttccagcagaaacaaggaaagagtcctcagttgctcgtatataatgctaaaactttggcagaaggcgtcccttctcgtttcagtggcggaggaagtgggactcaattctcactgaagatcaatagcctccagcccgccgactttgggagctactattgccaacatcattacaacataccattcacctttggctcaggtactaaactcgaaattaaa

SEQ ID NO: 72 - protein, 11 aa, synthetic construct
RASKNVYSYLA

SEQ ID NO: 73 - protein, 7 aa, synthetic construct
NAKTLAE

SEQ ID NO: 74 - protein, 9 aa, synthetic construct
QHHYNIPFT

SEQ ID NO: 75 - protein, 119 aa, synthetic construct
QIQLVQSGPELKKPGETVKISCKASGFTFTNYPMHWVKQAPGKGLKWMGWINTYSGVPTYADDFKGRFAFSLETSASTAYLQINNLKNEDMATYFCARGGYDGSREFAYWGQGTLVTVS

SEQ ID NO: 76 - DNA, 357 bp, synthetic construct
  source 1..357: mol_type = other DNA
cagatccagttggtgcagtctggacctgagctgaagaagcctggagagacagtcaagatctcctgcaaggcttctgggtttaccttcacaaactatccaatgcactgggtgaagcaggctccaggaaagggtttaaagtggatgggctggataaacacctactctggagtgccaacatatgcagatgacttcaagggacggtttgccttctctttggaaacctctgccagcactgcatatttgcagatcaacaacctcaaaaatgaggacatggctacatatttctgtgcaagagggggctacgatggtagcagggagtttgcttactggggccaagggactctggtcactgtctct

SEQ ID NO: 77 - DNA, 357 bp, synthetic construct
  source 1..357: mol_type = other DNA
cagatacaactcgtccagtcaggtccagagttgaagaaacccggagaaactgtgaagatatcctgtaaagccagcggctttactttcacaaactaccccatgcattgggtgaagcaggcccccggaaaaggactcaaatggatgggatggatcaacacatacagtggggtgcctacttacgcagacgatttcaaaggaaggttcgcatttagcttggaaactagcgcatctacagcatatctccagattaacaatcttaaaaatgaggatatggcaacatacttctgcgctaggggaggttacgatgggagcagggagttcgcttattgggggcaagggactcttgtgactgtaagt

SEQ ID NO: 78 - protein, 10 aa, synthetic construct
GFTFTNYPMH

SEQ ID NO: 79 - protein, 17 aa, synthetic construct
WINTYSGVPTYADDFKG

SEQ ID NO: 80 - protein, 11 aa, synthetic construct
GGYDGSREFAY

SEQ ID NO: 81 - protein, 110 aa, synthetic construct
DIVLTQSPASLAASLGQRATTSYRASKSVSTSGYSYMHWNQQKPGQPPRLLIYLVSNLESGVPARFSGSGSGTDFTLNIHPVEEEDAATYYCQHIRELTTFGGGTKLEIK

SEQ ID NO: 82 - DNA, 330 bp, synthetic construct
  source 1..330: mol_type = other DNA
gacattgtgctgacacagtctcctgcttccttagctgcatctctggggcagagggccaccacctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttaccacgttcggaggggggaccaagctggaaataaaa

SEQ ID NO: 83 - DNA, 330 bp, synthetic construct
  source 1..330: mol_type = other DNA
gacatagtgctcactcagagccctgcatcccttgccgcctccctcggacaacgagctactacaagctaccgggcatcaaagtccgttagcacatcaggatacagctatatgcactggaatcagcaaaagccaggccaaccaccccgtcttctcatctacctcgtaagtaatctggaatcaggcgtgccagcccgattcagtgggtcagggtctgggacagatttcaccctcaacatccatccagtagaggaagaggacgcagcaacatattactgccaacacattagagaacttaccactttcggaggaggaactaaattggagatcaaa

SEQ ID NO: 84 - protein, 15 aa, synthetic construct
RASKSVSTSGYSYMH

SEQ ID NO: 85 - protein, 7 aa, synthetic construct
LVSNLES

SEQ ID NO: 86 - protein, 7 aa, synthetic construct
QHIRELT

SEQ ID NO: 87 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 107 aa, synthetic construct [sequence omitted]

SEQ ID NO: 90 - protein, 450 aa, Homo sapiens
QVQLVQSGAEVKKPGASVKVSCKASGYTFTNYGVKWVRQAPGQDLEWMGWINTYTGNPIYAADFKGRVTMTTDTSTSTAFMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1362 bp, Homo sapiens
  source 1..1362: mol_type = other DNA
tctaccggacaggtgcagttggttcagtctggcgccgaagtgaagaaacctggcgcttctgtgaaggtgtcctgcaaggcctctggctacacctttaccaactacggcgtgaaatgggtccgacaggctcctggacaggatctggaatggatgggctggatcaacacctacaccggcaatcctatctacgccgccgacttcaagggcagagtgaccatgaccaccgacacctctacctccaccgccttcatggaactgcggtccctgagatctgacgacaccgccgtgtactactgcgtgcggtttcagtacggcgactaccggtactttgatgtgtggggccagggcacactggtcaccgtttcttccgcttctaccaagggacccagcgtgttccctctggctccttcctctaaatccacctctggcggaaccgctgctctgggctgtctggtcaaggattacttccctgagcctgtgaccgtgtcctggaactctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 92 - protein, 450 aa, Homo sapiens
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1353 bp, Homo sapiens
  source 1..1353: mol_type = other DNA
gaagtgcagttggtgcagtctggcgccgaagtgaagaaacctggcgcttctgtgaaggtgtcctgcaaggcctctggctacacctttaccaactacggcgtgaaatgggtccgacaggctcctggacaaggcctggaatggatgggctggatcaacacctacaccggcaatcctatctacgccgccgacttcaagggcagagtgaccatgaccaccgacacctctacctccaccgcctacatggaactgcggtccctgagatctgacgacaccgccgtgtactactgcgtgcggtttcagtacggcgactaccggtactttgatgtgtggggccagggcacactggtcaccgtttcttccgcttctaccaagggacccagcgtgttccctctggctccttcctctaaatccacctctggcggaaccgctgctctgggctgtctggtcaaggattacttccctgagcctgtgaccgtgtcctggaattctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 94 - protein, 10 aa, Homo sapiens
GYTFTNYGVK

SEQ ID NO: 95 - protein, 16 aa, Homo sapiens
WINTYTGNPIYAADFK

SEQ ID NO: 96 - protein, 11 aa, Homo sapiens
FQYGDYRYFDV

SEQ ID NO: 97 - protein, 213 aa, Homo sapiens
EIILTQSPATLSLSPGERATLSCRANSGISFINWYQQKPGQAPRLLIYGTANLASGIPARFGGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - DNA, 714 bp, Homo sapiens
  source 1..714: mol_type = other DNA
aagcttgccaccatggaaaccgacacactgctgctgtgggtgctgttgttgtgggtgccaggatctaccggagagatcatcctgacacagagccccgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagatttggcggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 99 - protein, 213 aa, Homo sapiens
EIVLTQSPATLSLSPGERATLSCRANSGISFINWYQQKPGQAPRLLIYGTANLASGIPARFSGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - DNA, 651 bp, Homo sapiens
  source 1..651: mol_type = other DNA
tctacaggcgagatcgtgctgacccagtctcctgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 101 - protein, 213 aa, Homo sapiens
EIVLTQSPATLSLSPGERATLSCRAQSGISFINWYQQKPGQAPRLLIYGTANLASGIPARFSGSGSGRDFTLTISSLEPEDFAVYYCQQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - DNA, 651 bp, Homo sapiens
  source 1..651: mol_type = other DNA
tctacaggcgagatcgtgctgacccagtctcctgccacattgtctctgagtcctggcgagagagctaccctgtcttgtagagcccagtccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtggtctagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtctggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 103 - protein, 213 aa, Homo sapiens
EIVLTQSPATLSLSPGERATLSCRANSGISFINWYQQKPGQAPRLLIYGTANLASGIPARFSGSGSGRDFTLTISSLEPEDFAVYYCQQYSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - DNA, 651 bp, Homo sapiens
  source 1..651: mol_type = other DNA
tctacaggcgagatcgtgctgacccagtctcctgccacattgtctctgagtcctggcgagagagctaccctgtcctgtagagccaactccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtacagcagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 105 - protein, 213 aa, Homo sapiens
EIVLTQSPATLSLSPGERATLSCRAQSGISFINWYQQKPGQAPRLLIYGTANLASGIPARFSGSGSGRDFTLTISSLEPEDFAVYYCQQYSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - DNA, 651 bp, Homo sapiens
  source 1..651: mol_type = other DNA
tctacaggcgagatcgtgctgacccagtctcctgccacattgtctctgagtcctggcgagagagctaccctgtcttgtagagcccagtccggcatctccttcatcaactggtatcagcagaagcccggccaggctcctagactgctgatctatggcaccgctaacctggcctctggcatccctgctagattttccggctctggctctggcagagacttcaccctgaccatctctagcctggaacctgaggacttcgccgtgtactactgccagcagtacagcagcaaccctctgacctttggcggaggcaccaaggtggaaatcaagagaaccgtggccgctccttccgtgttcatcttcccaccatctgacgagcagctgaagtctggcacagcctctgtcgtgtgcctgctgaacaacttctaccctcgggaagccaaggtgcagtggaaggtggacaatgccctgcagtctggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaaccggggcgagtgctga

SEQ ID NO: 107 - protein, 10 aa, Homo sapiens
RANSGISFIN

SEQ ID NO: 108 - protein, 10 aa, Homo sapiens
RAQSGISFIN

SEQ ID NO: 109 - protein, 7 aa, Homo sapiens
GTANLAS

SEQ ID NO: 110 - protein, 9 aa, Homo sapiens
QQWSSNPLT

SEQ ID NO: 111 - protein, 9 aa, Homo sapiens
QQYSSNPLT

SEQ ID NO: 112 - protein, 453 aa, Homo sapiens
EVQLVESGGGLVKPGGSLRLSCAASGFTFSSFAMSWVRQAPGKGLEWVSTISSGSIYIYYTDGVKGRFTISRDNAKNSLYLQMNSLRAEDTAVYYCARRGIYYGYDGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - DNA, 1362 bp, Homo sapiens
  source 1..1362: mol_type = other DNA
gaggtgcagctggttgaatctggcggaggacttgtgaagcctggcggctctctgagactgtcttgtgccgcctctggcttcaccttctccagctttgccatgtcctgggtccgacaggctcctggcaaaggactggaatgggtgtccaccatctcctccggctccatctacatctactacaccgacggcgtgaagggcagattcaccatcagcagagacaacgccaagaactccctgtacctgcagatgaacagcctgagagccgaggacaccgccgtgtactattgtgccagacggggcatctactatggctacgacggctacgctatggactattggggacagggcacactggtcaccgtgtcctctgcttctaccaagggacccagcgtgttccctctggctccttcctctaaatccacctctggcggaaccgctgctctgggctgtctggtcaaggattacttccctgagcctgtgaccgtgtcctggaactctggtgctctgacatccggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaagctgctggcgctccctctgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacaccctgcctccatctcgggaagagatgaccaagaaccaggtgtccctgacctgcctcgtgaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggcaagtga

SEQ ID NO: 114 - protein, 453 aa, Homo sapiens
EVQLVESGGGLVKPGGSLRLSCAASGFTFSSFAMSWVRQAPGKGLEWVSTISSGSIYIYYTDSVKGRFTISRDNAKNSLYLQMNSLRAEDTAVYYCARRGIYYGYEGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 10 aa, Homo sapiens
GFTFSSFAMS

SEQ ID NO: 116 - protein, 17 aa, Homo sapiens
TISSGSIYIYYTDGVKG

SEQ ID NO: 117 - protein, 17 aa, Homo sapiens
TISSGSIYIYYTDSVKG

SEQ ID NO: 118 - protein, 14 aa, Homo sapiens
RGIYYGYDGYAMDY

SEQ ID NO: 119 - protein, 14 aa, Homo sapiens
RGIYYGYEGYAMDY

SEQ ID NO: 120 - protein, 14 aa, Homo sapiens
RGIYYGYSGYAMDY

SEQ ID NO: 121 - protein, 14 aa, Homo sapiens
RGIYYGYAGYAMDY

SEQ ID NO: 122 - protein, 219 aa, Homo sapiens
AIVLTQSPGTLSLSPGERATLSCRSSQSLVHSNGNTYLHWYMQKPGQAPRVLIYKVSNRFSGIPDRFSGSGSGTDFTLTISRLEPDDFAIYYCSQSTHIPLAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 660 bp, Homo sapiens
  source 1..660: mol_type = other DNA
gccatcgtgttgacccagtctccaggcacattgtctctgagccctggcgagagagctaccctgtcctgcagatcttctcagtccctggtgcactccaacggcaacacctacctgcactggtacatgcagaagcccggacaggctcccagagtgctgatctacaaggtgtccaaccggttctctggcatccccgacagattttccggctctggctctggcaccgacttcaccctgaccatctctagactggaacccgacgacttcgccatctactactgctcccagtccacacacatccctctggcttttggccagggcaccaagctggaaatcaagagaaccgtggccgctccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgtgaagtgacccaccagggcctgtctagccctgtgaccaagtctttcaaccggggcgagtgttga

SEQ ID NO: 124 - protein, 16 aa, Homo sapiens
RSSQSLVHSNGNTYLH

SEQ ID NO: 125 - protein, 16 aa, Homo sapiens
RSSQSLVHSSGNTYLH

SEQ ID NO: 126 - protein, 16 aa, Homo sapiens
RSSQSLVHSTGNTYLH

SEQ ID NO: 127 - protein, 16 aa, Homo sapiens
RSSQSLVHSQGNTYLH

SEQ ID NO: 128 - protein, 7 aa, Homo sapiens
KVSNRFS

SEQ ID NO: 129 - protein, 9 aa, Homo sapiens
SQSTHIPLA

SEQ ID NO: 130 - protein, 120 aa, Homo sapiens
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSS

SEQ ID NO: 131 - protein, 106 aa, Homo sapiens
EIVLTQSPATLSLSPGERATLSCRAQSGISFINWYQQKPGQAPRLLIYGTANLASGIPARFSGSGSGRDFTLTISSLEPEDFAVYYCQQWSSNPLTFGGGTKVEIK

SEQ ID NO: 132 - protein, 120 aa, Homo sapiens
QVQLVQSGAEVKKPGASVKVSCKASGYTFTNYGVKWVRQAPGQDLEWMGWINTYTGNPIYAADFKGRVTMTTDTSTSTAFMELRSLRSDDTAVYYCVRFQYGDYRYFDVWGQGTLVTVSS

SEQ ID NO: 133 - protein, 106 aa, Homo sapiens
EIILTQSPATLSLSPGERATLSCRANSGISFINWYQQKPGQAPRLLIYGTANLASGIPARFGGSGSGRDFTLTISSLEPEDFAVYYCQQWSSNPLTFGGGTKVEIK

SEQ ID NO: 134 - protein, 202 aa, synthetic construct
MCYGKCARCIGHSLVGLALLCIAANILLYFPNGETKYASENHLSRFVWFFSGIVGGGLLMLLPAFVFIGLEQDDCCGCCGHENCGKRCAMLSSVLAALIGIAGSGYCVIVAALGLAEGPLCLDSLGQWNYTFASTEGQYLLDTSTWSECTEPKHIVEWNVSLFSILLALGGIEFILCLIQVINGVLGGICGFCCSHQQQYDC

SEQ ID NO: 135 - protein, 330 aa, synthetic construct; identical to SEQ ID NO: 87

SEQ ID NO: 136 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 330 aa, synthetic construct; identical to SEQ ID NO: 136

SEQ ID NO: 142 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 330 aa, synthetic construct; identical to SEQ ID NO: 138

SEQ ID NO: 144 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVAVSHEDPEVKFNWYVDGVEVHNAKTKPREEQYCSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 332 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYCSTYRVVSVLTVLHQDWLNGKEYKCAVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKKP

SEQ ID NO: 146 - protein, 447 aa, synthetic construct
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 447 aa, synthetic construct
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 148 - protein, 447 aa, synthetic construct
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 149 - protein, 447 aa, synthetic construct
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAEGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 150 - protein, 449 aa, synthetic construct
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFCSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKKP

SEQ ID NO: 151 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 153 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 154 - protein, 447 aa, synthetic construct
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 155 - protein, 447 aa, synthetic construct
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 156 - protein, 450 aa, synthetic construct
EVQLVQSGAEVKKPGASVKVSCKASGYTFTNYGVKWVRQAPGQGLEWMGWINTYTGNPIYAADFKGRVTMTTDTSTSTAYMELRSLRSDDTAVYYCVRFQYGDYRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 48 aa, Homo sapiens
EGPLCLDSLGQWNYTFASTEGQYLLDTSTWSECTEPKHIVEWNVSLFS

SEQ ID NO: 158 - protein, 12 aa, synthetic construct
REEQYCSTYRVV

SEQ ID NO: 159 - protein, 12 aa, synthetic construct
REEQFCSTYRVV

(19) United States
(12) Patent Application Publication
(10) Pub. No.: US 2025/0152730 A1
(43) Pub. Date: May 15, 2025

(54) ANTI-TM4SF1 ANTIBODY-DRUG CONJUGATES COMPRISING CLEAVABLE LINKERS AND METHODS OF USING SAME

(71) Applicant: Angiex, Inc., Cambridge, MA (US)

(72) Inventors: Paul A. Jaminet, Sudbury, MA (US); Shou-Ching S. Jaminet, Sudbury, MA (US); Manish Hudlikar, Brookline, MA (US); Edward H. Ha, New York, NY (US)

(21) Appl. No.: 18/971,926

(22) Filed: Dec. 6, 2024

Related U.S. Application Data
(63) Continuation of application No. PCT/US2023/024732, filed on Jun. 7, 2023.
(60) Provisional application No. 63/350,349, filed on Jun. 8, 2022.

(51) Int. Cl. A61K 47/68

(52) U.S. Cl. CPC A61K 47/6849; A61K 47/68033; A61K 47/6889

(57) ABSTRACT

Antibody-drug conjugates (ADCs) are described, comprising anti-TM4SF1 antibodies, antigen-binding fragments thereof, and a linker. Methods of use of said ADCs are also described.

### CROSS-REFERENCE

This application is a continuation of International Application No. PCT/US2023/024732, filed Jun. 7, 2023, which claims the benefit of U.S. Provisional Application No. 63/350,349, filed Jun. 8, 2022, all of which applications are entirely incorporated herein by reference.

### SEQUENCE LISTING STATEMENT

The instant application contains a Sequence Listing which has been submitted electronically in XML format and is hereby incorporated by reference in its entirety. Said XML copy, created on Dec. 6, 2024, is named 52628-723_301_SL.xml and is 170,252 bytes in size.

### BACKGROUND

There remains a need in the art for cancer therapeutics, and in particular therapeutics with improved therapeutic margins that can regress primary tumors as well as invasive tumor cells and metastases.

Cancer therapies designed to destroy tumor blood vessels have in the past failed in clinical trials due to toxicity. Examples include the vascular disrupting agents such as Combretastatin (CA4P). See, e.g., Grisham et al. Clinical trial experience with CA4P anticancer therapy: focus on efficacy, cardiovascular adverse events, and hypertension management. Gynecol Oncol Res Pract. 2018; 5:1. CA4P reduced overall survival from 16.2 to 13.6 months in the Phase II FALCON study, and seven patients have experienced heart attacks while being treated with CA4P. Id. As coronary heart disease and stroke are leading causes of death, any vascular targeted toxic therapy may lead to a risk of lethal toxicity.

TM4SF1 is an endothelial marker with a functional role in angiogenesis. See, e.g., Shih et al. The L6 protein TM4SF1 is critical for endothelial cell function and tumor angiogenesis. Cancer Res. 2009; 69(8):3272-7. Although antibody-drug conjugates targeting TM4SF1 have been considered previously, see, e.g., Visintin et al. Novel Anti-TM4SF1 Antibody-Drug Conjugates with Activity against Tumor Cells and Tumor Vasculature, Mol Cancer Ther 2015 (14) (8) 1868-1876, in order to enable anti-TM4SF1 ADCs to fulfill their promise as therapies for solid tumors, TM4SF1 targeted ADCs with reduced toxicity to normal vessels, especially arteries, are needed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

One embodiment provides an antibody drug conjugate comprising: (i) an anti-TM4SF1 antibody or an antigen binding fragment thereof; (ii) a therapeutic molecule; and (iii) a linker conjugated with the ani-TM4SF1 antibody and the therapeutic molecule, wherein the linker comprises a first fragment, wherein the first fragment comprises a moiety selected from the group consisting of

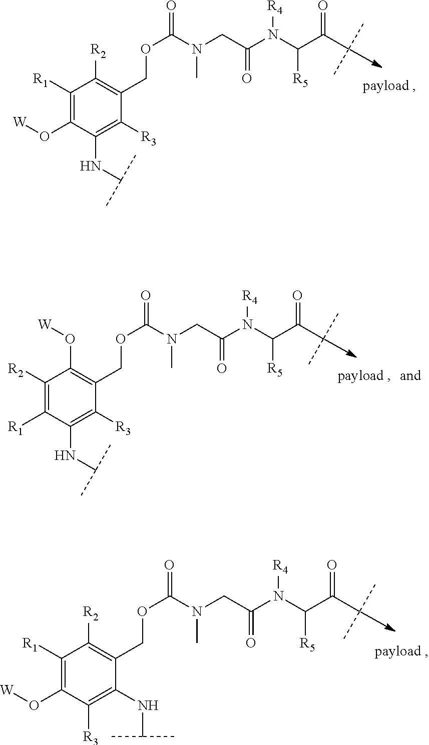

wherein:
payload indicates orientation of the moiety or the linker with respect to conjugation to the therapeutic molecule;
W is a sugar moiety, wherein W—O represents an O-glycosidic bond cleavable by beta-galactosidase;
each R₁, R₂, and R₃ is independently H, halide, —CN, or —NO₂; and
each R₄ and R₅ is independently H, C₁-C₆ alkyl, or C₃-C₆ cycloalkyl.

In some embodiments, the first fragment comprises a moiety selected from the group consisting of:

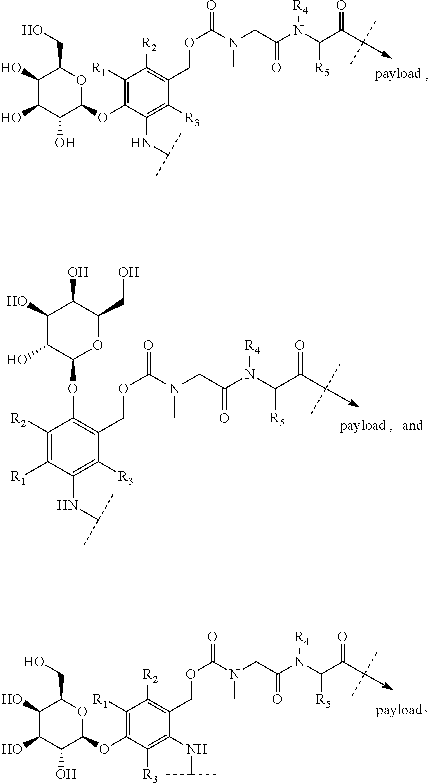

In some embodiments, the first fragment comprises a moiety selected from the group consisting of:

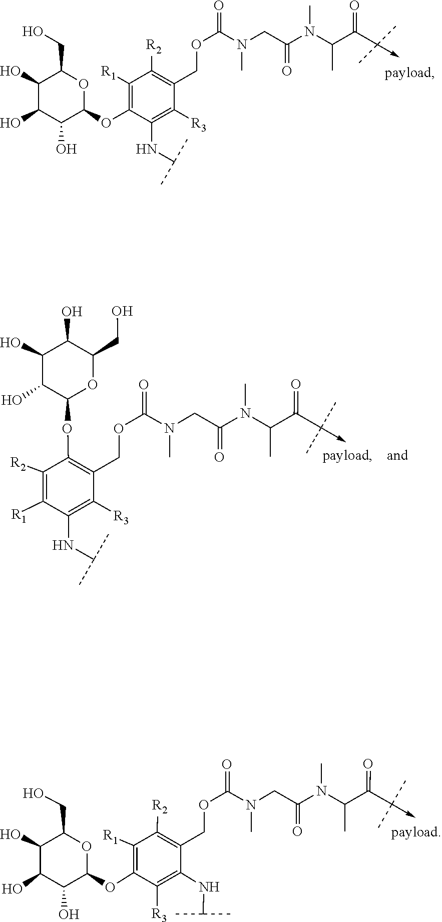

In some embodiments, the first fragment comprises a moiety selected from the group consisting of:

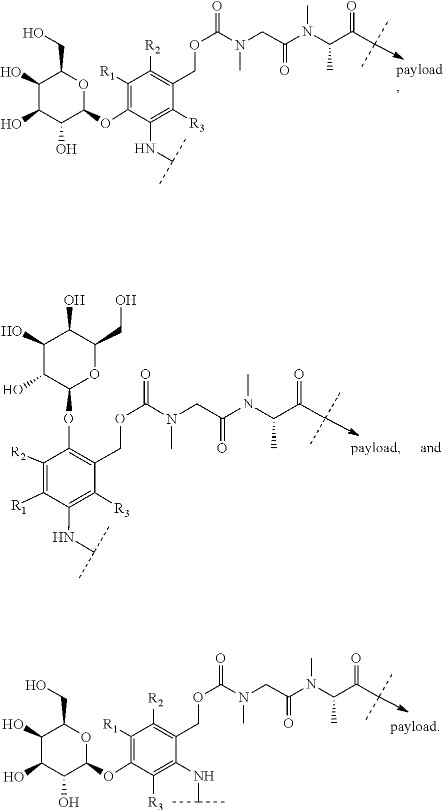

In some embodiments, the first fragment comprises a moiety selected from the group consisting of:

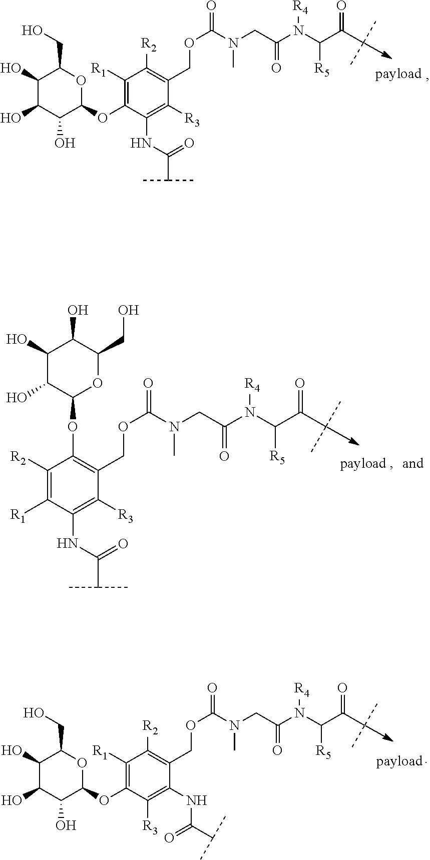

In some embodiments, the first fragment comprises a moiety selected from the group consisting of:

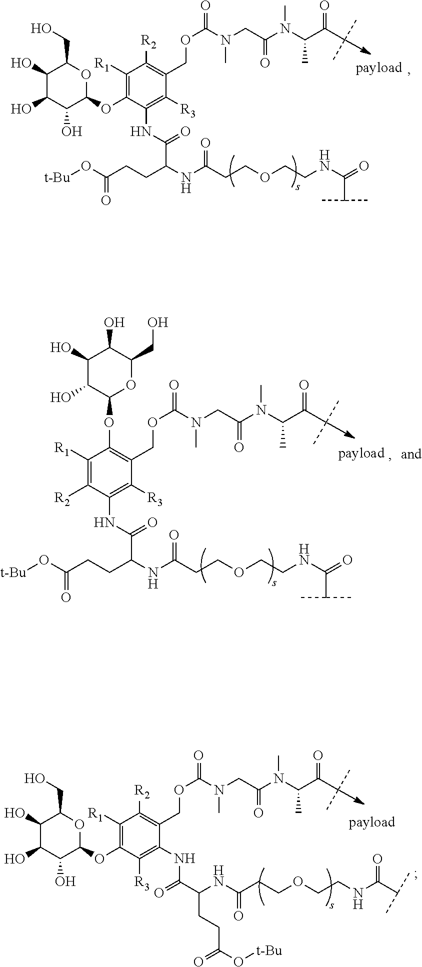

wherein s is 1, 2, 3, 4, 5, 6, 7 or 8.

In some embodiments, the first fragment comprises a moiety selected from the group consisting of:

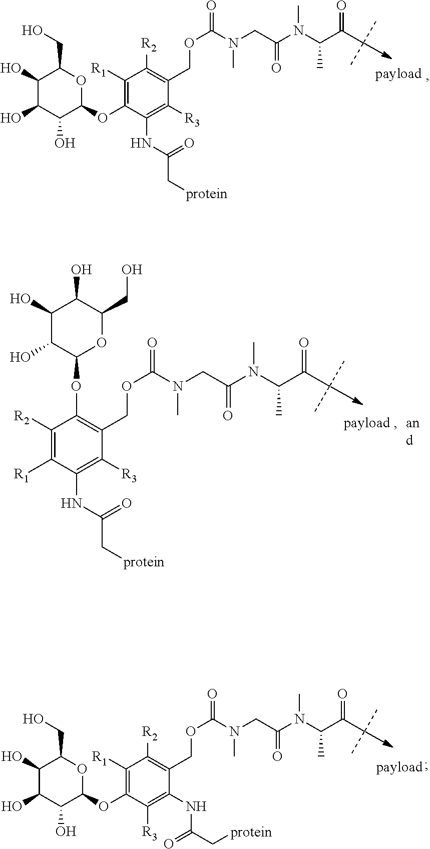

and wherein protein is the anti-TM4SF1 antibody or the antigen binding fragment thereof.

In some embodiments, each R₁, R₂, and R₃ is H.

In some embodiments, the first fragment comprises a moiety selected from the group consisting of:

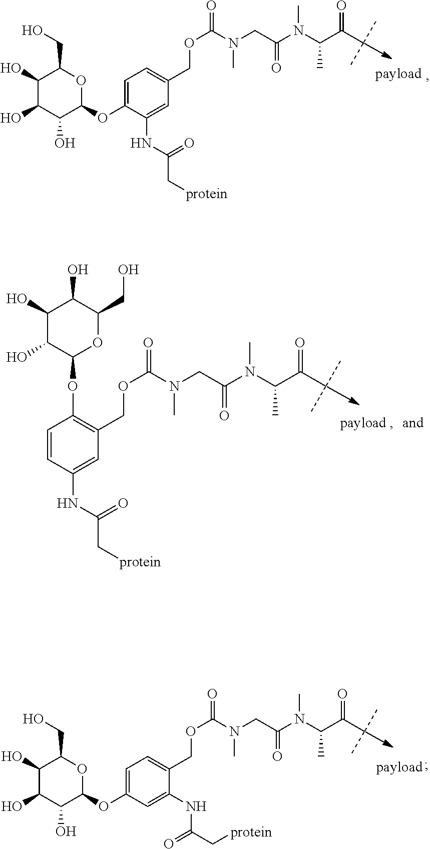

wherein protein is the anti-TM4SF1 antibody or the antigen binding fragment thereof.

In some embodiments, the antibody drug conjugate is:

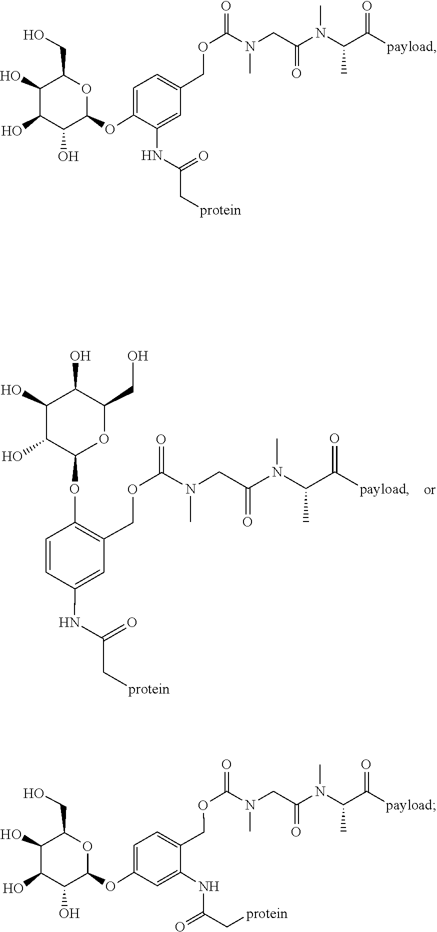

wherein protein is the anti-TM4SF1 antibody or the antigen binding fragment thereof; and wherein payload is the therapeutic molecule.

In some embodiments, the antibody drug conjugate is:

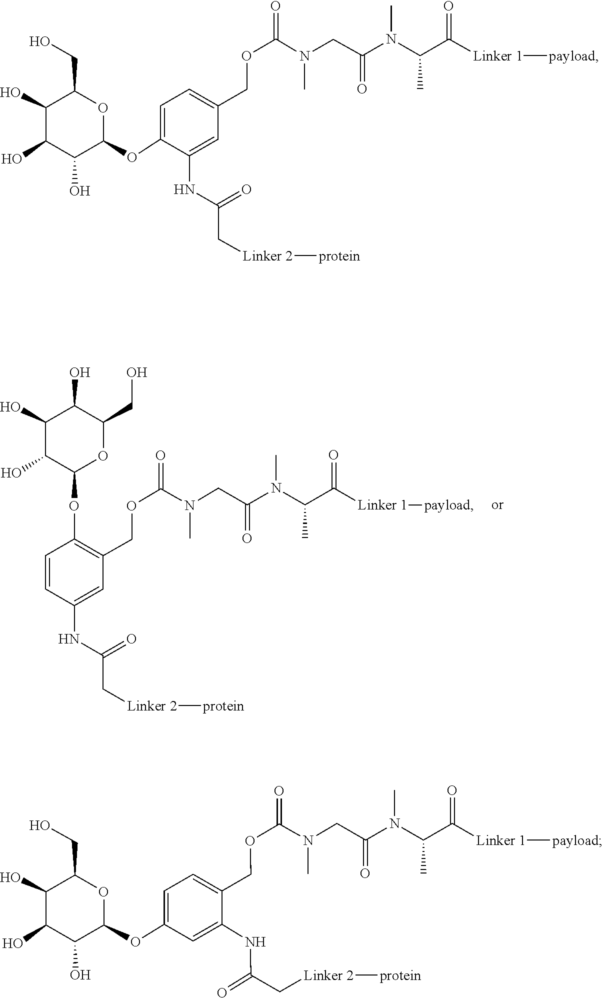

wherein protein is the anti-TM4SF1 antibody or the antigen binding fragment thereof; wherein payload is the therapeutic molecule, wherein each of Linker 1 and Linker 2 is independently alkylene, alkenylene, cycloalkylene with a 3-7 membered ring, alkynylene, arylene, heteroarylene, heterocyclene with a 5-12 membered ring comprising 1-3 atoms of N, O or S, —O—, —NH—, —S—, —N(C₁₋₆ alkyl)-, —C(═O)—, —C(═O)NH—, or combinations thereof, wherein each of the alkylene, alkenylene, cycloalkylene with the 3-7 membered ring, arylene, heteroarylene, and heterocyclene with a 5-12 membered ring comprising 1-3 atoms of N, O or S is independently unsubstituted or independently substituted with halide, amino, —CF₃, C₁-C₃ alkyl, C₃-C₆ cycloalkyl, C₁-C₃ alkoxy, C1-C₃ alkoxy, or C₁-C₃ alkylthio.

In some embodiments, the first fragment is a cleavable linker.

In some embodiments, the linker further comprise a second fragment, wherein the second fragment comprises alkylene, alkenylene, cycloalkylene with a 3-7 membered ring, alkynylene, arylene, heteroarylene, heterocyclene with a 5-12 membered ring comprising 1-3 atoms of N, O or S, —O—, —NH—, —S—, —N(C₁₋₆ alkyl)-, —C(═O)—, —C(═O)NH—, or combinations thereof, wherein the alkylene, alkenylene, cycloalkylene a 3-7 membered ring, arylene, heteroarylene, and heterocyclene with a 5-12 membered ring comprising 1-3 atoms of N, O or S is unsubstituted or substituted with halide, amino, —CF₃, C₁-C₃ alkyl, C₃-C₆ cycloalkyl, C₁-C₃ alkoxy, C₁-C₃ alkoxy, or C₁-C₃ alkylthio. In some embodiments, the second fragment is a cleavable linker or a non-cleavable linker. In some embodiments, the second fragment is a non-cleavable linker. In some embodiments, the second fragment is a cleavable linker.

In some embodiments, each of the second fragment, the Linker 1 and the Linker 2 is independently

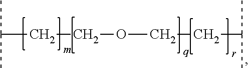

wherein m is 0-3, q is 0-12, and r is 1-3.

In some embodiments, each of the second fragment, the Linker 1 and the Linker 2 is independently:

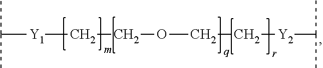

wherein each Y₁ and Y₂ is independently a bond, O, S, or NR₆; R₆ is independently H, deuterium, C₁-C₆ alkyl, C₃-C₆ cycloalkyl, C₁-C₆ alkyl; C₆-C₁₂ aryl, 5-12 membered heteroaryl, C₃-C₁₂ cycloalkyl or 3-12 membered heteroalicyclic, or R₄ together with the nitrogen to which R₆ is bound and another atom of the linker, the anti-TM4SF1 antibody or the antigen-binding fragment thereof, or the therapeutic molecule, be combined to form a 3 to 12 membered heteroalicyclic or 5-12 membered heteroaryl group optionally containing 1 to 3 additional heteroatoms selected from the group consisting of N, O, and S; and m is 0-3, q is 0-12, and r is 1-3.

In some embodiments, the first fragment is directly bonded with the second fragment. In some embodiments, the first fragment is not directly bonded with the second fragment.

In some embodiments, the therapeutic molecule comprises at least one of: a small molecule, a degrader, a nucleic acid molecule, a CRISPR-Cas9 gene editing system, and a lipid nanoparticle, or any combinations thereof. In some embodiments, the therapeutic molecule comprises at least one of: a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, a nucleic acid, a CRISPR enzyme, or any combinations thereof.

In some embodiments, the therapeutic molecule is maytansine or camptothecin. In some embodiments, the degrader comprises a proteolysis inducing chimera, an HSP90 inhibitor, a selective estrogen receptor degrader (SERD), or a selective androgen receptor degrader (SARD), or any combinations thereof.

In some embodiments, the lipid nanoparticle encapsulates one or more agents, wherein each of the one or more agents is independently a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, a nucleic acid, or a CRISPR enzyme.

In some embodiments, the nucleic acid molecule comprises a ribonucleic acid (RNA) molecule or a deoxyribonucleic acid (DNA) molecule. In some embodiments, the RNA molecule comprises an siRNA, an antisense-RNA, an miRNA, an antisense miRNA, an antagomir (anti-miRNA), an shRNA, or an mRNA. In some embodiments, the anti-TM4SF1 antibody or the antigen binding fragment thereof and the therapeutic molecule are conjugated by the linker in a single or a multistep protocol.

In some embodiments, the anti-TM4SF1 antibody or the antigen binding fragment thereof comprises a modified IgG Fc region, wherein the modified IgG Fc region comprises one or more substitutions relative to a wild-type IgG Fc region, wherein the wild-type IgG Fc region is a wild-type IgG1, IgG2, IgG3, or IgG4 Fc region, and wherein the modified IgG Fc region comprises an IgG1 Fc region comprising mutation at one or more positions selected from the group consisting of E233, L234, L235, G237, M252, S254, T250, T256, D265, N297, K322, P331, M428, and N434 of the wild-type IgG1 Fc region, as numbered by the EU index as set forth in Kabat.

In some embodiments, the IgG1 Fc region comprises one or more mutations of N297C, E233P, L234A, L235A, G237A, M252Y, S254T, T256E, M428L, N434S OR N434A, T250Q, D265A, K322A, P331G, or M428L.

In some embodiments, the IgG1 Fc region comprises:
(a) T250Q and M428L; or
(b) L234A, L235A, and G237A; or
(c) L234A, L235A, G237A, and P331G; or
(d) L234A, L235A, G237A, N297C, and P331G; or
(e) E233P, L234A, L235A, G237A, and P331G; or
(f) E233P, L234A, L235A, G237A, and N297C; or
(g) L234A, L235A, G237A, N297C, K322A, and P331G; or
(h) E233P, L234A, L235A, G237A, D265A, N297C, K322A, and P331G; or
(i) E233P and D265A; or
(j) M252Y, S254T, and T256E; or
(k) M252Y, S254T, T256E, and N297C; or
(l) a combination thereof.

In some embodiments, the anti-TM4SF1 antibody or the antigen binding fragment thereof comprises an IgG Fc region comprising a mutation at one or more positions selected from the group consisting of E233, L234, L235, G237, M252, S254, T250, T256, D265, N297, K322, P331, M428, and N434; as numbered by the EU index as set forth in Kabat. In some embodiments, the IgG Fc region comprises the mutation at position N297. In some embodiments, the mutation at position N297 comprises N297C. In some embodiments, the IgG Fc region further comprises an extended C-terminus that is positively charged, wherein the extended C-terminus comprises one or more amino acid residues after position K447, as numbered by the EU index as set forth in Kabat.

In some embodiments, the anti-TM4SF1 antibody or the antigen binding fragment thereof comprises an IgG Fc region comprising an extended C-terminus that is positively charged, wherein the extended C-terminus comprises one or more amino acid residues after position K447, as numbered by the EU index as set forth in Kabat. In some embodiments, the IgG Fc region further comprises a mutation at one or more positions selected from the group consisting of E233, L234, L235, G237, M252, S254, T250, T256, D265, N297, K322, P331, T356, M428, and N434; as numbered by the EU index as set forth in Kabat. In some embodiments, the IgG Fc region comprises the mutation at position N297. In some embodiments, the mutation at position N297 comprises N297C. In some embodiments, the IgG Fc region further comprises an extended C-terminus that is positively charged, wherein the extended C-terminus comprises one or more amino acid residues after position K447, as numbered by the EU index as set forth in Kabat.

In some embodiments, the anti-TM4SF1 antibody or the antigen binding fragment thereof comprises an IgG Fc region comprising a cysteine residue at position N297, as numbered by the EU index as set forth in Kabat. In some embodiments, the IgG Fc region further comprises a mutation at one or more positions selected from the group consisting of E233, L234, L235, G237, M252, S254, T250, T256, D265, N297, K322, P331, M428, and N434; as numbered by the EU index as set forth in Kabat.

In some embodiments, the IgG Fc region further comprises an extended C-terminus that is positively charged, wherein the extended C-terminus comprises one or more amino acid residues after position K447, as numbered by the EU index as set forth in Kabat.

In some embodiments, the anti-TM4SF1 antibody or the antigen binding fragment thereof comprises an IgG Fc region comprising a human IgG1 Fc region comprising a cysteine residue at position N297 and a mutation at one or more positions selected from the group consisting of E233, L234, L235, G237, M252, S254, T250, T256, D265, N297, K322, P331, M428, and N434; as numbered by the EU index as set forth in Kabat. In some embodiments, the IgG Fc region further comprises an extended C-terminus that is positively charged, wherein the extended C-terminus comprises one or more amino acid residues after position K447, as numbered by the EU index as set forth in Kabat.

In some embodiments, the anti-TM4SF1 antibody or the antigen binding fragment thereof comprises an IgG Fc region comprising a cysteine residue at position N297, as numbered by the EU index as set forth in Kabat, wherein the antibody-drug conjugate comprises a drug to antibody ratio (DAR) of greater than or equal to about 1. In some embodiments, the IgG Fc region further comprises a mutation at one or more positions selected from the group consisting of E233, L234, L235, G237, M252, S254, T250, T256, D265, N297, K322, P331, M428, and N434; as numbered by the EU index as set forth in Kabat. In some embodiments, the IgG Fc region further comprises an extended C-terminus that is positively charged, wherein the extended C-terminus comprises one or more amino acid residues after position K447, as numbered by the EU index as set forth in Kabat.

In some embodiments, the anti-TM4SF1 antibody or the antigen binding fragment thereof comprises an IgG Fc region comprising a cysteine residue at position N297 and an extended C-terminus that is positively charged, wherein the extended C-terminus comprises one or more amino acid residues after position K447, wherein numbering is according to the EU index as set forth in Kabat. In some embodiments, the IgG Fc region further comprises a mutation at one or more positions selected from the group consisting of E233, L234, L235, G237, M252, S254, T250, T256, D265, N297, K322, P331, M428, and N434; as numbered by the EU index as set forth in Kabat. In some embodiments, the one or more amino acid residues after position K447 are independently selected from the group consisting of a lysine, a proline, an arginine, or any combinations thereof.

In some embodiments, the one or more amino acid residues after position K447 are independently selected from the group consisting of: the lysine and the proline. In some embodiments, the IgG Fc region comprises the mutation at position E233. In some embodiments, the mutation at position E233 comprises E233P. In some embodiments, the IgG Fc region comprises the mutation at position L234. In some embodiments, the mutation at position L234 comprises L234A. In some embodiments, the IgG Fc region comprises the mutation at position L235. In some embodiments, the mutation at position L235 comprises L235A. In some embodiments, the IgG Fc region comprises the mutation at position G237. In some embodiments, the mutation at position G237 comprises G237A. In some embodiments, the IgG Fc region comprises the mutation at position M252. In some embodiments, the mutation at position M252 comprises M252Y. In some embodiments, the IgG Fc region comprises the mutation at position S254. In some embodiments, the mutation at position S254 comprises S254T. In some embodiments, the IgG Fc region comprises the mutation at position T256. In some embodiments, the mutation at position T256 comprises T256E. In some embodiments, the IgG Fc region comprises the mutation at position M428. In some embodiments, the mutation at position M428 comprises M428L. In some embodiments, the IgG Fc region comprises the mutation at position N434. In some embodiments, the mutation at position N434 comprises N434S or N434A. In some embodiments, the IgG Fc region comprises the mutation at position T250. In some embodiments, the mutation at position T250 comprises T250Q. In some embodiments, the IgG Fc region comprises the mutation at position D265. In some embodiments, the mutation at position D265 comprises D265A. In some embodiments, the IgG Fc region comprises the mutation at position K322. In some embodiments, the mutation at position K322 comprises K322A. In some embodiments, the IgG Fc region comprises the mutation at position P331. In some embodiments, the mutation at position P331 comprises P331G. In some embodiments, the IgG Fc region comprises T250Q and M428L. In some embodiments, the IgG Fc region comprises M428L. In some embodiments, the IgG Fc region comprises M428L and N434S.

In some embodiments, the IgG Fc region comprises N434A. In some embodiments, the IgG Fc region comprises L234A, L235A, and G237A. In some embodiments, the IgG Fc region comprises L234A, L235A, G237A, and P331G. In some embodiments, the IgG Fc region comprises L234A, L235A, G237A, N297C, and P331G. In some embodiments, the IgG Fc region comprises L234A, L235A, G237A, K322A, and P331G. In some embodiments, the IgG Fc region comprises E233P, L234A, L235A, G237A, and P331G. In some embodiments, the IgG Fc region comprises E233P, L234A, L235A, G237A, and N297C. In some embodiments, the IgG Fc region comprises E233P, L234A, L235A, G237A, and N297C. In some embodiments, the IgG Fc region comprises L234A, L235A, G237A, N297C, K322A, and P331G. In some embodiments, the IgG Fc region comprises E233P, L234A, L235A, G237A, D265A, N297C, K322A, and P331G. In some embodiments, the IgG Fc region comprises E233P, L234A, L235A, G237A, D265A, N297C, K322A, and P331G. In some embodiments, the IgG Fc region comprises E233P and D265A. In some embodiments, the IgG Fc region comprises M252Y, S254T, and T256E. In some embodiments, the IgG Fc region comprises M252Y, S254T, T256E, and N297C. In some embodiments, the IgG Fc region comprises K322A and P331G, and wherein the IgG Fc region further comprises an extended C-terminus that is positively charged, wherein the extended C-terminus comprises one or more amino acid residues after position K447. In some embodiments, the IgG Fc region comprises an amino acid sequence selected from the group consisting of SEQ ID Nos. 87-88, 135-145, and 151-153. In some embodiments, the IgG Fc region exhibits reduced or ablated binding with Clq. In some embodiments, the IgG Fc region exhibits reduced or ablated binding to an Fc receptor. In some embodiments, the anti-TM4SF1 antibody exhibits reduced or ablated ADCC or CDC effector function.

In some embodiments, the anti-TM4SF1 antibody or the antigen binding fragment thereof comprises a human IgG4 Fc region comprising a mutation at one or more positions selected from the group consisting of S228, F234, L235, G237, P238, F243, T250, M252, S254, T256, E258, D259, V264, D265, K288, T299, T307, V308, Q311, K322, L328, P329, A330, P331, T356, K370, A378, R₄₀₉, V427, M428, H433, N434, H435, and N297, as numbered by the EU index as set forth in Kabat. In some embodiments, the human IgG4 Fc region comprises the mutation at position N297. In some embodiments, the mutation at position N297 comprises N297C. In some embodiments, the human IgG4 Fc region further comprises an extended C-terminus that is positively charged, wherein the extended C-terminus comprises one or more amino acid residues after position K447, as numbered by the EU index as set forth in Kabat.

In some embodiments, the anti-TM4SF1 antibody or the antigen binding fragment thereof comprises a human IgG4 Fc region comprising an extended C-terminus that is positively charged, wherein the extended C-terminus comprises one or more amino acid residues after position K447, as numbered by the EU index as set forth in Kabat. In some embodiments, the human IgG4 Fc region further comprises a mutation at one or more positions selected from the group consisting of S228, F234, L235, G237, P238, F243, T250, M252, S254, T256, E258, D259, V264, D265, K288, T299, T307, V308, Q311, K322, L328, P329, A330, P331, T356, K370, A378, R409, V427, M428, H433, N434, H435, and N297, as numbered by the EU index as set forth in Kabat. In some embodiments, the human IgG4 Fc region comprises the mutation at position N297. In some embodiments, the mutation at position N297 comprises N297C.

In some embodiments, the anti-TM4SF1 antibody or the antigen binding fragment thereof comprises a human IgG4 Fc region comprising a cysteine residue at position N297, as numbered by the EU index as set forth in Kabat. In some embodiments, the human IgG4 Fc region further comprises a mutation at one or more positions selected from the group consisting of S228, F234, L235, G237, P238, F243, T250, M252, S254, T256, E258, D259, V264, D265, K288, T299, T307, V308, Q311, K322, L328, P329, A330, P331, T356, K370, A378, R409, V427, M428, H433, N434, and H435, as numbered by the EU index as set forth in Kabat. In some embodiments, the human IgG4 Fc region further comprises an extended C-terminus that is positively charged, wherein the extended C-terminus comprises one or more amino acid residues after position K447, as numbered by the EU index as set forth in Kabat.

In some embodiments, the anti-TM4SF1 antibody or the antigen binding fragment thereof comprises a human IgG4 Fc region comprising a cysteine residue at position N297 and a mutation at one or more positions selected from the group consisting of S228, F234, L235, G237, P238, F243, T250, M252, S254, T256, E258, D259, V264, D265, K288, T299, T307, V308, Q311, K322, L328, P329, A330, P331, T356, K370, A378, R409, V427, M428, H433, N434, and H435, as numbered by the EU index as set forth in Kabat. In some embodiments, the human IgG4 Fc region further comprises an extended C-terminus that is positively charged, wherein the extended C-terminus comprises one or more amino acid residues after position K447, as numbered by the EU index as set forth in Kabat.

In some embodiments, the anti-TM4SF1 antibody or the antigen binding fragment thereof comprises a human IgG4 Fc region comprising a cysteine residue at position N297, as numbered by the EU index as set forth in Kabat, wherein the antibody-drug conjugate comprises a drug to antibody ratio (DAR) of greater than or equal to 1. In some embodiments, the human IgG4 Fc region further comprises a mutation at one or more positions selected from the group consisting of S228, F234, L235, G237, P238, F243, T250, M252, S254, T256, E258, D259, V264, D265, K288, T299, T307, V308, Q311, K322, L328, P329, A330, P331, T356, K370, A378, R409, V427, M428, H433, N434, and H435, as numbered by the EU index as set forth in Kabat. In some embodiments, the human IgG4 Fc region further comprises an extended C-terminus that is positively charged, wherein the extended C-terminus comprises one or more amino acid residues after position K447, as numbered by the EU index as set forth in Kabat.

In some embodiments, the anti-TM4SF1 antibody or the antigen binding fragment thereof comprises a human IgG4 Fc region comprising a cysteine residue at position N297 and an extended C-terminus that is positively charged, wherein the extended C-terminus comprises one or more amino acid residues after position K447, wherein numbering is according to the EU index as set forth in Kabat. In some embodiments, the human IgG4 Fc region further comprises a mutation at one or more positions selected from the group consisting of S228, F234, L235, G237, P238, F243, T250, M252, S254, T256, E258, D259, V264, D265, K288, T299, T307, V308, Q311, K322, L328, P329, A330, P331, T356, K370, A378, R409, V427, M428, H433, N434, and H435, as numbered by the EU index as set forth in Kabat. In some embodiments, the one or more amino acid residues after position K447 is independently selected from the group consisting of: a lysine, a proline, an arginine, or any combinations thereof. In some embodiments, the one or more amino acid residues after position K447 is independently selected from the group consisting of the lysine and the proline. In some embodiments, the human IgG4 Fc region comprises the mutation at position 5228. In some embodiments, the mutation at position S228 comprises S228P. In some embodiments, the human IgG4 Fc region comprises the mutation at position F234. In some embodiments, the mutation at position F234 comprises F234A. In some embodiments, the human IgG4 Fc region comprises the mutation at position L235. In some embodiments, the mutation at position L235 comprises L235E. In some embodiments, the human IgG4 Fc region comprises S228P and L235E. In some embodiments, the human IgG4 Fc region comprises S228P, L235E, and N297C. In some embodiments, the human IgG4 Fc region comprises S228P, F234A, L235E, and N297C. In some embodiments, the human IgG4 Fc region comprises S228P, L235E, and N297C, and wherein the human IgG4 Fc region further comprises an extended C-terminus that is positively charged, wherein the extended C-terminus comprises one or more amino acid residues after position K447. In some embodiments, the human IgG4 Fc region comprises M428L and N434S. In some embodiments, the human IgG4 Fc region comprises mutations at L235 and F234. In some embodiments, the human IgG4 Fc region comprises mutations at positions L328, A330, and T299. In some embodiments, the human IgG4 Fc region comprises S228P, F234A, L235A, G237A, and P238S. In some embodiments, the human IgG4 Fc region comprises F243A and V264A. In some embodiments, the human IgG4 Fc region comprises S228P and L235A. In some embodiments, the human IgG4 Fc region comprises M252Y and M428L; D259I and V308F; or N434S. In some embodiments, the human IgG4 Fc region comprises T307Q and N434S; M428L and V308F; Q311V and N434S; H433K and N434F; E258F and V427T; or T256D, Q311V, and A378V. In some embodiments, the human IgG4 Fc region comprises one or more of the following properties: (i) reduced or ablated binding with Clq; (ii) reduced or ablated binding to an Fc receptor; and (iii) reduced or ablated ADCC or CDC effector function. In some embodiments, the anti-TM4SF1 antibody or the antigen binding fragment thereof comprising the human IgG4 Fc region comprises an amino acid sequence selected from the group consisting of SEQ ID Nos. 146-150, and 154-155.

In some embodiments, the anti-TM4SF1 antibody or the antigen binding fragment thereof comprises:
(a) a heavy chain comprising a CDR3 domain comprising an amino acid sequence that has at least 75% identity to a sequence selected from the group consisting of SEQ ID NO: 8, 20, 32, 44, 56, 68, 80, 96, 118, 119, 120, and 121; a CDR2 domain comprising an amino acid sequence that has at least 75% identity to a sequence selected from the group consisting of SEQ ID NO: 7, 19, 31, 43, 55, 67, 79, 95, 116, and 117; and a CDR1 domain comprising an amino acid sequence that has at least 75% identity to a sequence selected from the group consisting of SEQ ID NO: 6, 18, 30, 42, 54, 66, 78, 94, and 115; and
(b) a light chain comprising a CDR3 domain comprising an amino acid sequence that has at least 75% identity to a sequence selected from the group consisting of SEQ ID NO: 14, 26, 38, 50, 62, 74, 86, 110, and 129; a CDR2 domain comprising an amino acid sequence that has at least 75% identity to a sequence selected from the group consisting of SEQ ID NO: 13, 25, 37, 49, 61, 73, 85, 109, and 128; and a CDR1 domain comprising an amino acid sequence that has at least 75% identity to a sequence selected from the group consisting of SEQ ID NO: 12, 24, 36, 48, 60, 72, or 84, 107, 108, 124, 125, 126, and 127.

In some embodiments, the heavy chain comprises an amino acid sequence that has at least 75% identity to SEQ ID NO: 3, 15, 27, 39, 51, 63, 75, 90, 92, 112, 114, 130, or 132, and a light chain comprises an amino acid sequence that has at least 75% identity to SEQ ID NO: 9, 21, 33, 45, 57, 69, 81, 97, 99, 101, 122, 131, or 133. In some embodiments, the heavy chain comprises an amino acid sequence as set forth in any one of: SEQ ID NO: 3, 15, 27, 39, 51, 63, 75, 90, 92, 112, 114, 130, or 132, and wherein the light chain variable domain comprises an amino acid sequence as set forth in any one of: SEQ ID NO: 9, 21, 33, 45, 57, 69, 81, 97, 99, 101, 122, 131, or 133. In some embodiments, the heavy chain comprises a CDR3 domain comprising the amino acid sequence as set forth in SEQ ID NO: 8, a CDR2 domain comprising the amino acid sequence as set forth in SEQ ID NO: 7, and a CDR1 domain comprising the amino acid sequence as set forth in SEQ ID NO: 6; and wherein the light chain comprises a CDR3 domain comprising the amino acid sequence as set forth in SEQ ID NO: 14, a CDR2 domain comprising the amino acid sequence as set forth in SEQ ID NO: 13, and a CDR1 domain comprising the amino acid sequence as set forth in SEQ ID NO: 12. In some embodiments, the heavy chain comprises a CDR3 domain comprising the amino acid sequence se set forth in SEQ ID NO: 20, a CDR2 domain comprising the amino acid sequence as set forth in SEQ ID NO: 19, and a CDR1 domain comprising the amino acid sequence as set forth in SEQ ID NO: 18; and wherein the light chain comprises a CDR3 domain comprising the amino acid sequence as set forth in SEQ ID NO: 26, a CDR2 domain comprising the amino acid sequence as set forth in SEQ ID NO: 25, and a CDR1 domain comprising the amino acid sequence as set forth in SEQ ID NO: 24. In some embodiments, the heavy chain comprises a CDR3 domain comprising the amino acid sequence se set forth in SEQ ID NO: 32, a CDR2 domain comprising the amino acid sequence as set forth in SEQ ID NO: 31, and a CDR1 domain comprising the amino acid sequence as set forth in SEQ ID NO: 30; and wherein the light chain comprises a CDR3 domain comprising the amino acid sequence as set forth in SEQ ID NO: 38, a CDR2 domain comprising the amino acid sequence as set forth in SEQ ID NO: 37, and a CDR1 domain comprising the amino acid sequence as set forth in SEQ ID NO: 36.

In some embodiments, the heavy chain comprises a CDR3 domain comprising the amino acid sequence se set forth in SEQ ID NO: 44, a CDR2 domain comprising the amino acid sequence as set forth in SEQ ID NO: 43, and a CDR1 domain comprising the amino acid sequence as set forth in SEQ ID NO: 42; and wherein the light chain comprises a CDR3 domain comprising the amino acid sequence as set forth in SEQ ID NO: 50, a CDR2 domain comprising the amino acid sequence as set forth in SEQ ID NO: 49, and a CDR1 domain comprising the amino acid sequence as set forth in SEQ ID NO: 48. In some embodiments, the heavy chain comprises a CDR3 domain comprising the amino acid sequence se set forth in SEQ ID NO: 56, a CDR2 domain comprising the amino acid sequence as set forth in SEQ ID NO: 55, and a CDR1 domain comprising the amino acid sequence as set forth in SEQ ID NO: 54; and wherein the light chain comprises a CDR3 domain comprising the amino acid sequence as set forth in SEQ ID NO: 62, a CDR2 domain comprising the amino acid sequence as set forth in SEQ ID NO: 61, and a CDR1 domain comprising the amino acid sequence as set forth in SEQ ID NO: 60. In some embodiments, the heavy chain comprises a CDR3 domain comprising the amino acid sequence se set forth in SEQ ID NO: 68, a CDR2 domain comprising the amino acid sequence as set forth in SEQ ID NO: 67, and a CDR1 domain comprising the amino acid sequence as set forth in SEQ ID NO: 66; and wherein the light chain comprises a CDR3 domain comprising the amino acid sequence as set forth in SEQ ID NO: 74, a CDR2 domain comprising the amino acid sequence as set forth in SEQ ID NO: 73, and a CDR1 domain comprising the amino acid sequence as set forth in SEQ ID NO: 72. In some embodiments, the heavy chain comprises a CDR3 domain comprising the amino acid sequence se set forth in SEQ ID NO: 80, a CDR2 domain comprising the amino acid sequence as set forth in SEQ ID NO: 79, and a CDR1 domain comprising the amino acid sequence as set forth in SEQ ID NO: 78; and wherein the light chain comprises a CDR3 domain comprising the amino acid sequence as set forth in SEQ ID NO: 86, a CDR2 domain comprising the amino acid sequence as set forth in SEQ ID NO: 85, and a CDR1 domain comprising the amino acid sequence as set forth in SEQ ID NO: 84. In some embodiments, the heavy chain comprises a CDR3 domain comprising the amino acid sequence se set forth in SEQ ID NO: 96, a CDR2 domain comprising the amino acid sequence as set forth in SEQ ID NO: 95, and a CDR1 domain comprising the amino acid sequence as set forth in SEQ ID NO: 94; and wherein the light chain comprises a CDR3 domain comprising the amino acid sequence as set forth in SEQ ID NO: 111 or SEQ ID NO: 110, a CDR2 domain comprising the amino acid sequence as set forth in SEQ ID NO: 109, and a CDR1 domain comprising the amino acid sequence as set forth in SEQ ID NO: 107 or SEQ ID NO: 108.

In some embodiments, the heavy chain comprises a CDR3 domain comprising the amino acid sequence se set forth in SEQ ID NO: 96, a CDR2 domain comprising the amino acid sequence as set forth in SEQ ID NO: 95, and a CDR1 domain comprising the amino acid sequence as set forth in SEQ ID NO: 94; and wherein the light chain comprises a CDR3 domain comprising the amino acid sequence as set forth in SEQ ID NO: 110, a CDR2 domain comprising the amino acid sequence as set forth in SEQ ID NO: 109, and a CDR1 domain comprising the amino acid sequence as set forth in SEQ ID NO: 107. In some embodiments, the heavy chain comprises a CDR3 domain comprising the amino acid sequence se set forth in SEQ ID NO: 96, a CDR2 domain comprising the amino acid sequence as set forth in SEQ ID NO: 95, and a CDR1 domain comprising the amino acid sequence as set forth in SEQ ID NO: 94; and wherein the light chain comprises a CDR3 domain comprising the amino acid sequence as set forth in SEQ ID NO: 110, a CDR2 domain comprising the amino acid sequence as set forth in SEQ ID NO: 109, and a CDR1 domain comprising the amino acid sequence as set forth in SEQ ID NO: 108. In some embodiments, the heavy chain comprises a CDR3 domain comprising the amino acid sequence se set forth in SEQ ID NO: 96, a CDR2 domain comprising the amino acid sequence as set forth in SEQ ID NO: 95, and a CDR1 domain comprising the amino acid sequence as set forth in SEQ ID NO: 94; and wherein the light chain comprises a CDR3 domain comprising the amino acid sequence as set forth in SEQ ID NO: 111, a CDR2 domain comprising the amino acid sequence as set forth in SEQ ID NO: 109, and a CDR1 domain comprising the amino acid sequence as set forth in SEQ ID NO: 107. In some embodiments, the heavy chain comprises a CDR3 domain comprising the amino acid sequence se set forth in SEQ ID NO: 96, a CDR2 domain comprising the amino acid sequence as set forth in SEQ ID NO: 95, and a CDR1 domain comprising the amino acid sequence as set forth in SEQ ID NO: 94; and wherein the light chain comprises a CDR3 domain comprising the amino acid sequence as set forth in SEQ ID NO: 111, a CDR2 domain comprising the amino acid sequence as set forth in SEQ ID NO: 109, and a CDR1 domain comprising the amino acid sequence as set forth in SEQ ID NO: 108. In some embodiments, the heavy chain comprises a CDR3 domain comprising the amino acid sequence se set forth in SEQ ID NO: 118, a CDR2 domain comprising the amino acid sequence as set forth in SEQ ID NO: 116, and a CDR1 domain comprising the amino acid sequence as set forth in SEQ ID NO: 115; and wherein the light chain comprises a CDR3 domain comprising the amino acid sequence as set forth in SEQ ID NO: 129, a CDR2 domain comprising the amino acid sequence as set forth in SEQ ID NO: 128, and a CDR1 domain comprising the amino acid sequence as set forth in SEQ ID NO: 124.

In some embodiments, the heavy chain comprises a CDR3 domain comprising the amino acid sequence se set forth in SEQ ID NO: 118, SEQ ID NO: 119, SEQ IN NO: 120, or SEQ ID NO: 121, a CDR2 domain comprising the amino acid sequence as set forth in SEQ ID NO: 116 or SEQ ID NO: 117, and a CDR1 domain comprising the amino acid sequence as set forth in SEQ ID NO: 115; and wherein the light chain comprises a CDR3 domain comprising the amino acid sequence as set forth in SEQ ID NO: 129, a CDR2 domain comprising the amino acid sequence as set forth in SEQ ID NO: 128, and a CDR1 domain comprising the amino acid sequence as set forth in SEQ ID NO: 124, SEQ ID NO: 125, SEQ ID NO: 126, or SEQ ID NO: 127. In some embodiments, the antigen-binding fragment comprises an Fab, an Fab′, an F(ab′)2, an Fv, or an scFv.

In some embodiments, the anti-TM4SF1 binding protein comprises a modified human IgG1 Fc region, wherein the modified human IgG1 Fc region comprises one or more amino acid substitutions selected from the group consisting of E233, L234, L235, G237, M252, S254, T250, T256, D265, N297, K322, P331, M428, and N434, as numbered by the EU index as set forth in Kabat, wherein the anti-TM4SF1 binding protein demonstrates improved vascular safety compared to an otherwise identical binding protein that does not comprise an amino acid substitution selected from the group consisting of E233, L234, L235, G237, M252, S254, T250, T256, D265, N297, K322, P331, M428, and N434. In some embodiments the modified human IgG1 Fc region comprises a mutation at one or more positions selected from the group consisting of T250, M252, S254, T256, M428, and N434 as numbered by the EU index as set forth in Kabat. In some embodiments the modified human IgG1 Fc region comprises a mutation selected from the group consisting of T250Q, M252Y, S254T, T256E, M428L, and N434S, as numbered by the EU index as set forth in Kabat. In some embodiments, the modified human IgG1 Fc region comprises mutations T250Q and M428L. In some embodiments, the modified human IgG1 Fc region comprises mutations M252Y, S254T, and T256E. In some embodiments, the modified human IgG1 Fc region comprises mutations M428L and N434S.

In some embodiments, the anti-TM4SF1 binding protein comprises a modified human IgG4 Fc region, wherein the modified human IgG4 Fc region comprises one or more amino acid substitutions selected from the group consisting of S228, F234, L235, G237, P238, F243, T250, M252, S254, T256, E258, D259, V264, D265, K288, T299, T307, V308, Q311, K322, L328, P329, A330, P331, T356, K370, A378, R409, V427, M428, H433, N434, H435, and N297, as numbered by the EU index as set forth in Kabat, wherein the anti-TM4SF1 binding protein demonstrates improved vascular safety compared to an otherwise identical binding protein that does not comprise an amino acid substitutions selected from the group consisting of S228, F234, L235, G237, P238, F243, T250, M252, S254, T256, E258, D259, V264, D265, K288, T299, T307, V308, Q311, K322, L328, P329, A330, P331, T356, K370, A378, R409, V427, M428, H433, N434, H435, and N297. In some embodiments, the modified human IgG4 Fc region comprises a mutation at one or more positions selected from the group consisting of T250, M428, and N434 as numbered by the EU index as set forth in Kabat. In some embodiments, the modified human IgG4 Fc region comprises a mutation selected from the group consisting of T250Q, M428L, and N434S as numbered by the EU index as set forth in Kabat. In some embodiments, the modified human IgG4 Fc region comprises mutations T250Q and M428L. In some embodiments, the modified human IgG4 Fc region comprises M428L and N434S. In some embodiments, the binding protein exhibits increased affinity to FcRn as compared to a control anti-TM4SF1 binding protein comprising a wild type IgG1 Fc or IgG4 Fc. In some embodiments, the anti-TM4SF1 binding protein comprises an anti-TM4SF1 antibody or an antigen binding fragment thereof. In some embodiments, the anti-TM4SF1 antibody or the antigen binding fragment thereof is conjugated to a therapeutic molecule, wherein the therapeutic molecule comprises at least one of a small molecule, a degrader, a nucleic acid molecule, a CRISPR-Cas9 gene editing system, and a lipid nanoparticle, or any combinations thereof.

In some embodiments, the antibody-drug conjugate comprises (i) an anti-TM4SF1 antibody or an antigen binding fragment thereof and (ii) a therapeutic molecule, wherein the anti-TM4SF1 antibody or the antigen binding fragment thereof comprises a human IgG1 Fc region comprising a mutation at one or more positions selected from the group consisting of T250, M252, S254, T256, M428, and N434 as numbered by the EU index as set forth in Kabat. In some embodiments, the human IgG1 Fc region comprises a mutation selected from the group consisting of T250Q, M252Y, S254T, T256E, M428L, and N434S, as numbered by the EU index as set forth in Kabat. In some embodiments, the human IgG1 Fc region comprises mutations at positions T250 and M428. In some embodiments, the human IgG1 Fc region comprises mutations T250Q and M428L. In some embodiments, the human IgG1 Fc region comprises mutations at positions M252, S254, and T256. In some embodiments, the human IgG1 Fc region comprises mutations M252Y, S254T, and T256E. In some embodiments, the human IgG1 Fc region comprises mutations at positions M428 and N434. In some embodiments, the human IgG1 Fc region comprises mutations M428L and N434S. In some embodiments, the human IgG1 Fc region further comprises a mutation at position N297. In some embodiments, the mutation at position N297 is N297C. In some embodiments, the human IgG1 Fc region further comprises an extended C-terminus that is positively charged, wherein the extended C-terminus comprises one or more amino acid residues after position K447, as numbered by the EU index as set forth in Kabat. In some embodiments, the human IgG1 Fc region further comprises a mutation at one or more positions selected from the group consisting of E233, L234, L235, G237, D265, N297, K322, and P331; as numbered by the EU index as set forth in Kabat. In some embodiments, the human IgG1 Fc region comprises a mutation selected from the group consisting of E233P, L234A, L235A, G237A, D265A, N297C, K322A, and P331G.

In some embodiments, the human IgG1 Fc region comprises 2, 3, 4, 5, 6, or 7 mutations selected from the group consisting of E233P, L234A, L235A, G237A, D265A, N297C, K322A, and P331G. In some embodiments, the human IgG1 Fc region comprises mutations L234A, L235A, and G237A. In some embodiments, the human IgG1 Fc region comprises mutations L234A, L235A, G237A, and P331G. In some embodiments, the human IgG1 Fc region comprises mutations L234A, L235A, G237A, K322A, and P331G. In some embodiments, the human IgG1 Fc region comprises mutations L234A, L235A, G237A, E233P, and P331G. In some embodiments, the human IgG1 Fc region comprises mutations L234A, L235A, G237A, and N297C. In some embodiments, the human IgG1 Fc region comprises mutations L234A, L235A, G237A, N297C, and P331G. In some embodiments, the human IgG1 Fc region comprises mutations L234A, L235A, G237A, N297C, K322A, and P331G. In some embodiments, the human IgG1 Fc region comprises mutations L234A, L235A, G237A, N297C, E233P, and P331G. In some embodiments, the human IgG1 Fc region comprises mutations L234A, L235A, G237A, D265A, N297C, K322A, and P331G.

In some embodiments, the anti-TM4SF1 antibody or the antigen binding fragment thereof comprises a human IgG4 Fc region comprising a mutation at one or more positions selected from the group consisting of T250, M428, and N434 as numbered by the EU index as set forth in Kabat. In some embodiments, the human IgG4 Fc region comprises a mutation selected from the group consisting of T250Q, M428L, and N434S as numbered by the EU index as set forth in Kabat. In some embodiments, the human IgG4 Fc region comprises mutations at positions T250 and M428. In some embodiments, the human IgG4 Fc region comprises mutations T250Q and M428L. In some embodiments, the human IgG4 Fc region comprises mutations at positions M428 and N434. In some embodiments, the human IgG4 Fc region comprises mutations M428L and N434S. In some embodiments, the human IgG4 Fc region further comprises a mutation at position N297. In some embodiments, the mutation at position N297 is N297C. In some embodiments, the human IgG4 Fc region further comprises an extended C-terminus that is positively charged, wherein the extended C-terminus comprises one or more amino acid residues after position K447, as numbered by the EU index as set forth in Kabat. In some embodiments, the human IgG4 Fc region further comprises a mutation at one or more positions selected from the group consisting of S228, F234, and L235 as numbered by the EU index as set forth in Kabat. In some embodiments, the human IgG4 Fc region comprises a mutation selected from the group consisting of S228P, F234A, L235E, and N297C as numbered by the EU index as set forth in Kabat. In some embodiments, the human IgG4 Fc region comprises 2, 3, or 4, mutations selected from the group consisting of S228P, F234A, L235E, and N297C. In some embodiments, the IgG4 Fc region comprises a mutation at position S228. In some embodiments, the mutation at position S228 is S228P. In some embodiments, the IgG4 Fc region comprises mutations at positions S228 and L235. In some embodiments, the IgG4 Fc region comprises mutations S228P and L235E. In some embodiments, the IgG4 Fc region comprises mutations at positions S228, L235, and N297. In some embodiments, the IgG4 Fc region comprises mutations S228P, L235E, and N297C. In some embodiments, the antibody drug conjugate exhibits increased affinity to FcRn as compared to a control antibody drug conjugate comprising a wild type IgG1 Fc or IgG4 Fc.

In some embodiments, the lipid nanoparticle encapsulates one or more therapeutic molecules. In some embodiments, the linker comprises a cleavable linker, a non-cleavable linker, a hydrophilic linker, a pro-charged linker, or a dicarboxylic acid based linker. In some embodiments, the cleavable linker comprises a cleavable covalent or non-covalent linker. In some embodiments, the linker comprises a non-cleavable covalent or non-covalent linker. In some embodiments, the cleavable linker comprises an acid-labile linker, a protease-sensitive linker, a photo-labile linker, or a disulfide-containing linker. In some embodiments, the linker comprises a cysteine linker or a non-cysteine linker. In some embodiments, the non-cysteine linker comprises a lysine linker. In some embodiments, the linker comprises a MC (6-maleimidocaproyl), a MCC (a maleimidomethyl cyclohexane-1-carboxylate), a MP (maleimidopropanoyl), a val-cit (valine-citrulline), a val-ala (valine-alanine), an ala-phe (alanine-phenylalanine), a PAB (p-aminobenzyloxycarbonyl), a SPP (N-Succinimidyl 4-(2-pyridylthio) pentanoate), 2,5-dioxopyrrolidin-1-yl 4-(pyridin-2-ylthio)hexanoate, 2,5-dioxopyrrolidin-1-yl 5-methyl-4-(pyridin-2-ylthio)hexanoate, 2,5-dioxopyrrolidin-1-yl 5-methyl-4-(pyridin-2-ylthio)heptanoate, 2,5-dioxopyrrolidin-1-yl 5-ethyl-4-(pyridin-2-ylthio)heptanoate, 2,5-dioxopyrrolidin-1-yl 4-cyclopropyl-4-(pyridin-2-ylthio)butanoate, 2,5-dioxopyrrolidin-1-yl 4-cyclobutyl-4-(pyridin-2-ylthio)butanoate, 2,5-dioxopyrrolidin-1-yl 4-cyclopentyl-4-(pyridin-2-ylthio)butanoate, 2,5-dioxopyrrolidin-1-yl 4-cyclohexyl-4-(pyridin-2-ylthio)butanoate, a SMCC (N-Succinimidyl 4-(N-maleimidomethyl)cyclohexane-1 carboxylate), or a SIAB (N-Succinimidyl (4-iodo-acetyl)aminobenzoate). In some embodiments, the linker is derived from a cross-linking reagent, wherein the cross-linking reagent comprises N-succinimidyl-3-(2-pyridyldithio)propionate (SPDP), 2,5-dioxopyrrolidin-1-yl 3-cyclopropyl-3-(pyridin-2-yldisulfaneyl)propanoate, 2,5-dioxopyrrolidin-1-yl 3-cyclobutyl-3-(pyridin-2-yldisulfaneyl)propanoate, N-succinimidyl 4-(2-pyridyldithio)pentanoate (SPP), 2,5-dioxopyrrolidin-1-yl 4-cyclopropyl-4-(pyridin-2-yldisulfaneyl)butanoate, 2,5-dioxopyrrolidin-1-yl 4-cyclobutyl-4-(pyridin-2-yldisulfaneyl)butanoate, N-succinimidyl 4-(2-pyridyldithio)butanoate (SPDB), 2,5-dioxopyrrolidin-1-yl 4-cyclopropyl-4-(pyridin-2-yldisulfaneyl)butanoate, 2,5-dioxopyrrolidin-1-yl 4-cyclobutyl-4-(pyridin-2-yldisulfaneyl)butanoate, N-succinimidyl-4-(2-pyridyldithio)-2-sulfo-butanoate (sulfo-SPDB), N-succinimidyl iodoacetate (SIA), N-succinimidyl(4-iodoacetyl)aminobenzoate (SIAB), maleimide PEG NHS, N-succinimidyl 4-(maleimidomethyl) cyclohexanecarboxylate (SMCC), N-sulfosuccinimidyl 4-(maleimidomethyl) cyclohexanecarboxylate (sulfo-SMCC), or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

One embodiment provides a method of treating or preventing a disease or disorder in a subject, wherein the disease or disorder is characterized by abnormal endothelial cell (EC)-cell interaction, the method comprising administering to the subject an antibody-drug conjugate according to this disclosure. In some embodiments, the EC-cell interaction comprises one or more of EC-mesenchymal stem cell, EC-fibroblast, EC-smooth muscle cell, EC-tumor cell, EC-leukocyte, EC-adipose cell, and EC-neuronal cell interactions. In some embodiments, the disease or disorder comprises an inflammatory disease or a cancer. One embodiment provides a method of treating or preventing inflammation in a subject, the method comprising administering to the subject an antibody-drug conjugate according to this disclosure. One embodiment provides a method of treating or preventing metastasis in a subject, the method comprising administering to the subject an antibody-drug conjugate according to this disclosure, wherein the subject is in partial or complete remission from a cancer. One embodiment provides a method of treating a subject having a cancer which is associated with a high risk of metastasis, the method comprising administering an antibody-drug conjugate according to this disclosure, to the subject having the cancer which is associated with the high risk of metastasis. One embodiment provides a method of treating or preventing metastasis in a subject having a cancer, the method comprising administering an antibody-drug conjugate according to this disclosure, to the subject having the cancer. In some embodiments, the subject is undergoing a treatment which may induce metastasis. In some embodiments, the treatment comprises surgery, radiation treatment and chemotherapy. In some embodiments, the subject is a human. In some embodiments, the cancer is a carcinoma or a sarcoma. In some embodiments, the carcinoma comprises breast cancer, lung cancer, colon cancer, prostate cancer, pancreatic cancer, liver cancer, gastric cancer, renal cancer, bladder cancer, uterine cancer, cervical cancer, ovarian cancer. In some embodiments, the sarcoma comprises an angiosarcoma, an osteosarcoma, or a soft tissue sarcoma. In some embodiments, the cancer is a glioblastoma. One embodiment provides a method of treating or preventing lymphatic or hematogenous metastasis in a human subject comprising administering to the human subject an antibody-drug conjugate according to this disclosure. In some embodiments, the antibody drug conjugate exhibits longer serum half-life after administration as compared to a control antibody drug conjugate comprising a wild type IgG1 Fc or IgG4 Fc.

In some embodiments, the linker cleaves in lysosome. In some embodiments, the first fragment cleaves in lysosome. In some embodiments, the linker is galactoside linker, such as β-galactoside linker, which can be used for ADC to deliver cytotoxic agents. Either amine-containing or alcohol-containing cytotoxic agents can be delivered using the galactoside linker, optionally in the presence of additional spacers or linkers. Exposure of the galactoside linker to galactosidase, such as, for example, β-galactosidase, can result in drug release. In some embodiments, β-galactosidase can be found in lysosomes and tumor interstitium. In some embodiments, higher concentrations of β-galactosidase may be found in the serum and breast milk of diabetic mothers. In some embodiments, cancer may cause chronic inflammation, which in turn may lead to an increase in the concentration of extracellular β-galactosidase. Thus, the interstitial space of necrotic tumor tissue may display high levels of beta-galactosidase activity. The source of the excess beta-galactosidase at the interstitial space of necrotic tumor tissue may be inflammatory cells and may not be the tumor tissue. In some embodiments, there can be increased expression of β-galactosidase in necrotic areas and other body fluids of patients with cancers, such as, for example, breast cancer, cervical cancer colon cancer, lung cancer, renal carcinoma and leukemia, when compared to healthy people. In some embodiments, this overexpression may also be found in other disease states such as urinary tract infection, HIV, diabetes, neuropathy and rheumatoid arthritis.

In some embodiments, the ADC is made from the linker-payload shown in FIG. 1. In some embodiments, the ADCs are synthesized using the conjugation protocols shown in Schemes 3-5.

In some embodiments, the payload is maytansine (also known as maitansine) with a CAS number 35846-53-8. In some embodiments, the ADC comprising an antibody or an antigen binding fragment thereof and a galactoside linker is:

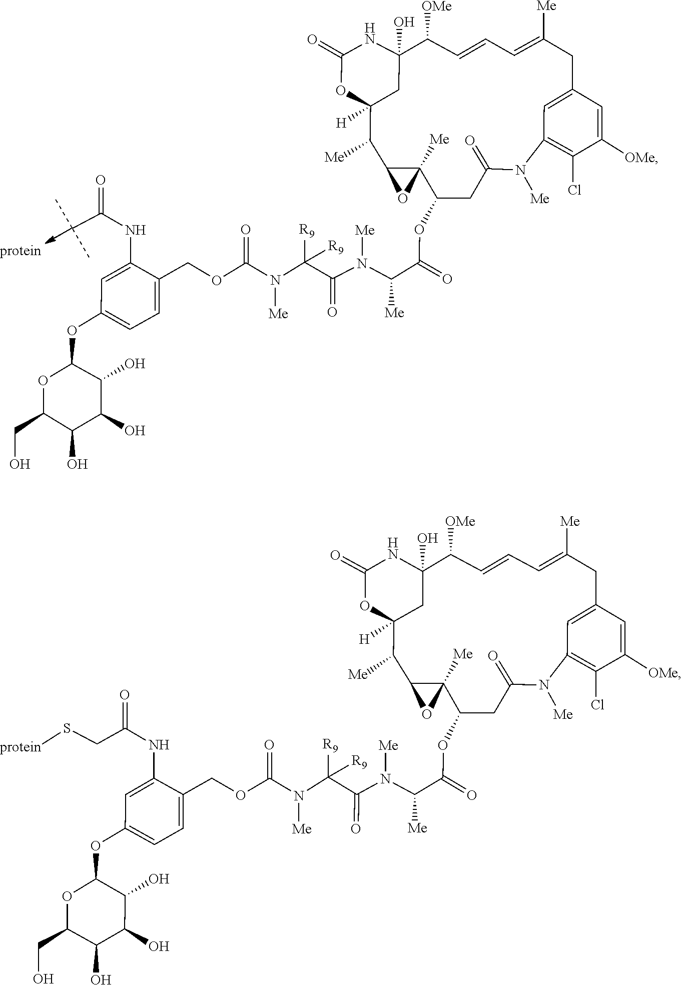

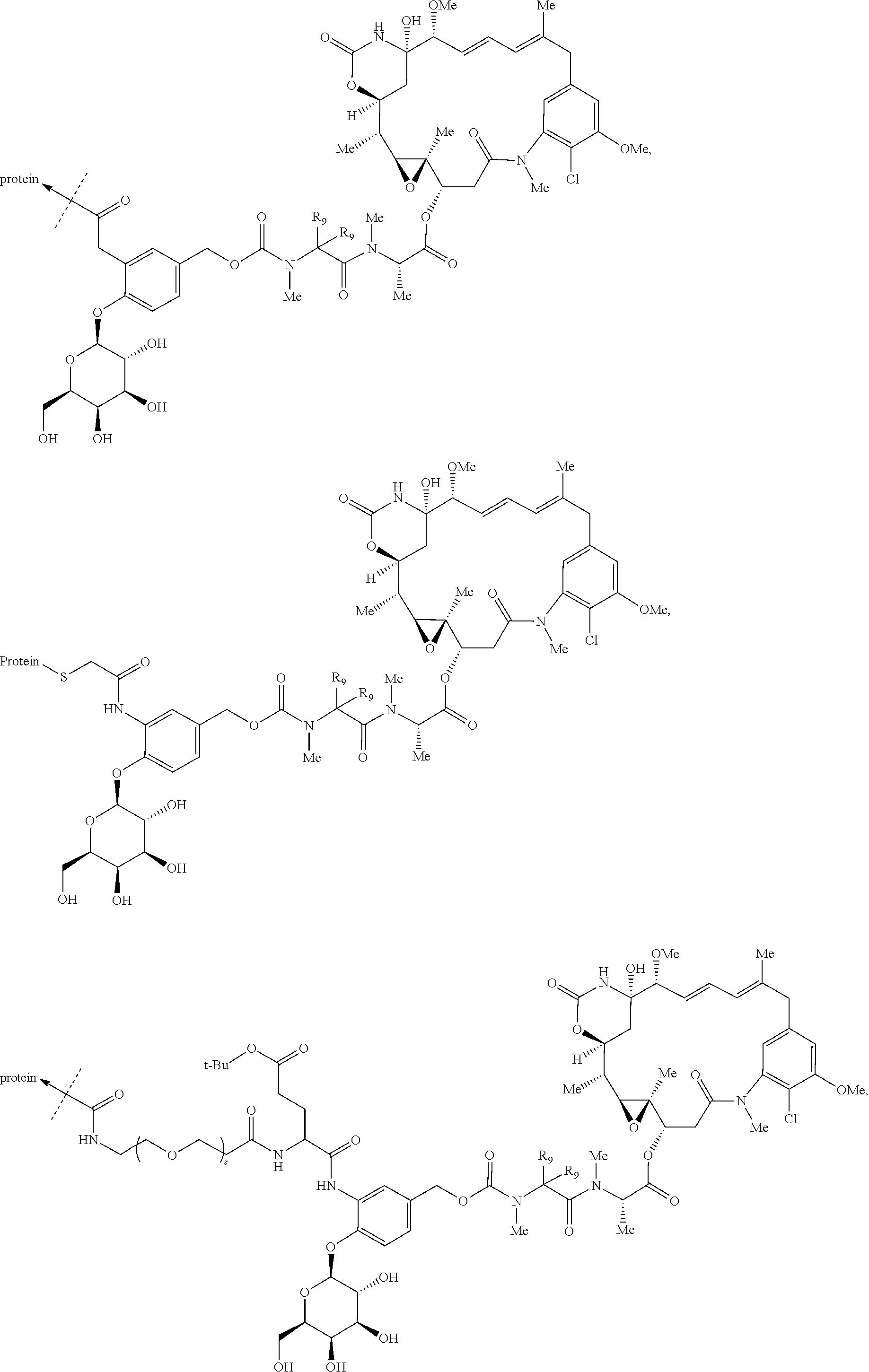

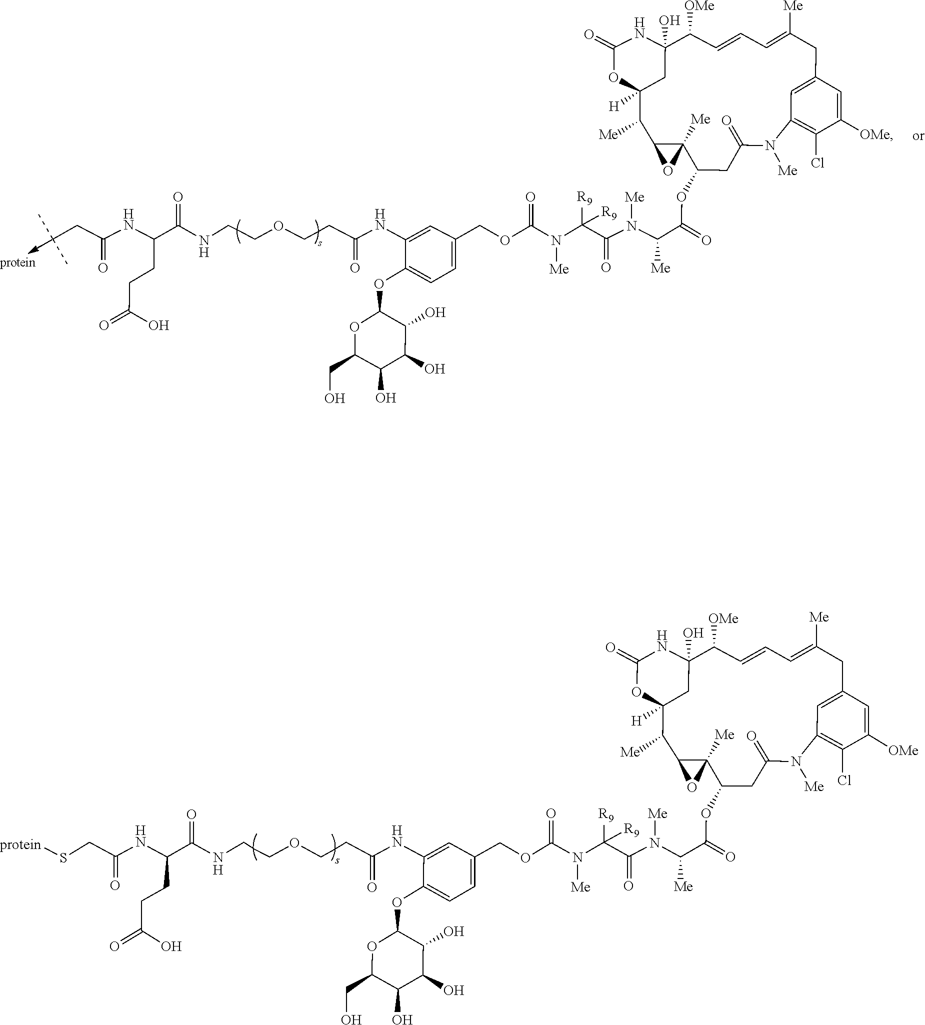

wherein R₉ is independently H or methyl; s is independently 1, 2, 3, 4, 5, 6, 7 or 8, and protein is an antibody or an antigen binding fragment thereof. In some embodiments, the protein is Anti-TM4SF1 antibody or an antigen binding fragment thereof. In some embodiments, R₉ is methyl. In some embodiments, R₉ is H. In some embodiments, s is 4. In some embodiments, the binding activities of the ADC are similar to or better than those of the naked antibody. In some embodiments, the killing activities (in vitro) against human cancer cell lines and/or rodent cancer cell lines have EC50 in the range from 0.01 nM to 300 nM, from 0.01 nM to 0.05 nM, from 0.05 nM to 0.1 nM, from 0.1 nM to 0.5 nM from 0.5 nM to 1 nM from 1 nM to 5 nM from 5 nM to 10 nM from 10 nM to 50 nM from 50 nM to 100 nM from 100 nM to 200 nM or from 200 nM to 300 nM. In some embodiments, the cancer cell lines are for pancreatic cancer, lung cancer, ovarian cancer, and melanoma, and endothelial cells are for umbilical vein (HUVEC) and microvascular (MS1). In some embodiments, the ADC is tolerated in mice models with survival rate no lower than 80% at about 60 mg per kg dosage via injection. In some embodiments, the ADC is tolerated in mice models with survival rate of about 100% at about 60 mg per kg dosage via injection. In some embodiments, the ADC is tolerated in mice models with body weight of tested mice no lower than 80% of the corresponding initial body weight when measured from day 5 to day 50 at about 60 mg per kg dosage via injection. In some embodiments, the ADC is tolerated in mice models with some mice gain body weight against the corresponding initial body weight when measured from day 5 to day 50 at about 60 mg per kg dosage via injection.

In some embodiments, the linker cleaves in lysosomes. In some embodiments, the first fragment cleaves in the lysosomes. In some embodiments, the first fragment is cleaved by a beta-galactosidase enzyme. In some embodiments, at least 50% of the antibody drug conjugate remains intact in extracellular milieu before entering lysosomes. In some embodiments, at least 60% of the antibody drug conjugate remains intact in extracellular milieu before entering lysosomes. In some embodiments, at least 70% of the antibody drug conjugate remains intact in extracellular milieu before entering lysosomes. In some embodiments, at least 80% of the antibody drug conjugate remains intact in extracellular milieu before entering a cytosolic environment. In some embodiments, at least 90% of the antibody drug conjugate remains intact in extracellular milieu before entering lysosomes. In some embodiments, at least 95% of the antibody drug conjugate remains intact in extracellular milieu before entering lysosomes. In some embodiments, at least 50% of the antibody drug conjugate is cleaved in extracellular milieu after entering lysosomes. In some embodiments, at least 60% of the antibody drug conjugate is cleaved in extracellular milieu after entering lysosomes. In some embodiments, at least 70% of the antibody drug conjugate is cleaved in extracellular milieu after entering lysosomes. In some embodiments, at least 80% of the antibody drug conjugate is cleaved in extracellular milieu after entering lysosomes. In some embodiments, at least 90% of the antibody drug conjugate is cleaved in extracellular milieu after entering lysosomes. In some embodiments, at least 95% of the antibody drug conjugate is cleaved in extracellular milieu after entering lysosomes. In some embodiments, at least 99% of the antibody drug conjugate is cleaved in extracellular milieu after entering lysosomes.

In some embodiments, the antibody drug conjugate is cleaved by a beta-galactosidase enzyme. In some embodiments, the antibody drug conjugate is cleaved in lysosome.

One embodiment provides a pharmaceutical composition comprising (i) an antibody-drug conjugate according to this disclosure and (ii) a pharmaceutically acceptable carrier. One embodiment provides a pharmaceutical composition comprising the binding protein according to this disclosure.

In some embodiments, the anti-TM4SF1 antibody or the antigen binding fragment thereof comprises a modified IgG Fc region comprising one or more mutations selected from the group consisting of: (i) S228, F234, L235, G237, P238, F243, T250, M252, S254, T256, E258, D259, V264, D265, K288, T299, T307, V308, Q311, K322, L328, P329, A330, P331, T356, K370, A378, R409, V427, M428, H433, N434, H435, and N297; or (ii) E233, L234, L235, G237, M252, S254, T250, T256, D265, N297, K322, P331, M428, and N434, conjugated to a therapeutic molecule via a linker, wherein portion of the linker including, for example, the second fragment, is derived from a compound of the following Formulae:

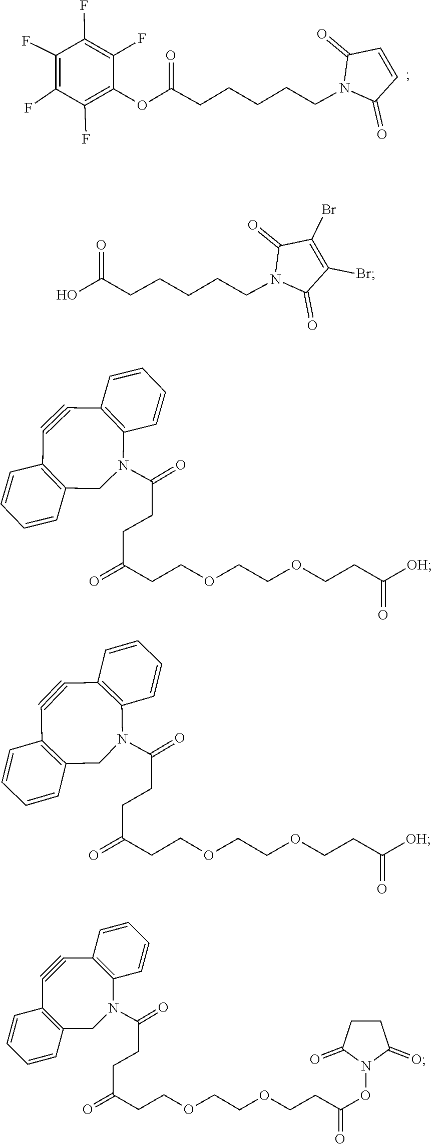 [SMILES: O=C(CCCCCN1C(=O)C=CC1=O)Oc1c(F)c(F)c(F)c(F)c1F ; O=C(O)CCCCCN1C(=O)C(Br)=C(Br)C1=O ; O=C(O)CCOCCOCCC(=O)CCC(=O)N1Cc2ccccc2C#Cc2ccccc21 ; O=C(O)CCOCCOCCC(=O)CCC(=O)N1Cc2ccccc2C#Cc2ccccc21 ; O=C(CCOCCOCCC(=O)ON1C(=O)CCC1=O)CCC(=O)N1Cc2ccccc2C#Cc2ccccc21]

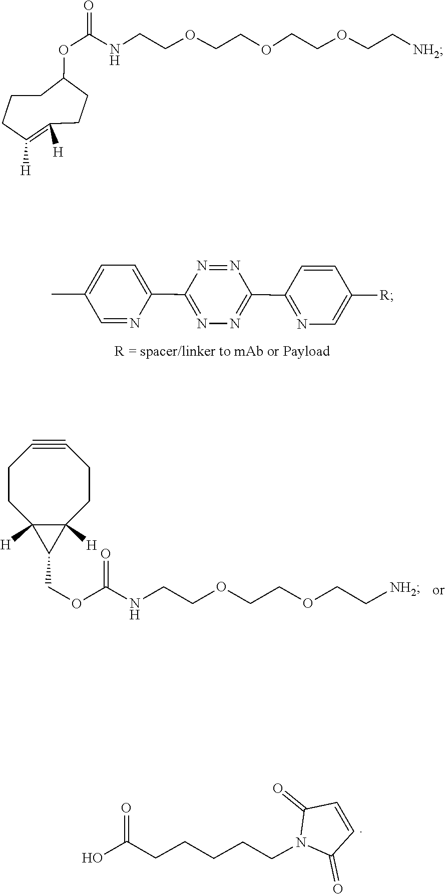

In some embodiments, the linker comprises one or more second fragments derived from molecules independently selected from the group consisting of the Formulae shown above. In some embodiments, the linker comprises one second fragment attached to the anti-TM4SF1 antibody or the antigen binding fragment thereof; the first fragment attached to the second fragment; and another second fragment attached to both the first fragment and the therapeutic molecule. In some embodiments, the linker comprises multiple second fragments in tandem. In some embodiments, the multiple second fragments are of the same molecular structure. In some embodiments, the multiple second fragments are of different molecular structures. In some embodiments, each of the multiple second fragments are independently selected. In some embodiments, some but not all of the multiple second fragments are of the same molecular structure.

### DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates an exemplary linker-payload (LP) comprising bromoacetamide for conjugation.

FIG. 2 shows a mass spectrum of an ADC synthesized from Compound 11 (ADC1) and Exemplary Antibody 1.

FIG. 3 shows a size exclusion chromatograph (SEC) of the ADC1.

FIG. 4 shows a mass spectrum of another ADC synthesized from Compound 11 (ADC2) and Exemplary Antibody 1.

FIG. 5 shows a size exclusion chromatograph (SEC) of the ADC2.

FIG. 6 shows a mass spectrum of an ADC synthesized from Compound 11 (ADC3) and Exemplary Antibody 2.

FIG. 7 shows a size exclusion chromatograph (SEC) of the ADC3.

FIG. 8 shows a mass spectrum of another ADC synthesized from Compound 11 and Exemplary Antibody 2 (ADC4).

FIG. 9 shows a size exclusion chromatograph (SEC) of the ADC4.

FIG. 10 shows a mass spectrum of an ADC synthesized from Compound 11 (ADC5) and Exemplary Antibody 3.

FIG. 11 shows a size exclusion chromatograph (SEC) of the ADC5.

FIG. 12 shows a mass spectrum of another ADC synthesized from Compound 11 and Exemplary Antibody 3 (ADC6).

FIG. 13 shows a size exclusion chromatograph (SEC) of the ADC6.

FIG. 14 shows a mass spectrum of an ADC synthesized from Compound 11 (ADC5) and Exemplary Antibody 4.

FIG. 15 shows a mass spectrum of an ADC synthesized from Compound 11 (ADC5) and Exemplary Antibody 5.

FIG. 16 illustrates the results of a study assessing the affinity of exemplary anti-TM4SF1 antibodies, in various endothelial cells.

FIG. 17 illustrates in vivo tissue distribution (large intestine, small intestine, stomach) of exemplary anti-TM4SF1 antibodies (murine surrogate, MS) containing various Fc mutations.

FIG. 18 illustrates in vivo tissue distribution (female reproductive tract, skin adjacent to a tumor, and tumor under the skin) of exemplary anti-TM4SF1 antibodies (murine surrogate, MS) containing various Fc mutations.

FIG. 19 illustrates hydrophobicity of exemplary anti-TM4SF1 antibodies (murine surrogate, MS; and anti-human AGX-A07), assessed by hydrophobic interaction chromatography (HIC).

### DETAILED DESCRIPTION OF THE INVENTION

Transmembrane-4 L six family member-1 (TM4SF1) is a small membrane glycoprotein with tetraspanin topology that is highly expressed on many human epithelial tumor cells and in endothelial cells, especially endothelial cells in angiogenic vessels or overgrowth of new blood vessels.

Provided herein in one embodiment, is an antibody-drug conjugate (ADC) for a vascular-targeted therapy that, e.g., can regress primary tumors by killing the endothelial cells of tumor blood vessels. This therapy may include various attractive features. Notably, (1) angiogenesis is a hallmark of cancer and a therapy that destroys angiogenic vessels can be a universal treatment for solid tumors; (2) the vascular endothelium is an unmutated host system and might be unable to evolve resistance to therapy. Thus, a vascular-targeted therapy may be able to overcome a common problem with tumor cell targeted therapies, wherein a target tissue evolves and becomes resistant to therapy; and (3) the vascular endothelium of tumors is directly exposed to intravenously (IV)-infused drugs and therefore can be accessible to drugs that cannot reach tumor cells. The inaccessibility of tumor cells can be a major problem in cancers such as pancreatic cancer which have a dense fibrotic stroma which limits access of drugs to tumor cells. A vascular targeted therapy, using an ADC that comprises an anti-TM4SF1 antibody, can advantageously reach the vascular endothelium of tumors.

In one embodiment, the disclosure provides antibody-drug conjugates (ADCs) comprising TM4SF1 binding proteins, such as anti-TM4SF1 antibodies, and antigen-binding fragments thereof. The disclosure includes, in some examples, methods of using the ADCs for treating or preventing cancer. The disclosure includes, in some embodiments, ADCs in which the drug payload conjugated to the antibody is comprised of a small molecule, RNA, DNA, degrader, protein, or combinations thereof.

### I. Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the present disclosure may be more readily understood, select terms are defined below. The terms “transmembrane-4 L six family member-1” or “TM4SFT”, as used herein refer to a polypeptide of the transmembrane 4 superfamily/tetraspanin family, which is highly expressed on tumor vasculature endothelial cells (ECs), tumor cells (TCs), ECs of developing retinal vasculature, and angiogenic blood vessels. TM4SF1 has two extracellular loops (ECL1 and ECL2) that are separated by four transmembrane domains (M1, M2, M3, and M4), the N- and C-termini, and the intracellular loop (ICL). ECL2 contains two N-glycosylation sites. The amino acid sequence of human TM4SF1 (hTM4SF1) is described in SEQ ID NO: 90 (see also NCBI Ref Seq No. NP_055035.1).

The term “antibody”, as used herein, means any antigen-binding molecule comprising at least one complementarity determining region (CDR) that specifically binds to or interacts with a particular antigen (e.g., TM4SF1). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-TMS4F1 antibody (or antigen-binding portion thereof) may be identical to the human germline sequences or may be naturally or artificially modified. An amino acid consensus sequence may be defined based on a side-by-side analysis of two or more CDRs.

The term “intact antibody” refers to an antibody comprising four polypeptide chains, two heavy (H) chains and two light (L) chains inter-connected by disulfide bonds. In one embodiment, the anti-TM4SF1 antibody is an intact antibody. In one embodiment, the intact antibody is an intact human IgG1, IgG2 or IgG4 isotype. In certain embodiments, the anti-TM4SF1 antibody, or antigen-binding fragment thereof, is a human IgG1, IgG2, or IgG4 isotype.

The terms “antigen-binding portion” of an antibody, “antigen-binding fragment,” or “antibody-fragment,” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from intact antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide.

The term “variable region” or “variable domain” of an antibody, or fragment thereof, as used herein refers to the portions of the light and heavy chains of antibody molecules that include amino acid sequences of complementarity determining regions (CDRs; i.e., CDR-1, CDR-2, and CDR-3), and framework regions (FRs). VH refers to the variable domain of the heavy chain. VL refers to the variable domain of the light chain. According to the methods used in this disclosure, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

The term “complementarity determining regions” or “CDRs” as used herein refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia et al., J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “framework regions” (hereinafter FR) as used herein refers to those variable domain residues other than the CDR residues. Each variable domain typically has four FRs identified as FR1, FR2, FR3 and FR4. Common structural features among the variable regions of antibodies, or functional fragments thereof, are well known in the art. The DNA sequence encoding a particular antibody can generally be found following well known methods such as those described in Kabat, et al. 1987 Sequence of Proteins of Immunological Interest, U.S. Department of Health and Human Services, Bethesda MD, which is incorporated herein as a reference. In addition, a general method for cloning functional variable regions from antibodies can be found in Chaudhary, V. K., et al., 1990 Proc. Natl. Acad. Sci. USA 87:1066, which is incorporated herein as a reference.

The term “Fc region” herein is used to define a C-terminal region of an antibody heavy chain, including, for example, native sequence Fc regions, recombinant Fc regions, and variant Fc regions. Although the boundaries of the Fc region of an antibody heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system as in Kabat et al.) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Further, a composition of intact antibodies in this disclosure may comprise antibody populations with extension of residues after the C-terminal lysine, K447.

The term “humanized antibody” as used herein refers to an antibody or a variant, derivative, analog or fragment thereof, which immunospecifically binds to an antigen of interest (e.g., human TM4SF1), and which comprises a framework (FR) region having substantially the amino acid sequence of a human antibody and a complementary determining region (CDR) having substantially the amino acid sequence of a non-human antibody. Humanized forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are hereby incorporated by reference in their entireties.

The term “monoclonal antibody” as used herein refers to an antibody obtained from a population of substantially homogeneous antibodies, i.e., the individual antibodies comprising the population are identical except for possible mutations, e.g., naturally occurring mutations that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. In contrast to polyclonal antibody preparations, which typically include different antibodies directed against different determinants (epitopes), each monoclonal antibody of a monoclonal-antibody preparation is directed against a single epitope on an antigen.

The term “chimeric antibody” as used herein refers to antibodies (immunoglobulins) that have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

The term “epitope” as used herein refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “an effective amount” as used herein refers to an amount effective, at dosages, and for periods of time necessary, to achieve the desired result with respect to the treatment of a disease. For example, in the treatment of dry eye disease, an agent (i.e., a compound, a pharmaceutical composition, an antibody-drug conjugate) which decrease, prevents, delays or suppresses or arrests any symptoms of cancer or canine hemangiosarcoma would be effective. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two or more doses in a suitable form to be administered at one, two or more times throughout a designated time period.

The terms “payload,” “drug payload,” “therapeutic molecule,” therapeutic payload”, “therapeutic agents,” “therapeutic moieties,” as used interchangeably herein, refers to a chemical or biological moiety that is conjugated to an anti-TMSF1 antibody or antigen binding fragment (e.g., an anti-TM4SF1 antibody or antigen binding fragment disclosed herein), and can include any therapeutic or diagnostic agent, for example, but not limited to, small molecules, both for cancer and for non-cancer angiogenic indications; a V-ATPase inhibitor; a pro-apoptotic agent; a Bcl2 inhibitor; an MCL1 inhibitor; a HSP90 inhibitor; an IAP inhibitor; an mTor inhibitor; a microtubule stabilizer; a microtubule destabilizer; an auristatin; a dolastatin; a maytansinoid; a MetAP (methionine aminopeptidase); an inhibitor of nuclear export of proteins CRM1; a DPPIV inhibitor; proteasome inhibitors; inhibitors of phosphoryl transfer reactions in mitochondria; a protein synthesis inhibitor; a kinase inhibitor (such as, a CDK2 inhibitor, a CDK9 inhibitor); a kinesin inhibitor, an HDAC inhibitor, a DNA damaging agent, a DNA alkylating agent, a DNA intercalator, a DNA minor groove binder, a DHFR inhibitor, a nucleic acid, a CRISPR enzyme; degraders (such as agents that induce protein degradation, (e.g., HSP90 inhibitor, selective estrogen receptor degraders (SERDs), selective androgen receptor degraders (SARDs); hydrophobic tags that can be used to recruit chaperones to a protein of interest, e.g., Adamantane, Arg-Boc3; E3 ligase recruiting ligands, e.g., Nutlin-3a (MDM2 ligand), Bestatin (cIAP ligand), VHL ligand, Pomalidomide (CRBN ligand); proteolysis-targeting chimeras (PROTACs) that may utilize different D3 ligases to target a protein of interest for degradation)) (see, e.g., Lai A C, Crews C M. Induced protein degradation: an emerging drug discovery paradigm. Nat Rev Drug Discov. 2016; 16(2):101-114); antisense oligonucleotides; RNAi agents (such as siRNA), CRISPR-Cas9 gene editing systems; RNA molecules; DNA e.g., plasmids; an anti-cancer agent, an anti-inflammatory agent, an anti-infective agent (e.g., anti-fungal, antibacterial, anti-parasitic, anti-viral), an anesthetic agent; RNA polymerase II inhibitor; a DNA intercalating agent, a DNA cross-linking agent; an anti-tubulin agent; a cytotoxic drug, a tumor vaccine, an antibody, a peptide, pepti-bodies, a chemotherapeutic agent, a cytotoxic agent; a cytostatic agent; an immunological modifiers, an interferon, an interleukin, an immuno stimulatory growth hormone, a cytokine, a vitamin, a mineral, an aromatase inhibitor, a Histone Deacetylase (HDAC), an HDAC inhibitor; a lipid nanoparticle to encapsulate one or more therapeutic molecules.

The term “polypeptide” or “peptide” can refer to two or more naturally or non-naturally-occurring amino acids joined by a covalent bond (e.g., an amide bond). Polypeptides or peptide as described herein include full length proteins (e.g., fully processed proteins) as well as shorter amino acid sequences (e.g., fragments of naturally-occurring proteins or synthetic polypeptide fragments). A tetrapeptide has four amino acid covalently joined by covalent bonds.

The term “substituent” can refer to a group replacing a second atom or group such as a hydrogen atom on any molecule, compound or moiety. Suitable substituents may include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

The term “drug-to-antibody ratio” or “DAR” can refer to the number of drugs (also referred to herein as therapeutic molecules, therapeutic agents, or therapeutic moieties), attached to an anti-TM4SF1 antibody or antigen binding fragments thereof, of the ADCs disclosed herein. The DAR of an ADC typically ranges from 1 to 12, although higher loads, e.g., 16, are also possible depending on the number of linkage sites on an antibody or the use of multivalent linkages in which multiple drug payloads are attached to one linkage site. The term DAR may be used in reference to the number of drug molecules loaded onto an individual antibody, or, alternatively, may be used in reference to the average or mean DAR of a group of ADCs to reflect average drug loading. Compositions, batches, and/or formulations of a plurality of ADCs may be characterized by an average DAR. DAR and average DAR can be determined by various conventional means such as UV spectroscopy, mass spectroscopy, ELISA assay, radiometric methods, hydrophobic interaction chromatography (HIC), electrophoresis and HPLC.

The term “binding affinity” generally refers to the strength of the sum total of noncovalent interactions between a single binding site of a molecule (e.g., a binding protein such as an antibody) and its binding partner (e.g., an antigen). The affinity of a binding molecule X (e.g., anti-TM4SF1 antibody) for its binding partner Y (e.g., human TM4SF1) can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} may also be measured by using FACS or surface plasmon resonance assays by BIACORE, using, for example, a BIACORE 2000 or a BIACORE 3000, or by biolayer interferometry using, for example, the OCTET QK384 system. In certain embodiments, the K_{D} of an anti-TM4SF1 antibody is determined using a standard flow cytometry assay with HUVEC cells. An “on-rate” or “rate of association” or “association rate” or “kₒₙ” and an “off-rate” or “rate of dissociation” or “dissociation rate” or “k_{off}” may also be determined with the same surface plasmon resonance or biolayer interferometry techniques described above using, for example, a BIACORE 2000 or a BIACORE 3000, or the OCTET QK384 system.

The term “kₒₙ”, as used herein, is intended to refer to the on rate constant for association of an antibody to the antigen to form the antibody/antigen complex, as is known in the art.

The term “k_{off}”, as used herein, is intended to refer to the off rate constant for dissociation of an antibody from the antibody/antigen complex, as is known in the art.

The term “inhibition” or “inhibit,” when used herein, refers to partial (such as, 1%, 2%, 5%, 10%, 20%, 25%, 50%, 75%, 90%, 95%, 99%) or complete (i.e., 100%) inhibition.

The term “cancer” as used herein, refers to or describes the physiological condition in mammals that is typically characterized by unregulated cell growth.

The term “cancer which is associated with a high risk of metastasis”, as used herein, refers to a cancer that is associated with at least one factor known to increase the risk that a subject having the cancer will develop metastatic cancer. Examples of factors associated with increased risk for metastasis include, but are not limited to, the number of cancerous lymph nodes a subject has at the initial diagnosis of cancer, the size of the tumor, histological grading, and the stage of the cancer at initial diagnosis.

The term “hematogenous metastasis” as used herein refers to the ability of cancer cells to penetrate the walls of blood vessels, after which they are able to circulate through the bloodstream (circulating tumor cells) to other sites and tissues in the body.

The term “lymphatic metastasis” as used herein refers to the ability of cancer cells to penetrate lymph vessels and drain into blood vessels.

In the context of the disclosure, the term “treating” or “treatment”, as used herein, means reversing, alleviating, inhibiting the progress of, or preventing the disorder or condition to which such term applies, or one or more symptoms of such disorder or condition. By the term “treating cancer” as used herein is meant the inhibition of the growth and/or proliferation of cancer cells. In one embodiment, the compositions and methods described herein are used to treat metastasis in a subject having metastatic cancer.

The term “preventing cancer” or “prevention of cancer” refers to delaying, inhibiting, or preventing the onset of a cancer in a mammal in which the onset of oncogenesis or tumorigenesis is not evidenced but a predisposition for cancer is identified whether determined by genetic screening, for example, or otherwise. The term also encompasses treating a mammal having premalignant conditions to stop the progression of, or cause regression of, the premalignant conditions towards malignancy. Examples of premalignant conditions include hyperplasia, dysplasia, and metaplasia. In some embodiments, preventing cancer is used in reference to a subject who is in remission from cancer.

A variety of cancers, including malignant or benign and/or primary or secondary, may be treated or prevented with a method according to the disclosure. Examples of such cancers are known to those skilled in the art and listed in standard textbooks such as the Merck Manual of Diagnosis and Therapy (published by Merck).

The term “subject” as used herein, refers to a mammal (e.g., a human).

The term “administering” as used herein refers to a method of giving a dosage of an antibody or fragment thereof, or a composition (e.g., a pharmaceutical composition) to a subject. The method of administration can vary depending on various factors (e.g., the binding protein or the pharmaceutical composition being administered, and the severity of the condition, disease, or disorder being treated).

The term “effective amount” as used herein refers to the amount of an antibody or pharmaceutical composition provided herein which is sufficient to result in the desired outcome.

The terms “about” and “approximately” mean within 20%, within 15%, within 10%, within 9%, within 8%, within 7%, within 6%, within 5%, within 4%, within 3%, within 2%, within 1%, or less of a given value or range.

The term “identity,” or “homology” as used interchangeable herein, may be to calculations of “identity,” “homology,” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=#of identical positions/total #of positions ×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

The term “manufacturability,” as used herein, refers to the stability of a particular protein during recombinant expression and purification of that protein. Manufacturability is believed to be due to the intrinsic properties of the molecule under conditions of expression and purification. Examples of improved manufacturability characteristics include uniform glycosylation of a protein, increased cell titer, growth and protein expression during recombinant production of the protein, improved purification properties, less propensity of aggregation or non-aggregation, and improved stability, including, but not limited to, thermal stability and stability at low pH. In some embodiments are provided TM4SF1 binding proteins that demonstrate the manufacturability, along with retention of in vitro and in vivo activity, compared with other TM4SF1 antibodies. In some embodiments, humanization of a parent TM4SF1 binding protein, by making amino acid substitutions in the CDR or framework regions, can confer additional manufacturability benefits.

In some embodiments are provided TM4SF1 binding proteins that demonstrate improved developability characteristics, including, but not limited to improved purification yield, for example, after protein A purification or size exclusion chromatography, improved homogeneity after purification, improved thermal stability. In some cases, the improvement is with respect to an anti-TM4SF1 antibody produced by a hybridoma mouse cell line 8G4-5-13-13F (PTA-120523), as determined by HLA molecule binding.

In some examples, binding affinity is determined by Scatchard analysis, which comprises generating a Scatchard plot, which is a plot of the ratio of concentrations of bound ligand to unbound ligand versus the bound ligand concentration.

The term “vascular toxicity” refers to any effect of an anti-TM4SF1 antibody-therapeutic molecule conjugate (also referred to herein as anti-TM4SF1 ADC or TM4SF1 targeted ADC) which leads to vascular injury either directly due to the antibody or the therapeutic molecule effects on antigen-bearing cells or indirectly through activation of the immune system and resulting inflammation. Such vascular injury may include, but is not limited to, damage or inflammation affecting vascular endothelial cells or underlying smooth muscle cells or pericytes or the basement membrane of any blood vessel, including the endocardium (lining of the heart). Such vascular injury may affect arteries, including major arteries such as the aorta, elastic arteries (such as the aorta), muscular arteries of varying sizes, such as coronary artery, pulmonary artery, carotid artery, arterioles, capillaries, arteries of the brain or retina; venues, veins; or it may affect angiogenic vessels including vessels serving hair follicles, the digestive tract, and bone marrow. Such vascular injury may include microvascular dysfunction or damage in the heart, lung, kidney, retina, brain, skin, liver, digestive tract, bone marrow, endocrine glands, testes or ovaries, endometrium, and other target organs and may include renal, retinal or cerebrovascular circulation dysfunction.

The term “antibody-dependent cell-mediated cytotoxicity (ADCC)” as used herein refers to the killing of an antibody-coated target cell by a cytotoxic effector cell through a nonphagocytic process, characterized by the release of the content of cytotoxic granules or by the expression of cell death-inducing molecules. ADCC is triggered through interaction of target-bound antibodies (belonging to IgG or IgA or IgE classes) with certain Fc receptors (FcRs), glycoproteins present on the effector cell surface that bind the Fc region of immunoglobulins (Ig). Effector cells that mediate ADCC include natural killer (NK) cells, monocytes, macrophages, neutrophils, eosinophils and dendritic cells. ADCC is a rapid effector mechanism whose efficacy is dependent on a number of parameters (density and stability of the antigen on the surface of the target cell; antibody affinity and FcR-binding affinity). PBMC-based ADCC assays and natural kill cell-based ADCC assays can be used to detect ADCC. The readout in these assays is endpoint-driven (target cell lysis).

The term “complement dependent cytotoxicity” or “CDC” refers to the lysis of a target cell in the presence of complement. Activation of the classical complement pathway is initiated by the binding of the first component of the complement system (Clq) to antibodies (of the appropriate subclass) which are bound to their cognate antigen. To assess complement activation, a CDC assay (See, e.g., Gazzano-Santoro et al., 1996, J. Immunol. Methods 202:163) may be performed. Polypeptide variants with altered Fc region amino acid sequences (polypeptides with a variant Fc region) and increased or decreased Cq binding capability have been described (see, e.g., U.S. Pat. No. 6,194,551; WO 1999/51642; Idusogie et al., 2000, J. Immunol. 164: 4178-84). Antibodies (or fragments) with little or no CDC activity may be selected for use.

The term “effector function” as used herein refers to a function contributed by an Fc effector domain(s) of an IgG (e.g., the Fc region of an immunoglobulin). Such function can be effected by, for example, binding of an Fc effector domain(s) to an Fc receptor on an immune cell with phagocytic or lytic activity or by binding of an Fc effector domain(s) to components of the complement system. Examples of antibody effector functions include: Clq binding and complement dependent cytotoxicity (CDC); Fc receptor binding; antibody-dependent cell-mediated cytotoxicity (ADCC); phagocytosis (ADCP); down regulation of cell surface receptors (e.g., B cell receptor); and B cell activation.

The terms “reduce” or “ablate” as used herein refers to the ability to cause an overall decrease preferably of 20% or greater, more preferably of 50% or greater, and most preferably of 75%, 85%, 90%, 95%, or greater. Reduce or ablate can refer to binding affinity of two molecules, for example the binding of immunoglobulins to C1q or to Fc receptors; or can refer to the symptoms of the disorder (e.g., cancer) being treated, such as the presence or size of metastases or the size of the primary tumor.

The term “reduced ADCC/CDC function,” as used herein refers to a reduction of a specific effector function, e.g., ADCC and/or CDC, in comparison to a control (for example an antibody with a Fc region not including the mutation(s)), by at least about 5%, at least about 10%, at least about 15%, at least about 20%, at least about 30%, at least about 40%, at least about 50%, at least about 60%, at least about 70%, at least about 80% at least, at least about 90% or more.

The term “sugar moiety,” as used herein refers to a cyclic hexose, such as a pyranose, or a cyclic pentose, such as a furanose. In some embodiments, the pyranose is a galactoside or hexose. In some embodiments, the sugar moiety is in the O-D conformation. In some embodiments, the pyranose is a β-D-galactoside moiety (i.e., β-D-galactoside with a glycosidic bond that is cleavable by beta-galactosidase). In some embodiments, the sugar moiety is unsubstituted (e.g., a naturally occurring cyclic hexose or cyclic pentose). In some embodiments, the sugar moiety can be a substituted 3-D-galactoside (i.e., galactoside substituted with one or more groups, including, for example, hydrogen, hydroxyl, halogen, sulfur, nitrogen or lower alkyl containing 1-6 carbons). The glycosidic bond (—O—) connecting the sugar moiety can be a beta-galactosidase-cleavage site and can be a bond cleavable by human, lysosomal beta-galactosidase.

The term “cytotoxin,” as used herein refers to a molecule that, when released within a cancer cell, is toxic to that cell. Cytotoxins of particular interest in this invention are the tubulysins (such as the natural tubulysins or their derivatives), the auristatins (such as monomethylauristatin E and monomethylauristatin F), the maytansinoids (such as mertansine), the cahcheamicins (such as calicheamicin γ).

For all amino acid positions discussed in the present disclosure, in the context of antibodies or antigen binding fragments thereof, numbering is according to the EU index. The “EU index” or “EU index as in Kabat et al.” or “EU numbering scheme” refers to the numbering of the EU antibody (See Edelman et al., 1969; Kabat et al., 1991).

### II. Antibody-Drug Conjugates Containing Anti-TM4SF1 Antibodies or Antigen Binding Fragments Thereof, with Modified Fc Regions and/or CDR Regions

One embodiment of the disclosure provides antibody-drug conjugates (ADCs) comprising an anti-TM4SF1 antibody or an antigen binding fragment thereof linked to a therapeutic molecule, wherein the anti-TM4SF1 antibody or the antigen binding fragment thereof comprises a modified Fc region, such as a modified IgG region (e.g., IgG1, IgG2, IgG3, IgG4) comprising one or more mutations. In some cases, the one or more mutations in the Fc region leads to improvements in a drug comprising such a modified Fc region, in areas of improvement such as: 1) reduction of effector functions, 2) half-life modulation, 3) stability, and 4) downstream processes. In some cases, the modified Fc region can comprise one or more mutations that will reduce or ablate interactions between the antibodies and the immune system. Key interactions may include interactions of the antibody Fc with Fcγ receptors on white blood cells and platelets, and with C1q of the complement system leading to complement dependent cytotoxicity.

The present disclosure provides, in some cases, an ADC comprising an anti-TM4SF1 antibody or an antigen binding fragment thereof that includes immune ablating mutations, for example, in the Fc region which in such cases is a modified Fc region, for example, a modified IgG Fc region. In some embodiments, the modified Fc region comprises a modification at position N297. In some embodiments, the modified Fc region comprises a modified IgG Fc region (e.g., a modified IgG1, IgG2, IgG3, or IgG4 Fc region) comprising one or more mutations at positions E233, L234 or F234, L235, G237, P238, F243, T250, M252, S254, T256, E258, D259, V264, D265, K288, N297, T299, T307, V308, Q311, K322, L328, P329, A330, P331, T356, K370, A378, R409, V427, M428, H433, N434, and H435, or any combinations thereof. In some embodiments, the Fc region comprises an extension of residues at its C-terminus, such that positive charge is maintained at the C-terminus (e.g., in some cases, if the anti-TM4SF1 antibody or the antigen binding fragment comprises two heavy chains then at least one heavy chain comprises an extension of residues at the C-terminus). Such extension of residues can comprise addition of one or more amino acids, such as, arginine, lysine, proline, or any combinations thereof. In some examples, the extended C-terminus of the Fc regions leads to reduced CDC function of the anti-TM4SF1 antibody or the antigen binding fragment thereof, and that of an ADC comprising the anti-TM4SF1 antibody or the antigen binding fragment thereof. Such an effect is seen, in some cases, by addition of KP residues after K447 of Fc in IgG1 or IgG4, alone or in combination with other mutations (e.g., K322A, P331G-IgG1).

In some embodiments, an anti-TM4SF1 antibody or an antigen binding fragment thereof can comprise an antibody with reduced effector function, including substitution of one or more of Fc region residues 238, 265, 269, 270, 297, 327 and 329 (See, e.g., U.S. Pat. No. 6,737,056). In some cases, such mutations in the Fc region may comprise substitutions at two or more of amino acid positions 265, 269, 270, 297 and 327, for example, substitution of residues 265 and 297 to alanine (DANA mutations, i.e., D265A and N297A) (See, e.g., U.S. Pat. No. 7,332,581). In some cases, mutations in the Fc region may comprises substitutions at one or more amino acid positions E233, L234, L235, G237, D265, N297, K322, and P331. In some cases, mutations in the Fc region may comprises at least one of E233P, L234A, L235A, G237A, D265A, N297A, K322A, and P331G, or any combinations thereof. For instance, the mutations in the Fc region can comprise L234A/L235A/G237A (IgG1), or F234A/L235E (IgG4), and an anti-TM4SF1 antibody or antigen binding fragment comprising such mutations may exhibit altered FcgRI interactions.

In some embodiments, an anti-TM4SF1 antibody or antigen binding fragment thereof may include an Fc variant comprising the following mutations: an amino acid substitution at position M428 and N434 (M428L, N434S) (See, e.g., U.S. Pat. No. 9,803,023). In some embodiments, an anti-TM4SF1 antibody or antigen binding fragment thereof may include an Fc variant comprising the following mutations: an amino acid substitution at position T250 and M428 (T250Q, M428L) (See, e.g., U.S. Pat. No. 9,803,023).

In some embodiments, the TM4SF1 antibody or antigen binding fragment thereof may comprise mutations D265A and N297A. In some cases, the proline at position 329 (P329) of a wild-type human Fc region may be substituted with glycine or arginine or an amino acid residue large enough to destroy the proline sandwich within the Fc/Fcy receptor interface, that is formed between the P329 of the Fc and tryptophan residues W87 and WHO of FcgRIII (See, e.g., Sondermann et al., Nature 406, 267-273 (20 Jul. 2000)). In a further embodiment, the mutations in the Fc region may comprise one or more amino acid substitutions such as S228P (IgG4), E233P, L234A, L235A, L235E, N297A, N297D, or P331S and in still in other embodiments: L234A and L235A of the human IgG1 Fc region or S228P and F234A, L235A, or L235E of the human IgG4 Fc region.

In some embodiments, an anti-TM4SF1 antibody or antigen binding fragment thereof may include a modified Fc region which is an Fc variant of a wild-type human IgG Fc region wherein P329 of the human IgG Fc region substituted with glycine and wherein the Fc variant comprises at least two further amino acid substitutions at L234A and L235A of the human IgG1 Fc region or S228P and L235E of the human IgG4 Fc region, and wherein the residues are numbered according to the EU numbering (See, e.g., U.S. Pat. No. 8,969,526). The polypeptide comprising the P329G, L234A and L235A substitutions may exhibit a reduced affinity to the human FeyRIIIA and FeyRIIA, for down-modulation of ADCC to at least 20% of the ADCC induced by the polypeptide comprising the wildtype human IgG Fc region, and/or for down-modulation of ADCP (See, e.g., U.S. Pat. No. 8,969,526).

In some embodiments, an anti-TM4SF1 antibody or antigen binding fragment thereof may include an Fc variant comprising triple mutations: an amino acid substitution at position P329, a L234A and a L235A mutation (P329/LALA) (See, e.g., U.S. Pat. No. 8,969,526).

Certain anti-TM4SF1 antibodies or antigen binding fragments of this disclosure, in some embodiments, can comprise mutations that exhibit improved or diminished binding to FcRs. (See, e.g. U.S. Pat. No. 6,737,056; WO 2004/056312, and Shields et al., J. Biol. Chem. 9(2): 6591-6604 (2001).)

In some instances, an anti-TM4SF1 antibody or antigen binding fragment may include an Fc region with one or more amino acid substitutions which improve ADCC, e.g., substitutions at positions 298, 333, and/or 334 of the Fc region. Alterations may be made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. (2000) J. Immunol. 164: 4178-4184.

Antibodies with increased half-lives and improved binding to the neonatal Fc receptor (FcRn). FcRn, named after its function for the transfer of maternal IgGs to the fetus, also serves to prevent antibodies from being degraded in lysosomes, by capturing them in endosomes and returning them to circulation. (See, e.g., Guyer et al., J. Immunol. 117:587 (1976) and Kim et al., J. Immunol. 24:249 (1994)), are described in US2005/0014934. Without being bound by any particular theory, it is contemplated that antibodies with improved binding to FcRn detach from TM4SF1 and bind to FcRn, which then recycles the ADC back to circulation, thus reducing vascular toxicity. In some embodiments herein are provided anti-TM4SF1 antibodies or antigen binding fragments that comprise an Fc region with one or more substitutions that enhance FcRn recycling. In some embodiments herein are provided anti-TM4SF1 antibodies or antigen binding fragments thereof that comprise an Fc region with one or more substitutions therein which improve binding of the Fc region to FcRn, such as, substitutions at one or more of positions: 238, 250, 252, 254, 256, 265, 272, 286, 303, 305, 307, 311, 312, 317, 340, 356, 360, 362, 376, 378, 380, 382, 413, 428, 424, 434, and 435, e.g., substitution of Fc region residue 434 (U.S. Pat. No. 7,371,826) according to EU numbering. See also Duncan &amp; Winter, Nature 322:738-40 (1988); U.S. Pat. Nos. 5,648,260; 5,624,821; US2005/0014934 and WO 94/29351 concerning other examples of Fc region variants, the entirety of which are incorporated herein by reference.

In some embodiments, provided herein are anti-TM4SF1 antibodies or antigen binding fragments thereof that have pH dependent FcRn binding affinities. Without being bound by any particular theory, it is contemplated that ADC antibodies or antigen binding fragments thereof with pH dependent FcRn binding affinity detach from FcRn at pH >7 and bind to FcRn at pH 6. Accordingly, FcRn in acidic pH subcellular organelles, e.g., endosomes, binds such antibodies and carries the antibodies back to the cell membrane, and release the antibodies into plasma at pH >7, recycling the antibody and avoiding lysosomal release of ADC payloads.

In certain embodiments, herein are provided anti-TM4SF1 antibodies or antigen binding fragments thereof that comprise an Fc region with one or more substitutions therein which modulate FcRn recycling. In some embodiments herein are provided anti-TM4SF1 antibodies or antigen binding fragments thereof that comprise one or more substitutions that enhance FcRn binding at acidic pH, e.g., pH 6, and does not affect FcRn binding at neutral or basic pH, e.g., pH 7. In some embodiments, an anti-TM4SF1 antibody or antigen binding fragment thereof may comprise substitutions at one or more of positions 250, 252, 254, 256, 428, and 434 according to EU numbering. In some embodiments, an anti-TM4SF1 antibody or antigen binding fragment thereof may include an Fc variant comprising one or more of substitutions T250Q, M252Y, S254T, T256E, M428L, and N434S. In some embodiments, an anti-TM4SF1 antibody or antigen binding fragment thereof may include an IgG1 Fc variant comprising substitutions T250Q and M428L (the “QL mutant”). In some embodiments, an anti-TM4SF1 antibody or antigen binding fragment thereof may include an IgG4 Fc variant comprising substitutions T250Q and M428L (the “QL mutant”). In some embodiments, an anti-TM4SF1 antibody or antigen binding fragment thereof may include an IgG1 Fc variant comprising substitutions M252Y, S254T, and T256E (the “YTE mutant”). In some embodiments, an anti-TM4SF1 antibody or antigen binding fragment thereof may include an IgG1 Fc variant comprising substitutions M428L and N434S (the “LS mutant”). In some embodiments, an anti-TM4SF1 antibody or antigen binding fragment thereof may include an IgG4 Fc variant comprising substitutions M428L and N434S (the “LS mutant”). Effects of amino acid substitutions in the Fc region that modulate FcRn recycling are described in, e.g., Hamblett et al., Mol. Pharm. 13(7): 2387-96 (2016); Dall'Acqua et al., J. Biol. Chem. 281(33): 23514-24 (2006), Hinton et al., J. Biol. Chem. 279(8): 6213-6 (2003), Hinton et al., J. Immunol., 176(1): 346-56 (2006), US20080181887, U.S. Pat. No. 7,361,740, and EP2235059, the entirety of which are incorporated herein by reference.

In certain embodiments, an anti-TM4SF1 antibody, or antigen-binding fragment thereof, is an IgG1 isotype and comprises an Fc region comprising one or more substitutions selected from the group consisting of T250Q, M252Y, S254T, T256E, M428L, and N434S. In some embodiments, an anti-TM4SF1 antibody, or antigen binding fragment thereof, is an IgG4 isotype and comprises an Fc region comprising one or more substitutions selected from the group consisting of T250Q, M252Y, S254T, T256E, M428L, and N434S. In some embodiments, an anti-TM4SF1 antibody or antigen binding fragment thereof is an IgG1 isotype and comprises an Fc region comprising substitutions T250Q and M428L. In some embodiments, an anti-TM4SF1 antibody or antigen binding fragment thereof is an IgG1 isotype and comprises an Fc variant comprising substitutions M252Y, S254T, and T256E. In some embodiments, an anti-TM4SF1 antibody or antigen binding fragment thereof is an IgG4 isotype and comprises an Fc variant comprising substitutions M252Y, S254T, and T256E. In some embodiments, an anti-TM4SF1 antibody or antigen binding fragment thereof is an IgG1 isotype and comprises an Fc variant comprising substitutions M428L and N434S. In some embodiments, an anti-TM4SF1 antibody or antigen binding fragment thereof is an IgG4 isotype and comprises an Fc variant comprising substitutions M428L and N434S.

In certain embodiments, the ADCs disclosed herein exhibit reduced vascular toxicity, reduced lysosomal toxicity, improved efficacy, and/or improved therapeutic margin. In some embodiments, the ADCs disclosed herein comprise anti-TM4SF1 antibodies or antigen binding fragments thereof comprising mutated Fc regions that have increased FcRn binding affinity and increased serum half-life. In certain embodiments, an anti-TM4SF1 antibody or antigen binding fragment thereof comprising mutated Fc regions have serum half-life of at least 10 days, at least 15 days, at least 20 days, at least 25 days, at least 30 days, at least 35 days, at least 40 days, at least 50 days, at least 60 days, at least 70 days, at least 80 days, at least 90 days, at least 100 days or more.

In certain embodiments, the ADCs of this disclosure exhibit reduced vascular toxicity, improved therapeutic margin, or both. In certain embodiments the ADCs of this disclosure comprise anti-TM4SF1 antibodies or antigen binding fragments thereof comprising mutated Fc regions that have reduced or ablated affinity for an Fc ligand responsible for facilitating effector function compared to an antibody having the same amino acid sequence as the antibody of the disclosure but not comprising the addition, substitution, or deletion of at least one amino acid residue to the Fc region (also referred to herein as an “unmodified antibody”).

In one embodiment, an anti-TM4SF1 antibody, or antigen-binding fragment thereof comprises an Fc region comprising at least two mutations that reduce or ablate ADCC and/or CDC effector function of the antibody, or antigen-binding fragment thereof. In further embodiments, the anti-TM4SF1 antibody, or antigen-binding fragment thereof, comprises an Fc region comprising at least three, at least four, at least five, at least six, at least seven, at least eight, at least nine, at least ten or more mutations that reduce or ablate ADCC and/or CDC effector function of the antibody, or antigen-binding fragment thereof.

In certain embodiments, an anti-TM4SF1 antibody, or antigen-binding fragment thereof, is an IgG1 isotype and comprises an Fc region comprising one or more mutations selected from the group consisting of E233P, L234V, L234A, L235A, G236Delta (deletion), G237A, V263L, N297A, N297D, N297G, N297Q, K322A, A327G, P329A, P329G, P329R, A330S, P331A, P331G, and P331S.

In one embodiment, an anti-TM4SF1 antibody, or antigen-binding fragment thereof, is an IgG1 isotype and comprises an Fc region comprising an L234A/L235A mutation, with or without a G237A mutation. In one embodiment, the anti-TM4SF1 antibody, or antigen-binding fragment thereof, is an IgG1 isotype and comprises an Fc region comprising L234A, L235A, and G237A mutations.

In one embodiment, an anti-TM4SF1 antibody, or antigen-binding fragment thereof, is an IgG1 isotype and comprises an Fc region comprising an A327G/A330S/P331S mutation.

In one embodiment, an anti-TM4SF1 antibody, or antigen-binding fragment thereof, is an IgG1 isotype and comprises an Fc region comprising an E233P/L234V/L235A/delta G236 (deletion) mutation, which provides reduced binding to FcγRI (also referred to herein as FcgRI), FcγRIIA (also referred to herein as FcgRIIA), FcγRIIIA (also referred to herein as FcgRIIIAI) and reduced ADCC and CDC effector function, as described, for example, in An Z et al. Mabs 2009 November-Ec; 1(6):572-9, incorporated by reference in its entirety herein.

In one embodiment, an anti-TM4SF1 antibody, or antigen-binding fragment thereof, is an IgG1 isotype and comprises an Fc region comprising an N297x mutation, where x=A, D, G, Q.

In one embodiment, an anti-TM4SF1 antibody, or antigen-binding fragment thereof, is an IgG1 isotype and comprises an Fc region comprising an A327G/A330S/P331S mutation.

In one embodiment, an anti-TM4SF1 antibody, or antigen-binding fragment thereof, is an IgG1 isotype and comprises an Fc region comprising a mutation in one or more of K322A, P329A, and P331A, which provides reduced binding to C1q, as described, for example, in Canfield &Morrison. J Exp Med (1991) 173(6):1483-91.10.1084, incorporated by reference in its entirety herein.

In one embodiment, an anti-TM4SF1 antibody, or antigen-binding fragment thereof, is an IgG1 isotype and comprises an Fc region comprising a V263L mutation, which provides enhanced binding to FcγRIIB (also referred to herein as FcgRIIB) and enhanced ADCC, as described in, for example, Hezareh et al. J Virol. 2001 December; 75(24):12161-8, incorporated by reference in its entirety herein.

In other embodiments, an anti-TM4SF1 antibody or antigen-binding fragment thereof is an IgG1 isotype and comprises an Fc region comprising a L234A/L235A, G237A or L235E mutation.

In other embodiments, an anti-TM4SF1 antibody, or antigen-binding fragment thereof, is an IgG1 isotype and comprises an Fc region comprising a L234F, L235E or P331S mutation.

In certain embodiments, an anti-TM4SF1 antibody, or antigen-binding fragment thereof, is an IgG2 isotype and comprises an Fc region comprising a one or more mutations selected from the group consisting of V234A, G237A, P238S, H268A or H268Q, V309L, A330S and P331S.

In one embodiment, an anti-TM4SF1 antibody, or antigen-binding fragment thereof, is an IgG2 isotype and comprises an Fc region comprising an A330S/P331S mutation.

In one embodiment, an anti-TM4SF1 antibody, or antigen-binding fragment thereof, is an IgG2 isotype and comprises an Fc region comprising an A330S/P331S, V234A/G237A /P238S/H268A/V309L/A330S/P331S or H268Q/V309L/A330S/P331S mutation.

In other embodiments, an anti-TM4SF1 antibody, or antigen-binding fragment thereof, is an IgG4 isotype and comprises an Fc region comprising a one or more mutations selected from the group consisting of S228P, E233P, F234A, F234V, L235E, L235A, G236Delta (deletion), N297A, N297D, N297G, N297Q, P329G, P329R.

In certain embodiments, an anti-TM4SF1 antibody, or antigen-binding fragment thereof, is an IgG4 isotype and comprises an Fc region comprising an S228P mutation, which provides reduced Fab-arm exchange and reduced aggregation, as described for example in Chappel et al. Proc Natl Acad Sci USA (1991) 88(20):9036-40, incorporated by reference in its entirety herein.

In one embodiment, an anti-TM4SF1 antibody, or antigen-binding fragment thereof, is an IgG4 isotype and comprises an Fc region comprising an S228P/L235E mutation.

In one embodiment, an anti-TM4SF1 antibody, or antigen-binding fragment thereof, is an IgG4 isotype and comprises an Fc region comprising an S228P/E233P/F234V/L235A/delta G236 (deletion) mutation.

In one embodiment, an anti-TM4SF1 antibody, or antigen-binding fragment thereof, is an IgG4 isotype and comprises an Fc region comprising an N297x mutation, where x=A, D, G, Q.

In one embodiment, an anti-TM4SF1 antibody, or antigen-binding fragment thereof, is an IgG4 isotype and comprises an Fc region comprising an S228P/F234A/L235A mutation.

In one embodiment, an anti-TM4SF1 antibody, or antigen-binding fragment thereof, is an IgG4 isotype and comprises an Fc region comprising a L235E mutation, which provides reduced binding to FeyRI, FeyRIIA, FeyRIIIA and reduced ADCC and CDC effector activity, as described in, for example, Saxena et al. Front Immunol. 2016 Dec. 12; 7:580.

In other embodiments, an anti-TM4SF1 antibody, or antigen-binding fragment thereof, is an IgG4 isotype and comprises an Fc region comprising a S228P/F234A/L235A or E233P/L235A/G236Delta mutation.

In one embodiment, an anti-TM4SF1 antibody, or antigen-binding fragment thereof, is an IgG4 isotype and comprises an Fc region comprising at least a S228P mutation. See, e.g., Angal et al. (Mol Immunol. 1993 January; 30(1):105-8) describe an analysis of the hinge sequences of human IgG4 heavy chains to determine that the presence of serine at residue 241 (according to EU numbering system, and now corresponding to residue 228 in Kabat numbering,) as the cause of heterogeneity of the inter-heavy chain disulfide bridges in the hinge region in a proportion of secreted human IgG4. Silva et al. (J Biol Chem. 2015 Feb. 27; 290(9):5462-9) describe the S228P mutation in human IgG4 that prevents in vivo and in vitro IgG4 Fab-arm exchange.

In other embodiments, an anti-TM4SF1 antibody, or antigen-binding fragment thereof, is an IgG4 isotype and comprises an Fc region comprising a L235E or S228P mutation.

In other embodiments, the anti-TM4SF1 antibody, or antigen-binding fragment thereof, is an IgG4 or IgG1 isotype and comprises an Fc region comprising a N297A, N297D or N297G mutation.

In other embodiments, an anti-TM4SF1 antibody, or antigen-binding fragment thereof, is an IgG4 or IgG1 isotype and comprises an Fc region comprising a P329G, P329R mutation.

In one exemplary embodiment, the mutated Fc region of any IgG isotype comprises one or more mutations at positions 234, 235, 236, 237, 297, 318, 320, 322 (as described in WO1988007089, incorporated by reference in its entirety herein). Other possible mutations in the Fc region, including substitutions, deletions and additions are also described in, for example, US20140170140, WO2009100309, US20090136494 and U.S. Pat. No. 8,969,526, incorporated by reference in their entireties herein.

In vitro and/or in vivo cytotoxicity assays can be conducted to confirm the reduction or ablation of CDC and/or ADCC activities. For example, Fc receptor (FcR) binding assays can be conducted to ensure that the antibody lacks FcγR binding (hence likely lacking ADCC activity) but retains FcRn binding ability. The primary cells for mediating ADCC, NK cells, express FcγRIII only, whereas monocytes express FcγRI, RII and RII. Non-limiting examples of in vitro assays to assess ADCC activity of a molecule of interest is described in U.S. Pat. No. 5,500,362 (see, e.g., Hellstrom, I., et al., Proc. Nat'l Acad. Sci. USA 83 (1986) 7059-7063) and Hellstrom, I., et al., Proc. Nat'l Acad. Sci. USA 82 (1985) 1499-1502; U.S. Pat. No. 5,821,337 (see Bruggemann, M., et al., J. Exp. Med. 166 (1987) 1351-1361). Alternatively, non-radioactive assays methods may be employed (see, for example, ACTI.TM. non-radioactive cytotoxicity assay for flow cytometry (CellTechnology, Inc. Mountain View, Calif; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes, et al., Proc. Nat'l Acad. Sci. USA 95 (1998) 652-656. C1q binding assays may also be carried out to confirm that the antibody is unable to bind C1q and hence lacks CDC activity. See, e.g., C1q and C₃c binding ELISA in WO 2006/029879 and WO 2005/100402. To assess complement activation, a CDC assay may be performed (see, for example, Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743). FcRn binding and in vivo clearance/half-life determinations can also be performed using methods known in the art (see, e.g., Petkova, S. B., et al., Int'l. Immunol. 18(12) (2006) 1759-1769).

In some embodiments, the mutated Fc region of any IgG isotype comprises a mutation at position L328, such as L328M, L328D, L328E, L328N, L328Q, L328F, L3281, L328V, L328T, L328H, L328A (see e.g., US20050054832).

In one embodiment, antibodies, or antigen-binding fragments thereof, of the disclosure exhibit reduced or ablated ADCC effector function as compared to unmodified antibodies. In another embodiment, antibodies, or antigen-binding fragments thereof, of the disclosure exhibit reduced ADCC effector function that is at least 2 fold, or at least 3 fold, or at least 5 fold or at least 10 fold or at least 50 fold or at least 100 fold less than that of an unmodified antibody. In still another embodiment, antibodies of the disclosure exhibit ADCC effector function that is reduced by at least 10%, or at least 20%, or by at least 30%, or by at least 40%, or by at least 50%, or by at least 60%, or by at least 70%, or by at least 80%, or by at least 90%, or by at least 100%, relative to an unmodified antibody. In a further aspect of the disclosure the reduction or down-modulation of ADCC effector function induced by the antibodies, or antigen-binding fragments thereof, of the present disclosure, is a reduction to 0, 2.5, 5, 10, 20, 50 or 75% of the value observed for induction of ADCC by unmodified antibodies. In certain embodiments, the reduction and/or ablation of ADCC activity may be attributed to the reduced affinity of the antibodies, or antigen-binding fragments thereof, of the disclosure for Fc ligands and/or receptors.

CDR Substitutions that Modulate pH-Dependent™-4SF1 Binding of an Anti-TM4SF1 Antibody or Antigen Binding Fragment Thereof

One embodiment of the disclosure provides ADCs comprising an anti-TM4SF1 antibody or an antigen binding fragment thereof linked to a therapeutic molecule or a payload, wherein the anti-TM4SF1 antibody or the antigen binding fragment thereof exhibit pH dependent binding affinity to TM4SF1. In some instances, an anti-TM4SF1 antibody or antigen binding fragment thereof binds to TM4SF1 with higher affinity at certain pH range as compared to other pH ranges. For example, an anti-TM4SF1 antibody or antigen binding fragment thereof may bind to TM4SF1 with different affinity at an acidic pH than at a neutral pH or a basic pH. In some embodiments, an anti-TM4SF1 antibody or antigen binding fragment thereof binds to TM4SF1 with higher affinity at an acidic pH than at a neutral or basic pH. In some embodiments, an anti-TM4SF1 antibody or antigen binding fragment thereof binds to TM4SF1 with lower affinity at an acidic pH than at a neutral or basic pH. In some embodiments, an anti-TM4SF1 antibody or antigen binding fragment thereof binds to TM4SF1 at acidic pH and dissociates from TM4SF1 at neutral or basic pH. In some embodiments, an anti-TM4SF1 antibody or antigen binding fragment thereof binds to TM4SF1 at pH7 or higher and detaches from TM4SF1 at pH6 or lower. In subcellular compartments such as plasma, cytosol, and nucleus, the pH is neutral or basic. In lysosomes or endosomes, the pH is acidic. Without being bound by any theory, an anti-TM4SF1 antibody or antigen binding fragment thereof bind to the antigen and subsequently internalized in the membrane of an endosome. A pH-dependent anti-TM4SF1 antibody or antigen binding fragment thereof can detach from TM4SF1 in an endosome and bind to FcRn receptors within the endosome and can be recycled by the FcRn receptor back into circulation rather than degraded in a lysosome that the endosome progresses to. Accordingly, a pH dependent anti-TM4SF1 antibody or antigen binding fragment thereof can bind to TM4SF1 antigen multiple times. Accordingly, a pH dependent anti-TM4SF1 antibody and the associated therapeutic molecule or payload therewith can be recycled by FcRn receptors, without releasing the payload in the lysosome.

Target-mediated drug disposition, or TMDD, occurs when an antigen carries a bound antibody and/or any associated ADC payload to the lysosome, wherein the ADC is degraded, and the payload is released. Lysosome toxicity related to TMDD as described in Grimm et al., J. Pharmacokinet. Pharmacodyn. 36(5): 407-20 (2009) is incorporated herein by reference in its entirety. In some embodiments, provided herein are ADCs comprising an anti-TM4SF1 antibody or antigen binding fragment thereof linked to a therapeutic molecule that exhibit reduced vascular toxicity, increased serum half-life, and/or improved therapeutic margin. In some embodiments, an anti-TM4SF1 antibody or antigen binding fragment thereof comprises one or more histidine amino acid residue substitutions in CDR residues. Not intended to be bound by any particular theory, the introduction of a histidine residue at a suitable position of an anti-TM4SF1 antibody may allow pH-regulatable binding affinity to TM4SF1. For example, an ADC with a pH-dependent anti-TM4SF1 antibody may dissociate from TM4SF1 in acidic lysosome or endosome environment, and subsequently be recycled into circulation via FcRn binding. As compared to an otherwise comparable wild type anti-TM4SF1 antibody or antigen binding fragment thereof, a pH-dependent ant-TM4SF1 antibody may exhibit increased serum half-life and reduced degradation rate or payload release rate in lysosomes. In some cases, the ADCs comprising a pH-dependent anti-TM4SF1 antibody or antigen binding fragment thereof may demonstrate increased half-life, reduced vascular toxicity, improved therapeutic window, and/or improved or at least about equivalent in vivo potency.

Disclosed herein are methods of making an ADC comprising an anti-TM4SF1 antibody or antigen binding fragment thereof that has increased half-life and/or pharmacodynamic effect by regulating antibody-TM4SF1 binding affinity in a pH dependent manner, comprising selecting for antibody CDR histidine residues or other residues that optimize the microenvironment affecting pKa of the antibody, such that the antibody-TM4SF1 binding has a Kd ratio and/or Koff ratio at pH6.0/pH7.4 that is at least 2, 3, 4, 8, 10, 16, or more, or ranges between 2, 3, 4, 8, 10, 16, or more. In some embodiments, the method comprises introducing amino acid substitutions into an anti-TM4SF1 antibody or antigen binding fragment thereof to achieve TM4SF1 affinity with a KD at pH 7.4 of at least 100 nM as measured at 25° C. In certain embodiments, the method comprises generating an antibody library enriched for histidines in CDR residues or other residues that optimize the microenvironment affecting pKa. In some embodiments, the antibody library comprises anti-TM4SF1 antibodies or antigen binding fragments thereof with histidine residues introduced into a CDR position. In some embodiments, the antibody library comprises a series of anti-TM4SF1 antibodies or antigen binding fragments thereof, wherein each anti-TM4SF1 antibody in the antibody library comprises a single histidine substitution at a different CDR position. In some embodiments, the antibody library comprises a series of anti-TM4SF1 antibodies or antigen binding fragments thereof, each comprising 1, 2, 3, 4, 5, 6, 7, 8, 9, 10, 11, 12, 13, 14, 15, 16, or 17 mutations to histidine residues. In some embodiments, every CDR position is mutated to histidine in at least one of the TM4SF1 antibodies or antigen fragments of the antibody library.

In some embodiments, an anti-TM4SF1 antibody or antigen binding fragment thereof comprises 1, 2, 3, 4, 5, or more histidine substitutions in a CDR region. A histidine residue can be engineered into different positions of an anti-TM4SF1 antibody light chain (LC) or heavy chain (HC) for pH dependent binding affinity. Accordingly, in some embodiments, provided herein are ADCs with histidine engineered anti-TM4SF1 antibody or antigen binding fragment thereof. In some embodiments, an anti-TM4SF1 antibody or antigen binding fragment thereof comprises one or more histidine residues in CDR1, CDR2, and/or CDR3 of the light chain variable region (VL). In some embodiments, an anti-TM4SF1 antibody or antigen binding fragment thereof comprises one or more histidine residues in CDR1 of the light chain variable region (VL). In some embodiments, an anti-TM4SF1 antibody or antigen binding fragment thereof comprises one or more histidine residues in CDR2 of the light chain variable region (VL). In some embodiments, an anti-TM4SF1 antibody or antigen binding fragment thereof comprises one or more histidine residues in CDR3 of the light chain variable region (VL). In some embodiments, an anti-TM4SF1 antibody or antigen binding fragment thereof comprises one or more histidine residues in CDR1, CDR2, and/or CDR3 of the heavy chain variable region (VH). In some embodiments, an anti-TM4SF1 antibody or antigen binding fragment thereof comprises one or more histidine residues in CDR1 of the heavy chain variable region (VH). In some embodiments, an anti-TM4SF1 antibody or antigen binding fragment thereof comprises one or more histidine residues in CDR2 of the heavy chain variable region (VH). In some embodiments, an anti-TM4SF1 antibody or antigen binding fragment thereof comprises one or more histidine residues in CDR3 of the heavy chain variable region (VH). Accordingly, in some embodiments, the ADCs of the present disclosure comprise a histidine engineered anti-TM4SF1 antibody or antigen binding fragment thereof.

In some embodiments, an anti-TM4SF1 antibody or antigen binding fragment thereof comprises one or more histidine residues in CDR1, CDR2, and/or CDR3 of the light chain, for instance, in one or more of positions 30 (S30H), 92 (S92H), and 93 (N93H) of SEQ ID No. 101 or SEQ ID No. 131. In some embodiments, an anti-TM4SF1 antibody or antigen binding fragment thereof comprises one or more histidine residues in CDR1, CDR2, and/or CDR3 of the heavy chain, for instance in one or more of positions 28 (T28H), 31 (N31H), 32 (Y₃₂H), 52 (N52H), 54 (Y₅₄H), 57 (N57H), 100 (Q100H), and 101 (Y₁₀₁H), of SEQ ID No. 92 or SEQ ID No. 130.

### Substitution at Position N297(Asn 297) and Conjugation of One or More Therapeutic Molecules to an Anti-TM4SF1 Antibody or Antigen Binding Fragment Thereof

Human IgG molecules have a conserved glycosylation site at each N297 residue in the CH2 domain, making these pendant N-glycans a convenient target for site-specific conjugation. This glycosylation site is sufficiently far from the variable region that conjugation of drug moieties to attached glycans should not impact antigen binding. In some embodiments of this disclosure, therapeutic molecules are linked to the glycans, using exemplary methods that include oxidative cleavage of the vicinal diol moieties contained in these glycans with periodate to generate aldehydes that can be reductively aminated and conjugated to hydrazide and aminooxy compounds. (See, e.g., O'Shannessy, et al. (1984) Immunol. Lett. 8:273-77).

Another method may include increasing the fucosylation of the N-acetylglucosamine residues in these glycans. Oxidation of these fucose residues can produce carboxylic acid and aldehyde moieties that can be used to link drugs and fluorophores to these specific sites on the antibody (See, e.g., Zuberbuhler, et al. (2012) Chem. Commun. 48:7100-02). Another method may include modifying sialic acid in these glycans (as well as increasing the sialic acid content in these glycans) followed by oxidation of the sialic acid and conjugation with aminooxy-drugs to form oxime-linked conjugates (See, e.g., Zhou, et al. (2014) Bioconjugate Chem. 25:510-20).

Alternatively, a sialyltransferase may be used to incorporate a modified sialic acid residue containing a bioorthogonal functional group into these glycans. The bioorthogonal functional group may then be modified to attach therapeutic molecules to the site of the glycan (See, e.g., Li, et al. (2014) Angew. Chem. Int. 53:7179-82). Another approach to modifying these glycan sites is the use of glycosyltransferases to link galactose, or galactose analogues containing ketones or azides, to the N-acetylglucosamine in these glycans, and linking drugs or radionucleotides to the galactose molecules (See, e.g., Khidekel, et al., (2003) J. Am. Chem. Soc. 125: 16162-63; Clark, et al., (2008) J. Am. Chem. Soc. 130: 11576-77; Boeggeman, et al. (2007) Bioconjugate Chem. 18:806-14). Another approach relies on the introduction of modified sugars into these glycans at the time of expression of the antibody by metabolic oligosaccharide engineering (See, e.g., Campbell, et al. (2007) Mol. BioSyst. 3: 187-94; Agard, et al., (2009) Acc. Chem. Res. 42:788-97).

In some embodiments, the anti-TM4SF1 antibody or the antigen binding fragment thereof is conjugated to a therapeutic molecule, by site-specific conjugation. Several native or engineered amino acids, including cysteines and glutamines, can be selected as the sites for conjugation.

In some instances, a cysteine residue can be engineered into different positions of antibody heavy chain (HC) or light chain (LC) for coupling, such as at position N297, i.e., N297C. Thus, in some embodiments, the ADCs of the present disclosure comprise a cysteine engineered anti-TM4SF1 antibody or an antigen binding fragment thereof.

The introduction of a cysteine residue at a suitable position of the anti-TM4SF1 antibody may allow control of the site of conjugation and the obtained site-specific conjugates may be more homogeneous than the conjugates obtained via wild-type conjugation, i.e., conjugation via reduced interchain cysteines. In some cases, the ADCs comprising at least one conjugation via cysteine may demonstrate at least equivalent in vivo potency, improved pharmacokinetics (PK), and an expanded therapeutic window compared to wild-type conjugates. The ADC, in some embodiments, comprises a cleavable dipeptide linker (i.e., valine-alanine) and a DNA-cross-linking pyrrolobenzodiazepine (PBD) dimer as the drug, which is linked to a cysteine at heavy chain position N297C in the Fc part of the anti-TM4SF1 antibody or the antigen binding fragment thereof. In some cases, the ADCs have an average drug-to-antibody ratio (DAR) of greater than or equal to 1, such as a DAR of about 2, 6, 10 etc.

Without being bound by any particular theory, it is contemplated that site-specific conjugation through unpaired cysteine can be relatively simple and scalable. For instance, the therapeutic molecule coupling can be done without the need of special reagents. In some cases, ADCs prepared through site-specific cysteines can show stronger in vivo antitumor activities and could be better tolerated than the conventional conjugates. In some embodiments, position N297 of the anti-TM4SF1 antibody or the antigen binding fragment thereof can be mutated to cysteine, i.e., N297C, and the cysteine residue can be conjugated to a therapeutic molecule. In some instances, the N297C mutation is combined with additional mutations in nearby residues, to add stabilizing residues (e.g., arginine, lysine) and/or remove glutamic acid. In some cases, one or more positions from residue 292-303 are modified, in addition to N297C. The sequence for positions 292-303 can be REEQYCSTYRVV (SEQ ID NO: 158) (in IgG1), and REEQFCSTYRVV (SEQ ID NO: 159) (in IgG4).

In some embodiments, the anti-TM4SF1 antibody or the antigen binding fragment thereof is conjugated to a therapeutic molecule, by site-specific conjugation through a glutamine residue. In some cases, microbial transglutaminase (mTG) can be used to transfer an amine containing drug-linker or a reactive spacer into Q295 residue in the heavy chain of an anti-TM4SF1 antibody or an antigen binding fragment thereof, for example, a deglycosylated anti-TM4SF1 antibody or an antigen binding fragment thereof. The conjugation can be optimized using a two-step chemoenzymatic approach whereby a reactive spacer containing a bioorthogonal azido or thiol functional linker is attached to the antibody by mTG and subsequently reacted with either dibenzocyclooctynes (DBCO) or maleimide containing MMAE. By using strain-promoted azide-alkyne cycloaddition (SPAAC) or thiol-maleimide chemistry, ADCs can be generated with DAR, for example, at about 2.

In some instances, the anti-TM4SF1 antibody or the antigen binding fragment thereof is conjugated to a therapeutic molecule, by site-specific conjugations through a glutamine residue (e.g., Q295) as well as cysteine at position 297, N297C. This combination of mutations can open up two conjugation handles in the anti-TM4SF1 antibody or the antigen binding fragment thereof, and ADCs of higher DAR can be obtained. Thus, in some embodiments of this disclosure, ADCs are provided wherein more than one therapeutic molecules (e.g., two therapeutic molecules) are conjugated to an anti-TM4SF1 antibody or antigen-binding fragment thereof via site specific conjugations at N297C and Q295. The cysteine conjugation can be, for example, to maleimide, haloacetamide, or another partner.

Increased DAR could lead to efficient ADC construction, minimal destabilization of the antibody structure, and enhanced ADC efficacy. A cysteine conjugation-based dual-loading linker enabling modular payload installation was recently developed (Levengood et al., 2017). Thus, there remains a need for ADCs capable of delivering multiple payloads.

In addition, the ADC linker structure and antibody-payload conjugation modality impact ADC homogeneity, cytotoxic potency, tolerability, and pharmacokinetics (PK). These key parameters may critically contribute to overall in vivo therapeutic efficacy (See, e.g., Lu et al., 2016, Hamblett et al., 2004, Junutula et al., 2008, and Behrens et al., 2015). Thus, refining linker and conjugation chemistries is of crucial importance to maximize the therapeutic potential and safety profiles of ADCs.

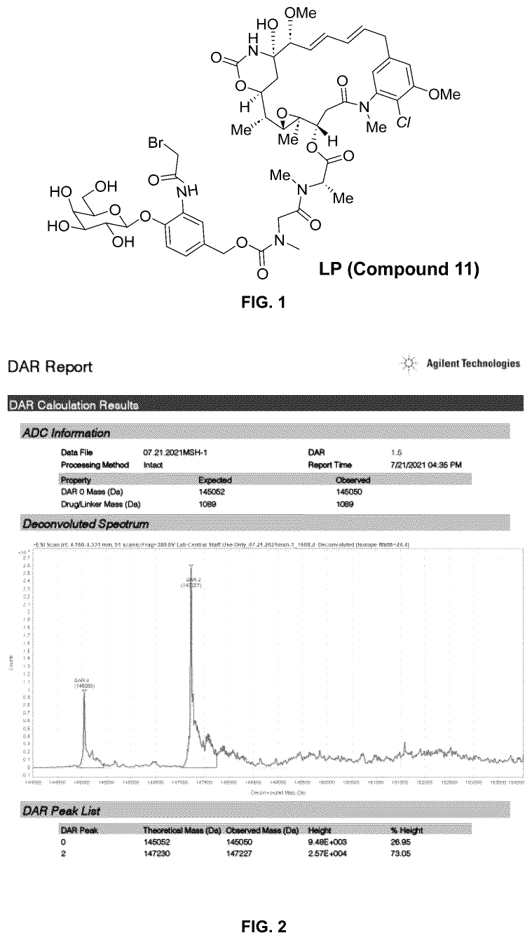

Bioconjugation modality and method may be optimized for improved ADC stability and efficacy. In some embodiments, one or more therapeutic agents and/or diagnostic agents are conjugated to anti-TM4SF1 antibodies or antigen binding fragments via maleimide, e.g., cysteine-maleimide conjugation. Other functional groups besides maleimide, which in some instances are reactive with an anti-TM4SF1 antibody, such as a thiol group of a cysteine engineered anti-TM4SF1 antibody, include iodoacetamide, bromoacetamide, vinyl pyridine, disulfide, pyridyl disulfide, isocyanate, and isothiocyanate. In some embodiments, the therapeutic agents and/or diagnostic agents are conjugated to anti-TM4SF1 antibodies or antigen binding fragments thereof via acetamide. For example, a therapeutic agent may be conjugated to an anti-TM4SF1 antibody or antigen binding fragment thereof via bromoacetamide conjugation. In some cases, an ADC comprising a bromoacetamide conjugated anti-TM4SF1 antibody or antigen binding fragment thereof exhibits increased stability, increased half-life, reduced toxicity, and/or improved therapeutic margin. Exemplary ADC structures are provided in FIGS. 1 and 2.

### III. Anti-TM4SF1 Antibody or Antigen Binding Fragments Thereof

TM4SF1 is a small plasma membrane glycoprotein (NCBI Ref Seq No. N P_055035.1) with tetraspanin topology but not homology (Wright et al. Protein Sci. 9: 1594-1600, 2000). It forms TM4SF1-enriched domains (TMED) on plasma membranes, where, like genuine tetraspanins, it serves as a molecular facilitator that recruits functionally related membrane and cytosolic molecules (Shih et al. Cancer Res. 69: 3272-3277, 2009; Zukauskas et al., Angiogenesis. 14: 345-354, 201 1), and plays important roles in cancer cell growth (Hellstrom et al. Cancer Res. 46: 391 7-3923, 1986), motility (Chang et al. Int J Cancer. 1 16: 243-252, 2005), and metastasis (Richman et al. Cancer Res. 5916s-5920s, 1995). The amino acid sequence of human TM4SF1 protein (NCBI RefSeq No. NP_055035.1) is shown below as SEQ ID NO: 134.

(SEQ ID NO: 134)
MCYGKCARCI GHSLVGLALL CIAANILLYF PNGETKYASE
NHLSRFVWFF SGIVGGGLLM LLPAFVFIGL EQDDCCGCCG
HENCGKRCAM LSSVLAALIG IAGSGYCVIV AALGLAEGPLCLD
SLGQWNYTFASTEGQYLLDTSTWSECTEPKHIVEWNVSLFSILLALG
GIEFILCLIQVINGVLGGIC GFCCSHQQQY DC

In some embodiments, the anti-TM4SF1 antibodies and antigen binding fragments thereof, of the disclosure are specific to the ECL2 domain of TM4SF1. The amino acid sequence of human TM4SF1 ECL2 domain is

(SEQ ID NO: 157)
EGPLCLDSLGQWNYTFASTEGQYLLDTSTWSECTEPKHIVEWNVSLFS

As described in Table 5 below, included in the disclosure are novel antibodies that are specific to TM4SF1. The antibodies described in Table 5 are monoclonal murine antibodies AGX-A03, AGX-A04, AGX-A05, AGX-A07, AGX-A08, AGX-A09, and AGX-A11, each of which were identified in the screen described in the Examples and bind the ECL2 region of TM4SF1. Further provided in Table 5 below are humanized antibodies h AGX-A07 and h AGX-A01.

In some embodiments, the anti-TM4SF1 antibodies or antigen-binding fragments thereof, comprise an IgG heavy chain constant region comprising an amino acid sequence set forth in SEQ ID NO: 87 or 88, or a sequence that is at least about 60%, at least about 65%, at least about 70%, at least about 75%, at least about, at least about 80%, at least about 85%, at least about 90%, at least about 95%, at least about 96%, at least about 97%, at least about 98%, at least about 99%, or 100% identical to SEQ ID NO: 73 or 74.

In another embodiment, the anti-TM4SF1 antibody or the antigen-binding fragment thereof, comprises a light chain constant region comprising the amino acid sequence set forth in SEQ ID NO: 89, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 89.

In another embodiment, the anti-TM4SF1 antibody or the antigen-binding fragment thereof, comprises a heavy chain variable domain comprising the amino acid sequence set forth in SEQ ID NO: 3, 15, 27, 39, 51, 63, or 75, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 3, 15, 27, 39, 51, 63, or 75.

In another embodiment, the anti-TM4SF1 antibody or the antigen-binding fragment thereof is humanized and, comprises a heavy chain comprising the amino acid sequence set forth in SEQ ID NO: 90 or 92 or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 90 or 92.

In another embodiment, the anti-TM4SF1 antibody or the antigen-binding fragment thereof is humanized and, comprises a heavy chain comprising the amino acid sequence set forth in SEQ ID NO: 112 or 114, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 112 or 114.

In another embodiment, the anti-TM4SF1 antibody or the antigen-binding fragment thereof, comprises a light chain variable domain comprising the amino acid sequence set forth in SEQ ID NO: 9, 21, 33, 45, 57, 69, or 81, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 9, 21, 33, 45, 57, 69, or 81.

In another embodiment, the anti-TM4SF1 antibody or the antigen-binding fragment thereof is humanized and, comprises a light chain variable domain comprising the amino acid sequence set forth in SEQ ID NO: 97, 99, 101, 103, or 105 or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 97, 99, 101, 103 or 105. In another embodiment, the antibody or antigen-binding fragment thereof is humanized and, comprises a light chain variable domain comprising the amino acid sequence set forth in SEQ ID NO: 97, 99, or 101 or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 97, 99, or 101.

In another embodiment, the anti-TM4SF1 antibody or the antigen-binding fragment thereof is humanized and, comprises a light chain variable domain comprising the amino acid sequence set forth in SEQ ID NO: 122, or a sequence that is at least about 60%, at least about 65%, at least about 70%, at least about 75%, at least about 80%, at least about 85%, at least about 90%, at least about 95%, at least about 96%, at least about 97%, at least about 98%, at least about 99% identical, or 100% identical to SEQ ID NO: 122.

In some embodiments, the anti-TM4SF1 antibody or the antigen binding fragment thereof comprises a heavy chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 6, 18, 30, 42, 54, 66, or 78. In some embodiments, the anti-TM4SF1 antibody or the antigen binding fragment thereof comprises a heavy chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 7, 19, 31, 43, 55, 67, or 79. In some embodiments, the anti-TM4SF1 antibody or the antigen binding fragment thereof comprises a heavy chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 8, 20, 32, 44, 56, 68, or 80.

In some embodiments, the anti-TM4SF1 antibody or the antigen binding fragment thereof comprises a light chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 12, 24, 36, 48, 60, 72, or 84. In some embodiments, the anti-TM4SF1 antibody or the antigen binding fragment thereof comprises a light chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 13, 25, 37, 49, 61, 73, or 85. In some embodiments, the anti-TM4SF1 antibody or the antigen binding fragment thereof comprises a light chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 14, 26, 38, 50, 62, 74, or 86.

In some embodiments, the anti-TM4SF1 antibody or the antigen binding fragment thereof is humanized and comprises a heavy chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 94 or SEQ ID NO: 115. In some embodiments, the anti-TM4SF1 antibody or the antigen binding fragment thereof is humanized and comprises a heavy chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 95, SEQ ID NO: 116, or SEQ ID NO: 117. In some embodiments, the anti-TM4SF1 antibody or the antigen binding fragment thereof is humanized and comprises a heavy chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 96, SEQ ID NO: 118, SEQ ID NO: 119, SEQ ID NO: 120, or SEQ ID NO: 121.

In some embodiments, the anti-TM4SF1 antibody or the antigen binding fragment thereof is humanized and comprises a light chain CDR1 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 107, SEQ ID NO: 108, SEQ ID NO: 124, SEQ ID NO: 125, SEQ ID NO: 126, or SEQ ID NO: 127. In some embodiments, the anti-TM4SF1 antibody or the antigen binding fragment thereof is humanized comprises a light chain CDR2 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 109 or SEQ ID NO: 128. In some embodiments, the anti-TM4SF1 antibody or the antigen binding fragment thereof is humanized and comprises a light chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 110, SEQ ID NO: 111, or SEQ ID NO: 129. In some embodiments, the anti-TM4SF1 antibody or the antigen binding fragment thereof is humanized and comprises a light chain CDR3 comprising an amino acid sequence that is from at least about 80% to at least about 85%, from at least about 85% to at least about 90%, from at least about 90% to at least about 91%, from at least about 91% to at least about 92%, from at least about 92% to at least about 93%, from at least about 93% to at least about 94%, from at least about 94% to at least about 95%, from at least about 95% to at least about 96%, from at least about 96% to at least about 97%, from at least about 97% to at least about 98%, from at least about 98% to at least about 99%, or from at least about 99% to 100% identical to SEQ ID NO: 110, or SEQ ID NO: 129.

The amino acid sequences of murine monoclonal antibody AGX-A03 are described in Table 5. Specifically, the heavy chain CDR sequences are set forth in SEQ ID Nos: 6, 7, and 8 (CDR1, CDR2, and CDR3), and the light chain CDR amino acid sequences are set forth in SEQ ID Nos: 12, 13, and 14 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 6, 7, and 8 and/or a light chain variable region comprising CDRs as set forth in the amino acid sequences of SEQ ID Nos: 12, 13, and 14. Included in the disclosure are humanized antibodies or antigen binding fragments comprising the CDRs of AGX-A03. Further, the heavy chain variable amino acid sequences and the light chain variable amino acid sequences of AGX-A03 are described in SEQ ID NOS: 3 and 9, respectively.

The amino acid sequences of murine monoclonal antibody AGX-A04 are described in Table 5. Specifically, the heavy chain CDR sequences are set forth in SEQ ID Nos: 18, 19, and 20 (CDR1, CDR2, and CDR3), and the light chain CDR amino acid sequences are set forth in SEQ ID Nos: 24, 25, and 26 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 18, 19, and 20 and/or a light chain variable region comprising CDRs as set forth in the amino acid sequences of SEQ ID Nos: 24, 25, and 26. Included in the disclosure are humanized antibodies or antigen binding fragments comprising the CDRs of AGX-A04. Further, the heavy chain variable amino acid sequences and the light chain variable amino acid sequences of AGX-A04 are described in SEQ ID NOS: 15 and 21, respectively.

The amino acid sequences of murine monoclonal antibody AGX-A05 are described in Table 5. Specifically, the heavy chain CDR sequences are set forth in SEQ ID Nos: 30, 31, and 32 (CDR1, CDR2, and CDR3), and the light chain CDR amino acid sequences are set forth in SEQ ID Nos: 36, 37, and 38 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 30, 31, and 32 and/or a light chain variable region comprising CDRs as set forth in the amino acid sequences of SEQ ID Nos: 36, 37, and 38. Included in the disclosure are humanized antibodies or antigen binding fragments comprising the CDRs of AGX-A05. Further, the heavy chain variable amino acid sequences and the light chain variable amino acid sequences of AGX-A05 are described in SEQ ID NOS: 27 and 33, respectively. The amino acid sequences of murine monoclonal antibody AGX-A07 are described in Table 5. Specifically, the heavy chain CDR sequences are set forth in SEQ ID Nos: 42, 43, and 44 (CDR1, CDR2, and CDR3), and the light chain CDR amino acid sequences are set forth in SEQ ID Nos: 48, 49, and 50 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 42, 43, and 44 and/or a light chain variable region comprising CDRs as set forth in the amino acid sequences of SEQ ID Nos: 48, 49, and 50. Included in the disclosure are humanized antibodies or antigen binding fragments comprising the CDRs of AGX-A07. Further, the heavy chain variable amino acid sequences and the light chain variable amino acid sequences of AGX-A07 are described in SEQ ID NOs: 39 and 45, respectively.

In one embodiment, a humanized AGX-A07 (h AGX-A07) antibody or antigen binding fragments thereof is provided, comprising a heavy chain sequence as forth in the amino acid sequence of SEQ ID NO: 90. In some embodiments, the humanized AGX-A07 antibody or antigen binding fragments thereof is a humanized mutated AGX-A07 (hm AGX-A07) antibody or antigen binding fragments thereof, comprising a heavy chain sequence comprising one or more substitutions in the sequence as set forth in the amino acid sequence of SEQ ID NO: 90. As shown in Table 5, the heavy chain sequence set forth in SEQ ID NO: 90 is also referred to herein as AGX-A07 H2. In some embodiments, the humanized AGX-A07 antibody or antigen binding fragments thereof is a humanized mutated AGX-A07 antibody or antigen binding fragments thereof, comprising a heavy chain sequence comprising one or more substitutions in the sequence as set forth in the amino acid sequence of SEQ ID NO: 90, wherein the one or more substitutions are in amino acid positions 1, 44, and 80 of SEQ ID NO: 90. In some cases, the humanized mutated AGX-A07 antibody or antigen binding fragments thereof comprises an E1Q (glutamic acid to glutamine substitution at position 1 of the heavy chain, SEQ ID NO: 90). In some cases, the humanized mutated AGX-A07 antibody or antigen binding fragments thereof comprises a D44G (aspartate to glycine substitution at position 44 of the heavy chain, SEQ ID NO: 90). In some cases, the humanized mutated AGX-A07 antibody or antigen binding fragments thereof comprises a F80Y (phenyl alanine to tyrosine substitution at position 80 of the heavy chain, SEQ ID NO: 90). In some embodiments, a humanized mutated AGX-A07 antibody or antigen binding fragments is provided, comprising a heavy chain sequence as forth in the amino acid sequence of SEQ ID NO: 92. As shown in Table 5, the heavy chain sequence set forth in SEQ ID NO: 92 is also referred to herein as AGX-A07 H2v1. In some embodiments, humanized AGX-A07 antibodies or antigen binding fragments are provided, comprising a light chain sequence as forth in the amino acid sequence of SEQ ID NO: 97. As shown in Table 5, the light chain sequence set forth in SEQ ID NO: 97 is also referred to herein as AGX-A07 L5. In some embodiments, the humanized AGX-A07 antibody or antigen binding fragments thereof is a humanized mutated AGX-A07 antibody or antigen binding fragments thereof, comprising a light chain sequence comprising one or more substitutions in the sequence as set forth in the amino acid sequence of SEQ ID NO: 97. In some embodiments, the humanized AGX-A07 antibodies or antigen binding fragments thereof is a humanized mutated AGX-A07 antibody or antigen binding fragments thereof, comprising a light chain sequence comprising one or more substitutions in the sequence as set forth in the amino acid sequence of SEQ ID NO: 97, wherein the one or more substitutions are in amino acid positions 3, 26, 62, and 90 of SEQ ID NO: 97. In some cases, the humanized mutated AGX-A07 antibody or antigen binding fragments thereof comprises an 13V (isoleucine to valine substitution at position 3 of the light chain, SEQ ID NO: 97). In some cases, the humanized mutated AGX-A07 antibody or antigen binding fragments thereof comprises a N26Q (asparagine to glutamine substitution at position 26 of the light chain, SEQ ID NO: 97). In some cases, the humanized mutated AGX-A07 antibody or antigen binding fragments thereof comprises a N26S (asparagine to serine substitution at position 26 of the light chain, SEQ ID NO: 97). In some cases, the humanized mutated AGX-A07 antibody or antigen binding fragments thereof comprises a G62S (glycine to serine substitution at position 62 of the light chain, SEQ ID NO: 97). In some cases, the humanized mutated AGX-A07 antibody or antigen binding fragments thereof comprises a W90Y (tryptophan to tyrosine substitution at position 90 of the light chain, SEQ ID NO: 97). In some embodiments, humanized mutated AGX-A07 antibodies or antigen binding fragments are provided, comprising a light chain sequence as forth in an amino acid sequence selected from the group consisting of SEQ ID NO: 99, SEQ ID NO: 101, SEQ ID NO: 103, and SEQ ID NO: 105. As shown in Table 5, the light chain sequence set forth in SEQ ID NO: 99 is also referred to herein as AGX-A07 L5v1, the light chain sequence set forth in SEQ ID NO: 101 is also referred to herein as AGX-A07 L5v2, the light chain sequence set forth in SEQ ID NO: 103 is also referred to herein as AGX-A07 L5v3, and the light chain sequence set forth in SEQ ID NO: 105 is also referred to herein as AGX-A07 L5v4. Exemplary coding sequence for the heavy chain of a humanized AGX-A07 antibody or antigen binding fragment thereof is provided in SEQ ID NO: 91. Exemplary coding sequence for the heavy chain of a humanized mutated AGX-A07 antibody or antigen binding fragment thereof is provided in SEQ ID NO: 93. Exemplary coding sequence for the light chain of a humanized AGX-A07 antibody or antigen binding fragment thereof is provided in SEQ ID NO: 98 (AGX-A07 L5). Exemplary coding sequences for the light chain of a humanized mutated AGX-A07 antibody or antigen binding fragment thereof are provided in SEQ ID NO: 100 (AGX-A07 L5v1), SEQ ID NO: 102 (AGX-A07 L5v2), SEQ ID NO: 104 (AGX-A07 L5v3), and SEQ ID NO: 106 (AGX-A07 L5v4).

In one embodiment, a humanized AGX-A07 antibody or antigen binding fragments thereof is provided, comprising a heavy chain variable domain sequence as forth in the amino acid sequence of SEQ ID NO: 130 or SEQ ID NO: 132. In some embodiments, the humanized AGX-A07 antibody or antigen binding fragments thereof is a humanized mutated AGX-A07 antibody or antigen binding fragments thereof, comprising a heavy chain variable domain sequence comprising one or more substitutions in the sequence as set forth in the amino acid sequence of SEQ ID NO: 130 or SEQ ID NO: 132. In one embodiment, a humanized AGX-A07 antibody or antigen binding fragments thereof is provided, comprising a light chain variable domain sequence as forth in the amino acid sequence of SEQ ID NO: 131 or SEQ ID NO: 133. In some embodiments, the humanized AGX-A07 antibody or antigen binding fragments thereof is a humanized mutated AGX-A07 antibody or antigen binding fragments thereof, comprising a light chain variable domain sequence comprising one or more substitutions in the sequence as set forth in the amino acid sequence of SEQ ID NO: 131 or SEQ ID NO: 133.

In some embodiments, the humanized AGX-A07 antibody or antigen binding fragment thereof is a humanized mutated AGX-A07 antibody or antigen binding fragment thereof comprising a light chain variable domain sequence comprising the sequence as set forth in the amino acid sequence of SEQ ID NO: 131 and a heavy chain variable domain sequence comprising the sequence as set forth in the amino acid sequence of SEQ ID NO: 130. In some embodiments, the humanized AGX-A07 antibody or antigen binding fragment thereof is a humanized mutated AGX-A07 antibody or antigen binding fragments thereof, comprising a light chain variable domain sequence comprising one or more substitutions in the sequence as set forth in the amino acid sequence of SEQ ID NO: 131 and a heavy chain variable domain sequence comprises one or more substitutions in the sequence as set forth in the amino acid sequence of SEQ ID NO: 130. In some embodiments, the humanized AGX-A07 antibody or antigen binding fragments thereof is a humanized mutated AGX-A07 antibody or antigen binding fragments thereof comprising a light chain variable domain sequence comprising the sequence as set forth in the amino acid sequence of SEQ ID NO: 133 and a heavy chain variable domain sequence comprising the sequence as set forth in the amino acid sequence of SEQ ID NO: 132. In some embodiments, the humanized AGX-A07 antibody or antigen binding fragments thereof is a humanized mutated AGX-A07 antibody or antigen binding fragments thereof, comprising a light chain variable domain sequence comprising one or more substitutions in the sequence as set forth in the amino acid sequence of SEQ ID NO: 133 and a heavy chain variable domain sequence comprises one or more substitutions in the sequence as set forth in the amino acid sequence of SEQ ID NO: 132. In some embodiments, the humanized AGX-A07 antibody or antigen binding fragments thereof is a humanized mutated AGX-A07 antibody or antigen binding fragments thereof comprising a heavy chain sequence comprising the sequence as set forth in the amino acid sequence of SEQ ID NO: 156, or a sequence comprising one of more substitutions in the amino acid sequence of SEQ ID NO: 156.

In some cases, the humanized AGX-A07 antibodies or antigen binding fragments thereof comprise heavy chain CDR sequences as set forth in SEQ ID Nos: 94, 95, and 96 (CDR1, CDR2, and CDR3), or CDR sequences comprising one or more substitutions in the sequences as set forth in SEQ ID Nos: 94, 95, and 96 (CDR1, CDR2, and CDR3). In some cases, the humanized mutated AGX-A07 antibodies or antigen binding fragments thereof comprises heavy chain CDR sequences as set forth in SEQ ID Nos: 94, 95, and 96 (CDR1, CDR2, and CDR3), or CDR sequences comprising one or more substitutions in the sequences as set forth in SEQ ID Nos: 94, 95, and 96 (CDR1, CDR2, and CDR3).

In some cases, the humanized mutated AGX-A07 antibodies or antigen binding fragments thereof comprise heavy chain CDR1 sequence as set forth in SEQ ID NO: 94, or a heavy chain CDR1 sequence comprising one or more substitutions in the sequences as set forth in SEQ ID NO: 94. In some cases, the humanized mutated AGX-A07 antibodies or antigen binding fragments thereof comprise a heavy chain CDR2 sequence as set forth in SEQ ID NO: 95, or a heavy chain CDR2 sequence comprising one or more substitutions in the sequences as set forth in SEQ ID NO: 95. In some cases, the humanized mutated AGX-A07 antibodies or antigen binding fragments thereof comprise a heavy chain CDR3 sequence as set forth in SEQ ID NO: 96, or a heavy chain CDR3 sequence comprising one or more substitutions in the sequences as set forth in SEQ ID NO: 96.

In some cases, the humanized AGX-A07 antibodies or antigen binding fragments thereof comprise light chain CDR sequences as set forth in SEQ ID Nos: 107, 109, and 110 (CDR1, CDR2, and CDR3), or CDR sequences comprising one or more substitutions in the sequences as set forth in SEQ ID Nos: 107, 109, and 110 (CDR1, CDR2, and CDR3). In some cases, the humanized AGX-A07 antibodies or antigen binding fragments thereof comprise light chain CDR sequences as set forth in SEQ ID Nos: 107, 109, and 111 (CDR1, CDR2, and CDR3), or CDR sequences comprising one or more substitutions in the sequences as set forth in SEQ ID Nos: 107, 109, and 111 (CDR1, CDR2, and CDR3). In some cases, the humanized AGX-A07 antibodies or antigen binding fragments thereof comprise light chain CDR sequences as set forth in SEQ ID Nos: 108, 109, and 110 (CDR1, CDR2, and CDR3), or CDR sequences comprising one or more substitutions in the sequences as set forth in SEQ ID Nos: 108, 109, and 110 (CDR1, CDR2, and CDR3). In some cases, the humanized AGX-A07 antibodies or antigen binding fragments thereof comprise light chain CDR sequences as set forth in SEQ ID Nos: 108, 109, and 111 (CDR1, CDR2, and CDR3), or CDR sequences comprising one or more substitutions in the sequences as set forth in SEQ ID Nos: 108, 109, and 111 (CDR1, CDR2, and CDR3).

In some cases, the humanized mutated AGX-A07 antibodies or antigen binding fragments thereof comprise light chain CDR1 sequence as set forth in SEQ ID Nos: 107 or 108, or light chain CDR1 sequence comprising one or more substitutions in the sequences as set forth in SEQ ID Nos: 107 or 108. In some cases, the humanized mutated AGX-A07 antibodies or antigen binding fragments thereof comprise light chain CDR2 sequence as set forth in SEQ ID NO: 109, or light chain CDR2 sequence comprising one or more substitutions in the sequences as set forth in SEQ ID NO: 109. In some cases, the humanized mutated AGX-A07 antibodies or antigen binding fragments thereof comprise light chain CDR3 sequence as set forth in SEQ ID Nos: 110 or 111, or light chain CDR1 sequence comprising one or more substitutions in the sequences as set forth in SEQ ID Nos: 110 or 111. In some cases, the humanized mutated AGX-A07 antibodies or antigen binding fragments thereof comprise light chain CDR3 sequence as set forth in SEQ ID NO: 110, or light chain CDR1 sequence comprising one or more substitutions in the sequences as set forth in SEQ ID Nos: 110.

In some embodiments, the humanized mutated AGX-A07 comprises a heavy chain variable region comprising the following amino acid substitutions: Q1E, D44G, F80Y in SEQ ID NO: 132 (also referred to herein as AGX-A07 H2), and a light chain variable region comprising the following amino acid substitutions: 13V, N26Q, G62S in SEQ ID NO: 133 (also referred to herein as AGX-A07 L5). In some embodiments, the humanized mutated AGX-A07 comprises a heavy chain variable region comprising the following amino acid substitutions: Q1E, D44G, F80Y in SEQ ID NO: 132, and a light chain variable region comprising the following amino acid substitutions: I3V, N26Q, G62S in SEQ ID NO: 133, wherein the heavy chain comprises CDR1 (SEQ ID NO: 94), CDR2 (SEQ ID NO: 95), and CDR3 (SEQ ID NO: 96), and the light chain comprises CDR1 (SEQ ID NO: 108), CDR2 (SEQ ID NO: 109), and CDR3 (SEQ ID NO: 110). In some embodiments, the humanized mutated AGX-A07 is AGX-A07 H2v1L5v2 and comprises a heavy chain comprising the amino acid sequence as set forth in SEQ ID NO: 130 (also referred to herein as AGX-A07 H2v1), and a light chain comprising the amino acid sequence as set forth in SEQ ID NO: 131 (also referred to herein as AGX-A07 L5v2). In some embodiments, the humanized mutated AGX-A07 comprises a heavy chain comprising the amino acid sequence as set forth in SEQ ID NO: 92, and a light chain comprising the amino acid sequence as set forth in SEQ ID NO: 101.

The amino acid sequences of murine monoclonal antibody AGX-A08 are described in Table 5. Specifically, the heavy chain CDR sequences are set forth in SEQ ID Nos: 54, 55, and 56 (CDR1, CDR2, and CDR3), and the light chain CDR amino acid sequences are set forth in SEQ ID Nos: 60, 61, and 62 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 54, 55, and 56 and/or a light chain variable region comprising CDRs as set forth in the amino acid sequences of SEQ ID Nos: 60, 61, and 62. Included in the disclosure are humanized antibodies or antigen binding fragments comprising the CDRs of AGX-A08. Further, the heavy chain variable amino acid sequences and the light chain variable amino acid sequences of AGX-A08 are described in SEQ ID NOs: 51 and 57, respectively.

The amino acid sequences of murine monoclonal antibody AGX-A09 are described in Table 5. Specifically, the heavy chain CDR sequences are set forth in SEQ ID Nos: 66, 67, and 68 (CDR1, CDR2, and CDR3), and the light chain CDR amino acid sequences are set forth in SEQ ID Nos: 72, 73, and 74 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 66, 67, and 68 and/or a light chain variable region comprising CDRs as set forth in the amino acid sequences of SEQ ID Nos: 72, 73, and 74. Included in the disclosure are humanized antibodies or antigen binding fragments comprising the CDRs of AGX-A09. Further, the heavy chain variable amino acid sequences and the light chain variable amino acid sequences of AGX-A09 are described in SEQ ID NOs: 63 and 69, respectively.

The amino acid sequences of murine monoclonal antibody AGX-A11 are described in Table 5. Specifically, the heavy chain CDR sequences are set forth in SEQ ID Nos: 78, 79, and 80 (CDR1, CDR2, and CDR3), and the light chain CDR amino acid sequences are set forth in SEQ ID Nos: 84, 85, and 86 (CDR1, CDR2, and CDR3). Included in the disclosure are anti-TM4SF1 antibodies, or antigen binding fragments comprising a heavy chain variable region comprising CDRs as set forth in the amino acid sequences of SEQ ID Nos: 78, 79, and 80 and/or a light chain variable region comprising CDRs as set forth in the amino acid sequences of SEQ ID Nos: 84, 85, and 862. Included in the disclosure are humanized antibodies or antigen binding fragments comprising the CDRs of AGX-A11. Further, the heavy chain variable amino acid sequences and the light chain variable amino acid sequences of AGX-A11 are described in SEQ ID NOS: 75 and 81, respectively.

The amino acid sequences of a humanized antibody AGX-A01 (h AGX-A01) are described in Table 5. As shown in Table 5, the heavy chain sequence set forth is SEQ ID NO: 112 is also referred to herein as AGX-A01 H1. Specifically, the heavy chain CDR sequences are set forth in SEQ ID Nos: 115, 116, and 118 (CDR1, CDR2, and CDR3) and the light chain CDR amino acid sequences are set forth in SEQ ID Nos: 124, 128, and 129 (CDR1, CDR2, and CDR3). Further, exemplary heavy chain amino acid sequence and the light chain amino acid sequence of the humanized AGX-A01 are described in SEQ ID Nos: 112 and 122, respectively. Exemplary coding sequences for the heavy chain and the light chain of the humanized AGX-A01 are described in SEQ ID Nos: 113 and 123, respectively.

In some embodiments, the humanized AGX-A01 antibody or antigen binding fragments thereof is a humanized mutated AGX-A01 (hm AGX-A01) antibody or antigen binding fragments thereof, comprising a heavy chain sequence comprising one or more substitutions in the sequence as set forth in the amino acid sequence of SEQ ID NO: 112. In some embodiments, the humanized AGX-A01 antibody or antigen binding fragments thereof is a humanized mutated AGX-A01 antibody or antigen binding fragments thereof, comprising a heavy chain sequence comprising one or more substitutions in the sequence as set forth in the amino acid sequence of SEQ ID NO: 112, wherein the one or more substitutions are in amino acid positions 63 and 106 of SEQ ID NO: 112. In some cases, the humanized mutated AGX-A01 antibody or antigen binding fragments thereof comprises a G63S (glycine to serine substitution at position 63 of the heavy chain, SEQ ID NO: 112). In some cases, the humanized mutated AGX-A01 antibody or antigen binding fragments thereof comprises a D106E (aspartate to glutamic acid substitution at position 106 of the heavy chain, SEQ ID NO: 112). In some cases, the humanized mutated AGX-A01 antibody or antigen binding fragments thereof comprises a D106S (aspartate to serine substitution at position 106 of the heavy chain, SEQ ID NO: 112). In some embodiments, a humanized mutated AGX-A01 antibody or antigen binding fragments is provided, comprising a heavy chain sequence as forth in the amino acid sequence of SEQ ID NO: 114. As shown in Table 5, the heavy chain sequence set forth is SEQ ID NO: 114 is also referred to herein as AGX-A01 Hlv1.

In some embodiments, humanized AGX-A01 antibodies or antigen binding fragments are provided, comprising a light chain sequence as forth in the amino acid sequence of SEQ ID NO: 122. As shown in Table 5, the light chain sequence set forth is SEQ ID NO: 122 is also referred to herein as AGX-A01 L10. In some embodiments, the humanized AGX-A01 antibody or antigen binding fragments thereof is a humanized mutated AGX-A01 antibody or antigen binding fragments thereof, comprising a light chain sequence comprising one or more substitutions in the sequence as set forth in the amino acid sequence of SEQ ID NO: 122. In some embodiments, the humanized mutated AGX-A01 antibody or antigen binding fragments thereof is a humanized mutated AGX-A01 antibody or antigen binding fragments thereof, comprising a light chain sequence comprising one or more substitutions in the sequence as set forth in the amino acid sequence of SEQ ID NO: 122, wherein the one or more substitutions are in one or more amino acid positions selected from amino acid positions 1, 33, 42, 51, 86, and 90 of SEQ ID NO: 122. In some embodiments, the humanized mutated AGX-A01 antibody or antigen binding fragments thereof is a humanized mutated AGX-A01 antibody or antigen binding fragments thereof, comprising a light chain sequence comprising one or more substitutions in the sequence as set forth in the amino acid sequence of SEQ ID NO: 122, wherein the one or more substitutions are in one or more amino acid positions selected from amino acid positions 1, 33, 42, 51, and 86 of SEQ ID NO: 122. In some cases, the humanized mutated AGX-A01 antibody or antigen binding fragments thereof comprises an AlE (alanine to glutamic acid substitution at position 1 of the light chain, SEQ ID NO: 122). In some cases, the humanized mutated AGX-A01 antibody or antigen binding fragments thereof comprises a N33S (asparagine to serine substitution at position 33 of the light chain, SEQ ID NO: 122). In some cases, the humanized mutated AGX-A01 antibody or antigen binding fragments thereof comprises a M42Q (methionine to glutamine substitution at position 42 of the light chain, SEQ ID NO: 122). In some cases, the humanized mutated AGX-A01 antibody or antigen binding fragments thereof comprises a V51L (valine to leucine substitution at position 51 of the light chain, SEQ ID NO: 122). In some cases, the humanized mutated AGX-A01 antibody or antigen binding fragments thereof comprises a D86E (aspartate to glutamic acid substitution at position 86 of the light chain, SEQ ID NO: 122). In some cases, the humanized mutated AGX-A01 antibody or antigen binding fragments thereof comprises an 190V (isoleucine to valine substitution at position 90 of the light chain, SEQ ID NO: 122).

In some cases, the humanized AGX-A01 antibodies or antigen binding fragments thereof comprise heavy chain CDR sequences as set forth in SEQ ID Nos: 115 (CDR1); 116 (CDR2); and 118 (CDR3), or CDR sequences comprising one or more substitutions in the sequences as set forth in SEQ ID Nos: 115 (CDR1); 116 (CDR2); and 118 (CDR3). In some cases, the humanized mutated AGX-A01 antibodies or antigen binding fragments thereof comprise heavy chain CDR sequences as set forth in SEQ ID Nos: 115 (CDR1); 116 or 117 (CDR2); and 118, 119, 120, or 121 (CDR3), or CDR sequences comprising one or more substitutions in the sequences as set forth in SEQ ID Nos: 115 (CDR1); 116 or 117 (CDR2); and 118, 119, 120, or 121 (CDR3).

In some cases, the humanized mutated AGX-A01 antibodies or antigen binding fragments thereof comprise heavy chain CDR1 sequence as set forth in SEQ ID NO: 115, or a heavy chain CDR1 sequence comprising one or more substitutions in the sequences as set forth in SEQ ID NO: 115. In some cases, the humanized mutated AGX-A01 antibodies or antigen binding fragments thereof comprise a heavy chain CDR2 sequence as set forth in SEQ ID NO: 116, or a heavy chain CDR2 sequence comprising one or more substitutions in the sequences as set forth in SEQ ID NO: 116. In some cases, the humanized mutated AGX-A01 antibodies or antigen binding fragments thereof comprise a heavy chain CDR2 sequence as set forth in SEQ ID NO: 117, or a heavy chain CDR2 sequence comprising one or more substitutions in the sequences as set forth in SEQ ID NO: 117. In some cases, the humanized mutated AGX-A01 antibodies or antigen binding fragments thereof comprise a heavy chain CDR3 sequence as set forth in a sequence selected from SEQ ID Nos: 118, 119, 120, and 121, or a heavy chain CDR3 sequence comprising one or more substitutions in a sequence selected from SEQ ID Nos: 118, 119, 120, and 121.

In some cases, the humanized AGX-A01 antibodies or antigen binding fragments thereof comprise light chain CDR sequences as set forth in SEQ ID Nos: 124 (CDR1); 128 (CDR2); and 129 (CDR3), or CDR sequences comprising one or more substitutions in the sequences as set forth in SEQ ID Nos: 124 (CDR1); 128 (CDR2); and 129 (CDR3). In some cases, the humanized mutated AGX-A01 antibodies or antigen binding fragments thereof comprise light chain CDR sequences as set forth in SEQ ID Nos: 124, 125, 126, or 127 (CDR1); 128 (CDR2); and 129 (CDR3), or CDR sequences comprising one or more substitutions in the sequences as set forth in SEQ ID Nos: 124, 125, 126, or 127 (CDR1); 128 (CDR2); and 129 (CDR3).

In some cases, the humanized mutated AGX-A01 antibodies or antigen binding fragments thereof comprise light chain CDR1 sequence as set forth in SEQ ID Nos: 125, 126, 127, or 128, or light chain CDR1 sequence comprising one or more substitutions in the sequences as set forth in SEQ ID Nos: 125, 126, 127, or 128. In some cases, the humanized mutated AGX-A01 antibodies or antigen binding fragments thereof comprise light chain CDR2 sequence as set forth in SEQ ID NO: 129, or light chain CDR2 sequence comprising one or more substitutions in the sequences as set forth in SEQ ID NO: 129. In some cases, the humanized mutated AGX-A01 antibodies or antigen binding fragments thereof comprise light chain CDR3 sequence as set forth in SEQ ID Nos: 130, or light chain CDR1 sequence comprising one or more substitutions in the sequences as set forth in SEQ ID Nos: 130.

In one embodiment, the disclosure provides an anti-TM4SF1 antibody, or antigen-binding fragment thereof, that comprises a heavy chain variable domain encoded by a nucleic acid sequence as set forth in SEQ ID NO: 3, and a light chain variable domain encoded by a nucleic acid sequence as set forth in SEQ ID NO: 9. In one embodiment, the disclosure provides an anti-TM4SF1 antibody, or antigen-binding fragment thereof, that comprises a heavy chain variable domain encoded by a nucleic acid sequence as set forth in SEQ ID NO: 15, and a light chain variable domain encoded by a nucleic acid sequence as set forth in SEQ ID NO: 21 In one embodiment, the disclosure provides an anti-TM4SF1 antibody, or antigen-binding fragment thereof, that comprises a heavy chain variable domain encoded by a nucleic acid sequence as set forth in SEQ ID NO: 27, and a light chain variable domain encoded by a nucleic acid sequence as set forth in SEQ ID NO: 33. In one embodiment, the disclosure provides an anti-TM4SF1 antibody, or antigen-binding fragment thereof, that comprises a heavy chain variable domain encoded by a nucleic acid sequence as set forth in SEQ ID NO: 39, and a light chain variable domain encoded by a nucleic acid sequence as set forth in SEQ ID NO: 45. In one embodiment, the disclosure provides an anti-TM4SF1 antibody, or antigen-binding fragment thereof, that comprises a heavy chain variable domain encoded by a nucleic acid sequence as set forth in SEQ ID NO: 51, and a light chain variable domain encoded by a nucleic acid sequence as set forth in SEQ ID NO: 57. In one embodiment, the disclosure provides an anti-TM4SF1 antibody, or antigen-binding fragment thereof, that comprises a heavy chain variable domain encoded by a nucleic acid sequence as set forth in SEQ ID NO: 63, and a light chain variable domain encoded by a nucleic acid sequence as set forth in SEQ ID NO: 69. In one embodiment, the disclosure provides an anti-TM4SF1 antibody, or antigen-binding fragment thereof, that comprises a heavy chain variable domain encoded by a nucleic acid sequence as set forth in SEQ ID NO: 75, and a light chain variable domain encoded by a nucleic acid sequence as set forth in SEQ ID NO: 81. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 97. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 99. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 101. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 103. In one embodiment, the disclosure provides an anti-TM4SF1 antibody, or antigen-binding fragment thereof, that comprises a heavy chain variable domain encoded by a nucleic acid sequence as set forth in SEQ ID NO: 90, and a light chain variable domain encoded by a nucleic acid sequence as set forth in SEQ ID NO: 105. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 97. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 99. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 101. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 103. In one embodiment, the disclosure provides an anti-TM4SF1 antibody, or antigen-binding fragment thereof, that comprises a heavy chain variable domain encoded by a nucleic acid sequence as set forth in SEQ ID NO: 92, and a light chain variable domain encoded by a nucleic acid sequence as set forth in SEQ ID NO: 105.

In one embodiment, the present disclosure provides an anti-TM4SF1 antibody, or antigen-binding fragment thereof, that has a heavy chain variable domain sequence that is at least 95% identical, at least 96% identical, at least 97% identical, at least 98% identical, at least 99% identical, or 100% identical to an amino acid sequence selected from SEQ ID NO: 3, SEQ ID NO: 15, SEQ ID NO: 27, SEQ ID NO: 39, SEQ ID NO: 51, SEQ ID NO: 63, SEQ ID NO: 75, SEQ ID NO: 90, SEQ ID NO: 92, SEQ ID NO: 112, or SEQ ID NO: 114; and that has a light chain variable domain sequence that is at least 95% identical, at least 96% identical, at least 97% identical, at least 98% identical, at least 99% identical, or 100% identical to an amino acid sequence selected from SEQ ID NO: 9, SEQ ID NO: 21, SEQ ID NO: 33, SEQ ID NO: 45, SEQ ID NO: 57, SEQ ID NO: 69, SEQ ID NO: 81, SEQ ID NO: 97, SEQ ID NO: 99, SEQ ID NO: 101, SEQ ID NO: 103, SEQ ID NO: 105, or SEQ ID NO: 122. In one embodiment, the present disclosure provides an anti-TM4SF1 antibody, or antigen-binding fragment thereof, that has a heavy chain variable domain sequence that is at least 95% identical, at least 96% identical, at least 97% identical, at least 98% identical, at least 99% identical, or 100% identical to an amino acid sequence selected from SEQ ID NO: 3, SEQ ID NO: 15, SEQ ID NO: 27, SEQ ID NO: 39, SEQ ID NO: 51, SEQ ID NO: 63, SEQ ID NO: 75, SEQ ID NO: 90, SEQ ID NO: 92, SEQ ID NO: 112, or SEQ ID NO: 114; and that has a light chain variable domain sequence that is at least 95% identical, at least 96% identical, at least 97% identical, at least 98% identical, at least 99% identical, or 100% identical to an amino acid sequence selected from SEQ ID NO: 9, SEQ ID NO: 21, SEQ ID NO: 33, SEQ ID NO: 45, SEQ ID NO: 57, SEQ ID NO: 69, SEQ ID NO: 81, SEQ ID NO: 97, SEQ ID NO: 99, SEQ ID NO: 101, or SEQ ID NO: 122.

In one embodiment, the disclosure includes an anti-TM4SF1 antibody which is an IgG and comprises four polypeptide chains including two heavy chains each comprising a heavy chain variable domain and heavy chain constant regions CH1, CH2 and CH3, and two light chains each comprising a light chain variable domain and a light chain constant region (CL). In certain embodiments, the antibody is a human IgG1, IgG2, or an IgG4. In certain embodiments, the antibody is a human IgG1. In other embodiments, the antibody is an IgG2. The heavy and light chain variable domain sequences may contain CDRs as set forth in Table 5.

Complementarity determining regions (CDRs) are known as hypervariable regions both in the light chain and the heavy chain variable domains. The more highly conserved portions of variable domains are called the framework (FR). CDRs and framework regions (FR) of a given antibody may be identified using the system described by Kabat et al. supra; Lefranc et al., supra and/or Honegger and Pluckthun, supra. Also familiar to those in the art is the numbering system described in Kabat et al. (1991, NIH Publication 91-3242, National Technical Information Service, Springfield, Va.). In this regard Kabat et al. defined a numbering system for variable domain sequences, including the identification of CDRs, that is applicable to any antibody.

One or more CDRs may be incorporated into a molecule either covalently or noncovalently to make it an antigen binding protein.

An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest. The CDR3, in particular, is known to play an important role in antigen binding of an antibody or antibody fragment.

In one embodiment, the disclosure provides an anti-TM4SF1 antibody, or an antigen-binding fragment thereof, comprising a heavy chain comprising a CDR3 domain as set forth in any one of SEQ ID NO: 8, SEQ ID NO: 20, SEQ ID NO: 32, SEQ ID NO: 44, SEQ ID NO: 56, SEQ ID NO: 68, or SEQ ID NO: 80 and comprising a variable domain comprising an amino acid sequence that has at least about 60%, at least about 65%, at least about 70%, at least about 75%, at least about 80%, at least about 85%, at least about 90%, at least about 95%, at least about 96%, at least about 97%, at least about 98%, at least about 99%, or 100% identical to a sequence as set forth in any one of SEQ ID NO: 3, SEQ ID NO: 15, SEQ ID NO: 27, SEQ ID NO: 39, SEQ ID NO: 51, SEQ ID NO: 63, or SEQ ID NO: 75. In one embodiment, the disclosure provides an anti-TM4SF1 antibody, or an antigen-binding fragment thereof, comprising a light chain comprising a CDR3 domain as set forth in any one of SEQ ID NO: 14, SEQ ID NO: 26, SEQ ID NO: 38, SEQ ID NO: 50, SEQ ID NO: 62, SEQ ID NO: 74, or SEQ ID NO: 86, and having a light chain variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 9, SEQ ID NO: 21, SEQ ID NO: 33, SEQ ID NO: 45, SEQ ID NO: 57, SEQ ID NO: 69, or SEQ ID NO: 81. Thus, in certain embodiments, the CDR3 domain is held constant, while variability may be introduced into the remaining CDRs and/or framework regions of the heavy and/or light chains, while the antibody, or antigen binding fragment thereof, retains the ability to bind to TM4SF1 and retains the functional characteristics, e.g., binding affinity, of the parent, or has improved functional characteristic, e.g., binding affinity, compared to the parent.

In one embodiment, the disclosure provides an anti-TM4SF1 antibody, or an antigen-binding fragment thereof, comprising a heavy chain comprising a CDR2 domain as set forth in any one of SEQ ID NO: 7, SEQ ID NO: 19, SEQ ID NO: 31, SEQ ID NO: 43, SEQ ID NO: 55, SEQ ID NO: 67, or SEQ ID NO: 79 and comprising a variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 3, SEQ ID NO: 15, SEQ ID NO: 27, SEQ ID NO: 39, SEQ ID NO: 51, SEQ ID NO: 63, or SEQ ID NO: 75. In one embodiment, the disclosure provides an anti-TM4SF1 antibody, or an antigen-binding fragment thereof, comprising a light chain comprising a CDR2 domain as set forth in any one of SEQ ID NO: 13, SEQ ID NO: 25, SEQ ID NO: 37, SEQ ID NO: 49, SEQ ID NO: 61, SEQ ID NO: 73, or SEQ ID NO: 85, and having a light chain variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 9, SEQ ID NO: 21, SEQ ID NO: 33, SEQ ID NO: 45, SEQ ID NO: 57, SEQ ID NO: 69, or SEQ ID NO: 81. Thus, in certain embodiments, the CDR2 domain is held constant, while variability may be introduced into the remaining CDRs and/or framework regions of the heavy and/or light chains, while the antibody, or antigen binding fragment thereof, retains the ability to bind to TM4SF1 and retains the functional characteristics, e.g., binding affinity, of the parent, or has improved functional characteristic, e.g., binding affinity, compared to the parent.

In one embodiment, the disclosure provides an anti-TM4SF1 antibody, or an antigen-binding fragment thereof, comprising a heavy chain comprising a CDR1 domain as set forth in any one of SEQ ID NO: 6, SEQ ID NO: 18, SEQ ID NO: 30, SEQ ID NO: 42, SEQ ID NO: 54, SEQ ID NO: 66, or SEQ ID NO: 78 and comprising a variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence as set forth in any one of SEQ ID NO: 3, SEQ ID NO: 15, SEQ ID NO: 27, SEQ ID NO: 39, SEQ ID NO: 45, SEQ ID NO: 69, or SEQ ID NO: 81. In one embodiment, the disclosure provides an anti-TM4SF1 antibody, or an antigen-binding fragment thereof, comprising a light chain comprising a CDR1 domain as set forth in any one of SEQ ID NO: 12, SEQ ID NO: 24, SEQ ID NO: 36, SEQ ID NO: 48, SEQ ID NO: 60, SEQ ID NO: 72, or SEQ ID NO: 84, and having a light chain variable domain comprising an amino acid sequence that has at least at least about 60%, at least about 65%, at least about 70%, at least about 75%, at least about 80%, at least about 85%, at least about 90%, at least about 95%, at least about 96%, at least about 97%, at least about 98%, or at least about 99%, or 100% identical to a sequence a set forth in any one of SEQ ID NO: 9, SEQ ID NO: 21, SEQ ID NO: 33, SEQ ID NO: 45, SEQ ID NO: 57, SEQ ID NO: 69, or SEQ ID NO: 81. Thus, in certain embodiments, the CDR1 domain is held constant, while variability may be introduced into the remaining CDRs and/or framework regions of the heavy and/or light chains, while the antibody, or antigen binding fragment thereof, retains the ability to bind to TM4SF1 and retains the functional characteristics, e.g., binding affinity, of the parent.

In some embodiments, an anti-TM4SF1 antibody of this disclosure comprises a heavy chain comprising an Fc region, wherein said Fc region comprises a sequence selected from the group consisting of: SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 151, SEQ ID NO: 152, and SEQ ID NO: 153; or wherein said Fc region comprises a sequence comprising one or more substitutions in a sequence selected from the group consisting of: SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 151, SEQ ID NO: 152, and SEQ ID NO: 153. For instance, in some embodiments, an anti-TM4SF1 antibody of this disclosure comprises an Fc region, wherein said Fc region comprises a sequence that is at least about 70% to about 100%, such as at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to a sequence selected from the group consisting of: SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 151, SEQ ID NO: 152, and SEQ ID NO: 153.

In some embodiments, an anti-TM4SF1 antibody of this disclosure comprises a heavy chain comprising a sequence selected from the group consisting of SEQ ID NO: 146, SEQ ID NO: 147, SEQ ID NO: 148, SEQ ID NO: 149, SEQ ID NO: 150, SEQ ID NO: 154, SEQ ID NO: 155, and SEQ ID NO: 156; or wherein said heavy chain comprises a sequence comprising one or more substitutions in a sequence selected from the group consisting of: SEQ ID NO: 146, SEQ ID NO: 147, SEQ ID NO: 148, SEQ ID NO: 149, SEQ ID NO: 150, SEQ ID NO: 154, SEQ ID NO: 155, and SEQ ID NO: 156. For instance, in some embodiments, an anti-TM4SF1 antibody of this disclosure comprises a heavy chain comprising a sequence that is at least about 70% to about 100%, such as at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to a sequence selected from the group consisting of: SEQ ID NO: 146, SEQ ID NO: 147, SEQ ID NO: 148, SEQ ID NO: 149, SEQ ID NO: 150, SEQ ID NO: 154, SEQ ID NO: 155, and SEQ ID NO: 156.

The anti-TM4SF1 antibodies and fragments described in Table 5 may also be humanized. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (See, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6 I) and VH subgroup III (VHIII). In another method, human germline genes are used as the source of the framework regions.

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human framework regions that may be used for humanization include but are not limited to: framework regions selected using the “best-fit” method (see, e.g., Sims, et al., J. Immunol. 151 (1993) 2296); framework regions derived from the consensus sequence of human antibodies of a particular subgroup of light or heavy chain variable regions (see, e.g., Carter, et al., Proc. Natl. Acad. Sci. USA, 89 (1992) 4285; and Presta, et al., J. Immunol., 151 (1993) 2623); human mature (somatically mutated) framework regions or human germline framework regions (see, e.g., Almagro, and Fransson, Front. Biosci. 13 (2008) 1619-1633); and framework regions derived from screening FR libraries (see, e.g., Baca, et al., J. Biol. Chem. 272 (1997) 10678-10684 and Rosok, et al., J. Biol. Chem. 271 (1996) 22611-22618).

Humanized antibodies and methods of making them are reviewed, e.g., in Almagro, and Fransson, Front. Biosci. 13 (2008) 1619-1633, and are further described, e.g., in Riechmann, et al., Nature 332 (1988) 323-329; Queen, et al., Proc. Nat'l Acad. Sci. USA 86 (1989) 10029-10033; U.S. Pat. Nos. 5,821,337, 7,527,791, 6,982,321, and 7,087,409; Kashmiri, et al., Methods 36 (2005) 25-34 (describing SDR (a-CDR) grafting); Padlan, Mol. Immunol. 28 (1991) 489-498 (describing “resurfacing”); Dall'Acqua, et al., Methods 36 (2005) 43-60 (describing “FR shuffling”); and Osbourn, et al., Methods 36 (2005)61-68 and Klimka, et al., Br. J. Cancer, 83 (2000) 252-260 (describing the “guided selection” approach to FR shuffling).

In one embodiment, an anti-TM4SF1 antibody, or antigen-binding fragment thereof, of the disclosure binds to cynomolgus TM4SF1 with a K_{D} about 1×10⁻⁶ M or less.

An anti-TM4SF1 antibody, or antigen-binding fragment thereof, of the disclosure, in certain embodiments, binds to an epitope on the ECL2 loop of human TM4SF1 with a K_{D} about 5×10⁻⁸ M or less as determined in a standard flow cytometry assay using HUVEC cells.

An anti-TM4SF1 antibody, or antigen-binding fragment thereof, of the disclosure, in certain embodiments, binds to human TM4SF1 with a K_{D} of about 1×10⁻⁸ M or less in a standard flow cytometry assay using HUVEC cells.

An anti-TM4SF1 antibody, or antigen-binding fragment thereof, of the disclosure, in certain embodiments, binds to human TM4SF1 with a K_{D} of about 1×10⁻³ M to about 1×10⁻⁴ M, about 1×10⁻⁴ M to about 1×10⁻⁵ M, about 1×10⁻⁵ M to about 1×10⁻⁶ M, about 1×10⁻⁶ to about 1×10⁻⁷ M, about 1×10⁻⁷ to about 1×10⁻⁸ M, about 1×10⁻⁸ M to about 1×10⁻⁹ M, about 1×10⁻⁹ M to about 1×10⁻¹⁰ M, about 1×10⁻¹⁰M to about 1×10⁻¹¹ M, about 1×10⁻¹¹ M to about 1×10⁻¹² M, about 2×10⁻³ M to about 2×10⁻⁴ M, about 2×10⁻⁴ M to about 2×10⁻⁵ M, about 2×10⁻⁵ M to about 2×10⁻⁶ M, about 2×10⁻⁶ to about 2×10⁻⁷ M, about 2×10⁻⁷ to about 2×10⁻⁸ M, about 2×10⁻⁸ M to about 2×10⁻⁹ M, about 2×10⁻⁹ M to about 2×10⁻¹⁰ M, about 2×10⁻¹⁰M to about 2×10⁻¹¹ M, about 2×10⁻¹¹ M to about 2×10⁻¹² M, about 3×10⁻³ M to about 3×10⁻⁴ M, about 3×10⁻⁴ M to about 3×10⁻⁵ M, about 3×10⁻⁵ M to about 3×10⁻⁶ M, about 3×10⁻⁶ to about 3×10⁻⁷ M, about 3×10⁻⁷ to about 3×10⁻⁸ M, about 3×10⁻⁸ M to about 3×10⁻⁹ M, about 3×10⁻⁹ M to about 3×10⁻¹⁰ M, about 3×10⁻¹⁰M to about 3×10¹¹ M, about 3×10⁻¹¹ M to about 3×10⁻¹² M, about 4×10⁻³ M to about 4×10⁻⁴ M, about 4×10⁻⁴ M to about 4×10⁻⁵ M, about 4×10⁻⁵ M to about 4×10⁻⁶ M, about 4×10⁻⁶ to about 4×10⁻⁷ M, about 4×10⁻⁷ to about 4×10⁻⁸ M, about 4×10⁻⁸ M to about 4×10⁻⁹ M, about 4×10⁻⁹ M to about 4×10⁻¹⁰ M, about 4×10⁻¹⁰ M to about 4×10⁻¹¹ M, about 4×10⁻¹¹ M to about 4×10⁻¹² M, about 5×10⁻³ M to about 5×10⁻⁴ M, about 5×10⁻⁴ M to about 5×10⁻⁵ M, about 5×10⁻⁵ M to about 5×10⁻⁶ M, about 5×10⁻⁶ to about 5×10⁻⁷ M, about 5×10⁻⁷ to about 5×10⁻⁸ M, about 5×10⁻⁸ M to about 5×10⁻⁹ M, about 5×10⁻⁹ M to about 5×10⁻¹⁰ M, about 5×10⁻¹⁰ M to about 5×10⁻¹¹ M, about 5×10⁻¹¹ M to about 5×10⁻¹² M, about 5×10⁻⁷ M to about 5×10⁻¹¹ M, about 5×10⁻⁷ M, about 1×10⁻⁷ M, about 5×10⁻⁸ M, about 1×10⁻⁸ M, about 5×10⁻⁹ M, about 1×10⁻⁹ M, about 5×10⁻¹⁰ M, about 1×10⁻¹⁰ M, about 5×10⁻¹¹ M or about 1×10⁻¹¹ M. In some embodiments, the K_{D} is determined in a standard flow cytometry assay using HUVEC cells.

An anti-TM4SF1 antibody, or antigen-binding fragment thereof, of the disclosure, in certain embodiments, binds to human TM4SF1 with a K_{D} of about 5×10⁻¹ M or less in a standard flow cytometry assay using HUVEC cells.

An anti-TM4SF1 antibody, or antigen-binding fragment thereof, of the disclosure, in certain embodiments, binds to cynomolgus TM4SF1 with a K_{D} about 1×10⁻⁶ M or less in a standard flow cytometry assay using HEK293 overexpressing cells. In one embodiment, the HEK293 cells are transfected to express cynomolgus TM4SF1. In a further embodiment, HEK293 cells express cynomolgus TM4SF1 at about 600 mRNA copies per 10⁶ copies 18S rRNA.

Methods of determining the K_{D} of an antibody or antibody fragment are known in the art. For example, surface plasmon resonance may be used to determine the K_{D} of the antibody to the antigen (e.g., using a BIACORE 2000 or a BIACORE 3000 (BIAcore, Inc., Piscataway, N.J.) at 25° C. with immobilized antigen or Fc receptor CM5 chips at about 10 response units (RU)). In certain embodiments FACS or flow cytometry is used to determine the K_{D}, whereby cells, such as HEK293 cells or HUVEC cells, that express TM4SF1 are used to bind the antibody or fragment and measure the K_{D} according to standard methods. Affinity determination of antibodies using flow cytometry is described, for example, in Geuijen et al (2005) J Immunol Methods. 302(1-2):68-77. In certain embodiments, FACS is used to determine affinity of antibodies.

In one embodiment, the disclosure features an anti-TM4SF1 antibody or antigen binding fragment thereof, having CDR amino acid sequences described herein with conservative amino acid substitutions, such that the anti-TM4SF1 antibody or the antigen binding fragment thereof comprises an amino acid sequence of a CDR that is at least 95% identical (or at least 96% identical, or at least 97% identical, or at least 98% identical, or at least 99% identical) to a CDR amino acid sequence set forth in Table 5. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

The disclosure further features in one aspect an anti-TM4SF1 antibody, or antigen-binding fragment thereof, that binds to an epitope on the ECL2 loop of human TM4SF1 with a K_{D} of about 5×10⁻⁸ M or less as determined in a standard flow cytometry assay using HUVEC cells, wherein the anti-TM4SF1 antibody, or antigen-binding fragment thereof, comprises a light chain variable region comprising a human IgG framework region and comprises a heavy chain variable region comprising a human IgG framework region. In one embodiment, the anti-TM4SF1 antibody, or antigen-binding fragment thereof, is humanized. In one embodiment, the anti-TM4SF1 antibody, or antigen-binding fragment thereof, cross reacts with cynomolgus TM4SF1.

In another aspect of the disclosure, the anti-TM4SF1 antibody, or antigen-binding fragment thereof, is a humanized anti-TM4SF1 antibody, or antigen-binding fragment thereof, that binds to an epitope on the ECL2 loop of human TM4SF1 with a K_{D} about 5×10⁻⁸ M or less as determined in a standard flow cytometry assay using HUVEC cells. In one embodiment, the anti-TM4SF1 antibody, or antigen-binding fragment thereof, binds to cynomolgus TM4SF1 with a K_{D} about 1×10⁻⁶ M or less in a standard flow cytometry assay using HEK293 overexpressing cells. In one embodiment, the anti-TM4SF1 antibody, or antigen-binding fragment thereof, binds to human TM4SF1 with a K_{D} of about 1×10⁻⁸ M or less in a standard flow cytometry assay using HUVEC cells. In one embodiment, the anti-TM4SF1 antibody, or antigen-binding fragment thereof, binds to human TM4SF1 with a K_{D} of 1×10⁻³ M to about 1×10⁻⁴ M, about 1×10⁻⁴ M to about 1×10⁻⁵ M, about 1×10⁻⁵ M to about 1×10⁻⁶ M, about 1×10⁻⁶ to about 1×10⁻⁷ M, about 1×10⁻⁷ to about 1×10⁻⁸ M, about 1×10⁻⁸ M to about 1×10⁻⁹ M, about 1×10⁻⁹ M to about 1×10⁻¹⁰ M, about 1×10⁻¹⁰M to about 1×10⁻¹¹ M, about 1×10⁻¹¹ M to about 1×10⁻¹² M, about 2×10⁻³ M to about 2×10⁻⁴ M, about 2×10⁻⁴ M to about 2×10⁻⁵ M, about 2×10⁻⁵ M to about 2×10⁻⁶ M, about 2×10⁻⁶ to about 2×10⁻⁷ M, about 2×10⁻⁷ to about 2×10⁻⁸ M, about 2×10⁻⁸ M to about 2×10⁻⁹ M, about 2×10⁻⁹ M to about 2×10⁻¹⁰ M, about 2×10⁻¹⁰M to about 2×10⁻¹¹ M, about 2×10⁻¹¹ M to about 2×10⁻¹² M, about 3×10⁻³ M to about 3×10⁻⁴ M, about 3×10⁻⁴ M to about 3×10⁻⁵ M, about 3×10⁻⁵ M to about 3×10⁻⁶ M, about 3×10⁻⁶ to about 3×10⁻⁷ M, about 3×10⁻⁷ to about 3×10⁻⁸ M, about 3×10⁻⁸ M to about 3×10⁻⁹ M, about 3×10⁻⁹ M to about 3×10⁻¹⁰ M, about 3×10⁻¹⁰M to about 3×10⁻¹¹ M, about 3×10⁻¹¹ M to about 3×10⁻¹² M, about 4×10⁻³ M to about 4×10⁻⁴ M, about 4×10⁻⁴ M to about 4×10⁻⁵ M, about 4×10⁻⁵ M to about 4×10⁻⁶ M, about 4×10⁻⁶ to about 4×10⁻⁷ M, about 4×10⁻⁷ to about 4×10⁻⁸ M, about 4×10⁻⁸ M to about 4×10⁻⁹ M, about 4×10⁻⁹ M to about 4×10⁻¹⁰ M, about 4×10⁻¹⁰ M to about 4×10⁻¹¹ M, about 4×10⁻¹¹ M to about 4×10⁻¹² M, about 5×10⁻³ M to about 5×10⁻⁴ M, about 5×10⁻⁴ M to about 5×10⁻⁵ M, about 5×10⁻⁵ M to about 5×10⁻⁶ M, about 5×10⁻⁶ to about 5×10⁻⁷ M, about 5×10⁻⁷ to about 5×10⁻⁸ M, about 5×10⁻⁸ M to about 5×10⁻⁹ M, about 5×10⁻⁹ M to about 5×10⁻¹⁰ M, about 5×10⁻¹⁰M to about 5×10¹¹ M, about 5×10⁻¹¹ M to about 5×10⁻¹² M, about 5×10⁻⁷ M to about 5×10⁻¹¹ M, about 5×10⁻⁷ M, about 1×10⁻⁷ M, about 5×10⁻⁸ M, about 1×10⁻⁸ M, about 5×10⁻⁹ M, about 1×10⁻⁹ M, about 5×10⁻¹⁰ M, about 1×10⁻¹⁰ M, about 5×10⁻¹¹ M or about 1×10⁻¹¹ M. In some embodiments, the K_{D} is determined in a standard flow cytometry assay using HUVEC cells. In one embodiment, the anti-TM4SF1 antibody, or antigen-binding fragment thereof, binds to human TM4SF1 with a K_{D} of about 5×10⁻¹⁰ M or less in a standard flow cytometry assay using TM4SF1 expressing HUVEC cells.

In one embodiment, binding of an anti-TM4SF1 antibody, or antigen binding fragment, of the disclosure to human TM4SF1 is not dependent on glycosylation of the ECL2 loop of human TM4SF1, i.e., binding of the antibody is independent of glycosylation of TM4SF1 within the ECL2 loop (SEQ ID NO: 77).

The anti-TM4SF1 antibodies, or antigen-binding fragments thereof, of the disclosure may be any of any isotype (for example, but not limited to IgG, IgM, and IgE). In certain embodiments, antibodies, or antigen-binding fragments thereof, of the disclosure are IgG isotypes. In a specific embodiment, antibodies, or antigen-binding fragments thereof, of the disclosure are of the IgG1, IgG2 or IgG4 isotype. In certain embodiments, the anti-TM4SF1 antibody, or antigen-binding fragment thereof, are human IgG1, human IgG2, or human IgG4 isotype.

IgG2 is naturally the lowest in ADCC and/or CDC activity (An et al., MAbs. 2009 November-December; 1(6): 572-579). Accordingly, in certain embodiments it IgG2 is advantageously used. However, IgG2 has two extra cysteines (leading to 4 inter-hinge disulfide bonds) which make it prone to aggregation via formation of inter-antibody disulfide bonds. In a related embodiment, mutations to the IgG2 cysteines are made to decrease aggregation.

The present disclosure provides antibody fragments that bind to TM4SF1. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., 1992, Bio/Technology 10:163-67). According to another approach, F(ab′)2 fragments can be isolated directly from recombinant host cell culture. Fab and F(ab′)2 fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

In certain embodiments, the antigen binding fragment is selected from the group consisting of a Fab, a Fab′, a F(ab′)2, an Fv, and an scFv.

Anti-TM4SF1 antibodies (and fragments) that, for example, have a high affinity for human TM4SF1, can be identified using screening techniques known in the art. For example, monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized using, for example, the ECL2 loop of human TM4SF1 or cells expressing TM4SF1 (whereby the ECL2 loop is expressed on the cell surface), to elicit lymphocytes that produce or are capable of producing antibodies that will specifically bind to the protein used for immunization.

Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, Va.), and those derived from MOPC-21 and MPC-11 mouse tumors available from the Salk Institute Cell Distribution Center (San Diego, Calif.). Human myeloma and mouse-human heteromyeloma cell lines also have been described for the production of human monoclonal antibodies (Kozbor, 1984, Immunol. 133:3001-05; and Brodeur et al., Monoclonal Antibody Production Techniques and Applications 51-63 (1987)).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxyapatites chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Pluckthun, 1992, Immunol. Revs. 130:151-88.

In a further embodiment, monoclonal antibodies or antibody fragments can be isolated from antibody phage libraries generated using the techniques described in, for example, Antibody Phage Display: Methods and Protocols (O'Brien and Aitken eds., 2002). In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self-antigens without any immunization as described by Griffiths et al., 1993, EMBO J 12:725-34. Finally, naive libraries can also be made synthetically by cloning the unrearranged V-gene segments from stem cells and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, TM4SF1 (e.g., a soluble form of the ECL2 loop or cells expressing the loop) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Anti-TM4SF1 antibodies can be obtained by designing a suitable antigen screening procedure to select for the phage clone of interest followed by construction of a full length anti-TM4SF1 antibody clone using VH and/or VL sequences (e.g., the Fv sequences), or various CDR sequences from VH and VL sequences, from the phage clone of interest and suitable constant region (e.g., Fc) sequences described in Kabat et al., supra.

Screening of anti-TM4SF1 antibodies can be performed using binding assays known in the art and described herein for determining whether the antibody has a therapeutic affinity for the ECL2 loop of TM4SF1. The ability of the antibody to inhibit or decrease metastatic cell activity can be measured using standard assays in the art, as well as those described herein. Preclinical assays require use of an animal model of metastasis, commonly of one of three types: (i) injection of metastatic mouse tumor cells such as B16F10 melanoma TCs into mice, commonly via tail vein injection to generate lung metastases, via portal vein or intrasplenic injection to generate liver metastases, or via left ventricular cardiac injection to generate bone and other metastases; (ii) orthotopic transplantation of metastatic tumor cells or intact tumor fragments into mice, which methods often require later surgical resection of the primary tumor to prevent morbidity associated with primary tumor growth; and (iii) genetically engineered mouse models of spontaneous metastasis, of which the most common is the MMTV-Pyt (mouse mammary tumor virus-polyomavirus middle T Antigen) mouse mammary carcinoma model which provides a highly realistic mouse model of human cancer metastasis; greater than 85% of hemizygous MMTV-PyMT females spontaneously develop palpable mammary tumors which metastasize to the lung at age to 8-16 weeks. Quantifying the metastatic burden in the lung, either by live animal imaging or direct counting of metastatic nodules in the lungs of sacrificed animals, as a function of the degree of TM4SF1 immunoblockade and achieving a therapeutic level, e.g., at least a 50% reduction in lung metastasis, would be indicative, for example, of a therapeutic antibody that could be used in the methods of the disclosure. Further, cross-species reactivity assays are known in the art. Examples of assays that can be used are described, for example, in Khanna and Hunter (Carcinogenesis. 2005 March; 26(3):513-23) and Saxena and Christofori (Mol Oncol. 2013 April; 7(2):283-96), incorporated by reference in their entireties herein.

In some embodiments, an anti-TM4SF1 antibody or an antigen binding fragment thereof is cysteine engineered for conjugation by reduction and reoxidation. Cysteine engineered antibodies, in some embodiments, are made reactive for conjugation with linker-degrader intermediates described herein, by treatment with a reducing agent such as DTT (Cleland's reagent, dithiothreitol) or TCEP (tris(2-carboxyethyl)phosphine hydrochloride; Getz et al (1999) Anal. Biochem. Vol 273:73-80; Soltec Ventures, Beverly, MA) followed by re-formation of the inter-chain disulfide bonds (reoxidation) with a mild oxidant such as dehydroascorbic acid.

In some instances, the cysteine engineered anti-TM4SF1 antibodies are reduced, for example, with about a 50 fold excess of DTT overnight in 50 mM Tris, pH 8.0 with 2 mM EDTA at room temperature, which removes Cys and glutathione adducts as well as reduces interchain disulfide bonds in the antibody. Removal of the adducts is in some instances monitored by reverse-phase LCMS using a PLRP-S column. The reduced cysteine engineered antibody can then be diluted and acidified by addition to at least about four volumes of 10 mM sodium succinate, pH 5 buffer. Alternatively, the antibody is diluted and acidified by adding to at least four volumes of 10 mM succinate, pH 5 and titration with 10% acetic acid until pH is approximately five. The pH-lowered and diluted cysteine engineered antibody is subsequently loaded onto a HiTrap S cation exchange column, washed with several column volumes of 10 mM sodium acetate, pH 5 and eluted with 50 mM Tris, pH 8.0, 150 mM sodium chloride. Disulfide bonds are reestablished between cysteine residues present in the parent Mab by carrying out reoxidation. The eluted reduced cysteine engineered antibody described above is treated with 15× dehydroascorbic acid (DHAA) for about 3 hours or, alternatively, with 200 nM to 2 mM aqueous copper sulfate (CuSO4) at room temperature overnight. Other oxidants, i.e., oxidizing agents, and oxidizing conditions, which are known in the art may be used. Ambient air oxidation may also be effective. This mild, partial reoxidation step forms intrachain disulfides efficiently with high fidelity. Reoxidation can be monitored by reverse-phase LCMS using a PLRP-S column. The reoxidized cysteine engineered antibody can be diluted with succinate buffer as described above to reach pH of approximately 5 and purification on an S column may be carried out as described above with the exception that elution was performed with a gradient of 10 mM succinate, pH 5, 300 mM sodium chloride (buffer B) in 10 mM succinate, pH 5 (buffer A). To the eluted antibody, EDTA is added to a final concentration of 2 mM and concentrated, if necessary, to reach a final concentration of more than 5 mg/mL. The resulting cysteine engineered antibody, ready for conjugation, can be stored at −20° C. or −80° C. in aliquots. Liquid chromatography/Mass Spectrometric Analysis was performed on a 6200 series TOF or QTOF Agilent LC/MS. Samples are, in some instances, chromatographed on a PRLP-S®, 1000 A, microbore column (50 mm×2.1 mm, Polymer Laboratories, Shropshire, UK) heated to 80° C. A linear gradient from 30-40% B (solvent A: 0.05% TFA in water, solvent B: 0.04% TFA in acetonitrile) was used and the eluent was directly ionized using the electrospray source. Data were collected and deconvoluted by the MassHunter software (Agilent). Prior to LC/MS analysis, antibodies or conjugates (50 micrograms) were treated with PNGase F (2 units/ml; PROzyme, San Leandro, CA) for 2 hours at 37° C. to remove N-linked carbohydrates.

Alternatively, antibodies or conjugates are partially digested with LysC (0.25 μg per 50 μg (microgram) antibody or conjugate) for 15 minutes at 37° C. to give a Fab and Fc fragment for analysis by LCMS. Peaks in the deconvoluted LCMS spectra are assigned and quantitated. Degrader-to-antibody ratios (DAR) are calculated by calculating the ratio of intensities of the peak or peaks corresponding to Degrader-conjugated antibody relative to all peaks observed.

In some embodiments, the anti-TM4SF1 antibodies and antigen binding fragments thereof can be used, e.g., to treat or prevent cancer. In certain embodiments, the anti-TM4SF1 antibodies and antigen binding fragments of the disclosure can be used to prevent tumor cells from metastasizing. The anti-TM4SF1 antibodies and antigen binding fragments thereof, of this disclosure, in some examples, prevent tumor cell metastasis by interfering with the interaction between tumor cells and blood vessel endothelial cells.

### IV. Therapeutic Molecules in the ADCs

In some embodiments, the ADCs of this disclosure comprise one or more therapeutic (also referred to herein as a therapeutic molecule or a therapeutic agent) conjugated to an anti-TM4SF1 antibody or an antigen binding fragment thereof. In some embodiments, the agent is a therapeutic agent or a diagnostic agent. In some embodiments, the therapeutic agent is a biologically active moiety. In some embodiments, the biologically active moiety comprises a radioactive isotope, a cytotoxic agent, a chemotherapeutic agent, a protein, a peptide, an antibody, a growth inhibitory agent, a prodrug activating enzyme, and an anti-hormonal agent. In some embodiments, a therapeutic molecule can be a small molecule (e.g., both for cancer and for non-cancer angiogenic indications); a V-ATPase inhibitor; a pro-apoptotic agent; a Bcl2 inhibitor; an MCL1 inhibitor; a HSP90 inhibitor; an IAP inhibitor; an mTor inhibitor; a microtubule stabilizer; a microtubule destabilizer; an auristatin; a dolastatin; a maytansinoid; a MetAP (methionine aminopeptidase); an inhibitor of nuclear export of proteins CRM1; a DPPIV inhibitor; proteasome inhibitors; inhibitors of phosphoryl transfer reactions in mitochondria; a protein synthesis inhibitor; a kinase inhibitor (such as, a CDK2 inhibitor, a CDK9 inhibitor); a kinesin inhibitor, an HDAC inhibitor, a DNA damaging agent, a DNA alkylating agent, a DNA intercalator, a DNA minor groove binder, a DHFR inhibitor, a nucleic acid, a CRISPR enzyme; degraders (such as agents that induce protein degradation, (e.g., HSP90 inhibitor, selective estrogen receptor degraders (SERDs), selective androgen receptor degraders (SARDs); hydrophobic tags that can be used to recruit chaperones to a protein of interest, e.g., Adamantane, Arg-Boc3; E3 ligase recruiting ligands, e.g., Nutlin-3a (MDM2 ligand), Bestatin (cIAP ligand), VHL ligand, Pomalidomide (CRBN ligand); proteolysis-targeting chimeras (PROTACs) that may utilize different D3 ligases to target a protein of interest for degradation)) (see, e.g., Lai A C, Crews C M. Induced protein degradation: an emerging drug discovery paradigm. Nat Rev Drug Discov. 2016; 16(2):101-114); antisense oligonucleotides; RNAi agents (such as siRNA), CRISPR-Cas9 gene editing systems; RNA molecules; DNA e.g., plasmids; an anti-cancer agent, an anti-inflammatory agent, an anti-infective agent (e.g., anti-fungal, antibacterial, anti-parasitic, anti-viral), an anesthetic agent; RNA polymerase II inhibitor; a DNA intercalating agent, a DNA cross-linking agent; an anti-tubulin agent; a cytotoxic drug, a tumor vaccine, an antibody, a peptide, pepti-bodies, a chemotherapeutic agent, a cytotoxic agent; a cytostatic agent; an immunological modifiers, an interferon, an interleukin, an 96rotea stimulatory growth hormone, a cytokine, a vitamin, a mineral, an aromatase inhibitor, a Histone Deacetylase (HDAC), an HDAC inhibitor; a lipid nanoparticle to encapsulate one or more therapeutic molecule.

In some embodiments, the radioactive isotope may be one or more kinds selected from the group consisting of ²¹¹At, ¹³¹I, ¹²⁵I, ⁹⁰Y, ¹⁸⁶Re, ¹⁸⁸Re, ¹⁵³Sm, ²¹²Bi, ³²P, and radioactive isotopes of Lu, but not limited thereto. In some embodiments, the prodrug-activating enzyme is one or more kinds selected from the group consisting of: an alkaline phosphatase, an arylsulfatase, a cytosine deaminase, a protease, a D-alanylcarboxy-peptidase, a carbohydrate-cleaving enzyme, a P-lactamase and a penicillin amidase, but not limited thereto.

The cytotoxic agent, ins some embodiments, comprises one or more selected from the group consisting of: ricin, saporin, gelonin, momordin, debouganin, diphtheria toxin, pseudomonas toxin, etc., but not limited thereto. The cytotoxic agent, in some instances is one or more kinds selected from the group consisting of: cisplatin, carboplatin, oxaliplatin, paclitaxel, melphalan, doxorubicin, methotrexate, 5-fluorouracil, etoposide, mechlorethamine, cyclophosphamide, bleomycin, a calicheamicin, a maytansine, a trichothene, CC1065, diphtheria A chain, Pseudomonas aeruginosa exotoxin A chain, ricin A chain, abrin A chain, modeccin A chain, alpha-sarcin, Aleuritesfordii proteins, dianthin proteins, Phytolaca 96roteasom proteins, 96roteasom charantia inhibitors, curcin, crotin, Sapaonaria officinalis inhibitors, gelonin, mitogellin, restrictocin, phenomycin, 96roteaso, tricothecenes, ribonucleases and deoxyribonucleases, but not limited thereto. In some embodiments, the cytotoxic agent is one or more kinds selected from the group consisting of: duocarmycin, monomethyl auristatin E (MMAE), monomethyl auristatin F (MMAF), N2′-deacetyl-N2′-(3-mercapto-1-oxopropyl)maytansine (DM1), PBD (Pyrrolobenzodiazepine) dimer, duocarmycin, monomethyl auristatin E (MMAE), monomethyl auristatin F (MMAF), but not limited thereto. In some embodiments, the cytotoxic agent comprises a ribosome inactivating protein, a histone deacetylase (HDAC) inhibitor, a tubulin inhibitor, an alkylating agent, an antibiotic, an antineoplastic agent, an antiproliferative agent, an antimetabolite, a topoisomerase I or II inhibitor, a hormonal agonist or antagonist, an immunomodulator, a DNA minor groove binder, and a radioactive agent. In certain embodiments, the ribosome inactivating protein is saporin. In some embodiments, the diagnostic agent is a label. In some embodiments, the label is a fluorescent label, a chromogenic label, or a radiolabel. In some embodiments, the agent is directly conjugated to the anti-TM4SF1 antibody or the antigen binding fragment thereof. In other embodiments, the agent is indirectly conjugated to the anti-TM4SF1 antibody or the antigen binding fragment thereof, optionally by a linker.

In some embodiments, an ADC of this disclosure comprises an anti-TM4SF1 antibody or antigen binding fragment thereof and one or more agents (e.g., 1, 2, 3, or 4 or more agents), such as therapeutic agents, that act additively or synergistically with the anti-TM4SF1 antibody or the antigen binding fragment thereof, for example, to kill or inhibit tumor cells (TCs) and/or tumor vasculature endothelial cells (ECs) in the treatment of a disorder associated with pathological angiogenesis, such as cancer. The therapeutic agent, for example, can be a biologically active moiety, such as a cytotoxic agent, a chemotherapeutic agent, a protein, a peptide, an antibody, a growth inhibitory agent, and/or an anti-hormonal agent.

Examples of tubulin inhibitors that can be conjugated, either directly or indirectly, to an anti-TM4SF1 antibody or antigen binding fragment thereof, can include, without limitation, polymerization inhibitors (e.g., vinblastine, vincristine, vinorelbine, vinflunine, cryptophycin 52, hallchondrins, dolastatins, hemiasterlins that can bind to the vinca domain of tubulin; colchine, combretastatins, 2-methoxy-estradiol, E7010 that can bind to the cholchicine domain of tubulin; depolymerization inhibitors, such as paclitaxel, docetaxel, 97roteasome, discodermolide that can bind to the taxane site).

Exemplary chemotherapeutic agents include, but are not limited to, methotrexate, adriamicin, vinca alkaloids (vincristine, vinblastine, etoposide), doxorubicin, melphalan, mitomycin C, chlorambucil, daunorubicin or other intercalating agents; enzymes and fragments thereof such as nucleolytic enzymes, antibiotics, and toxins such as small molecule toxins or enzymatically active toxins of bacterial, fungal, plant or animal origin, including fragments and/or variants thereof.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca 97roteasom proteins (PAPI, PAPII, and PAP-S), 97roteasom charantia inhibitor, curcin, crotin, Sapaonaria officinalis inhibitor, gelonin, mitogellin, restrictocin, phenomycin, 97roteaso, and the tricothecenes.

In addition, a variety of radionuclides can be used for conjugation of the anti-TM4SF1 antibodies or antigen binding fragments to the therapeutic agents, to generate the ADCs of this disclosure. Examples include At²¹¹, I¹³¹, I¹²⁵, Y⁹⁰, Re¹⁸⁶, Sm¹⁵³, Bi²¹², P³², and radioactive isotopes of Lu. Alternatively, the anti-TM4SF1 antibodies or antigen binding fragments can be conjugated to one or more smaller molecule toxins, such as a calicheamicin, maytansinoids, dolastatins, aurostatins, a trichothecene, and CC1065, and the derivatives of these toxins that have toxin activity, are also contemplated herein. Other therapeutic agents that can be conjugated to TM4SF1 binding protein of the disclosure include, in various examples, BCNU, streptozoicin, vincristine and 5-fluorouracil etc.

The diagnostic agent for conjugation, in some embodiments, is a label, such as a fluorescent label, a chromogenic label, or a radiolabel. Accordingly, the label may be used for detection purposes, and may be a fluorescent compound, an enzyme, a prosthetic group, a luminescent material, a bioluminescent material, or a radioactive material. The radiolabel, for example, may comprise a radioactive atom for scintigraphic studies, for example Tc^{99m} or I¹²³, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

The one or more agents (e.g., therapeutic agents and/or diagnostic agents) may be directly conjugated to anti-TM4SF1 antibodies or antigen binding fragments (e.g., by way of a direct covalent or non-covalent interaction), such that the agent is immediately conjugated to the protein. An agent may be directly conjugated to a binding protein of the disclosure, for example, by a direct peptide bond. In other instances, the direct conjugation is by way of a direct non-covalent interaction, such as an interaction between the anti-TM4SF1 antibodies or antigen binding fragments and an agent that specifically binds to the anti-TM4SF1 antibodies or antigen binding fragments.

### V. Linkers

The one or more agents (e.g., therapeutic agents and/or diagnostic agents) may be indirectly conjugated to anti-TM4SF1 antibodies or antigen binding fragments (e.g., by way of a linker with direct covalent or non-covalent interactions). Linkers can be chemical linking agents, such as homobifunctional and heterobifunctional cross-linkers, which are available from many commercial sources. Regions available for cross-linking may be found on the binding protein (e.g., anti-TM4SF1 antibodies) of the disclosure. The linker may comprise a flexible arm, e.g., 2, 3, 4, 5, 6, 7, 8, 9, 10, 11, 12, 13, 14, or 15 carbon atoms. The linker may comprise multiple fragments, including but not limited to, a first fragment and a second fragment. The first fragment may be cleavable. The second fragment may be cleavable. The second fragment may be non-cleavable. The first fragment and the second fragment may be cleavable. The first fragment may be cleavable, and the second fragment may be non-cleavable. The linker may comprise multiple first fragments that are cleavable. Each of the multiple first fragments may be the same or different. The linker may comprise multiple second fragments that are non-cleavable. Each of the multiple second fragments may be the same or different. The first fragment may directly conjugate to anti-TM4SF1 antibodies or antigen binding fragments. The first fragment may directly conjugate to one or more agents (e.g., therapeutic agents and/or diagnostic agents). The first fragment may indirectly conjugate to anti-TM4SF1 antibodies or antigen binding fragments, e.g., by way of a second fragment or another first fragment. The first fragment may indirectly conjugate to one or more agents (e.g., therapeutic agents and/or diagnostic agents), e.g., by way of a second fragment or another first fragment. The second fragment may directly conjugate to anti-TM4SF1 antibodies or antigen binding fragments. The second fragment may directly conjugate to one or more agents (e.g., therapeutic agents and/or diagnostic agents). The second fragment may indirectly conjugate to anti-TM4SF1 antibodies or antigen binding fragments, e.g., by way of another second fragment or another first fragment. The second fragment may indirectly conjugate to one or more agents (e.g., therapeutic agents and/or diagnostic agents), e.g., by way of another second fragment or a first fragment.

### Exemplary Linkers or Fragments Thereof can Include BS3

([Bis(sulfosuccinimidyl)suberate];BS3 is a homobifunctional N-hydroxysuccinimide ester that targets accessible primary amines), NHS/EDC (N-hydroxysuccinimide and N-ethyl-(dimethylaminopropyl)carbodiimide); NHS/EDC allows for the conjugation of primary amine groups with carboxyl groups), sulfo-EMCS ([N-e-Maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-S-acetylthioacetate; SATA is reactive towards amines and adds protected sulfhydryls groups). To form covalent bonds, a chemically reactive group a wide variety of active carboxyl groups (e.g., esters) where the hydroxyl moiety is physiologically acceptable at the levels required to modify the peptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA) maleimido hexanoic acid (MHA), and maleimido undecanoic acid (MUA). Primary amines are the principal targets for NHS esters. Accessible a-amino groups present on the N-termini of proteins and the ε-amine of lysine react with NHS esters. An amide bond is formed when the NHS ester conjugation reaction reacts with primary amines releasing N-hydroxysuccinimide. These succinimide containing reactive groups are herein referred to as succinimidyl groups. In certain embodiments of the disclosure, the functional group on the protein will be a thiol group and the chemically reactive group will be a maleimido-containing group such as gamma-maleimide-butrylamide (GMBA or MPA). Such maleimide containing groups are referred to herein as maleimido groups. The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl can be formed.

Further exemplary linker or a fragment thereof and linker chemistry that in some embodiments is used for conjugation of an anti-TM4SF1 antibody or an antigen binding fragment thereof, as described herein, include moieties that can be used in a click conjugation, e.g., in a two-step conjugation in which a first moiety is conjugated to an engineered cysteine (e.g., at position N297 with an N297C mutation), the first moiety containing a reactive handle, and a second moiety containing the linker-payload which reacts with the first moiety. An example of a possible reaction between the first moiety's reactive handle and the second moiety is a metal free click reaction that utilizes strain-promoted azide-alkyne cycloaddition. Examples of moieties include, but are not limited to, bicyclononyne (BCN) reacting with an azide or tetrazine, dibenzocyclooctyne (DBCO) reacting with an azide, also denoted as aza-dibenzocyclooctyne (DIBAC), a transcyclooctene (TCO) reacting with a tetrazine (such as methyl tetrazine), or a methyl cycloprene click handle reacting with tetrazine. Specific examples of such moieties are as follows, but not limited to: dibenzocyclooctyne-PEGx-carboxylic acid (X is 1-8), perfluorophenyl 6-(2,5-dioxo-2,5-dihydro-1H-pyrrol-1-yl)hexanoate Chemical Formula: C16H12F5NO4 Molecular Weight: 377.27; 6-(3,4-dibromo-2,5-dioxo-2,5-dihydro-1H-pyrrol-1-yl)hexanoic acid Chemical Formula: C10H11Br2NO4 Molecular Weight: 369.01; (2-methylcycloprop-2-en-1-yl)methyl carbamate (E)-cyclooct-4-en-1-yl (2-(2-(2-(2-aminoethoxy)ethoxy)ethoxy)ethyl)carbamate 3-(5-methylpyridin-2-yl)-6-(100roteaso-2-yl)-1,2,4,5-tetrazine; ((1R,8S,9s)-bicyclo[6.1.0]non-4-yn-9-yl)methyl (2-(2-(2-aminoethoxy)ethoxy)ethyl)carbamate Chemical Formula: C17H28N2O4 Molecular Weight: 324.42; ((1R,8S,9s)-bicyclo[6.1.0]non-4-yn-9-yl)methyl (2-(2-(2-aminoethoxy)ethoxy)ethyl)carbamate Chemical Formula: C17H28N2O4 Molecular Weight: 324.42.

In other embodiments, the linker or a fragment thereof includes at least one amino acid (e.g., a peptide of at least 2, 3, 4, 5, 6, 7, 10, 15, 20, 25, 40, or 50 amino acids). In certain embodiments, the linker or a fragment thereof is a single amino acid (e.g., any naturally occurring amino acid such as Cys). In other embodiments, a glycine-rich peptide such as a peptide can be used. In some cases, the linker or fragments thereof can be a single amino acid (e.g., any amino acid, such as Gly or Cys). Examples of suitable linkers or fragments thereof are succinic acid, Lys, Glu, and Asp, or a dipeptide such as Gly-Lys. When the linker or a fragment thereof is succinic acid, one carboxyl group thereof may form an amide bond with an amino group of the amino acid residue, and the other carboxyl group thereof may, for example, form an amide bond with an amino group of the peptide or substituent. When the linker or a fragment thereof is Lys, Glu, or Asp, the carboxyl group thereof may form an amide bond with an amino group of the amino acid residue, and the amino group thereof may, for example, form an amide bond with a carboxyl group of the substituent. When Lys is used as the linker or fragments thereof, a further linker or fragments thereof may be inserted between the ε-amino group of Lys and the substituent. In one particular embodiment, the further linker or a fragment thereof is succinic acid which, e.g., forms an amide bond with the ε-amino group of Lys and with an amino group present in the substituent. In one embodiment, the further linker or a fragment thereof is Glu or Asp (e.g., which forms an amide bond with the ε-amino group of Lys and another amide bond with a carboxyl group present in the substituent), that is, the substituent is a NE-acylated lysine residue.

In some embodiments, the anti-TM4SF1 antibody or the antigen binding fragment thereof as described herein and an oligonucleotide (e.g., a nucleic acid molecule, such as an RNA molecule or a DNA molecule) can be conjugated using various approaches, such as a genetic conjugation, an enzymatic conjugation, a chemical conjugation, or any combination thereof.

In some embodiments, the RNA molecules within the ADCs may be conjugated to the anti-TM4SF1 antibody or the antigen binding fragment thereof using an enzymatic site-specific conjugation method which involves the use of a mammalian or bacterial transglutaminase enzyme. Microbial transglutaminases (mTGs) are versatile tools in modern research and biotechnology. The availability of large quantities of relatively pure enzymes, ease of use, and lack of regulation by calcium and guanosine-5′-triphosphate (GTP) has propelled mTG to be the main cross-linking enzyme used in both the food industry and biotechnology. Currently, mTGs are used in many applications to attach proteins and peptides to small molecules, polymers, surfaces, DNA, as well as to other proteins. See, e.g., Pavel Strp, Veracity of microbial transglutaminase, Bioconjugate Chem. 25, 5, 855-862).

In some embodiments, the RNA molecules within the conjugates may be conjugated to the anti-TM4SF1 antibody or the antigen binding fragment thereof by way of a linker or a fragment thereof with direct covalent or non-covalent interactions. Linkers or fragments thereof can be amino acid or peptide based linkers, or chemical linking agents, such as homobifunctional and heterobifunctional cross-linkers, which are available from many commercial sources. Regions available for cross-linking may be found on the anti-TM4SF1 antibody or the antigen binding fragment thereof of the disclosure. The linker or fragments thereof may comprise a flexible arm, e.g., 2, 3, 4, 5, 6, 7, 8, 9, 10, 11, 12, 13, 14, or 15 carbon atoms. Exemplary linkers or fragments thereof include cleavable, non-cleavable, covalent, or non-covalent linkers, or any combinations thereof. The cleavable linker, in some embodiments, comprises an acid-labile linker, a protease-sensitive linker, a photo-labile linker, or a disulfide-containing linker. In some embodiments, the linker or a fragment thereof comprises a cysteine linker or a non-cysteine linker, such as a lysine linker. In some embodiments, the anti-TM4SF1 antibody or the antigen binding fragment thereof comprises an unnatural amino acid, wherein the antibody or antibody fragment and the oligonucleotide are linked/conjugated via the unnatural amino acid.

In some embodiments, the anti-TM4SF1 antibody or the antigen binding fragment thereof comprises a natural amino acid, wherein the antibody or antibody fragment and the oligonucleotide are linked/conjugated via the natural amino acid. The unnatural amino acid may be inserted between two naturally occurring amino acids in the antibody or antibody fragment. The one or more unnatural amino acids may replace one or more naturally occurring amino acids in the antibody or antibody fragment. The one or more unnatural amino acids may be incorporated at the N terminus of the antibody or antibody fragment. The one or more unnatural amino acids may be incorporated at the C terminus of the antibody or antibody fragment. The unnatural amino acid may be incorporated distal to the binding region of antibody or antibody fragment. The unnatural amino acid may be incorporated near the binding region of the antibody or antibody fragment. The unnatural amino acid may be incorporated in the binding region of the antibody or antibody fragment.

The one or more unnatural amino acids may be encoded by a codon that does not code for one of the twenty natural amino acids. The one or more unnatural amino acids may be encoded by a nonsense codon (stop codon). The stop codon may be an amber codon. The amber codon may comprise a UAG sequence. The stop codon may be an ochre codon. The ochre codon may comprise a UAA sequence. The stop codon may be an opal or umber codon. The opal or umber codon may comprise a UGA sequence. The one or more unnatural amino acids may be encoded by a four-base codon.

The one or more unnatural amino acids may be p-acetylphenylalanine (pAcF or pAcPhe). The one or more unnatural amino acids may be selenocysteine. The one or more unnatural amino acids may be p-fluorophenylalanine (pFPhe). The one or more unnatural amino acids may be selected from the group comprising p-azidophenylalanine (pAzF),p-azidomethylphenylalanine(pAzCH2F), p-benzoylphenylalanine (pBpF), p-propargyloxyphenylalanine (pPrF), p-iodophenylalanine (pIF), p-cyanophenylalanine (pCNF), p-carboxylmethylphenylalanine (pCmF), 3-(2-naphthyl)alanine (NapA), p-boronophenylalanine (pBoF), o-nitrophenylalanine (oNiF), (8-hydroxyquinolin-3-yl)alanine (HQA), selenocysteine, and (2,2′-bipyridin-5-yl)alanine (BipyA). The one or more unnatural amino acids may be 4-(6-methyl-s-tetrazin-3-yl)aminopheynlalanine.

The one or more unnatural amino acids may be n-amino acids (03 and 02), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, N-methyl amino acids, or a combination thereof.

Additional examples of unnatural amino acids include, but are not limited to, 1) various substituted tyrosine and phenylalanine analogues such as O-methyl-L-tyrosine, p-amino-L-phenylalanine, 3-nitro-L-tyrosine, p-nitro-L-phenylalanine, m-methoxy-L-phenylalanine and p-isopropyl-L-phenylalanine; 2) amino acids with aryl azide and benzophenone groups that may be photo-cross-linked; 3) amino acids that have unique chemical reactivity including acetyl-L-phenylalanine and m-acetyl-L-phenylalanine, O-allyl-L-tyrosine, 0-(2-propynyl)-L-tyrosine, p-ethylthiocarbonyl-L-phenylalanine and p-(3-oxobutanoyl)-L-phenylalanine; 4) heavy-atom-containing amino acids for phasing in X-ray crystallography including p-iodo and p-bromo-L-phenylalanine; 5) the redox-active amino acid dihydroxy-L-phenylalanine; 6) glycosylated amino acids including b-N-acetylglucosamine-O-serine and a-N-acetylgalactosamine-O-threonine; 7) fluorescent amino acids with naphthyl, dansyl, and 7-aminocoumarin side chains; 8) photocleavable and photoisomerizable amino acids with azobenzene and nitrobenzyl Cys, Ser, and Tyr side chains; 9) the phosphotyrosine mimetic p-carboxymethyl-L-phenylalanine; 10) the glutamine homologue homoglutamine; and 11) 2-aminooctanoic acid. The unnatural amino acid may be modified to incorporate a chemical group. The unnatural amino acid may be modified to incorporate a ketone group.

The one or more unnatural amino acids may comprise at least one oxime, carbonyl, dicarbonyl, hydroxylamine group or a combination thereof. The one or more unnatural amino acids may comprise at least one carbonyl, dicarbonyl, alkoxy-amine, hydrazine, acyclic alkene, acyclic alkyne, cyclooctyne, cyclopropene, aryl/alkyl azide, norbomene, 103roteasome103r, trans-cyclooctene, or tetrazine functional group or a combination thereof.

The one or more unnatural amino acids may be incorporated into the antibody or antibody fragment by methods known in the art. Cell-based or cell-free systems may be used to alter the genetic sequence of antibody or antibody fragment, thereby producing the antibody or antibody fragment with one or more unnatural amino acids. Auxotrophic strains may be used in place of engineered tRNA and synthetase. The one or more unnatural amino acids may be produced through selective reaction of one or more natural amino acids. The selective reaction may be mediated by one or more enzymes. In one non-limiting example, the selective reaction of one or more cysteines with formylglycine generating enzyme (FGE) may produce one or more formylglycines as described in Rabuka et al., Nature Protocols 7:1052-1067 (2012).

The one or more unnatural amino acids may take part in a chemical reaction to form a linker or fragments thereof. The chemical reaction to form the linker or fragments thereof may be a proteasome reaction or a bioorthogonal reaction. The chemical reaction to form the linker or fragments thereof may be click chemistry.

Additional unnatural amino acids are disclosed in Liu et al. (Annu Rev Biochem, 79:413-44, 2010), Wang et al. (Angew Chem Int Ed, 44:34-66, 2005) and PCT application numbers PCT/US2012/039472, PCT/US2012/039468, PCT/US2007/088009, PCT/US2009/058668, PCT/US2007/089142, PCT/US2007/088011, PCT/US2007/001485, PCT/US2006/049397, PCT/US2006/047822 and PCT/US2006/044682, all of which are incorporated by reference in their entireties.

The one or more unnatural amino acids may replace one or more amino acids in the antibody or antibody fragment. The one or more unnatural amino acids may replace any natural amino acid in the antibody or antibody fragment.

The one or more unnatural amino acids may be incorporated in a light chain of the antibody or antibody fragment. The one or more unnatural amino acids may be incorporated in a heavy chain of the antibody or antibody fragment. The one or more unnatural amino acids may be incorporated in a heavy chain and a light chain of antibody or antibody fragment. The one or more unnatural amino acids may replace an amino acid in the light chain of the antibody or antibody fragment. The one or more unnatural amino acids may replace an amino acid in a heavy chain of the antibody or antibody fragment. The one or more unnatural amino acids may replace an amino acid in a heavy chain and a light chain of the antibody or antibody fragment.

anti-TM4SF1 antibody or an antigen binding fragment thereof anti-TM4SF1 antibody or an antigen binding fragment thereof anti-TM4SF1 antibody or an antigen binding fragment thereof anti-TM4SF1 antibody or an antigen binding fragment thereof anti-TM4SF1 antibody or an antigen binding fragment thereof anti-TM4SF1 antibody or an antigen binding fragment thereof anti-TM4SF1 antibody or an antigen binding fragment thereof. In some embodiments, the linker or a fragment thereof comprises a small molecule fragment, a spacer, a non-covalent linker, or a combination thereof. In some embodiments, the linker or a fragment thereof comprises one or more of small molecule fragments. In some embodiments, the linker or a fragment thereof comprises a spacer.

In some embodiments, a linker or a fragment thereof comprises one or more of reactive moieties. In some embodiments, a linker or a fragments thereof comprise a reactive moiety selected from a Michael acceptor moiety, a leaving group moiety, or a moiety capable of forming a covalent bond with the antibody fragment and/or the therapeutic agent.

In some embodiments, a small anti-TM4SF1 antibody or an antigen binding fragment thereof anti-TM4SF1 antibody or an antigen binding fragment thereof comprises a reactive moiety. In some embodiments, a small molecule fragment comprises a reactive moiety selected from a Michael acceptor moiety, a leaving group moiety, or a moiety capable of forming a covalent bond with the thiol group of a cysteine residue.

In some embodiments, the Michael acceptor moiety comprises an alkene or an alkyne moiety. In some embodiments, a small molecule fragment is obtained from a compound library. In some embodiments, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

In some embodiments, a small molecule fragment comprises a carbodiimide, N-hydroxysuccinimide (NHS) ester, imidoester, pentafluorophenyl ester, hydroxymethyl phosphine, maleimide, haloacetyl, pyridyl disulfide, thiosulfonate, vinylsulfone, hydrazide, alkoxyamine, alkyne, azide, or isocyanate group. In some embodiments, a small molecule fragment comprises an alkyne or an azide group. In some embodiments, a small molecule fragment comprises an alkyne group. In some embodiments, a small molecule fragment comprises an azide group.

In some embodiments, a small molecule fragment covalently interacts with a spacer. In some embodiments, the spacer comprises an amide moiety, an ester moiety, an ether moiety, substituted or unsubstituted C₁-C₆ alkylene moiety, substituted or unsubstituted C₁-C₆ haloalkylene moiety, substituted or unsubstituted C₁-C₆ heteroalkylene moiety, substituted or unsubstituted C₃-C₈ cycloalkylene moiety, substituted or unsubstituted C₂-C₇ heterocycloalkylene moiety, substituted or unsubstituted arylene moiety, a substituted or unsubstituted heteroarylene moiety or any combination thereof.

In some embodiments, the linker or a fragments thereof comprises MC (6-maleimidocaproyl), MCC (a maleimidomethyl cyclohexane-1-carboxylate), MP (maleimidopropanoyl), val-cit (valine-citrulline), val-ala (valine-alanine), ala-phe (alanine-phenylalanine), PAB (p-aminobenzyloxycarbonyl), SPP (N-Succinimidyl 4-(2-pyridylthio) pentanoate), SMCC (N-Succinimidyl 4-(N-maleimidomethyl)cyclohexane-1 carboxylate), SIAB (N-Succinimidyl (4-iodo-acetyl)aminobenzoate. Further examples of linkers or fragments thereof include: BS3 ([Bis(sulfosuccinimidyl)suberate]; BS3 is a homobifunctional N-hydroxysuccinimide ester that targets accessible primary amines), NHS/EDC (N-hydroxysuccinimide and N-ethyl-(dimethylaminopropyl)carbodiimide); NHS/EDC allows for the conjugation of primary amine groups with carboxyl groups), sulfo-EMCS ([N-e-Maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-S-acetylthioacetate; SATA is reactive towards amines and adds protected sulfhydryls groups). To form covalent bonds, a chemically reactive group a wide variety of active carboxyl groups (e.g., esters) where the hydroxyl moiety is physiologically acceptable at the levels required to modify the peptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA) maleimido hexanoic acid (MHA), and maleimido undecanoic acid (MUA). Primary amines are the principal targets for NHS esters. Accessible a-amino groups present on the N-termini of proteins and the ε-amine of lysine react with NHS esters. An amide bond is formed when the NHS ester conjugation reaction reacts with primary amines releasing N-hydroxysuccinimide. These succinimide containing reactive groups are herein referred to as succinimidyl groups. In certain embodiments of the disclosure, the functional group on the protein will be a thiol group and the chemically reactive group will be a maleimido-containing group such as gamma-maleimide-butrylamide (GMBA or MPA). Such maleimide containing groups are referred to herein as maleimido groups. The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl can be formed.

In other embodiments, the linker or a fragment thereof includes at least one amino acid (e.g., a peptide of at least 2, 3, 4, 5, 6, 7, 10, 15, 20, 25, 40, or 50 amino acids). In certain embodiments, the linker or a fragment thereof is a single amino acid (e.g., any naturally occurring amino acid such as Cys or Lys). In other embodiments, a glycine-rich peptide such as a peptide can be used. In some cases, the linker or fragments thereof can be a single amino acid (e.g., any amino acid, such as Gly or Cys or Lys). Examples of suitable linkers or fragments thereof are succinic acid, Lys, Glu, and Asp, or a dipeptide such as Gly-Lys. When the linker or a fragment thereof is succinic acid, one carboxyl group thereof may form an amide bond with an amino group of the amino acid residue, and the other carboxyl group thereof may, for example, form an amide bond with an amino group of the peptide or substituent. When the linker or a fragment thereof is Lys, Glu, or Asp, the carboxyl group thereof may form an amide bond with an amino group of the amino acid residue, and the amino group thereof may, for example, form an amide bond with a carboxyl group of the substituent. When Lys is used as the linker or fragments thereof, a further linker or fragments thereof may be inserted between the ε-amino group of Lys and the substituent. In one particular embodiment, the further linker or a fragment thereof is succinic acid which, e.g., forms an amide bond with the ε-amino group of Lys and with an amino group present in the substituent. In one embodiment, the further linker or a fragment thereof is Glu or Asp (e.g., which forms an amide bond with the ε-amino group of Lys and another amide bond with a carboxyl group present in the substituent), that is, the substituent is a NE-acylated lysine residue. In some embodiments, a linker or a fragment thereof comprises a single-amino acid peptide consisting of a lysine. In some embodiments, a linker or a fragment thereof comprises a LysLys dipeptide. In some embodiments, a linker or a fragment thereof comprises a *Lys and/or Lys* dipeptide. In some embodiments, a linker or a fragment thereof comprises a LysLys* and/or*LysLys, Lys*Lys tripeptide. In some embodiments, a linker or a fragment thereof comprises a LysLysLys tripeptide.

The linker comprises a first fragment. In some embodiments, the first fragment is -Phe-Lys-, -Gly-Gly-Gly-Gly-, -Gly-Gly-Phe-Gly-, —X—X—, —X—X—X—, —X—X—X—X—, wherein each of Phe, Lys, and Gly is independently of a D- or L-configuration, and wherein each X is independently a natural amino acid of a D- or L-configuration. In some embodiments, each X is independently a natural amino acid of a D- or L-configuration, or an unnatural amino acid. The linker can be a di-, tri- or tetra-peptide. Each of the amino acid in the linker can be D- or L-configuration.

In some embodiments, the first fragment is

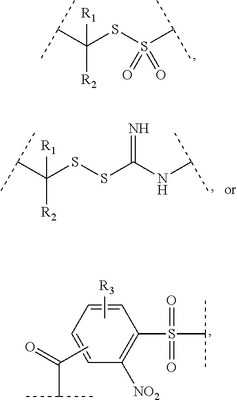

wherein:
each of Phe, Lys, and Gly is independently of a D- or L-configuration;
each X is independently a natural amino acid of a D- or L-configuration;
R₁ is H, deuterium, C₁-C₆ alkyl or C₃-C₆ cycloalkyl;
R₂ is H, deuterium, C₁-C₆ alkyl or C₃-C₆ cycloalkyl; and
R₃ is H, halide, —CN, —CF₃, amino, —OH, —SH, C₁-C₆ alkyl, C₃-C₆ cycloalkyl, C₁-C₆ alkoxy, C₁-C₆ alkylthio, C₂-C₆ alkenyl, C₂-C₆ alkynyl, C₆-C₁₂ aryl, 3-12 membered heteroalicyclic, 5-12 membered heteroaryl, —NR¹⁰R¹¹, —(CR¹²R¹³)ₙOR¹⁰, —C(O)R¹⁰, —O(CO)R¹⁰, —O(CR¹²R¹³)ₙR¹⁰, —OCR¹²R¹³(CR¹²R¹³)ₙNR¹R₁₁, —OCR¹²R¹³(CR¹²R¹³)ₙOR¹⁰, —NR¹⁰C(O)R¹¹, —(CR¹²R¹³)ₙC(O)OR¹⁰, —(CR¹²R¹³)ₙC(O)NR¹⁰R₁₁, —(CR¹²R¹³)ₙNR¹⁰R¹¹, —NR¹⁰ (CO)NR¹⁰R¹¹, —NR¹⁰S(O)ₚR¹¹, —C(O)NR¹⁰, —S(O)ₜR¹⁰, or —S(O)₂NR¹⁰R¹¹;
each R¹⁰, R¹¹, R¹², and R¹³ is independently H, C₁-C₆ alkyl; C₆-C₁₂ aryl, 5-12 membered heteroaryl, C₃-C₁₂ cycloalkyl or 3-12 membered heteroalicyclic; or any two of R¹⁰, R¹¹, R¹², and R¹³ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from the group consisting of N, O, and S; or any two of R¹⁰, R¹¹, R¹², and R¹³ bound to the same carbon atom may, together with the carbon to which they are bound, be combined to form a C₆-C₁₂ aryl, 5-12 membered heteroaryl, C₃-C₁₂ cycloalkyl, or 3-12 membered heteroalicyclic group;
each n is independently 0, 1, 2, 3, or 4;
each p is independently 1 or 2; and
each t is independently 0, 1, or 2.

In some embodiments, the first fragment is

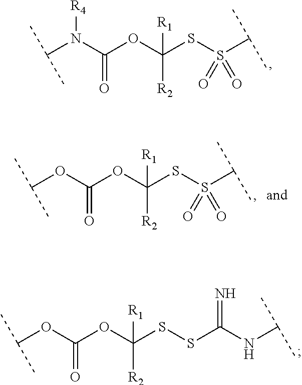

wherein R₄ is H, deuterium, C₁-C₆ alkyl, C₃-C₆ cycloalkyl, C₁-C₆ alkyl; C₆-C₁₂ aryl, 5-12 membered heteroaryl, C₃-C₁₂ cycloalkyl or 3-12 membered heteroalicyclic, or R₄ together with the nitrogen to which they are bound and another atom of the linker, be combined to form a 3 to 12 membered heteroalicyclic or 5-12 membered heteroaryl group optionally containing 1 to 3 additional heteroatoms selected from the group consisting of N, O, and S. In some embodiments, the first fragment is

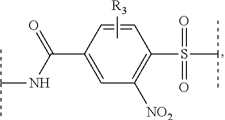

In some embodiments, the first fragment is cleavable.

In some embodiments, the linker comprises a second fragment. In some embodiments, the second fragment is non-cleavable.

In some embodiments, the conjugation of anti-TM4SF1 antibody or an antigen binding fragment thereof and the RNA molecules is carried out in a manner to produce a ring threaded molecule. In some embodiments, the spacer additionally comprises a macrocycle. In some embodiments, the macrocycle comprises a non-covalent macrocycle. In some embodiments, the macrocycle comprises a covalent macrocycle.

In some embodiments, the macrocycle comprises cucurbit[X]uril, wherein X is 5, 6, 7, 8, 9, 10, 11, 12, 13, 14, 15, 16, 17, 18, 19, or 20. In some embodiments, the macrocycle comprises cucurbit[X]uril, wherein X is 5, 6, 7, 8, 9, 10, 11, 12, 13, 14, or 15. In some embodiments, the macrocycle comprises cucurbit[X]uril, wherein X is 5, 6, 7, or 8. In some embodiments, the cucurbit[X]uril has a structure represented by:

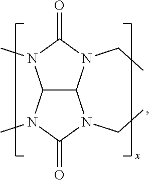

wherein x is 5, 6, 7, or 8.

In some embodiments, x is 5. In some embodiments, x is 6. In some embodiments, x is 7. In some embodiments, x is 8.

In some embodiments, the macrocycle comprises cucurbit[6]uril (CB6). In some embodiments, the macrocycle comprises cucurbit[7]uril (CB7). In some embodiments, the cucurbit[7]uril has a structure represented by:

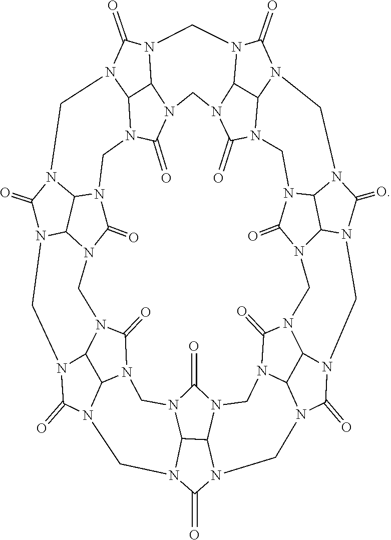 [SMILES: O=C1N2CN3C(=O)N4CN5C(=O)N6CN7C(=O)N8CN9C(=O)N%10CN%11C(=O)N%12CN%13C(=O)N%14CN1C1C2N2CN%15C(=O)N(CN%16C(=O)N(CN%17C(=O)N(CN%18C(=O)N(CN%19C(=O)N(CN%20C(=O)N(CN1C2=O)C%14C%13%20)C%12C%11%19)C%10C9%18)C8C7%17)C6C5%16)C4C3%15]

In some embodiments, the macrocycle comprises a cyclodextrin (CD). In some embodiments, the cyclodextrin has a structure represented by:

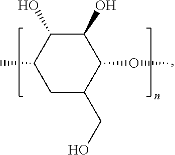

wherein n is 5, 6, 7, or 8.

In some embodiments, the macrocycle comprises a beta-cyclodextrin (n=7). In some embodiments, macrocycle comprises a gamma-cyclodextrin (n=8). In some embodiments, the beta-cyclodextrin has a structure represented by:

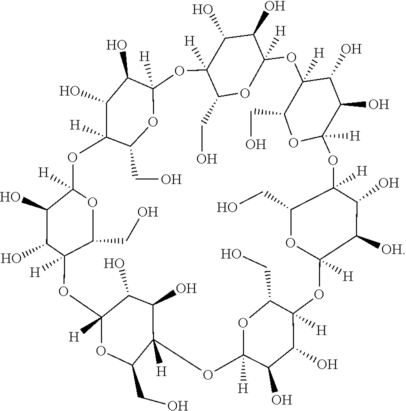 [SMILES: OC[C@H]1O[C@@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO]

In some embodiments, the macrocycle comprises a polypeptide. In some embodiments, the polypeptide has a structure represented by:

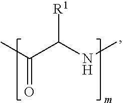

wherein
R¹ is H, D, F, —CN, substituted or unsubstituted C₁-C₆ alkyl, substituted or unsubstituted C₁-C₆fluoroalkyl, substituted or unsubstituted C₁-C₆heteroalkyl, substituted or unsubstituted C₃-C₈ cycloalkyl, substituted or unsubstituted aryl, and substituted or unsubstituted heteroaryl; and m is 5, 6, 7, or 8.

In some embodiments, the macrocycle comprises a cycloglycine. In some embodiments, the macrocycle comprises cyclo(glycylglycylglycylglycyglycyllglycyl). In some embodiments, the macrocycle comprises cyclo(glycylglycylglycylglycylglycylglycylglycyl). In some embodiments, the cyclo(glycylglycylglycylglycylglycylglycylglycyl) has a structure represented by:

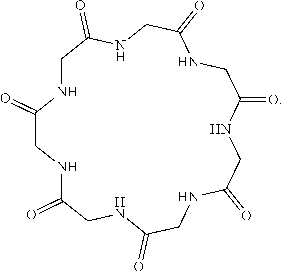 [SMILES: O=C1CNC(=O)CNC(=O)CNC(=O)CNC(=O)CNC(=O)CNC(=O)CN1]

In some embodiments, the macrocycle comprises a crown ether. In some embodiments, the crown ether is a 15-crown-5, 18-crown-6, dibenzo-18-crown-6, or diaza-18-crown-6.

In some embodiments, the macrocycle comprises a cycloalkane. In some embodiments, the cycloalkane is a cyclopentadecane, cyclohexadecane, cycloheptadecane, or cyclooctadecane.

In some embodiments, the macrocycle comprises cyclobis(paraquat-p-phenylene) (CBPQT⁴). In some embodiments, the cyclobis(paraquat-p-phenylene) (CBPQT⁴*) has a structure represented by:

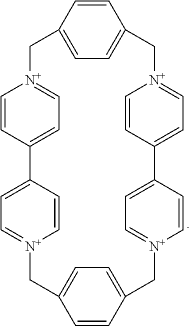 [SMILES: c1cc2ccc1C[n+]1ccc(cc1)-c1cc[n+](cc1)Cc1ccc(cc1)C[n+]1ccc(cc1)-c1cc[n+](cc1)C2]

In some embodiments, a linker or a fragments thereof comprise quaternary nitrogen. In some embodiments, the linker or a fragment thereof is:

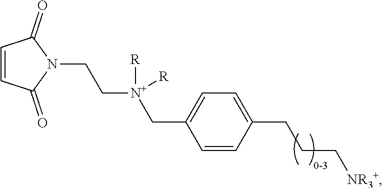

wherein each R is independently H or C1-C6 alkyl. In some embodiments, the linker or a fragment thereof is:

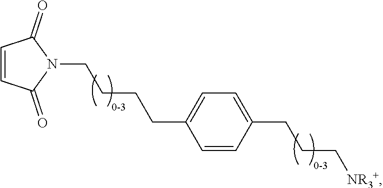

wherein each R is independently H or C1-C6 alkyl. In some embodiments, the linker or a fragment thereof is:

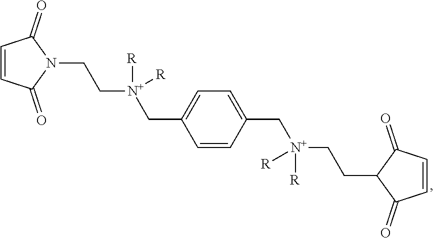

wherein
each R is independently H or C1-C6 alkyl.

In some embodiments, the linker or a fragment thereof is:

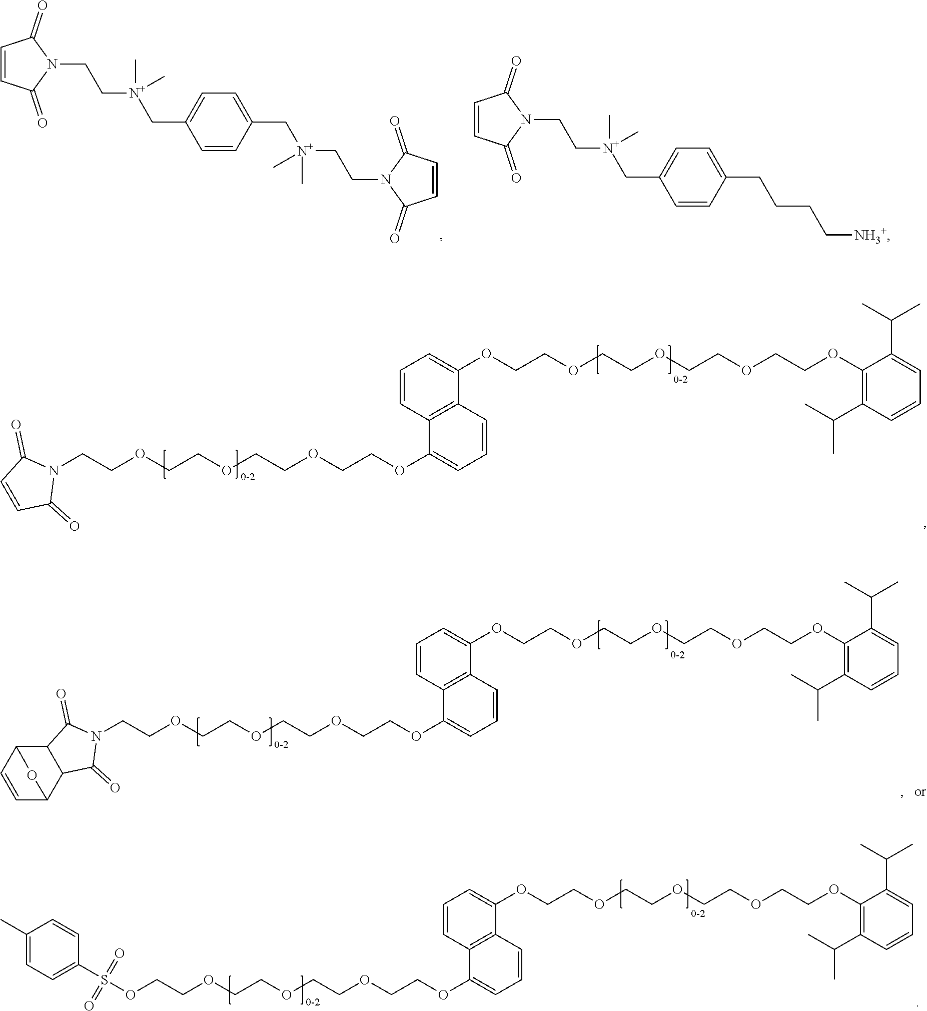

In some embodiments, the conjugates are produced by linking a first portion of the linker to the anti-TM4SF1 antibody or the antigen binding fragment thereof and a second portion of the linker to the oligonucleotide. Conjugating the linker to anti-TM4SF1 antibody or an antigen binding fragment thereof or the therapeutic molecule may comprise production of an ionic bond, a covalent bond, a non-covalent bond or a combination thereof between the linker and the antibody, antigen binding fragment thereof or therapeutic agent. Conjugating the linker to the anti-TM4SF1 antibody or the antigen binding fragment thereof or the oligonucleotide may, in some cases, be performed as described in Roberts et al., Advanced Drug Delivery Reviews 54:459-476 (2002). The linker may be selected from a bifunctional linker, a cleavable linker, a non-cleavable linker, an ethylene glycol linker, a bifunctional ethylene glycol linker, a flexible linker, or an inflexible linker. The linker may comprise a chemical group selected from a cyclooctyne, a cyclopropene, an aryl/alkyl azide, a trans-cyclooctene, a norborene, and a tetrazine. In some embodiments, a terminus of the linker comprises an alkoxy-amine. In some embodiments, a terminus of the linker comprises an azide or cyclooctyne group. In some embodiments, the antibody or antibody fragment or therapeutic agent may be coupled to the linker by a chemical group selected from a cyclooctyne, a cyclopropene, aryl/alkyl azide, trans-cyclooctene, norborene, and tetrazine. Linking anti-TM4SF1 antibody or an antigen binding fragment thereof or an oligonucleotide to the linker may comprise conducting one or more copper-free reactions. Linking the antibody or antibody fragment or an oligonucleotide to the linker may comprise conducting one or more copper-containing reactions. Linking the anti-TM4SF1 antibody or the antigen binding fragment thereof or an oligonucleotide to the linker may comprise one or more cycloadditions. Linking anti-TM4SF1 antibody or an antigen binding fragment thereof or an oligonucleotide to the linker may comprise one or more Huisgen-cycloadditions. Linking the anti-TM4SF1 antibody or the antigen binding fragment thereof or an oligonucleotide to the linker may comprise one or more Diels Alder reactions. Linking anti-TM4SF1 antibody or an antigen binding fragment thereof or an oligonucleotide to the linker may comprise one or more Hetero Diels Alder reaction. In some embodiments, a terminus of the linker comprises a leaving group. Linking fragments of the linker may rely on the same or similar chemical reactions as well.

In some embodiments, a first portion of the linker covalently interacts with a cysteine containing anti-TM4SF1 antibody or an antigen binding fragment thereof, as described herein. In some embodiments, a first portion of the linker covalently interacts with a cysteine containing TM4SF1 antibody or an antigen binding fragment thereof, as described herein. In some embodiments, an oligonucleotide described herein covalently interacts with a second portion of the linker. In some embodiments, an oligonucleotide described herein non-covalently interacts with a second portion of the linker.

In some embodiments, the conjugated ADCs can be de-activated by lysosomal de-activation as shown in Scheme 1.

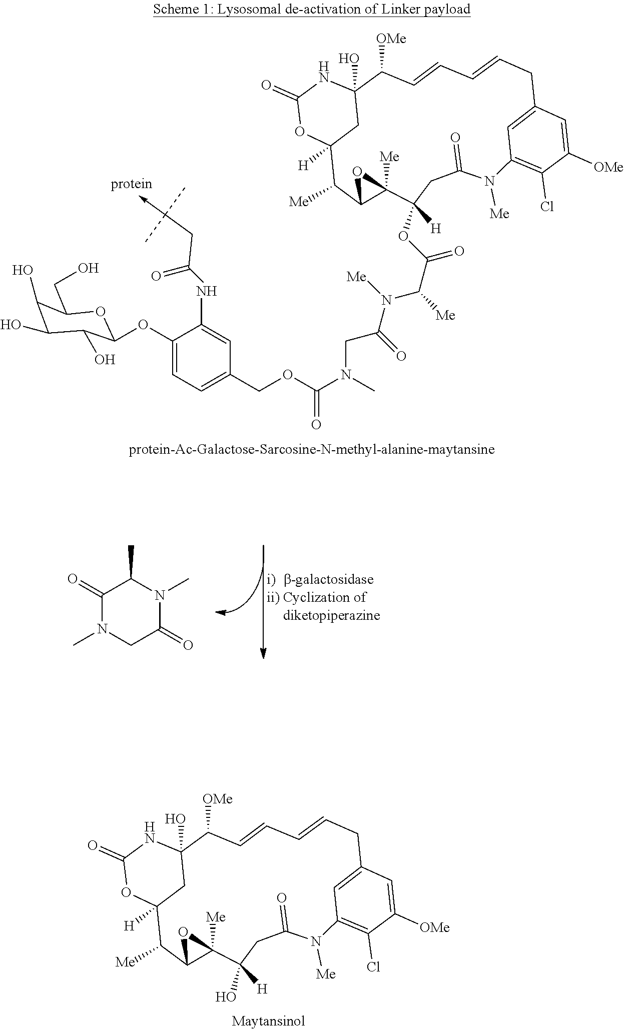

β-Glycosidic bond can be cleaved by β-galactosidase, which is present mainly or substantially, if not only, in the lysosomes. The bromoacetamide-conjugated-Galactose-Maytansine linker payload is designed to cleave upon lysosomal internalization, thereby leading to the release of maytansinol which is a less potent, known metabolite of maytansine and maytansine-containing ADCs. β-galactosidase expression is upregulated in cancer cells over healthy cells. If potency is retained, then the maximum tolerated dose (MTD) can be increased. MTD is the highest dose of a drug or treatment that does not cause unacceptable side effects. The MTD is determined in clinical trials by testing increasing doses on different groups of people until the highest dose with acceptable side effects is found. Maytansinol is a known metabolite of trastuzumab emtansine or Kadcyla. It has been reported 7% found in plasma every 2 weeks.

In some embodiments, a viral protein p19 based siRNA carrier is contemplated, which protein has been shown to have a high affinity for siRNA. See, e.g., Yang et al. Cytosolic delivery of siRNA by ultra-high affinity dsRNA binding proteins, Nucleic Acids Res. 2017 Jul. 27; 45(13): 7602-7614. In some examples, a p19-siRNA complex is generated and fused to an anti-TM4SF1 antibody or antigen-binding fragment thereof. In additional embodiments, a statistical or random conjugation methods via Cys, Lys, or Arginine residues within the antibody or antigen binding fragment thereof.

Synthesis of an ADC Comprising an Anti-TM4SF1 Antibody or an Antigen Bindingfragment Thereof and an siRNA

In one embodiment, a conjugate comprising an anti-TM4SF1 antibody or an antigen binding fragment thereof and an oligonucleotide is developed by covalent conjugation of the antibody or antigen binding fragment and the RNA molecule (e.g., siRNA). As a first step of such an exemplary process, an engineered anti-TM4SF1 antibody is generated, in which a cysteine residue had been introduced in the heavy chain (thereby producing an anti-TM4SF1 HC THIOMAB). The anti-TM4SF1 thiomab, in some examples, provides at least two discrete positions for coupling with an RNA molecule, such as with an siRNA. For instance, one siRNA molecule can be coupled to each heavy chain of the anti-TM4SF1 thiomab. In a separate or subsequent step in the conjugation process, a chemically stabilized siRNA (synthesized, e.g., using siSTABLE chemistry) modified with a 3′-amine for coupling to the passenger strand with a sequence targeting 116roteasomel 16rro isomerase B (PPIB, cyclophilin B) is generated. The conjugation, in some embodiments, further involves a reducible N-succinimidyl-4-(2-pyridyldithio)butyrate (SPDB) or a non-reducible succinimidyl-4-[N-maleimidomethyl]cyclohexane-1-carboxylate) (SMCC) NHS (N-hydroxysuccinimide) linkers. In some embodiments, using the anti-TM4SF1 thiomab, an exemplary conjugate molecule according to this disclosure is generated in a multi-step process involving at least two primary steps: (i) reaction of an amine-tagged siRNA with an NHS-linker to form a thiol-reactive siRNA-linker adduct, and (ii) reacting the adduct with thiol groups on the THIOMAB to covalently link the siRNA via a thio-ester bond. The exemplary ADC is subsequently purified using anion exchange chromatography to remove free siRNA and then by size-exclusion chromatography to remove un-coupled antibody. Further techniques, such as gel electrophoresis and electrospray TOF mass spectrometry can then be used to assess the yield of the exemplary ADC, as well characteristics such as monomeric conjugates with one or two
linked siRNAs per antibody. Additional methods that can be employed for the conjugation involve the use or chemical or peptide based linkers, chemical or enzymatic conjugation methods (e.g., using mammalian or bacterial transglutaminase), or any combinations thereof Δny of the linkers and/or methods described above can be used to couple the anti-TM4SF1 antibody or the antigen binding fragment thereof and the oligonucleotides of the conjugate.

Using appropriate coupling methods, it is possible to generate ADCs of this disclosure, which comprise, for example, an anti-TM4SF1 antibody or an antigen binding fragment thereof to oligonucleotide ratio of about 1:2, 1:3, 1:4, 1:5, 1:6, 1:7, 1:8, 1:9, or 1:10, or higher. In some embodiments, the ADC comprises an anti-TM4SF1 antibody or an antigen binding fragment thereof to oligonucleotide ratio of 1:1. This can be achieved, for example, by using an antigen binding fragment or a portion of an antibody, e.g., a half-antibody, Fab, or other fragments that comprise a THIOMAB engineered cysteine. In some examples, the ADC can be designed to comprise 1:1 ratios of an anti-TM4SF1 antibody or an antigen binding fragment thereof to oligonucleotide using a whole antibody which is conjugated to an oligonucleotide by a conjugation method that utilize a multimetallic protein (e.g., a hexa-rhodium metallopeptide) to enable modification of proteins, on the basis of molecular recognition. For example, the anti-TM4SF1 antibody or the antigen binding fragment thereof and the oligonucleotide can be conjugated using a site-specific antibody functionalization, based on molecular recognition of the Fc domain constant region of the antibody by the multimetallic protein. In some embodiments, the multimetallic protein comprises three rhodium complexes attached to specific sites of a protein that binds to the Fc domain of an antibody. Upon binding, the multimetallic protein can catalyze site-specific conjugation of the oligonucleotide to the antibody. An advantage of using the multimetallic protein can be that the antibody is minimally disrupted, such as by avoiding engineering residues within the antibody, during the conjugation.

### Non-Cleavable Linkers

In some embodiments, a non-cleavable linker is between the anti-TM4SF1 antibody or the antigen-binding fragment thereof and the therapeutic agent. In some embodiments, the non-cleavable linker is a covalent linker. In some embodiments, the non-cleavable linker is attached to the N-terminus, C-terminus or an internal amino acid position of the anti-TM4SF1 antibody or an antigen-binding fragment thereof. In some embodiments, the non-cleavable linker is covalent attached to the therapeutic agent. In some embodiments, the non-cleavable linker is covalent attached to a heteroatom comprising N, O, or S or a carbon atom of a C₁-C₆ alkyl, alkenyl, alkynyl, C₃-C₁₂ cycloalkyl, C₅-C₁₂ cycloalkenyl, C₈-C₁₆ cycloalkynyl, C₆-C₁₂ aryl, 5-12 membered heteroaryl, or 3-12 membered heteroalicyclic group of the therapeutic agent.

In some embodiments, the non-cleavable linker comprises C₁-C₆ alkylene, alkenylene, cycloalkylene with a 3-7 membered ring, alkynylene, arylene, heteroarylene, heterocyclene with a 5-12 membered ring comprising 1-3 atoms of N, O or S, —O—, —NH—, —S—, —N(C₁₋₆ alkyl)-, —C(═O)—, —C(═O)NH—, or combinations thereof, wherein the C₁-C₆ alkylene, alkenylene, cycloalkylene a 3-7 membered ring, arylene, heteroarylene, and heterocyclene with a 5-12 membered ring comprising 1-3 atoms of N, O or S is unsubstituted or substituted with halide, amino, —CF₃, C₁-C₃ alkyl, C₃-C₆ cycloalkyl, C₁-C₃ alkoxy, C₁-C₃ alkoxy, or C₁-C₃ alkylthio.

In some embodiments, the non-cleavable linker comprises

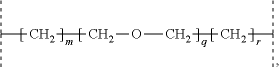

wherein m is 0-3, q is 0-12, and r is 1-3.

In some embodiments, the non-cleavable linker comprises

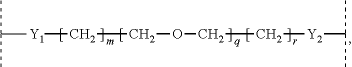

wherein:
each Y₁ and Y₂ is independently a bond, O, S, or NR₆;
R₆ is independently H, deuterium, C₁-C₆ alkyl, C₃-C₆ cycloalkyl, C₁-C₆ alkyl; C₆-C₁₂ aryl, 5-12 membered heteroaryl, C₃-C₁₂ cycloalkyl or 3-12 membered heteroalicyclic, or R₆ together with the nitrogen to which R₆ is bound and another atom of the non-cleavable linker, the anti-TM4SF1 antibody or the antigen-binding fragment thereof, or the therapeutic agent/molecule, be combined to form a 3 to 12 membered heteroalicyclic or 5-12 membered heteroaryl group optionally containing 1 to 3 additional heteroatoms selected from the group consisting of N, O, and S; and m is 0-3, q is 0-12, and r is 1-3.

### VI. Polynucleotides

Also provided, in some embodiments, are polynucleotides encoding an anti-TM4SF1 antibody or an antigen binding fragment thereof. In some embodiments, the polynucleotide molecules are provided as a DNA construct. In other embodiments, the polynucleotide molecules are provided as a messenger RNA transcript.

In some examples, an anti-TM4SF1 antibody of the present disclosure comprises a heavy chain variable domain encoded by a nucleic acid sequence as set forth in any one of SEQ ID NOs: 4, 16, 28, 40, 52, 64, or 76. In some examples, an anti-TM4SF1 antibody of the present disclosure comprises a light chain variable domain encoded by a nucleic acid sequence as set forth in any one of SEQ ID NOs: 10, 22, 34, 46, 58, 70, or 82.

In some embodiments are provided nucleic acid sequences that are codon optimized for expression in a host cell, e.g., a bacterium, such as E. coli, or a eukaryotic cell, such as a CHO cell. In some examples, the nucleic acid sequences are codon optimized for expression in CHO cells. In some examples, an anti-TM4SF1 antibody of the present disclosure comprises a heavy chain variable domain encoded by a codon optimized nucleic acid sequence as set forth in any one of SEQ ID NOs: 5, 17, 29, 41, 53, 65, or 77. In some examples, an anti-TM4SF1 antibody of the present disclosure comprises a light chain variable domain encoded by a codon optimized nucleic acid sequence as set forth in any one of SEQ ID NOs: 11, 23, 35, 47, 59, 71, or 83. In certain instances, the nucleic acid sequence of any one of SEQ ID NOs: 5, 17, 29, 41, 53, 65, or 77 is a nucleic acid sequence codon optimized for expression in CHO cell. In certain instances, the nucleic acid sequence of any one of SEQ ID NOs: 11, 23, 35, 47, 59, 71, or 83 is a nucleic acid sequence codon optimized for expression in CHO cell.

The polynucleotide molecules are constructed by known methods such as by incorporating the genes encoding the binding proteins into a genetic construct linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and inducible promoters, may be used. The promoter is selected such that it drives the expression of the polynucleotide in the respective host cell.

In some embodiments, a polynucleotide as described herein is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described TM4SF1 binding protein. Examples of expression vectors for expression in E. coli are pSKK (Le Gall et al., J Immunol Methods. (2004) 285(1):111-27) or pcDNA5 (Invitrogen) for expression in mammalian cells.

Thus, the TM4SF1 binding proteins as described herein, in some embodiments, are produced by introducing a vector encoding the protein as described above into a host cell and culturing said host cell under conditions whereby the protein domains are expressed, may be isolated and, optionally, further purified.

### VI. Methods of Treatment

The disclosure further provides a method for inhibiting cell-cell interactions that are endothelial cell (EC) specific, for example, but not limited to EC-EC, EC-mesenchymal stem cell, EC-fibroblast, EC-smooth muscle cell, EC-tumor cell, EC-leukocyte, EC-adipose cell and EC-neuronal cell interactions. In certain embodiments, the ADCs containing the anti-TM4SF1 antibodies and fragments of the present disclosure, can be used to treat any human disease or disorder with a pathology that is characterized by abnormal EC-cell interactions. In certain embodiments, the EC-cell interaction is an EC-leukocyte interaction, where inhibition of the EC-leukocyte interaction is used to prevent inflammation.

In other embodiments, the disclosure features a method of treating or preventing a disease or disorder in a subject, wherein the disease or disorder is characterized by abnormal endothelial cell (EC)-cell interactions, the method comprising administering the antibody, or antigen-binding fragment thereof, as described herein. In certain embodiments, the EC-cell interactions include one or more of EC-mesenchymal stem cell, EC-fibroblast, EC-smooth muscle cell, EC-tumor cell, EC-leukocyte, EC-adipose cell and EC-neuronal cell interactions. In exemplary embodiments, the disease is an inflammatory disease or disorder, and the antibodies and fragments of the disclosure are used to inhibit EC-leukocyte interactions. In another exemplary embodiment, the disease or disorder is selected from an inflammatory disease or cancer. The adhesion of leukocytes to vascular endothelium is a hallmark of the inflammatory process. Accordingly, in one embodiment, an ADC containing an anti-TM4SF1 antibody, or an antigen binding fragment thereof, of the present disclosure is used to treat an inflammatory disease in which inhibiting leukocyte attachment to endothelial cells, or leukocyte transmigration across the endothelium is helpful for treatment (see, e.g., Rychly et al., Curr Pharm Des. 2006; 12(29):3799-806, incorporated by reference in its entirety herein). Examples include, but are not limited to, sepsis, inflammatory bowel disease, psoriasis or multiple sclerosis.

Each year approximately half a million patients die from cancer in the United States alone. Tumor metastasis is responsible for ˜90% of these deaths. No therapy that blocks metastasis is known. The present disclosure provides antibodies, and antigen-binding fragments thereof, that can treat cancer and inhibit metastatic cells based on immunoblockade of tumor cell (TC)-endothelial cell (EC) interactions mediated by a novel target, TM4SF1.

As described above, TM4SF1 is a small, tetraspanin-like, cell surface glycoprotein originally discovered as a TC antigen with roles in TC invasion and metastasis. TM4SF1 is selectively expressed by TCs and ECs. TM4SF1 is expressed at low levels on the vascular ECs supplying normal tissues in both mice and humans. It has been shown that TM4SF1 is expressed at ˜10-20 fold higher levels on the vascular ECs lining the blood vessels supplying many human cancers, and at equivalent high levels on cultured ECs. TM4SF1-enriched microdomains (TMED) recruit cell surface proteins like integrins to assist the formation of nanopodia, thin membrane channels that extend from the cell surface and mediate cell-cell interactions. Thus, in certain instances, ADCs containing anti-TM4SF1 antibodies and fragments described herein interfere with nanopodia-mediated interactions and inhibit TC interactions with EC that are necessary for TC extravasation.

ADCs of this disclosure may be formulated for treating a subject (e.g., a human) having a disorder associated with pathological angiogenesis (e.g., cancer, such as breast cancer, ovarian cancer, renal cancer, colorectal cancer, liver cancer, gastric cancer, and lung cancer; obesity; macular degeneration; diabetic retinopathy; psoriasis; rheumatoid arthritis; cellular immunity; and rosacea.

TM4SF1 is highly expressed on the surface of most epithelial TCs, and is also highly expressed on the EC lining tumor blood vessels and on cultured EC. It is expressed at ˜10-20 fold lower levels on the surface of normal vascular ECs. In mouse models, tumor metastasis to lungs is related to TM4SF1 expression on both ECs and TCs. Metastasis requires initial attachment of TC to vascular EC and their subsequent migration across ECs to enter the lung or other metastatic sites. The examples below show that, in some instances, the anti-TM4SF1 antibodies of the present disclosure interfere with TC-EC interactions in culture and can also inhibit tumor metastasis in vivo.

Thus, the ADCs of the present disclosure can be used to block one or both of the earliest steps in metastasis, namely, TC attachment to vascular ECs and/or transmigration of TCs across ECs, and thereby prevent or substantially reduce the number of metastases in at risk cancer patients.

The present disclosure further provides a method for preventing metastasis. Human tumors typically shed TCs into the blood and lymphatics at early stages of growth; hence, early treatment of primary tumors provides no guarantee that metastasis has not already taken place. Thus, immunoblockade of TM4SF1 can be used to treat or prevent hematogenous metastases or to treat or prevent lymphatic metastases.

The methods of this disclosure are, in some embodiments, directed to inhibiting metastatic cells in a subject. In one embodiment, the subject has a cancer, e.g., a cancer that is associated with metastasis or a cancer that has already metastasized. In other embodiments, the subject was already treated for cancer and is in remission or partial remission, wherein the benefits of administering ADCs containing the anti-TM4SF1 antibodies or fragments described herein are that they work to prevent metastasis and maintain remission or partial remission.

In certain embodiments, the disclosure provides a method of treating a person having a greater risk of developing metastasis, wherein administration of the ADCs containing the anti-TM4SF1 antibodies and fragments described herein can be used to inhibit or delay onset of metastasis.

Included in the disclosure is a method of blocking tumor metastasis, particularly metastasis to the lung, by administering an anti-TM4SF1 antibody to a subject in need thereof. In some examples, the anti-TM4SF1 antibody is a human anti-TM4SF1 antibody, also referred to herein as anti-hTM4SFT. In certain embodiments, the methods can include administration of an effective amount of an ADC containing an anti-hTM4SF1 antibody to a subject in need thereof, wherein the effective amount of the antibody prevents tumor cell (TC) attachment to and migration across vascular endothelial cells (ECs).

In certain embodiments, an ADC containing an anti-TM4SF1 antibody is administered to a subject having cancer or at risk of having metastasis such that the dose amount and frequency maintains long term TM4SF1 immunoblockade. The dosing regimen will maximally inhibit TM4SF1-mediated metastasis by administering an ADC containing an anti-TM4SF1 antibody to a subject in an amount sufficient to saturate TM4SF1 expressed on normal vascular ECs of the subject.

In certain embodiments, the effective amount of an ADC containing an anti-TM4SF1 antibody, or an antigen binding fragment thereof, that is administered is an amount sufficient to, at one week, achieve circulating antibody concentrations >1 μg/ml.

In certain embodiments, the effective amount of an ADC containing an anti-TM4SF1 antibody, or an antigen binding fragment thereof that is administered is an amount sufficient to maintain serum concentrations of the antibody at or above 1 μg/ml continuously for about 1 month.

In one embodiment, the disclosure provides a method of treating or preventing metastasis in a human subject comprising administering to the subject an effective amount of an ADC containing an anti-TM4SF1 antibody, or an antigen binding fragment thereof, wherein the effective amount of the antibody, or antigen binding fragment thereof, comprises 1 to 80 mg/kg of the amount of the antibody, or antigen binding fragment thereof.

The mode of administration for therapeutic use of the ADCs of the disclosure may be any suitable route that delivers the antibody to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intra-articular, intrabronchial, intraabdominal, intracapsular, intracartilaginous, intracavitary, intracellular, intracerebellar, intracerebroventricular, intracolic, intracervical, intragastric, intrahepatic, intracardial, intraosteal, intraosseous, intrapelvic, intrapericardial, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

In some embodiments, the ADCs of the disclosure may be administered to a subject by any suitable route, for example parentally by intravenous (i.v.) infusion or bolus injection, intramuscularly or subcutaneously or intraperitoneally. i.v. infusion may be given over for example 15, 30, 60, 90, 120, 180, or 240 minutes, or from 1, 2, 3, 4, 5, 6, 7, 8, 9, 10, 11 or 12 hours. The dose given to a subject in some embodiments is about 0.005 mg to about 100 mg/kg, e.g., about 0.05 mg to about 30 mg/kg or about 5 mg to about 25 mg/kg, or about 4 mg/kg, about 8 mg/kg, about 16 mg/kg or about 24 mg/kg, or for example about 0.1, 0.2, 0.3, 0.4, 0.5, 0.6, 0.7, 0.8, 0.9, 1, 2, 3, 4, 5, 6, 7, 8, 9 or 10 mg/kg. In certain embodiments, the dose given to a subject is, for example about 15, 16, 17, 18, 19, 20, 21, 22, 23, 24, 25, 30, 40, 50, 60, 70, 80, 90 or 100 mg/kg. In some instances, the dose of the antibodies of the disclosure given to a subject may be about 0.1 mg/kg to 10 mg/kg via intravenous administration. In some instances, the dose of the antibodies of the disclosure given to a subject is about 0.1 mg/kg to 10 mg/kg via subcutaneous administration. In some instances, the dose of the antibodies of the disclosure given to a subject is about 0.1 mg/kg via intravenous administration. In some instances, the dose of the antibodies of the disclosure given to a subject is about 0.1 mg/kg via subcutaneous administration. In some embodiments, the dose of the antibodies of the disclosure given to a subject is about 0.3 mg/kg via intravenous administration. In some examples, the dose of the antibodies of the disclosure given to a subject is about 0.3 mg/kg via subcutaneous administration. In some examples, the dose of the antibodies of the disclosure given to a subject is about 1.0 mg/kg via intravenous administration. In some examples, the dose of the antibodies of the disclosure given to a subject is about 1.0 mg/kg via subcutaneous administration. In some examples, the dose of the antibodies of the disclosure given to a subject is about 3.0 mg/kg via intravenous administration. In some examples, the dose of the antibodies of the disclosure given to a subject is about 3.0 mg/kg via subcutaneous administration. In some examples, the dose of the antibodies of the disclosure given to a subject may be about 10.0 mg/kg via intravenous administration. In some examples, the dose of the antibodies of the disclosure given to a subject is about 10.0 mg/kg via subcutaneous administration.

In certain embodiments, a fixed unit dose of the antibodies of the disclosure is given, for example, 50, 100, 200, 500 or 1000 mg, or the dose may be based on the patient's surface area, e.g., 500, 400, 300, 250, 200, or 100 mg/m². In some instances, between 1 and 8 doses, (e.g., 1, 2, 3, 4, 5, 6, 7 or 8) is administered to treat the patient, but 9, 10, 11, 12, 13, 14, 15, 16, 17, 18, 19, 20 or more doses are given.

The administration of the ADCs of the disclosure described herein, in some embodiments, is repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration is at the same dose or at a different dose. In some examples, the ADCs of the disclosure described herein is administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion. Alternatively, in some embodiments, the ADCs of the disclosure described herein are administered at between 0.1 mg/kg to about 10 mg/kg at weekly interval for 17 weeks. For example, in some cases the antibodies of the disclosure are provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

In some embodiments, the antibodies of the disclosure described herein is administered prophylactically in order to reduce the risk of developing an inflammatory disease such as RA, psoriatic arthritis or psoriasis, delay the onset of the occurrence of an event in progression of the inflammatory disease such as RA, psoriatic arthritis or psoriasis. In some examples, the ADCs of the disclosure are lyophilized for storage and reconstituted in a suitable carrier prior to use. In some cases, the antibodies of the disclosure are supplied as a sterile, frozen liquid in a glass vial with stopper and aluminum seal with flip-off cap. In some examples, each vial might contain ADC containing 3.3 mL of a 50 mg/mL solution of the antibody (including a 10% overfill) in a formulation of 10 mM histidine, 8.5% (w/v) sucrose, and 0.04% (w/v) Polysorbate 80 at pH 5.8. In some examples, the vials contain no preservatives and are for single use. Vials may be stored frozen and protected from light. To prepare for IV administration, the ADC formulations, in some examples, are filtered with a 0.22 micron filter before being diluted in sterile diluent. In some examples, diluted ADCs at volumes up to approximately 100 mL are administered by IV infusion over a period of at least 30 minutes using an in-line 0.22 micron filter. Alternatively, in some embodiments, the ADCs are administered as 1 or 2 subcutaneous injections containing about 50 mg/mL antibody in about 3.3 mL. The subcutaneous injection site may be, for example, within the abdominal area.

### VIII. Pharmaceutical Compositions

The ADCs of this disclosure, can, in some embodiments, be included in compositions (e.g., pharmaceutical compositions). The pharmaceutical compositions of the disclosure may further include a pharmaceutically acceptable carrier, excipient, or diluent.

The term “pharmaceutical composition” as used herein refers to a composition containing a TM4SF1 binding protein described herein formulated with a pharmaceutically acceptable carrier and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable carrier” as used herein refers to a carrier which is physiologically acceptable to a treated mammal (e.g., a human) while retaining the therapeutic properties of the protein with which it is administered. One exemplary pharmaceutically acceptable carrier is physiological saline. Other physiologically acceptable carriers and their formulations are known to one skilled in the art and described, for example, in Remington's Pharmaceutical Sciences (18^{th} edition, A. Gennaro, 1990, Mack Publishing Company, Easton, PA), incorporated herein by reference.

Pharmaceutical compositions containing an ADC containing an TM4SF1 antibody or antigen-binding fragment thereof, are, in some embodiments, prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof.

A pharmaceutically acceptable excipient is, in some examples, an excipient described in the Handbook of Pharmaceutical Excipients, American Pharmaceutical Association (1986). Non-limiting examples of suitable excipients include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient is a buffering agent. Non-limiting examples of suitable buffering agents include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium gluconate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof is used, in some embodiments, in a pharmaceutical composition of the present disclosure.

In some embodiments an excipient comprises a preservative. Non-limiting examples of suitable preservatives include antioxidants, such as alpha-tocopherol and ascorbate, and antimicrobials, such as parabens, chlorobutanol, and phenol. In some examples, antioxidants further include but are not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical composition as described herein comprises a binder as an excipient. Non-limiting examples of suitable binders include starches, pregelatinized starches, gelatin, 126roteasome126rrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof. The binders used in a pharmaceutical formulation are, in some examples, selected from starches such as potato starch, corn starch, wheat starch; sugars such as sucrose, glucose, dextrose, lactose, maltodextrin; natural and synthetic gums; 126roteas; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or any combinations thereof.

In some embodiments a pharmaceutical composition as described herein comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that are used in a pharmaceutical formulation, in some embodiments, are be selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments a pharmaceutical formulation comprises a dispersion enhancer as an excipient. Non-limiting examples of suitable dispersants include, in some examples, starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical composition as described herein comprises a disintegrant as an excipient. In some embodiments a disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments a disintegrant is an effervescent disintegrant. Non-limiting examples of suitable effervescent disintegrants include sodium bicarbonate in combination with citric acid, and sodium bicarbonate in combination with tartaric acid.

In some embodiments an excipient comprises a flavoring agent. Flavoring agents incorporated into an outer layer are, in some examples, chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical composition as described herein comprises a coloring agent. Non-limiting examples of suitable color agents include food, drug and cosmetic colors (FD&C), drug and cosmetic colors (D&C), and external drug and cosmetic colors (Ext. D&C). A coloring agents can be used as dyes or their corresponding lakes.

In some instances, a pharmaceutical composition as described herein comprises a chelator. In some cases, a chelator is a fungicidal chelator. Examples include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); 0,0′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11]pentatriacontane hexahydrobromide; or triethylenetetramine-N,N,N′,N″,N′″,N′″-hexaacetic acid.

Also contemplated are combination products that include an anti-TM4SF1 antibody as disclosed herein and one or more other antimicrobial or antifungal agents, for example, polyenes such as amphotericin B, amphotericin B lipid complex (ABCD), liposomal amphotericin B (L-AMB), and liposomal nystatin, azoles and triazoles such as voriconazole, fluconazole, ketoconazole, itraconazole, pozaconazole and the like; glucan synthase inhibitors such as caspofungin, micafungin (FK463), and V-echinocandin (LY303366); griseofulvin; allylamines such as terbinafine; flucytosine or other antifungal agents, including those described herein. In addition, it is contemplated that a peptide can be combined with topical antifungal agents such as ciclopirox olamine, haloprogin, tolnaftate, undecylenate, topical 128roteaso, amorolfine, butenafine, naftifine, terbinafine, and other topical agents. In some instances, a pharmaceutical composition comprises an additional agent. In some cases, an additional agent is present in a therapeutically effective amount in a pharmaceutical composition.

Under ordinary conditions of storage and use, the pharmaceutical compositions as described herein comprise a preservative to prevent the growth of microorganisms. In certain examples, the pharmaceutical compositions as described herein do not comprise a preservative. The pharmaceutical forms suitable for injectable use include sterile aqueous solutions or dispersions and sterile powders for the extemporaneous preparation of sterile injectable solutions or dispersions. The pharmaceutical compositions comprise a carrier which is a solvent or a dispersion medium containing, for example, water, ethanol, polyol (e.g., glycerol, propylene glycol, and liquid polyethylene glycol, and the like), and/or vegetable oils, or any combinations thereof. Proper fluidity is maintained, for example, by the use of a coating, such as lecithin, by the maintenance of the required particle size in the case of dispersion and by the use of surfactants. The prevention of the action of microorganisms is brought about by various antibacterial and antifungal agents, for example, parabens, chlorobutanol, phenol, sorbic acid, thimerosal, and the like. In many cases, isotonic agents are included, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the liquid dosage form is suitably buffered if necessary and the liquid diluent rendered isotonic with sufficient saline or glucose. The liquid dosage forms are especially suitable for intravenous, intramuscular, subcutaneous, intratumoral, and intraperitoneal administration. In this connection, sterile aqueous media that can be employed will be known to those of skill in the art in light of the present disclosure. For example, one dosage is dissolved, in certain cases, in 1 mL to 20 mL of isotonic NaCl solution and either added to 100 mL to 1000 mL of a fluid, e.g., sodium-bicarbonate buffered saline, or injected at the proposed site of infusion.

In certain embodiments, sterile injectable solutions is prepared by incorporating a immunotherapy agent, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. The compositions disclosed herein are, in some instances, formulated in a neutral or salt form. Pharmaceutically-acceptable salts include, for example, the acid addition salts (formed with the free amino groups of the protein), and which are formed with inorganic acids such as, for example, hydrochloric or phosphoric acids, or such organic acids as acetic, oxalic, tartaric, mandelic, and the like. Salts formed with the free carboxyl groups are, in some cases, derived from inorganic bases such as, for example, sodium, potassium, ammonium, calcium, or ferric hydroxides, and such organic bases as isopropylamine, trimethylamine, histidine, procaine and the like. Upon formulation, the pharmaceutical compositions are administered, in some embodiments, in a manner compatible with the dosage formulation and in such amount as is therapeutically effective.

In certain embodiments, a pharmaceutical composition of this disclosure comprises an effective amount of an anti-TM4SF1 antibody, as disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically compatible carriers can include gels, bioadsorbable matrix materials, implantation elements containing the immunotherapeutic agents or any other suitable vehicle, delivery or dispensing means or material. Such carriers are formulated, for example, by conventional methods and administered to the subject at an effective amount.

### IX. Combination Therapies

In certain embodiments, the methods of this disclosure comprise administering an ADC as disclosed herein, followed by, preceded by or in combination with one or more further therapy. Examples of the further therapy can include, but are not limited to, chemotherapy, radiation, an anti-cancer agent, or any combinations thereof. The further therapy can be administered concurrently or sequentially with respect to administration of the immunotherapy. In certain embodiments, the methods of this disclosure comprise administering an immunotherapy as disclosed herein, followed by, preceded by, or in combination with one or more anti-cancer agents or cancer therapies. Anti-cancer agents include, but are not limited to, chemotherapeutic agents, radiotherapeutic agents, cytokines, immune checkpoint inhibitors, anti-angiogenic agents, apoptosis-inducing agents, anti-cancer antibodies and/or anti-cyclin-dependent kinase agents. In certain embodiments, the cancer therapies include chemotherapy, biological therapy, radiotherapy, immunotherapy, hormone therapy, anti-vascular therapy, cryotherapy, toxin therapy and/or surgery or combinations thereof. In certain embodiments, the methods of this disclosure include administering an immunotherapy, as disclosed herein, followed by, preceded by or in combination with one or more further immunomodulatory agents. An immunomodulatory agent includes, in some examples, any compound, molecule or substance capable of suppressing antiviral immunity associated with a tumor or cancer. Non-limiting examples of the further immunomodulatory agents include an agent that binds to a protein selected from the group consisting of A2AR, B7-H3, B7-H4, BTLA, CD27, CD137, 2B4, TIGIT, CD155, ICOS, HVEM, CD40L, LIGHT, TIM-1, OX40, DNAM-1, PD-L1, PD1, PD-L2, CTLA-4, CD8, CD40, CEACAMI, CD48, CD70, A2AR, CD39, CD73, B7-H3, B7-H4, BTLA, IDO1, ID02, TDO, KIR, LAG-3, TIM-3, and VISTA; an anti-CD33 antibody or variable region thereof, an anti-CDIIb antibody or variable region thereof, a COX2 inhibitor, e.g., celecoxib, cytokines, such as IL-12, GM-CSF, IL-2, IFN3 and 1FNy, and chemokines, such as MIP-1, MCP-1 and IL-8.

In certain examples, where the further therapy is radiation exemplary doses are 5,000 Rads (50 Gy) to 100,000 Rads (1000 Gy), or 50,000 Rads (500 Gy), or other appropriate doses within the recited ranges. Alternatively, the radiation dose are about 30 to 60 Gy, about 40 to about 50 Gy, about 40 to 48 Gy, or about 44 Gy, or other appropriate doses within the recited ranges, with the dose determined, example, by means of a dosimetry study as described above. “Gy” as used herein can refer to a unit for a specific absorbed dose of radiation equal to 100 Rads. Gy is the abbreviation for “Gray.”

In certain examples, where the further therapy is chemotherapy, exemplary chemotherapeutic agents include without limitation alkylating agents (e.g., nitrogen mustard derivatives, ethylenimines, alkylsulfonates, hydrazines and triazines, nitrosureas, and metal salts), plant alkaloids (e.g., vinca alkaloids, taxanes, podophyllotoxins, and camptothecan analogs), antitumor antibiotics (e.g., anthracyclines, chromomycins, and the like), antimetabolites (e.g., folic acid antagonists, pyrimidine antagonists, purine antagonists, and adenosine deaminase inhibitors), topoisomerase I inhibitors, topoisomerase II inhibitors, and miscellaneous antineoplastics (e.g., ribonucleotide reductase inhibitors, adrenocortical steroid inhibitors, enzymes, antimicrotubule agents, and retinoids).

Exemplary chemotherapeutic agents can include, without limitation, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), Ibrutinib, idelalisib, and brentuximab vedotin.

In some embodiments, the topoisomerase I inhibitor is camptothecin.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune®, ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary anthracyclines can include, without limitation, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteasome inhibitors can, but are not limited to, bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoac etamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

“In combination with,” as used herein, means that the anti-TM4SF1 antibody and the further therapy are administered to a subject as part of a treatment regimen or plan. In certain embodiments, being used in combination does not require that the anti-TM4SF1 antibody and the further therapy are physically combined prior to administration or that they be administered over the same time frame. For example, and not by way of limitation, the anti-TM4SF1 antibody and the one or more agents are administered concurrently to the subject being treated or are administered at the same time or sequentially in any order or at different points in time.

### X. Kits

In some embodiments, the disclosure provides kits that include a composition (e.g., a pharmaceutical composition) of the disclosure (e.g., a composition including an ADC containing an anti-TM4SF1 antibody or antigen binding fragment thereof). The kits include instructions to allow a clinician (e.g., a physician or nurse) to administer the composition contained therein to a subject to treat a disorder associated with pathological angiogenesis (e.g., cancer).

In certain embodiments, the kits include a package of a single-dose pharmaceutical composition(s) containing an effective amount of an antibody of the disclosure. Optionally, instruments or devices necessary for administering the pharmaceutical composition(s) may be included in the kits. For instance, a kit of this disclosure may provide one or more pre-filled syringes containing an effective amount of a vaccine, vector, stabilized trimer, or optimized viral polypeptide of the disclosure. Furthermore, the kits may also include additional components such as instructions regarding administration schedules for a subject having a disorder associated with pathological angiogenesis (e.g., cancer) to use the pharmaceutical composition(s) containing a TM4SF1 binding protein or polynucleotide of the disclosure.

The application may be better understood by reference to the following non-limiting examples, which are provided as exemplary embodiments of the application. The following examples are presented in order to illustrate embodiments more fully and should in no way be construed, however, as limiting the broad scope of the application.

Antibody drug conjugates (ADCs) containing exemplary anti-TM4SF1 antibodies as described in Table 5. FIG. 1 provides the structures of the intermediates leading to ADCs. They use different conjugation methods: maleimide conjugation or bromoacetamide conjugation (FIG. 1).

### EXAMPLES

### Example 1: Characterization of Exemplary Anti-TM4SF1 Antibodies

### Affinity

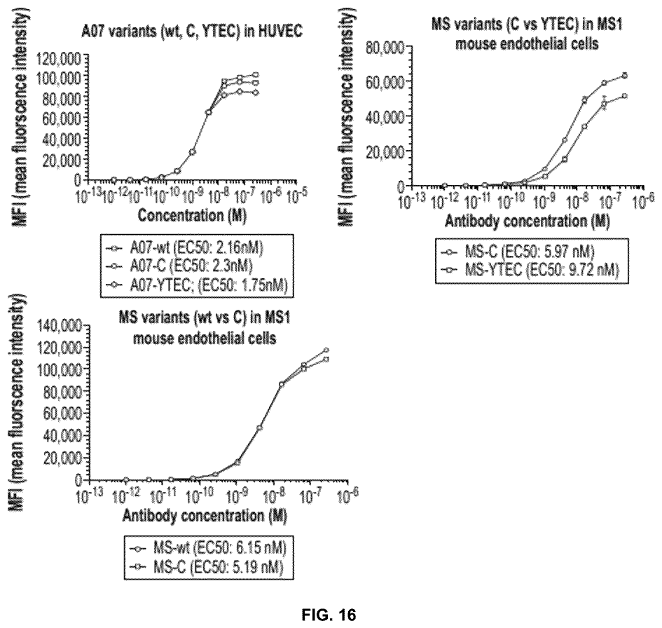

Antigen binding affinities of anti-TM4SF1 antibodies comprising various Fc mutations were tested, via a cell-based flow cytometry assay. Variants of an exemplary anti-TM4SF1 antibody AGX-A07, comprising Fc region mutation N297C (the “C” variant) or N297C in combination with the mutations M252Y, S254T, and T256E (the “YTEC” variant), were tested using HUVEC cells (Primary Umbilical Vein Endothelial Cells; ATCC® PCS-100-010™) The EC₅₀ values for binding are shown in FIG. 16 (top left panel), where A07-wt corresponds to the AGX-A07 antibody without Fc region mutations. Similarly, a “C” variant and an “YTEC” variant of a murine surrogate (referred to as “MS” in the figures), were tested in immortalized mouse endothelial cell MS-1 cells (MILE SVEN 1; ATCC® CRL-2279™) The EC₅₀ values for binding are shown in FIG. 16 (top right panel and bottom right panel), where MS-wt corresponds to the murine surrogate antibody without the Fc region mutations.

### Tissue Distribution

In this study, in vivo tissue distribution of the murine surrogate (MS) anti-TM4SF1 antibody “C” and “YTEC” variants was determined, in mice. Murine surrogate “C” variant conjugated to Alexa Fluor™ 647 (MS-C-647) and murine surrogate “YTEC” variant conjugated to Alexa Fluor™ 488 (MS-YTEC-488) were intraperitoneally co-injected to LLC (Lewis lung carcinoma) tumor bearing C57BL/6 (8 weeks old) mice, at a dose of 30 mg/kg (30 mpk). Major organs were harvest 24 or 48 hours after the injection and were fixed in 4% paraformaldehyde for embedding in OCT mounting media and sectioning. The MS-C-647 and MS-YTEC-488 antibody signals in tissue sections were captured via confocal microscope and the tissue distribution differences were examined. The results are shown in Table 1.

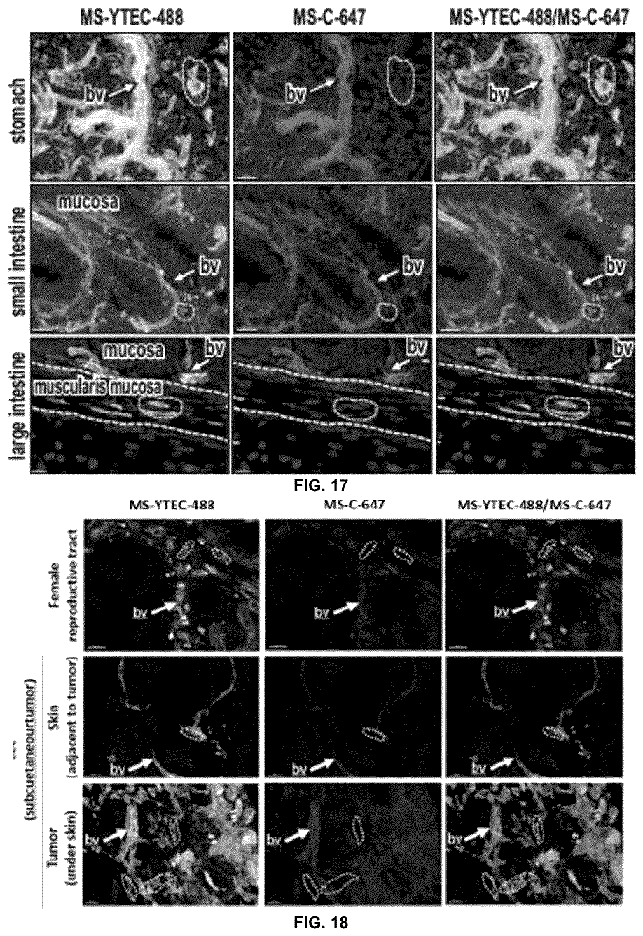

All blood vessels were found to be positive for both MS-C-647 and MS-YTEC-488 signals. In some organs, tissue resident mast cells and/or pericytes also strongly interacted with the MS-YTEC-488 but not with the MS-C-647. These results suggested that the MS-YTEC-488 can readily be transcytosed from endothelium to tissues for their interaction with leukocyte via antibody constant region or pericytes via antigen binding. The overall tissue distribution observations are summarized in below table and also shown in FIGS. 17 and 18 (bv=blood vessel).

TABLE 1
Tissue distribution of MS-C and MS-YTEC

| | | Tissue resident |
|---|---|---|
| | | mast cell or |
| Mouse organs | Blood vessel staining | pericyte staining |
| Brain | Comparable between MS-C | no |
| Stomach | and MS-YTEC | Yes for MS-YTEC |
| Small Intestine | | Yes for MS-YTEC |
| Large Intestine | | Yes for MS-YTEC |
| Eye | | No |
| Female Reproductive | | Yes for MS-YTEC |
| System | | |
| Heart | | No |
| Kidney | | No |
| Liver | | No |
| Lung | | No |
| Pancreas | | No |
| Skin + tumor | | Yes for MS-YTEC |

### Hydrophobicity

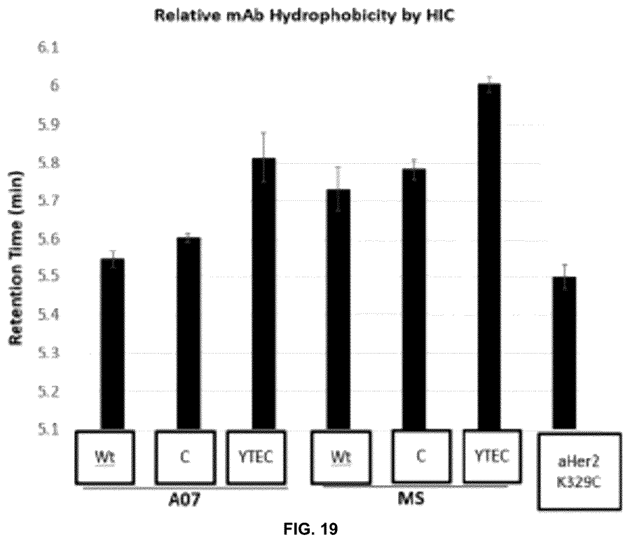

The hydrophobicity of exemplary anti-TM4SF1 antibodies, and their Fc mutation containing variants were assessed in this study, using hydrophobic interaction chromatography. The tested antibodies were AGX-A07 (the “wt,” “C,” and “YTEC”); MS (the “wt,” “C,” and “YTEC”). An anti-Her2 antibody with an Fc mutation was used as a control. Results are plotted in FIG. 19 and also summarized in Table 2. Both in case of AGX-A07 and the murine surrogate anti-TM4SF1 antibody, it was observed that the hydrophobicity increased with the “C” and “YTEC” mutations.

TABLE 2
Hydrophobicity summary

| | Run 1 | Run 2 | Run 3 | Average | StD |
|---|---|---|---|---|---|
| A07-wt | 5.547 | 5.523 | 5.568 | 5.55 | 0.023 |
| A07C | 5.614 | 5.591 | 5.606 | 5.60 | 0.0012 |
| A07-YTEC | 5.781 | 5.77 | 5.888 | 5.81 | 0.065 |
| MS-wt | 5.697 | 5.7 | 5.797 | 5.73 | 0.057 |
| MS-C | 5.807 | 5.784 | 5.752 | 5.78 | 0.028 |
| MS-YTEC | 6.017 | 5.978 | 6.017 | 6.00 | 0.023 |
| anti-Her2 | 5.496 | 5.467 | 5.532 | 5.5 | 0.033 |
| K392C | | | | | |

### Example 2: Antibody Drug Conjugates Containing Exemplary Anti-TM4SF1 Antibodies

Antibody drug conjugates (ADCs) containing exemplary anti-TM4SF1 antibodies are prepared and tested in in vitro and in vivo studies. ADCs are prepared using maleimide conjugation or bromoacetamide conjugation; 1⁴ S,1⁶ S,3² S,3³ S,2R,4S,10E,12E,14R)-8⁶-chloro-14-hydroxy-8⁵,14-dimethoxy-3³, 2,7,10-tetramethyl-1²,6-dioxo-7-aza-1(6,4)-oxazinana-3(2,3)-oxirana-8(1,3)-benzenacyclotetradecaphane-10,12-dien-4-yl N-(6-(2,5-dioxo-2,5-dihydro-1H-pyrrol-1-yl)hexanoyl)-N-methyl-L-alaninate, and 14 S,1⁶ S,3² S,3³ S,2R,4S,10E,12E,14R)-8⁶-chloro-1⁴-hydroxy-8⁵, 14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl (S)-1-bromo-26,27-dimethyl-2,18,25-trioxo-6,9,12,15-tetraoxa-3,19,26-triazaoctacosan-28-oate.

### In Vivo Tolerance

Eight weeks old C57Bl/6 mice ae administered an ADC at various doses (40 mg/kg, 50 mg/kg, and 60 mg/kg). The DAR for the ADC are about 2.0 and can vary from about 1 to about 4.

In other studies, mice of different ages are administered ADCs prepared using bromoacetamide conjugation, maleimide-conjugation, or other conjugation methods. Through these studies, conjugation method that shows the better potency and/or more toleration and/or fewer side effects can be determined. For example, using dose escalating experiments, survival rate at 40 mg/kg or 60 mg/kg doses can be used to compare the safety aspects of the ADCs.

### Toxicity Studies in Cynomolgus Monkeys

In this study the animals are randomly divided into various groups and administered various ADCs. Then macroscopic and microscopic pathological observations from the monkey study can be obtained at termination, for example, 42 days after 1 injection of test article.

### Pharmacokinetic Studies in Mice and Cynomolgus Monkeys

In this study, various concentrations of exemplary anti-TM4SF1 antibodies and ADCs containing the same are assessed, in mouse and cynomolgus monkeys. Pharmacokinetic data are obtained after testing how fast each ADC is cleared.

Different injection routes, intravenous (iv) and intraperitoneal (ip), show either similar or different levels of the antibodies in circulation in mice.

### Efficacy

In this study, the efficacy of ADCs in reducing tumor volume are tested.

Briefly, eight weeks old C57/B16 mice that are previously implanted with B16-F10 tumor cells (ATCC® CRL-6475™—mouse skin melanoma cells) are randomized into groups and injected with a control or ADCs at different dosages, for example, 12 mg/kg or 20 mg/kg.

Tumor volumes for a period of about 16 days, following injection, are measured and tumor regression efficacy is calculated and/or observed.

A further efficacy study is carried out using a MiaPaca 2 (ATCC® CRL-1420™—pancreatic carcinoma) xenograft tumor model. Briefly, eight weeks old athymic nude mice are randomized into groups and injected with a control or ADCs at different dosages and with different dosing regimens. Effects of the ADCs on the efficacy of MiaPaca2 tumor regression are observed and recorded.

### Conjugates Comprising Galactoside Linkers and In Vitro Activities of the Same in Cultured Cells

Various conjugates comprising exemplary anti-TM4SF1 antibodies and a cytotoxic payload (e.g., maytansine), conjugated using different linkers, are evaluated for cell killing potential, using multiple cancer cell lines.

Cells are incubated with antibody conjugate at various concentrations. The concentrations evaluated include a control, and five-fold dilution starting from 333.335 nM (333.335 nM, 66.667 nM, 13.334 nM, 2.667 nM, 0.533 nM (533.3360 pM), 106.6672 pM, 21.3334 pM, 4.2667 pM. Briefly, cells are seeded into 96-well plates a day before the antibody conjugates are added, whereupon cell viability is measured by PrestoBlue (ThermoFisher Scientific) through a plate reader (Varioskan™ LUX multimode microplate reader) five days after treatment with serial dilutions of the antibody conjugates. Cells are treated with media only or isotype matched control antibodies, as a negative control. The EC₅₀ values are generated via GraphPad.

### Example 3: BrAc-Galactose-Sar-N-Me-Alanine-DM1 (11)

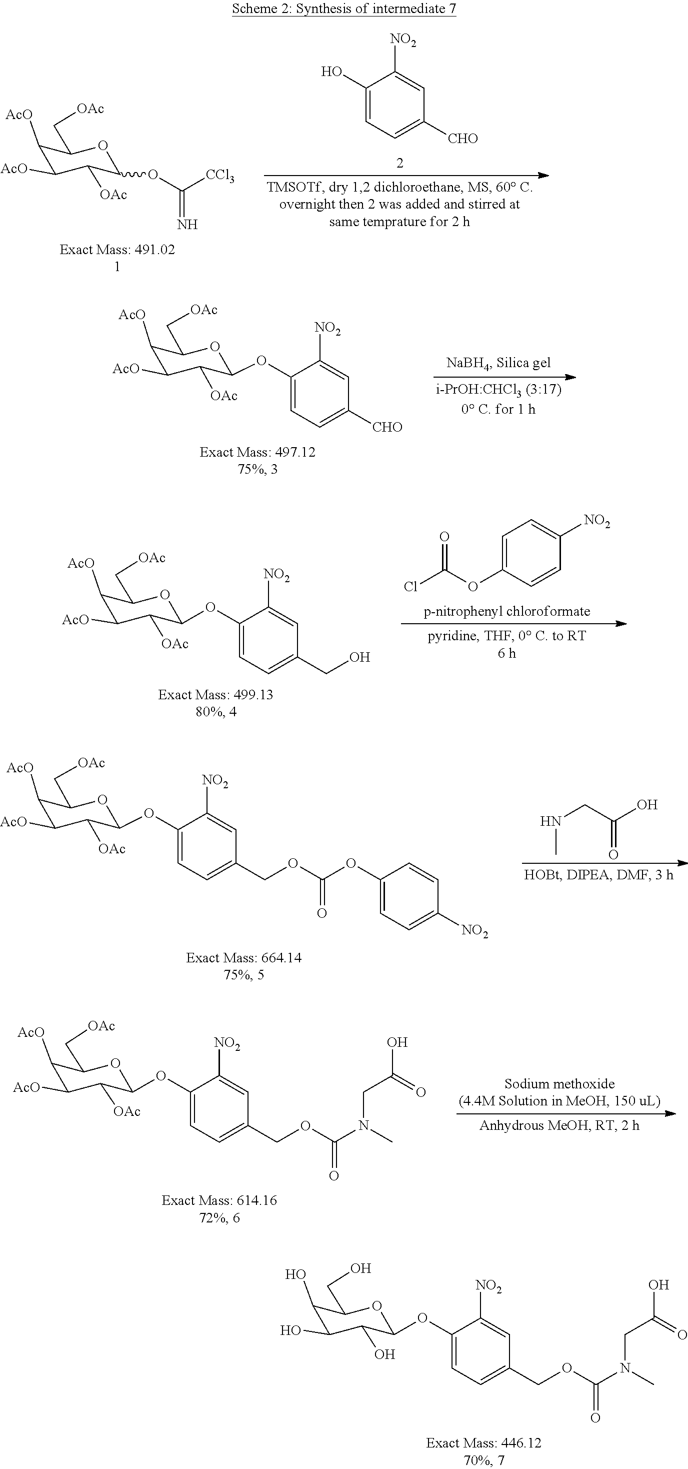

### 1) Compound 3

2,3,4,6-Tetra-O-acetyl-D-galactopyranosyl trichloroacetimidate (Synthose, 5 g, 10.18 mmol) was dissolved in anhydrous 1,2 dichloroethane (100 ml) under the atmosphere of argon. To this solution were added TMSOTf (2.8 mL, 15.27 mmol) at 0° C., and the mixture was stirred at 60° C. overnight. To this solution were added 4-hydroxy-3-nitrobenzaldehyde 2 (Oakwood, 8.5 g, 50.9 mmol) and stirring was continued for 2 h at same temperature till TLC showed complete conversion of starting material. The mixture was then neutralized with Et₃N (1 mL) and concentrated to dryness affording the crude product as yellowish-white solid. The solution was then concentrated to dryness, applied onto a column of silica gel, and purified using EtOAc: Hexane (30-50% ethyl acetate in hexane) to give compound 3 as a white solid (3.75 g, 75%).

### 2) Compound 4

The mixture of intermediate 3 (3.5 g, 7.04 mmol), NaBH₄ (781.5 mg, 21.12 mmol), and silica gel (7.5 g) in isopropanol/CHCl₃ (3:17) (100 ml) was stirred at 0° C. for 1 h. The reaction was quench with water, and the mixture was filtered to remove silica gel. The organic layer was dried over MgSO₄ and evaporated under reduced pressure to give a residue, which was washed with ethanol to give desired product 4 (2.8 g, 80%).

### 3) Compound 5

To a solution of compound 4 (2.8 g, 5.61 mmol) in THF (100 ml) was added pyridine (4 ml) and p-nitrophenyl chloroformate (2.25 g, 11.22 mmol) at 0° C. and warmed up to RT and stirred for 6 h. The mixture was purified by flash chromatography (EtOAc/Hexane, 2/1, silica gel) to give compound 5 as a white solid (2.79 g, 75%).

### 4) Compound 6

To a mixture of 5 (2.5 g, 3.765 mmol) and sarcosine (505 mg, 5.647 mmol) in DMF (25 ml) was added HOBt (350 mg, 2.6 mmol) and DIPEA (3.765 mmol, 655 μL), and the reaction was stirred at room temperature for 3 h. The crude reaction was purified by reverse phase prep-HPLC to give desired product 6 as a white powder after lyophilization (1.72 g, 72%).

### 5) Compound 7

To a stirred solution of compound 6 (1.72 g, 2.80 mmol) in 75 ml of anhydrous methanol was added sodium methoxide (4.4 M solution in MeOH, 621 μL) and the mixture was stirred at room temperature for 2-3 h. The reaction was then acidified (HOAc, 289 μL) and concentrated, and purified by reverse phase prep-HPLC to give compound 7 as a white powder after lyophilization (868 mg, 70%).

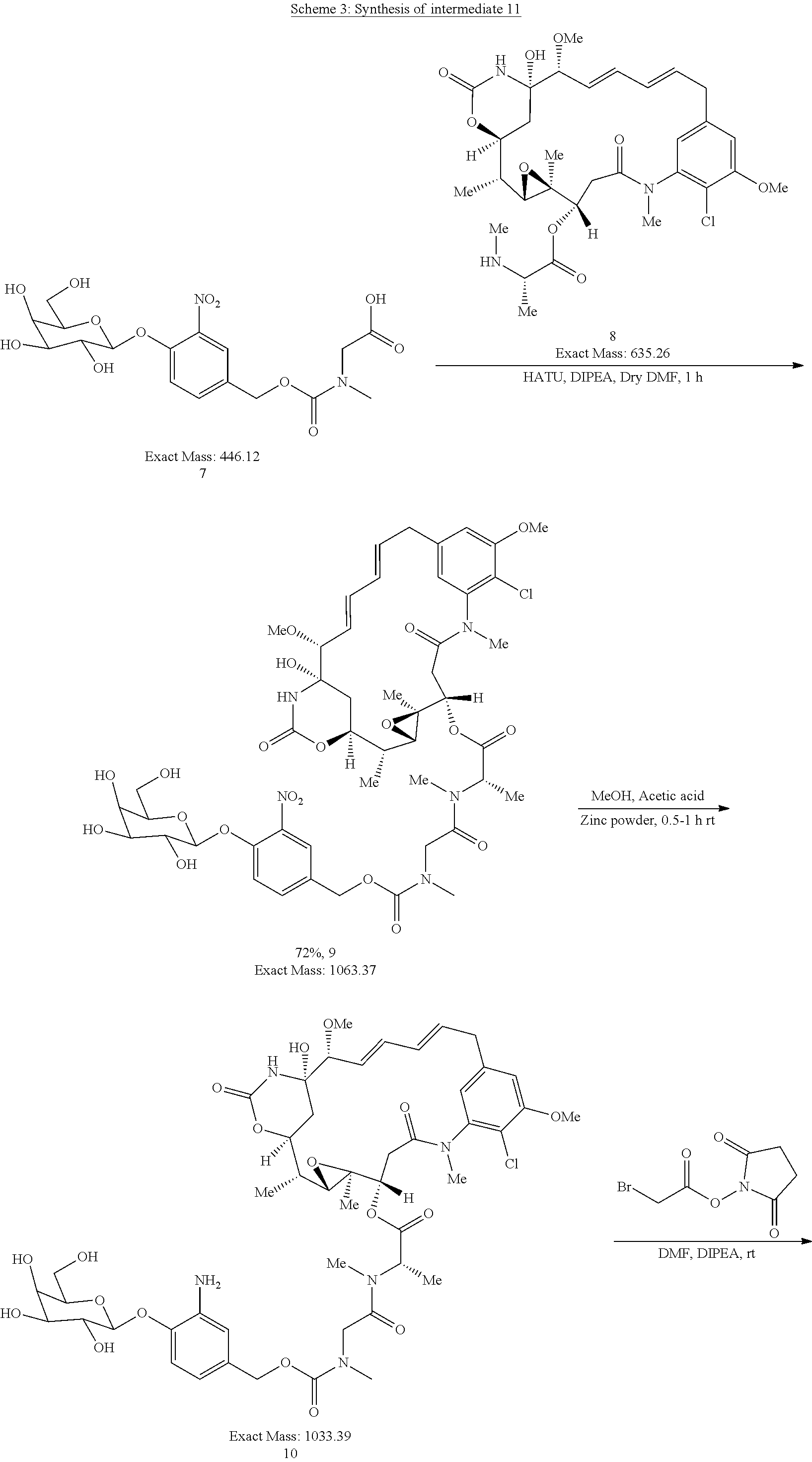

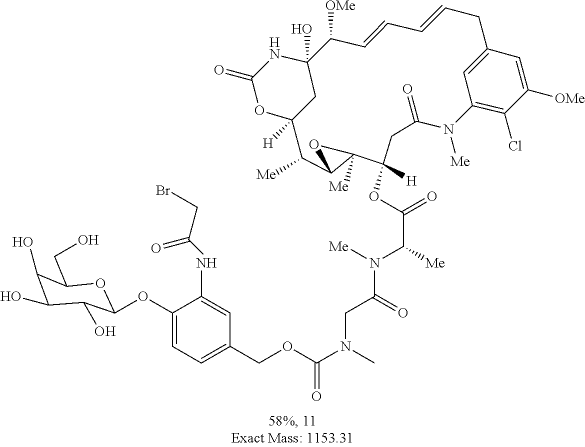 [SMILES: COc1cc2cc(c1Cl)N(C)C(=O)C[C@H](OC(=O)[C@H](C)N(C)C(=O)CN(C)C(=O)OCc1ccc(OC3OC(CO)C(O)C(O)C3O)c(NC(=O)CBr)c1)[C@]1(C)O[C@H]1[C@H](C)[C@@H]1C[C@@](O)(NC(=O)O1)[C@H](OC)/C=C/C=C/C2]

### 6) Compound 9

To a solution of 7 (200 mg, 0.448 mmol) and compound 8 (341.75 mg, 0.537 mmol) in anhydrous DMF (9 mL) was added DIEA (234 μL, 1.344 mmol) and HATU (171 mg, 0.448 mmol), and the mixture was stirred at room temperature for 30 min. The mixture was purified directly by RP-HPLC to give compound 9 as a white powder after lyophilization (342 mg, 72%).

### 7) Compound 10

Compound 9 (300 mg, 0.2821 mmol) was dissolved in MeOH (7.5 mL), and acetic acid (1.2 mL) was added, followed by zinc powder (60 mg). The mixture was stirred at room temperature for 30 mins. The reaction was then filtered, and the filtrate was concentrated under reduced pressure. The residue was purified by RP-HPLC to resulting compound 10 as white powder (198 mg, 68%).

### 8) Compound 11

To a solution of 10 (100 mg, 0.096 mmol) in acetonitrile/water (6.6 mL, 10/6.6, v/v) was added 1N aqueous NaOH solution (148.5 μL) and the mixture was stirred at room temperature for 20 min. LC/MS confirmed the complete saponification. Bromoacetic anhydride was added, followed by 66 μL of 1N NaOH. After 20 min, the reaction was acidified with 330 μL of aqueous citric acid (10% in water) and purified by RP-HPLC to give compound 11 (64 mg, 58%) as a white powder after lyophilization.

### Example 4: General Protocol for the Conjugation of Compound 11 to an Exemplary Antibody

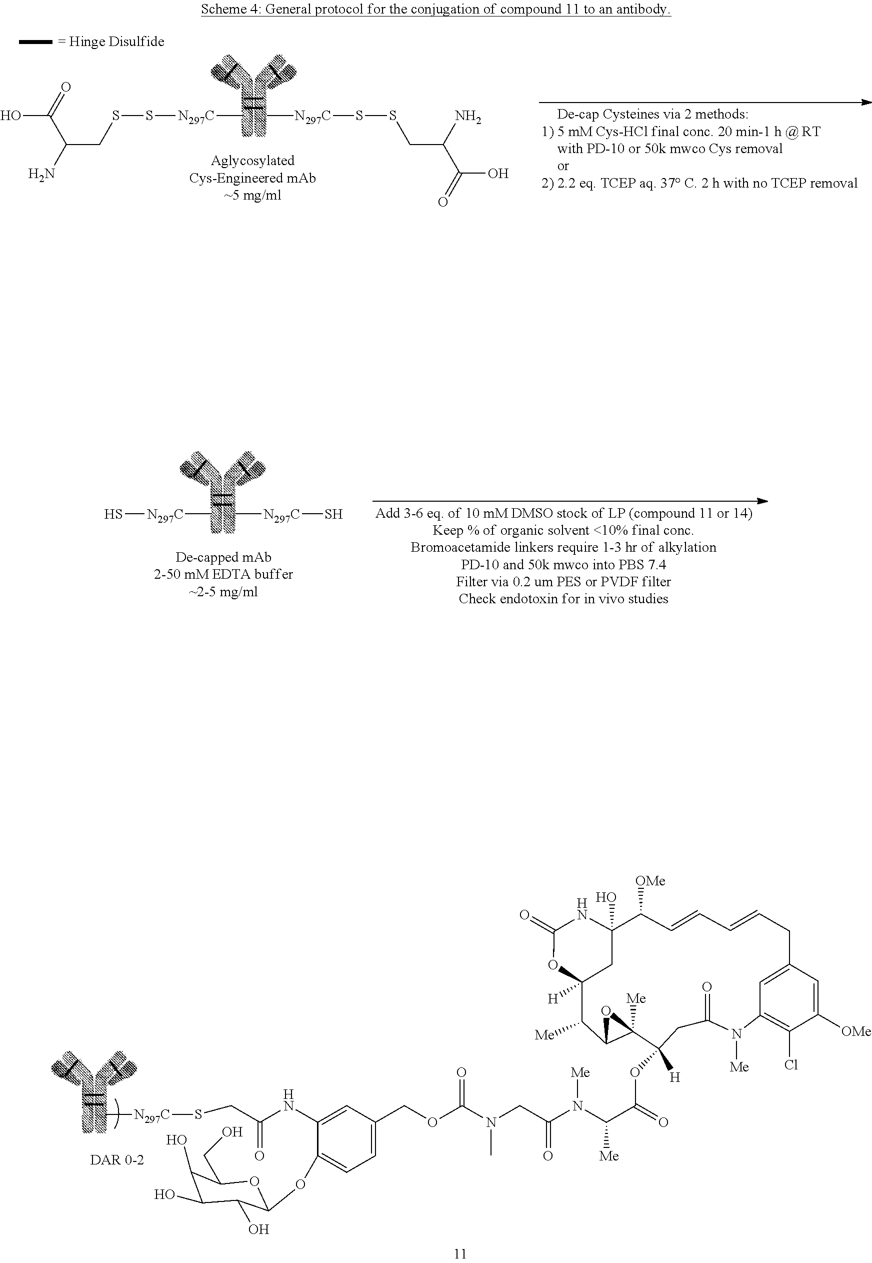

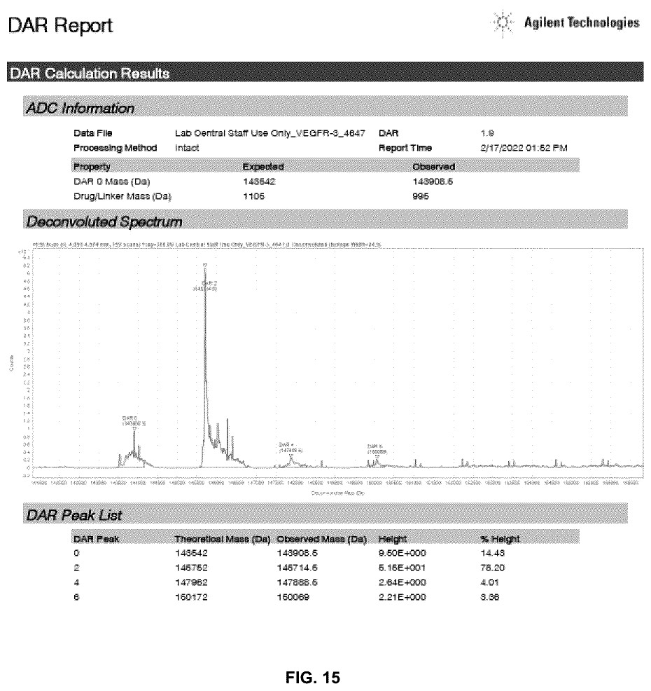

Exemplary Antibody Drug Conjugates ADC1-ADC8 target mouse cells or human cells and comprise Exemplary Antibody 1, Exemplary Antibody 2, Exemplary Antibody 3, Exemplary Antibody 4, and Exemplary Antibody 5. The intact Mass Spec analysis of DAR calculation results for Exemplary Antibody Drug Conjugates ADCT-ADC8 can be found in FIGS. 2, 4, 6, 8, 10, 12, 14, and 15, respectively. Some of the corresponding size exclusion chromatograph of the same Exemplary Antibody Drug Conjugate ADCT-ADC8 are shown in FIGS. 3, 5, 7, 9, 11, and 13.

### Example 5: Characterization of Anti-TM4SF1 Antibodies

### In Vitro Cell Proliferation Inhibition Assay

The effects of antibody drug-conjugates (drug to antibody ratio (DAR) varying from 1.8 to 4.2) containing a drug (maytansine) were assessed in cultured mouse (MS1) and human (HUVEC, A549, MiaPaCa2 and SKOV3) cells. Characteristics of exemplary ADCs (ADClto ADC8) are shown in Table 3. Characteristics of the Exemplary Antibodies 1-5, each of which contains human IgG1 constant region with YTEC mutation, can be found in Table 3A. The killing effect of these ADC are shown in Table 4. Cells were incubated for 5 days with the antibody drug conjugate before assessing cell viability on day 5. The in vitro cytotoxicity results of the free drug/linker payload is shown in Table 4 as well.

Conjugation of Exemplary Antibody 1 to Exemplary linker-payload (LP) as shown in Scheme 6. An exemplary structure of the LP used is shown in FIG. 1.

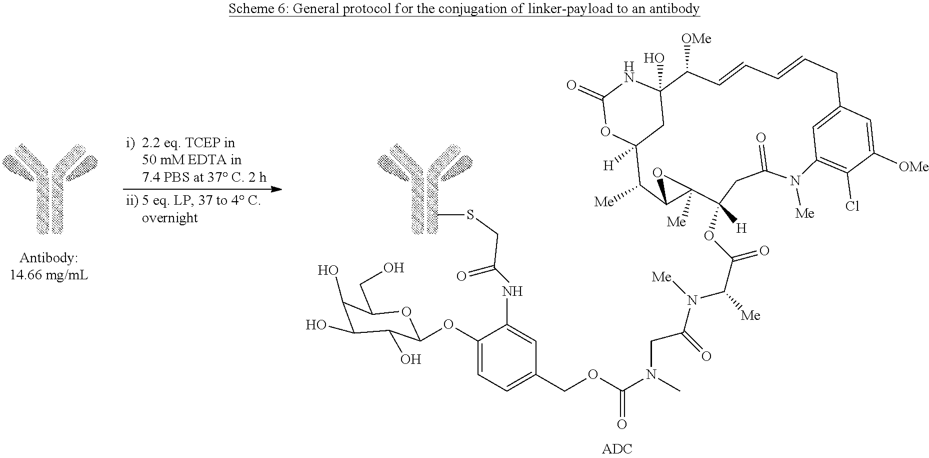 [SMILES: COc1cc2cc(c1Cl)N(C)C(=O)C[C@H](OC(=O)[C@H](C)N(C)C(=O)CN(C)C(=O)OCc1ccc(OC3OC(CO)C(O)C(O)C3O)c(NC(=O)CSC)c1)[C@]1(C)O[C@H]1[C@H](C)[C@@H]1C[C@@](O)(NC(=O)O1)[C@H](OC)/C=C/C=C/C2]

By varying the antibodies used, the concentrations of the antibodies, the equivalents of LP used, and other conditions, ADCs having different DAR are obtained.

FIG. 2 shows the intact Mass Spec analysis of DAR calculation results for ADC1, the conjugated ADC made from Exemplary Antibody 1 and Compound 11. The corresponding size exclusion chromatograph of the same ADC is shown in FIG. 3.

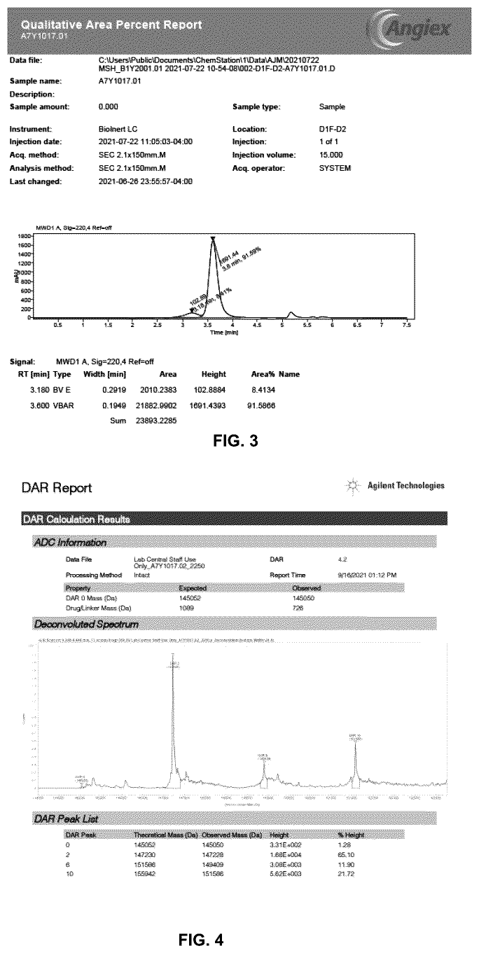

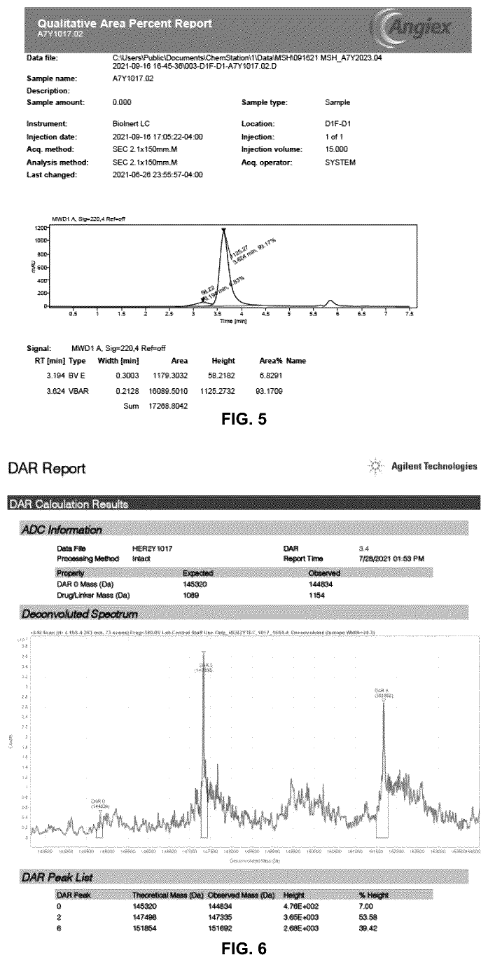

FIG. 4 shows the intact Mass Spec analysis of DAR calculation results for ADC1, the conjugated ADC made from Exemplary Antibody 1 and Compound 11. The corresponding size exclusion chromatograph of the same ADC is shown in FIG. 5.

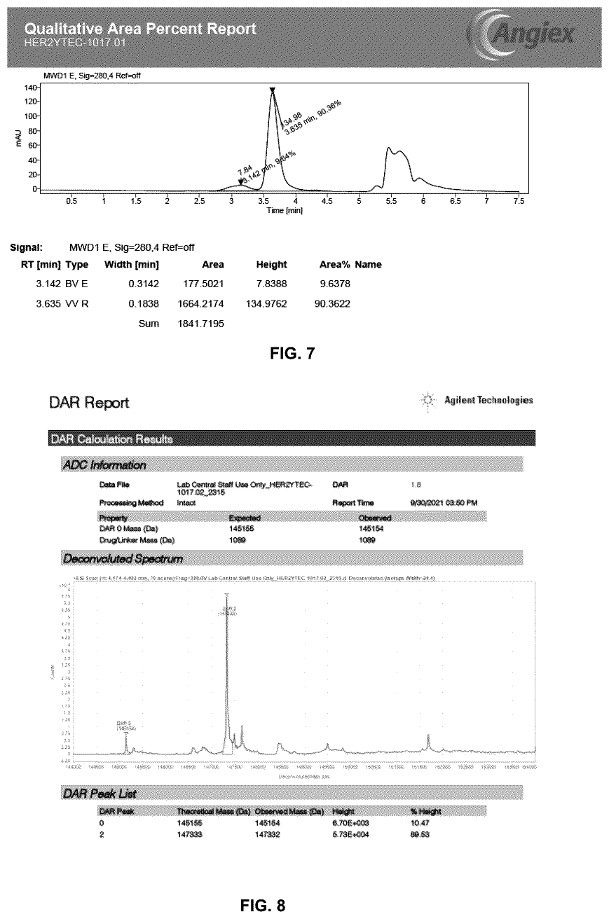

FIG. 6 shows the intact Mass Spec analysis of DAR calculation results for ADC1, the conjugated ADC made from Exemplary Antibody 2 and Compound 11. The corresponding size exclusion chromatograph of the same ADC is shown in FIG. 7.

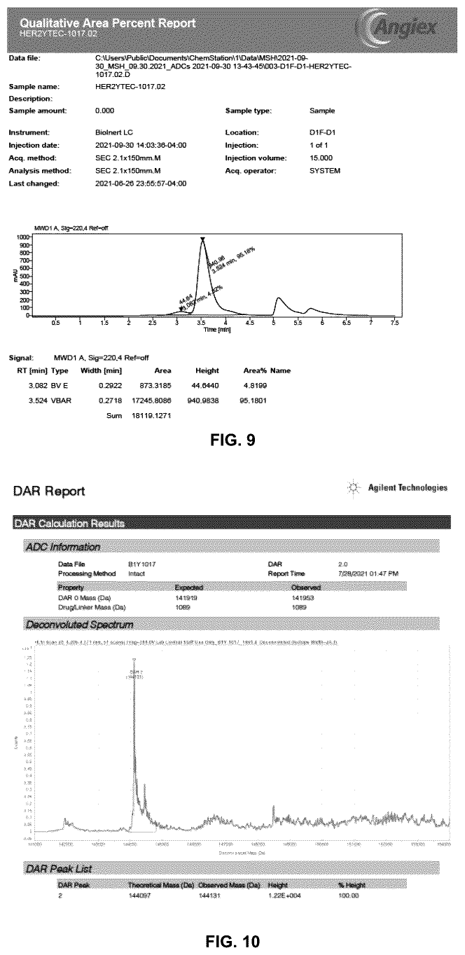

FIG. 8 shows the intact Mass Spec analysis of DAR calculation results for ADC1, the conjugated ADC made from Exemplary Antibody 2 and Compound 11. The corresponding size exclusion chromatograph of the same ADC is shown in FIG. 9.

FIG. 10 shows the intact Mass Spec analysis of DAR calculation results for ADC1, the conjugated ADC made from Exemplary Antibody 3 and Compound 11. The corresponding size exclusion chromatograph of the same ADC is shown in FIG. 11.

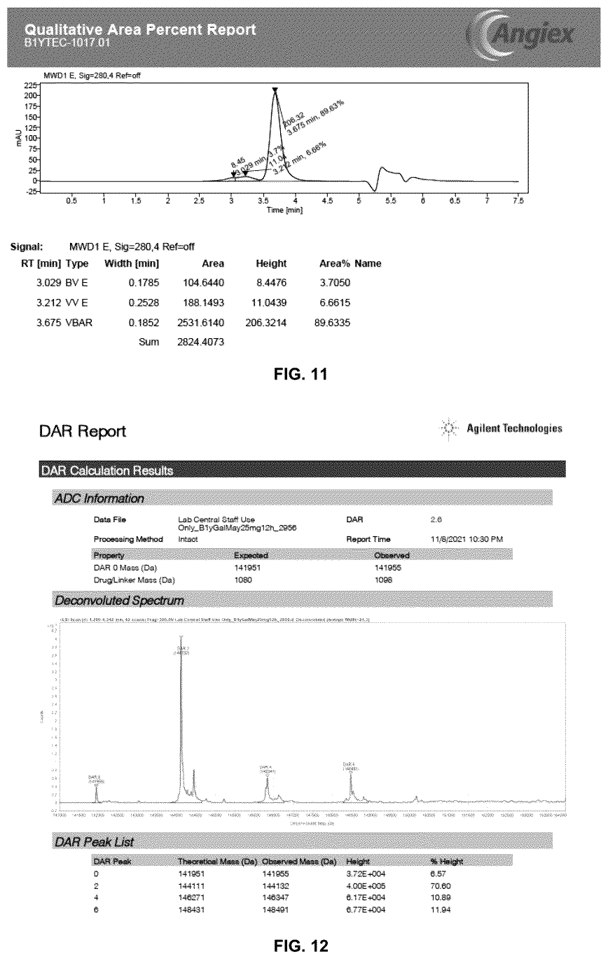

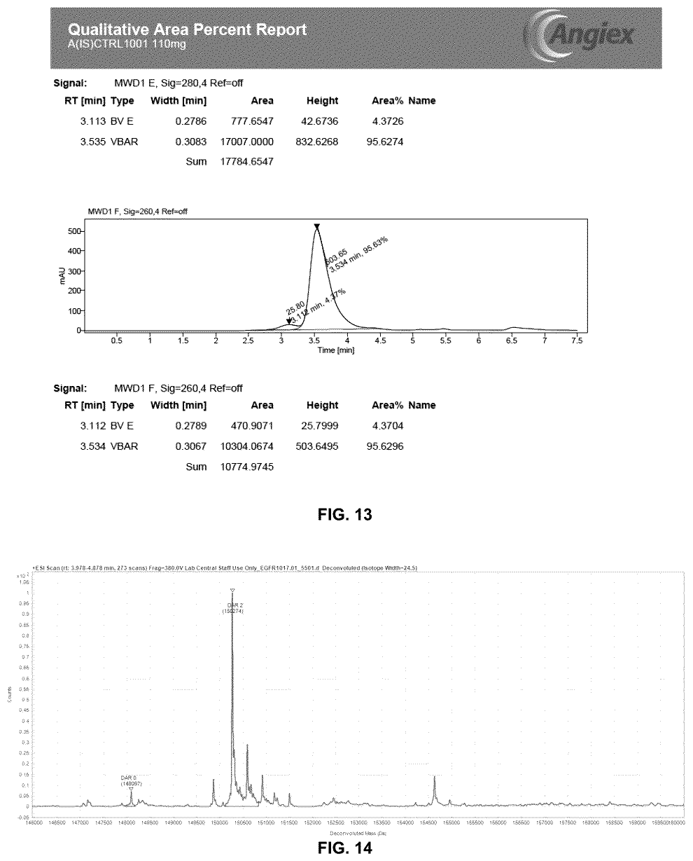

FIG. 12 shows the intact Mass Spec analysis of DAR calculation results for ADC1 the conjugated ADC made from Exemplary Antibody 3 and Compound 11. The corresponding size exclusion chromatograph of the same ADC is shown in FIG. 13.

FIG. 14 shows the intact Mass Spec analysis of DAR calculation results for ADC1, the conjugated ADC made from Exemplary Antibody 4 and Compound 11.

TABLE 3
ADCs made and tested

| | | | | | | conjugation |
|---|---|---|---|---|---|---|
| ADC | Antibody | DAR | linker | spacer | payload | site |
| ADC1 | Exemplary | 1.5 | Bromoacetamide | Gal-Sar-N- | Maytansine | N297C |
| | Antibody 1 | | | Methyl- | | |
| | | | | Alanine | | |
| ADC2 | Exemplary | 4.2 | Bromoacetamide | Gal-Sar-N- | Maytansine | N297C |
| | Antibody 1 | | | Methyl- | | |
| | | | | Alanine | | |
| ADC3 | Exemplary | 2.0 | Bromoacetamide | Gal-Sar-N- | Maytansine | N297C |
| | Antibody 2 | | | Methyl- | | |
| | | | | Alanine | | |
| ADC4 | Exemplary | 2.6 | Bromoacetamide | Gal-Sar-N- | Maytansine | N297C |
| | Antibody 2 | | | Methyl- | | |
| | | | | Alanine | | |
| ADC5 | Exemplary | 3.4 | Bromoacetamide | Gal-Sar-N- | Maytansine | N297C |
| | Antibody 3 | | | Methyl- | | |
| | | | | Alanine | | |
| ADC6 | Exemplary | 1.8 | Bromoacetamide | Gal-Sar-N- | Maytansine | N297C |
| | Antibody 3 | | | Methyl- | | |
| | | | | Alanine | | |
| ADC7 | Exemplary | 1.9 | Bromoacetamide | Gal-Sar-N- | Maytansine | N297C |
| | Antibody 4 | | | Methyl- | | |
| | | | | Alanine | | |
| ADC8 | Exemplary | 2 | Bromoacetamide | Gal-Sar-N- | Maytansine | N297C |
| | Antibody 5 | | | Methyl- | | |
| | | | | Alanine | | |

TABLE 3A
Exemplary Antibody Characteristics

| Antibody | Antigen |
|---|---|
| Exemplary Antibody 1 | TM4SF1 (human) |
| Exemplary Antibody 2 | TM4SF1 (mouse) |
| Exemplary Antibody 3 | Her2 |
| Exemplary Antibody 4 | EGFR |
| Exemplary Antibody 5 | VEGFR2 |

TABLE 4
In vitro cell proliferation/inhibition activity (EC50; nM) of ADCs

| | Killing activity in vitro (EC50; nM) | | | | |
|---|---|---|---|---|---|
| | HUVEC | | | MiaPaca2 | SKOV3 |
| | (endothelial | | A549 | (pancreatic | (ovarian |
| ADC | cell) | MS1 | (lung cancer) | cancer) | cancer) |
| ADC1 | 32 | | | | |
| ADC2 | 0.402/0.148 | | | | |
| ADC3 | No killing | 0.088/0.184 | | | |
| ADC4 | No killing | 0.04/0.084 | | | |
| ADC5 | 2.9 | 20.91 | | | 17.87 |
| ADC6 | 149.7 | | | | 3.252 |
| ADC7 | | | No Killing | 90.4 | 71,000 |
| ADC8 | No Killing | | | | |
| Free LP | 905.8 | | | | |

LP: linker-payload

The linker-payload (LP) portion of Exemplary Antibody Drug Conjugates ADC, ADC2, ADC3, ADC4, ADC5, ADC6, ADC7, ADC8 was tested in HUVEC cell line. See Table 4.

It will be apparent to those skilled in the art that various modifications and variations can be made in the compositions, methods, and kits of the present disclosure without departing from the spirit or scope of the disclosure. Thus, it is intended that the present disclosure cover the modifications and variations of this disclosure provided they come within the scope of the appended claims and their equivalents.

TABLE 5
SEQUENCE DESCRIPTION

| | | |
|---|---|---|
| SEQ | | |
| ID NO | Description | Sequence |

Antibody AGX-A01

| | | |
|---|---|---|
| 1 | AGX-A01 | EVILVESGGGLVKPGGSLKLSCAASGFTFSSF |
| | Variable heavy (VH) chain- | AMSWVRQTPEKRLEWVATISSGSIYIYYTDG |
| | amino acid | VKGRFTISRDNAKNTVHLQMSSLRSEDTAM |
| | | YYCARRGIYYGYDGYAMDYWGQGTSVTVS |
| 2 | AGX-A01 | AVVMTQTPLSLPVSLGDQASISCRSSQSLVHS |
| | Variable light (VL) chain-amino | NGNTYLHWYMQKPGQSPKVLIYKVSNRFSG |
| | acid | VPDRFSGSGSGTDFTLKISRVEADDLGIYFCS |
| | | QSTHIPLAFGAGTKLELK |

Antibody AGX-A03

| | | |
|---|---|---|
| 3 | AGX-A03 | QIQLVQSGPELKKPGETVKISCKASGYSFRD |
| | Variable heavy (VH) chain- | YGMNWVKQAPGRTFKWMGWINTYTGAPVY |
| | amino acid | AADFKGRFAFSLDTSASAAFLQINNLKNEDT |
| | | ATYFCARWVSYGNNRNWFFDFWGAGTTVT |
| | | VSS |
| 4 | AGX-A03 | CAGATCCAGTTGGTGCAGTCTGGACCTGAG |
| | Variable heavy (VH) chain- | CTGAAGAAGCCTGGAGAGACAGTCAAGAT |
| | nucleic acid | CTCCTGCAAGGCTTCTGGGTATTCCTTCAG |
| | | AGACTATGGAATGAACTGGGTGAAGCAGG |
| | | CTCCAGGAAGGACTTTTAAGTGGATGGGCT |
| | | GGATAAACACCTACACTGGAGCGCCAGTA |
| | | TATGCTGCTGACTTCAAGGGACGGTTTGCC |
| | | TTCTCTTTGGACACCTCTGCCAGCGCTGCC |
| | | TTTTTGCAGATCAACAACCTCAAAAATGAA |
| | | GACACGGCTACATATTTCTGTGCAAGATGG |
| | | GTCTCCTACGGTAATAACCGCAACTGGTTC |
| | | TTCGATTTTTGGGGCGCAGGGACCACGGTC |
| | | ACCGTCTCCTCA |
| 5 | AGX-A03 | CAAATTCAGTTGGTTCAATCCGGCCCTGAG |
| | Variable heavy (VH) chain- | CTCAAGAAGCCTGGAGAGACAGTGAAGAT |
| | codon optimized nucleic acid | AAGTTGTAAGGCTAGTGGCTATTCATTTCG |
| | | AGATTATGGGATGAATTGGGTCAAGCAGG |
| | | CCCCAGGGCGGACCTTCAAATGGATGGGG |
| | | TGGATCAATACTTACACTGGCGCACCAGTA |
| | | TATGCAGCTGATTTTAAGGGTCGCTTTGCA |
| | | TTTTCACTTGATACTTCAGCCAGTGCCGCT |
| | | TTTTTGCAAATCAACAATCTCAAAAATGAA |
| | | GACACTGCTACATATTTCTGCGCCAGGTGG |
| | | GTGAGCTATGGCAATAACAGAAATTGGTT |
| | | CTTTGACTTTTGGGGCGCAGGCACCACCGT |
| | | CACTGTCTCATCA |
| 6 | VH-CDR1 | GYSFRDYGMN |
| 7 | VH-CDR2 | WINTYTGAPVYAADFKG |
| 8 | VH-CDR3 | WVSYGNNRNWFFDF |
| 9 | AGX-A03 | DVLMTQTPLSLPVRLGDQASISCRSSQTLVHS |
| | Variable light (VL) chain-amino | NGNTYLEWYLQKPGQSPKLLIYKVSNRLSG |
| | acid | VPDRFSGSGSGTDFTLKISRVETEDLGVYYCF |
| | | QGSHGPWTFGGGTKLEIK |
| 10 | AGX-A03 | GATGTTTTGATGACCCAAACTCCACTCTCC |
| | Variable light (VL) chain- | CTGCCTGTCCGTCTTGGAGATCAGGCCTCC |
| | nucleic acid | ATCTCTTGTAGATCTAGTCAGACCCTTGTA |
| | | CATAGTAATGGAAACACCTATTTAGAATG |
| | | GTACCTGCAGAAACCAGGCCAGTCTCCAA |
| | | AACTCTTGATCTACAAAGTTTCCAATCGAC |
| | | TTTCTGGGGTCCCAGACAGGTTCAGTGGCA |
| | | GTGGATCAGGGACAGATTTCACACTCAAG |
| | | ATCAGCAGAGTGGAGACTGAGGATCTGGG |
| | | AGTTTATTACTGCTTTCAAGGTTCACATGG |
| | | TCCGTGGACGTTCGGTGGAGGCACCAAGC |
| | | TGGAAATCAAA |
| 11 | AGX-A03 | GACGTACTTATGACACAAACTCCCTTGAGC |
| | Variable light | TTGCCAGTACGGCTTGGCGATCAAGCTTCA |
| | (VL) chain-codon | ATTTCATGTCGTTCTTCTCAAACACTTGTCC |
| | optimized nucleic acid | ACTCAAATGGGAATACATATTTGGAATGGT |
| | | ATCTCCAAAAGCCCGGCCAATCCCCAAAA |
| | | TTGTTGATTTACAAGGTGTCTAATCGACTC |
| | | TCAGGCGTCCCCGACCGATTCTCCGGGAGC |
| | | GGGTCCGGTACAGACTTCACCTTGAAAATC |
| | | TCCAGGGTAGAAACTGAAGACCTCGGAGT |
| | | CTACTATTGTTTCCAGGGGTCACACGGCCC |
| | | CTGGACATTTGGAGGAGGAACTAAGCTCG |
| | | AGATCAAA |
| 12 | VL-CDR1 | RSSQTLVHSNGNTYLE |
| 13 | VL-CDR2 | KVSNRLS |
| 14 | VL-CDR3 | FQGSHGPWT |

Antibody AGX-A04

| | | |
|---|---|---|
| 15 | AGX-A04 | EVQLQQSGPELVKPGASVKISCKTSGYTFTD |
| | Variable heavy (VH) chain- | YTMHWVRQSHGKSLEWIGSFNPNNGGLTNY |
| | amino acid | NQKFKGKATLTVDKSSSTVYMDLRSLTSEDS |
| | | AVYYCTRIRATGFDSWGQGTTLTVSS |
| 16 | AGX-A04 | GAGGTCCAGCTGCAACAGTCTGGACCTGA |
| | Variable heavy (VH) chain- | GCTGGTGAAGCCTGGGGCTTCAGTGAAGA |
| | nucleic acid | TATCCTGCAAGACTTCTGGATACACATTCA |
| | | CTGATTACACCATGCACTGGGTGAGGCAG |
| | | AGCCATGGAAAGAGCCTTGAGTGGATTGG |
| | | AAGTTTTAATCCTAACAATGGTGGTCTTAC |
| | | TAACTACAACCAGAAGTTCAAGGGCAAGG |
| | | CCACATTGACTGTGGACAAGTCTTCCAGCA |
| | | CAGTGTACATGGACCTCCGCAGCCTGACAT |
| | | CTGAGGATTCTGCAGTCTATTACTGTACAA |
| | | GAATCCGGGCTACGGGCTTTGACTCCTGGG |
| | | GCCAGGGCACCACTCTCACAGTCTCCTCA |
| 17 | AGX-A04 | GAGGTACAACTGCAACAGAGTGGACCTGA |
| | Variable heavy (VH) chain- | ACTTGTCAAACCTGGAGCAAGTGTGAAGA |
| | codon optimized nucleic acid | TTAGCTGTAAAACCAGTGGCTACACATTTA |
| | | CCGATTATACTATGCACTGGGTAAGACAG |
| | | AGCCACGGAAAATCACTGGAGTGGATTGG |
| | | TAGTTTCAATCCTAACAACGGAGGATTGAC |
| | | AAATTACAACCAGAAGTTCAAAGGGAAAG |
| | | CCACCTTGACAGTTGATAAGTCCTCAAGTA |
| | | CCGTGTATATGGATCTGCGTTCTCTCACAA |
| | | GTGAAGATAGCGCAGTTTACTACTGTACCC |
| | | GCATCCGAGCCACCGGGTTCGATTCATGGG |
| | | GTCAGGGGACAACACTGACTGTTTCTTCT |
| 18 | VH-CDR1 | GYTFTDYTMH |
| 19 | VH-CDR2 | SFNPNNGGLTNYNQKFKG |
| 20 | VH-CDR3 | IRATGFDS |
| 21 | AGX-A04 | DIVMSQSPSSLAVSAGEKVTMSCKSSQSLLN |
| | Variable light (VL) chain- | SRTRKNYLAWYQQKPGQSPKLLIYWASTRE |
| | amino acid | SGVPDRFTGSGSGTDFTLTISNVQAEDLTVY |
| | | YCKQSYNPPWTFGGGTKLEIK |
| 22 | AGX-A04 | GACATTGTGATGTCACAGTCTCCATCCTCC |
| | Variable light (VL) chain- | CTGGCTGTGTCAGCAGGAGAGAAGGTCAC |
| | nucleic acid | TATGAGCTGCAAATCCAGTCAGAGTCTGCT |
| | | CAACAGTAGAACCCGAAAGAACTACTTGG |
| | | CTTGGTACCAGCAGAAACCAGGGCAGTCT |
| | | CCTAAACTGCTGATCTACTGGGCATCCACT |
| | | AGGGAATCTGGGGTCCCTGATCGCTTCACA |
| | | GGCAGTGGATCTGGGACAGATTTCACTCTC |
| | | ACCATCAGCAATGTGCAGGCTGAAGACCT |
| | | GACAGTTTATTACTGCAAGCAATCTTATAA |
| | | TCCTCCGTGGACGTTCGGTGGAGGCACCAA |
| | | GCTGGAAATCAAA |
| 23 | AGX-A04 | GACATAGTTATGTCCCAGTCTCCATCCAGC |
| | Variable light (VL) | TTGGCTGTCAGCGCCGGAGAGAAAGTGAC |
| | chain-codon | TATGAGTTGTAAATCTTCCCAGTCCCTGCT |
| | optimized nucleic acid | TAACTCACGTACTCGGAAGAATTATCTTGC |
| | | CTGGTATCAACAAAAGCCAGGTCAAAGTC |
| | | CTAAGCTCCTTATTTACTGGGCCTCAACAC |
| | | GGGAGTCAGGTGTCCCCGATCGCTTCACAG |
| | | GTAGTGGGAGTGGTACTGACTTCACTCTCA |
| | | CCATTTCAAATGTCCAAGCAGAAGACTTGA |
| | | CTGTGTATTACTGTAAGCAGAGTTACAACC |
| | | CTCCTTGGACCTTTGGTGGGGGGACCAAAC |
| | | TGGAGATCAAG |
| 24 | VL-CDR1 | KSSQSLLNSRTRKNYLA |
| 25 | VL-CDR2 | WASTRES |
| 26 | VL-CDR3 | KQSYNPPWT |

Antibody AGX-A05

| | | |
|---|---|---|
| 27 | AGX-A05 | EVQVQQSGPELVKPGASVKMSCKASGYTFT |
| | Variable heavy (VH) chain- | SYVMHWVKQKPGQGLEWIGYINPNNDNINY |
| | amino acid | NEKFKGKASLTSDKSSNTVYMELSSLTSEDS |
| | | AVYYCAGYGNSGANWGQGTLVTVSA |
| 28 | AGX-A05 | GAGGTCCAGGTACAGCAGTCTGGACCTGA |
| | Variable heavy (VH) chain- | ACTGGTAAAGCCTGGGGCTTCAGTGAAGA |
| | nucleic acid | TGTCCTGTAAGGCTTCTGGATACACATTCA |
| | | CTAGCTATGTCATGCACTGGGTGAAGCAG |
| | | AAGCCTGGGCAGGGCCTTGAGTGGATTGG |
| | | ATATATTAATCCTAACAATGATAATATTAA |
| | | CTACAATGAGAAGTTCAAAGGCAAGGCCT |
| | | CACTGACTTCAGACAAATCCTCCAACACAG |
| | | TCTACATGGAGCTCAGCAGCCTGACCTCTG |
| | | AGGACTCTGCGGTCTATTACTGTGCAGGCT |
| | | ATGGTAACTCCGGAGCTAACTGGGGCCAA |
| | | GGGACTCTGGTCACTGTCTCTGCA |
| 29 | AGX-A05 | GAAGTTCAAGTTCAGCAAAGCGGGCCTGA |
| | Variable heavy (VH) chain- | GCTTGTCAAGCCAGGCGCATCAGTCAAAA |
| | codon optimized nucleic acid | TGAGCTGTAAGGCTTCCGGGTACACCTTCA |
| | | CCAGTTATGTCATGCATTGGGTAAAACAAA |
| | | AGCCAGGACAGGGACTCGAGTGGATAGGA |
| | | TACATTAACCCAAATAACGACAACATTAA |
| | | CTACAACGAGAAATTCAAGGGCAAAGCAT |
| | | CATTGACTTCCGATAAATCCTCTAACACCG |
| | | TGTACATGGAGCTGAGTTCATTGACCAGCG |
| | | AGGATTCTGCCGTGTACTACTGTGCAGGTT |
| | | ATGGCAACTCTGGTGCTAACTGGGGGCAG |
| | | GGGACTCTGGTCACAGTCAGCGCA |
| 30 | VH-CDR1 | GYTFTSYVMH |
| 31 | VH-CDR2 | YINPNNDNINYNEKFKG |
| 32 | VH-CDR3 | YGNSGAN |
| 33 | AGX-A05 | DIQMTQSPASLSASVGETVTITCRTSKNIFNFL |
| | Variable light (VL)chain-amino | AWYHQKQGRSPRLLVSHTKTLAAGVPSRFS |
| | acid | GSGSGTQFSLKINSLQPEDFGIYYCQHHYGTP |
| | | WTFGGGTKLEIK |
| 34 | AGX-A05 | GACATCCAGATGACTCAGTCTCCAGCCTCC |
| | Variable light (VL) chain- | CTATCTGCATCTGTGGGAGAAACTGTCACC |
| | nucleic acid | ATCACATGTCGAACAAGTAAAAATATTTTC |
| | | AATTTTTTAGCATGGTATCACCAGAAACAG |
| | | GGAAGATCTCCTCGACTCCTGGTCTCTCAT |
| | | ACAAAAACCTTAGCAGCAGGTGTGCCATC |
| | | AAGGTTCAGTGGCAGTGGCTCAGGCACAC |
| | | AGTTTTCTCTGAAGATCAACAGCCTGCAGC |
| | | CTGAAGATTTTGGGATTTATTACTGTCAAC |
| | | ATCATTATGGTACTCCGTGGACGTTCGGTG |
| | | GAGGCACCAAACTGGAAATCAAA |
| 35 | AGX-A05 | GACATTCAGATGACCCAGTCACCAGCATCT |
| | Variable light (VL) chain-codon | TTGAGCGCATCCGTTGGGGAGACTGTGAC |
| | optimized nucleic acid | AATCACATGCCGAACCAGTAAGAACATCT |
| | | TCAACTTCCTCGCATGGTACCATCAAAAGC |
| | | AGGGCAGGTCTCCCAGACTGCTTGTCTCTC |
| | | ACACCAAGACACTGGCAGCAGGCGTCCCC |
| | | AGCCGGTTTAGTGGTAGTGGATCTGGCACA |
| | | CAGTTTAGTTTGAAAATCAATTCCCTGCAA |
| | | CCCGAAGACTTCGGCATATACTATTGCCAG |
| | | CACCACTATGGGACACCTTGGACTTTCGGA |
| | | GGTGGTACTAAACTTGAGATTAAA |
| 36 | VL-CDR1 | RTSKNIFNFLA |
| 37 | VL-CDR2 | HTKTLAA |
| 38 | VL-CDR3 | QHHYGTPWT |

Antibody AGX-A07

| | | |
|---|---|---|
| 39 | AGX-A07 | QIQLVQSGPELKKPGETVKISCKASGYTFTNY |
| | Variable heavy (VH) chain- | GVKWVKQAPGKDLKWMGWINTYTGNPIYA |
| | amino acid | ADFKGRFAFSLETSASTAFLQINNLKNEDTAT |
| | | YFCVRFQYGDYRYFDVWGAGTTVTVSS |
| 40 | AGX-A07 | CAGATCCAGTTGGTGCAGTCTGGACCTGAG |
| | Variable heavy (VH) chain- | CTGAAGAAGCCTGGAGAGACAGTCAAGAT |
| | nucleic acid | CTCCTGCAAGGCTTCTGGGTATACCTTCAC |
| | | AAACTATGGAGTGAAGTGGGTGAAGCAGG |
| | | CTCCAGGAAAGGATTTAAAGTGGATGGGC |
| | | TGGATAAACACCTACACTGGAAATCCAATT |
| | | TATGCTGCTGACTTCAAGGGACGGTTTGCC |
| | | TTCTCTTTGGAGACCTCTGCCAGCACTGCC |
| | | TTTTTGCAGATCAACAACCTCAAAAATGAG |
| | | GACACGGCTACATATTTCTGTGTAAGATTC |
| | | CAATATGGCGATTACCGGTACTTCGATGTC |
| | | TGGGGCGCAGGGACCACGGTCACCGTCTC |
| | | CTCA |
| 41 | AGX-A07 | CAAATCCAACTTGTCCAGAGCGGTCCCGA |
| | Variable heavy (VH) chain- | GTTGAAGAAGCCTGGCGAAACCGTGAAAA |
| | codon optimized nucleic acid | TCTCATGCAAGGCCAGTGGATATACATTTA |
| | | CAAACTATGGCGTCAAGTGGGTGAAACAA |
| | | GCCCCAGGTAAAGACTTGAAATGGATGGG |
| | | ATGGATCAACACATACACAGGGAATCCTA |
| | | TCTATGCAGCCGACTTTAAAGGCAGATTTG |
| | | CCTTCAGTTTGGAGACATCTGCCTCCACCG |
| | | CTTTCCTGCAAATAAATAACCTGAAAAATG |
| | | AAGATACCGCTACATACTTCTGTGTACGGT |
| | | TCCAGTACGGAGATTACCGCTATTTCGATG |
| | | TGTGGGGCGCAGGTACCACAGTAACCGTC |
| | | TCCTCA |
| 42 | VH-CDR1 | GYTFTNYGVK |
| 43 | VH-CDR2 | WINTYTGNPIYAADFKG |
| 44 | VH-CDR3 | FQYGDYRYFDV |
| 45 | AGX-A07 | QIILSQSPAILSASPGEKVTMTCRANSGISFIN |
| | Variable light (VL) chain-amino | WYQQKPGSSPKPWIYGTANLASGVPARFGG |
| | acid | SGSGTSYSLTISRVEAEDAATYYCQQWSSNP |
| | | LTFGAGTKLELR |
| 46 | AGX-A07 | CAAATTATTCTCTCCCAGTCTCCAGCAATC |
| | Variable light (VL) chain- | CTGTCTGCATCTCCAGGGGAGAAGGTCAC |
| | nucleic acid | GATGACTTGCAGGGCCAACTCAGGTATTA |
| | | GTTTCATCAACTGGTACCAGCAGAAGCCA |
| | | GGATCCTCCCCCAAACCCTGGATTTATGGC |
| | | ACAGCCAACCTGGCTTCTGGAGTCCCTGCT |
| | | CGCTTCGGTGGCAGTGGGTCTGGGACTTCT |
| | | TACTCTCTCACAATCAGCAGAGTGGAGGCT |
| | | GAAGACGCTGCCACTTATTACTGCCAGCAG |
| | | TGGAGTAGTAACCCGCTCACGTTCGGTGCT |
| | | GGGACCAAGCTGGAGTTGAGA |
| 47 | AGX-A07 | CAAATAATTCTGTCACAGTCCCCCGCTATA |
| | Variable light (VL) chain-codon | CTTAGTGCTTCACCAGGAGAAAAAGTGAC |
| | optimized nucleic acid | CATGACTTGTAGAGCTAATTCTGGCATATC |
| | | ATTCATCAACTGGTATCAACAAAAGCCAG |
| | | GTTCCTCCCCCAAGCCATGGATTTACGGGA |
| | | CCGCCAACCTTGCTTCTGGGGTACCCGCTC |
| | | GTTTCGGCGGATCAGGTTCAGGAACTTCCT |
| | | ATAGCCTCACTATCAGTCGGGTTGAAGCTG |
| | | AGGATGCCGCTACATATTACTGCCAGCAAT |
| | | GGTCTAGTAATCCACTTACCTTTGGAGCTG |
| | | GCACCAAATTGGAACTTCGT |
| 48 | VL-CDR1 | RANSGISFIN |
| 49 | VL-CDR2 | GTANLAS |
| 50 | VL-CDR3 | QQWSSNPLT |

Antibody AGX-A08

| | | |
|---|---|---|
| 51 | AGX-A08 | EVQLQQSGPELVKPGASVKLSCKASGYTVTS |
| | Variable heavy (VH) chain- | YVMHWVKQKPGQGLEWIGYINPYSDVTNC |
| | amino acid | NEKFKGKATLTSDKTSSTAYMELSSLTSEDS |
| | | AVYYCSSYGGGFAYWGQGTLVTVSA |
| 52 | AGX-A08 | GAGGTCCAGCTGCAGCAGTCTGGACCTGA |
| | Variable heavy (VH) chain- | GCTGGTAAAGCCTGGGGCTTCAGTGAAGC |
| | nucleic acid | TGTCCTGCAAGGCTTCTGGATACACAGTCA |
| | | CTAGCTATGTTATGCACTGGGTGAAGCAGA |
| | | AGCCTGGGCAGGGCCTTGAGTGGATTGGA |
| | | TATATTAATCCTTACAGTGATGTTACTAAC |
| | | TGCAATGAGAAGTTCAAAGGCAAGGCCAC |
| | | ACTGACTTCAGACAAAACCTCCAGCACAG |
| | | CCTACATGGAGCTCAGCAGCCTGACCTCTG |
| | | AGGACTCTGCGGTCTATTACTGTTCCTCCT |
| | | ACGGTGGGGGGTTTGCTTACTGGGGCCAA |
| | | GGGACTCTGGTCACTGTCTCTGCA |
| 53 | AGX-A08 | GAAGTCCAGCTTCAGCAATCCGGCCCAGA |
| | Variable heavy (VH) chain- | ACTGGTAAAACCAGGCGCAAGTGTTAAGT |
| | codon optimized nucleic acid | TGAGTTGCAAAGCCAGTGGTTATACCGTTA |
| | | CTTCATACGTCATGCATTGGGTAAAACAAA |
| | | AGCCCGGCCAAGGGCTTGAATGGATCGGC |
| | | TACATCAACCCTTACTCTGACGTCACCAAC |
| | | TGCAACGAGAAATTCAAAGGGAAAGCCAC |
| | | ATTGACCTCTGACAAGACAAGCAGTACCG |
| | | CCTACATGGAGCTTTCTAGTTTGACTTCTG |
| | | AAGACTCTGCTGTCTACTACTGTAGCAGCT |
| | | ACGGCGGCGGCTTTGCTTACTGGGGCCAG |
| | | GGTACATTGGTGACTGTGAGTGCA |
| 54 | VH-CDR1 | GYTVTSYVMH |
| 55 | VH-CDR2 | YINPYSDVTNCNEKFKG |
| 56 | VH-CDR3 | YGGGFAY |
| 57 | AGX-A08 | DIQMTQSPASLSASVGEPVTITCRASKNIYTY |
| | Variable light chain(VL)-amino | LAWYHQKQGKSPQFLVYNARTLAGGVPSRL |
| | acid | SGSGSVTQFSLNINTLHREDLGTYFCQHHYD |
| | | TPYTFGGGTNLEIK |
| 58 | AGX-A08 | GACATCCAGATGACTCAGTCTCCAGCCTCC |
| | Variable light (VL) chain- | CTATCTGCATCTGTGGGAGAACCTGTCACC |
| | nucleic acid | ATCACATGTCGAGCAAGTAAGAATATTTAC |
| | | ACATATTTAGCATGGTATCACCAGAAACA |
| | | GGGAAAATCTCCTCAGTTCCTGGTCTATAA |
| | | TGCAAGAACCTTAGCAGGAGGTGTGCCAT |
| | | CAAGGCTCAGTGGCAGTGGATCAGTCACG |
| | | CAGTTTTCTCTAAACATCAACACCTTGCAT |
| | | CGAGAAGATTTAGGGACTTACTTCTGTCAA |
| | | CATCATTATGATACTCCGTACACGTTCGGA |
| | | GGGGGGACCAACCTGGAAATAAAA |
| 59 | AGX-A08 | GACATCCAGATGACACAGTCACCAGCATC |
| | Variable light (VL) chain-codon | CCTGTCCGCCTCAGTTGGGGAGCCTGTTAC |
| | optimized nucleic acid | CATAACTTGTCGGGCAAGCAAAAACATAT |
| | | ACACCTATTTGGCTTGGTATCACCAAAAGC |
| | | AAGGTAAGTCACCTCAGTTTCTTGTATATA |
| | | ATGCCCGCACACTTGCTGGCGGAGTACCCT |
| | | CTCGATTGTCTGGATCTGGCAGCGTTACCC |
| | | AATTCAGCCTGAACATCAACACCCTCCATC |
| | | GGGAAGATTTGGGTACCTATTTCTGTCAAC |
| | | ATCACTACGACACCCCATACACCTTCGGAG |
| | | GCGGCACAAATTTGGAAATTAAA |
| 60 | VL-CDR1 | RASKNIYTYLA |
| 61 | VL-CDR2 | NARTLAG |
| 62 | VL-CDR3 | QHHYDTPYT |

Antibody AGX-A09

| | | |
|---|---|---|
| 63 | AGX-A09 | EVQLQQSGPELVKPGASVKMSCKASGYTFSS |
| | Variable heavy (VH) chain- | YVMHWVKQKPGQGLEWIGYINPYSDVTNY |
| | amino acid | NEKFKGKATLTSDRSSNTAYMELSSLTSEDS |
| | | AVYYCARNYFDWGRGTLVTVSA |
| 64 | AGX-A09 | GAGGTCCAGCTGCAGCAGTCTGGACCTGA |
| | Variable heavy (VH) chain- | GCTGGTAAAGCCTGGGGCTTCAGTGAAGA |
| | nucleic acid | TGTCCTGCAAGGCTTCTGGATACACATTCT |
| | | CTAGCTATGTTATGCACTGGGTGAAGCAGA |
| | | AGCCTGGGCAGGGCCTTGAGTGGATTGGA |
| | | TATATTAATCCTTACAGTGATGTCACTAAC |
| | | TACAATGAGAAGTTCAAAGGCAAGGCCAC |
| | | ACTGACTTCAGACAGATCCTCCAACACAGC |
| | | CTACATGGAACTCAGCAGCCTGACCTCTGA |
| | | GGACTCTGCGGTCTATTACTGTGCAAGAAA |
| | | TTACTTCGACTGGGGCCGAGGGACTCTGGT |
| | | CACAGTCTCTGCA |
| 65 | AGX-A09 | GAGGTACAGCTTCAGCAGAGTGGTCCAGA |
| | Variable heavy (VH) chain- | ACTCGTCAAGCCTGGGGCAAGCGTTAAGA |
| | codon optimized nucleic acid | TGAGTTGTAAAGCATCCGGTTACACATTCA |
| | | GTAGCTATGTTATGCACTGGGTCAAACAGA |
| | | AGCCTGGGCAGGGGTTGGAGTGGATCGGA |
| | | TATATAAATCCCTATTCAGACGTAACTAAT |
| | | TATAATGAAAAGTTCAAGGGGAAAGCAAC |
| | | CTTGACAAGTGACCGGTCATCTAATACCGC |
| | | ATACATGGAGCTGAGCTCATTGACAAGTG |
| | | AGGACTCTGCTGTGTATTACTGTGCCCGGA |
| | | ACTACTTCGACTGGGGTAGGGGCACACTG |
| | | GTAACTGTTAGTGCA |
| 66 | VH-CDR1 | GYTFSSYVMH |
| 67 | VH-CDR2 | YINPYSDVTNYNEKFKG |
| 68 | VH-CDR3 | NYFD |
| 69 | AGX-A09 | DIQMTQSPASLSASVGETVTITCRASKNVYS |
| | Variable light (VL) chain-amino | YLAWFQQKQGKSPQLLVYNAKTLAEGVPSR |
| | acid | FSGGGSGTQFSLKINSLQPADFGSYYCQHHY |
| | | NIPFTFGSGTKLEIK |
| 70 | AGX-A09 | GACATCCAGATGACTCAGTCTCCAGCCTCC |
| | Variable light (VL) chain- | CTATCTGCATCTGTGGGAGAAACTGTCACC |
| | nucleic acid | ATCACATGTCGAGCAAGTAAAAATGTTTAC |
| | | AGTTATTTAGCATGGTTTCAACAGAAACAG |
| | | GGGAAATCTCCTCAGCTCCTGGTCTATAAT |
| | | GCTAAAACCTTAGCAGAAGGTGTGCCATC |
| | | AAGGTTCAGTGGCGGGGGATCAGGCACAC |
| | | AGTTTTCTCTGAAGATCAACAGCCTGCAGC |
| | | CTGCAGATTTTGGGAGTTATTACTGTCAAC |
| | | ATCATTATAATATTCCATTCACGTTCGGCT |
| | | CGGGGACAAAGTTGGAAATAAAA |
| 71 | AGX-A09 | GACATACAAATGACACAAAGTCCCGCTAG |
| | Variable light (VL) chain-codon | TCTTTCAGCCAGTGTTGGTGAGACTGTGAC |
| | optimized nucleic acid | AATAACCTGTAGAGCTAGCAAAAATGTCT |
| | | ACTCCTATCTGGCTTGGTTCCAGCAGAAAC |
| | | AAGGAAAGAGTCCTCAGTTGCTCGTATATA |
| | | ATGCTAAAACTTTGGCAGAAGGCGTCCCTT |
| | | CTCGTTTCAGTGGCGGAGGAAGTGGGACT |
| | | CAATTCTCACTGAAGATCAATAGCCTCCAG |
| | | CCCGCCGACTTTGGGAGCTACTATTGCCAA |
| | | CATCATTACAACATACCATTCACCTTTGGC |
| | | TCAGGTACTAAACTCGAAATTAAA |
| 72 | VL-CDR1 | RASKNVYSYLA |
| 73 | VL-CDR2 | NAKTLAE |
| 74 | VL-CDR3 | QHHYNIPFT |

Antibody AGX-A11

| | | |
|---|---|---|
| 75 | AGX-A11 | QIQLVQSGPELKKPGETVKISCKASGFTFTNY |
| | Variable heavy (VH) chain- | PMHWVKQAPGKGLKWMGWINTYSGVPTY |
| | amino acid | ADDFKGRFAFSLETSASTAYLQINNLKNEDM |
| | | ATYFCARGGYDGSREFAYWGQGTLVTVS |
| 76 | AGX-A11 | CAGATCCAGTTGGTGCAGTCTGGACCTGAG |
| | Variable heavy (VH) chain- | CTGAAGAAGCCTGGAGAGACAGTCAAGAT |
| | nucleic acid | CTCCTGCAAGGCTTCTGGGTTTACCTTCAC |
| | | AAACTATCCAATGCACTGGGTGAAGCAGG |
| | | CTCCAGGAAAGGGTTTAAAGTGGATGGGC |
| | | TGGATAAACACCTACTCTGGAGTGCCAAC |
| | | ATATGCAGATGACTTCAAGGGACGGTTTGC |
| | | CTTCTCTTTGGAAACCTCTGCCAGCACTGC |
| | | ATATTTGCAGATCAACAACCTCAAAAATG |
| | | AGGACATGGCTACATATTTCTGTGCAAGAG |
| | | GGGGCTACGATGGTAGCAGGGAGTTTGCT |
| | | TACTGGGGCCAAGGGACTCTGGTCACTGTC |
| | | TCT |
| 77 | AGX-A11 | CAGATACAACTCGTCCAGTCAGGTCCAGA |
| | Variable heavy (VH) chain- | GTTGAAGAAACCCGGAGAAACTGTGAAGA |
| | codon optimized nucleic acid | TATCCTGTAAAGCCAGCGGCTTTACTTTCA |
| | | CAAACTACCCCATGCATTGGGTGAAGCAG |
| | | GCCCCCGGAAAAGGACTCAAATGGATGGG |
| | | ATGGATCAACACATACAGTGGGGTGCCTA |
| | | CTTACGCAGACGATTTCAAAGGAAGGTTC |
| | | GCATTTAGCTTGGAAACTAGCGCATCTACA |
| | | GCATATCTCCAGATTAACAATCTTAAAAAT |
| | | GAGGATATGGCAACATACTTCTGCGCTAG |
| | | GGGAGGTTACGATGGGAGCAGGGAGTTCG |
| | | CTTATTGGGGGCAAGGGACTCTTGTGACTG |
| | | TAAGT |
| 78 | VH-CDR1 | GFTFTNYPMH |
| 79 | VH-CDR2 | WINTYSGVPTYADDFKG |
| 80 | VH-CDR3 | GGYDGSREFAY |
| 81 | AGX-A11 | DIVLTQSPASLAASLGQRATTSYRASKSVSTS |
| | Variable light (VL) chain-amino | GYSYMHWNQQKPGQPPRLLIYLVSNLESGV |
| | acid | PARFSGSGSGTDFTLNIHPVEEEDAATYYCQ |
| | | HIRELTTFGGGTKLEIK |
| 82 | AGX-A11 | GACATTGTGCTGACACAGTCTCCTGCTTCC |
| | Variable light (VL) chain- | TTAGCTGCATCTCTGGGGCAGAGGGCCACC |
| | nucleic acid | ACCTCATACAGGGCCAGCAAAAGTGTCAG |
| | | TACATCTGGCTATAGTTATATGCACTGGAA |
| | | CCAACAGAAACCAGGACAGCCACCCAGAC |
| | | TCCTCATCTATCTTGTATCCAACCTAGAAT |
| | | CTGGGGTCCCTGCCAGGTTCAGTGGCAGTG |
| | | GGTCTGGGACAGACTTCACCCTCAACATCC |
| | | ATCCTGTGGAGGAGGAGGATGCTGCAACC |
| | | TATTACTGTCAGCACATTAGGGAGCTTACC |
| | | ACGTTCGGAGGGGGGACCAAGCTGGAAAT |
| | | AAAA |
| 83 | AGX-A11 | GACATAGTGCTCACTCAGAGCCCTGCATCC |
| | Variable light (VL) chain-codon | CTTGCCGCCTCCCTCGGACAACGAGCTACT |
| | optimized nucleic acid | ACAAGCTACCGGGCATCAAAGTCCGTTAG |
| | | CACATCAGGATACAGCTATATGCACTGGA |
| | | ATCAGCAAAAGCCAGGCCAACCACCCCGT |
| | | CTTCTCATCTACCTCGTAAGTAATCTGGAA |
| | | TCAGGCGTGCCAGCCCGATTCAGTGGGTCA |
| | | GGGTCTGGGACAGATTTCACCCTCAACATC |
| | | CATCCAGTAGAGGAAGAGGACGCAGCAAC |
| | | ATATTACTGCCAACACATTAGAGAACTTAC |
| | | CACTTTCGGAGGAGGAACTAAATTGGAGA |
| | | TCAAA |
| 84 | VL-CDR1 | RASKSVSTSGYSYMH |
| 85 | VL-CDR2 | LVSNLES |
| 86 | VL-CDR3 | QHIRELT |

Constant Region Sequences

| | | |
|---|---|---|
| 87 | IgG1 Glm17* (heavy chain | ASTKGPSVFPLAPSSKSTSGGTAALGCLVKD |
| | constant region) | YFPEPVTVSWNSGALTSGVHTFPAVLQSSGL |
| | * with L234A/L235A/G237A | YSLSSVVTVPSSSLGTQTYICNVNHKPSNTKV |
| | mutations | DKKVEPKSCDKTHTCPPCPAPEAAGAPSVFL |
| | SEQ ID NO: 88 is sequence | FPPKPKDTLMISRTPEVTCVVVDVSHEDPEV |
| | without the terminal lysine | KFNWYVDGVEVHNAKTKPREEQYNSTYRV |
| | | VSVLTVLHQDWLNGKEYKCKVSNKALPAPI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 88 | IgG1 Glm17* (heavy chain | ASTKGPSVFPLAPSSKSTSGGTAALGCLVKD |
| | constant region) | YFPEPVTVSWNSGALTSGVHTFPAVLQSSGL |
| | * with L234A/L235A/G237A | YSLSSVVTVPSSSLGTQTYICNVNHKPSNTKV |
| | mutations | DKKVEPKSCDKTHTCPPCPAPEAAGAPSVFL |
| | | FPPKPKDTLMISRTPEVTCVVVDVSHEDPEV |
| | | KFNWYVDGVEVHNAKTKPREEQYNSTYRV |
| | | VSVLTVLHQDWLNGKEYKCKVSNKALPAPI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPG |
| 89 | IgG1 Km3 (light chain constant | RTVAAPSVFIFPPSDEQLKSGTASVVCLLNNF |
| | region) | YPREAKVQWKVDNALQSGNSQESVTEQDSK |
| | | DSTYSLSSTLTLSKADYEKHKVYACEVTHQG |
| | | LSSPVTKSFNRGEC |

Humanized AGX-A07 sequences

| | | |
|---|---|---|
| 90 | AGX-A07 (humanized) H2 | QVQLVQSGAEVKKPGASVKVSCKASGYTFT |
| | Heavy chain amino acid | NYGVKWVRQAPGQDLEWMGWINTYTGNPI |
| | | YAADFKGRVTMTTDTSTSTAFMELRSLRSD |
| | | DTAVYYCVRFQYGDYRYFDVWGQGTLVTV |
| | | SSASTKGPSVFPLAPSSKSTSGGTAALGCLVK |
| | | DYFPEPVTVSWNSGALTSGVHTFPAVLQSSG |
| | | LYSLSSVVTVPSSSLGTQTYICNVNHKPSNTK |
| | | VDKKVEPKSCDKTHTCPPCPAPEAAGAPSVF |
| | | LFPPKPKDTLMISRTPEVTCVVVDVSHEDPE |
| | | VKFNWYVDGVEVHNAKTKPREEQYNSTYR |
| | | VVSVLTVLHQDWLNGKEYKCKVSNKALPAP |
| | | IEKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 91 | AGX-A07 (humanized) H2 | TCTACCGGACAGGTGCAGTTGGTTCAGTCT |
| | Heavy chain nucleic acid | GGCGCCGAAGTGAAGAAACCTGGCGCTTC |
| | | TGTGAAGGTGTCCTGCAAGGCCTCTGGCTA |
| | | CACCTTTACCAACTACGGCGTGAAATGGGT |
| | | CCGACAGGCTCCTGGACAGGATCTGGAAT |
| | | GGATGGGCTGGATCAACACCTACACCGGC |
| | | AATCCTATCTACGCCGCCGACTTCAAGGGC |
| | | AGAGTGACCATGACCACCGACACCTCTAC |
| | | CTCCACCGCCTTCATGGAACTGCGGTCCCT |
| | | GAGATCTGACGACACCGCCGTGTACTACTG |
| | | CGTGCGGTTTCAGTACGGCGACTACCGGTA |
| | | CTTTGATGTGTGGGGCCAGGGCACACTGGT |
| | | CACCGTTTCTTCCGCTTCTACCAAGGGACC |
| | | CAGCGTGTTCCCTCTGGCTCCTTCCTCTAA |
| | | ATCCACCTCTGGCGGAACCGCTGCTCTGGG |
| | | CTGTCTGGTCAAGGATTACTTCCCTGAGCC |
| | | TGTGACCGTGTCCTGGAACTCTGGTGCTCT |
| | | GACATCCGGCGTGCACACCTTTCCAGCTGT |
| | | GCTGCAGTCCTCTGGCCTGTACTCTCTGTC |
| | | CTCTGTCGTGACCGTGCCTTCTAGCTCTCT |
| | | GGGCACCCAGACCTACATCTGCAACGTGA |
| | | ACCACAAGCCTTCCAACACCAAGGTGGAC |
| | | AAGAAGGTGGAACCCAAGTCCTGCGACAA |
| | | GACCCACACCTGTCCTCCATGTCCTGCTCC |
| | | AGAAGCTGCTGGCGCTCCCTCTGTGTTCCT |
| | | GTTTCCTCCAAAGCCTAAGGACACCCTGAT |
| | | GATCTCTCGGACCCCTGAAGTGACCTGCGT |
| | | GGTGGTGGATGTGTCTCACGAGGACCCAG |
| | | AAGTGAAGTTCAATTGGTACGTGGACGGC |
| | | GTGGAAGTGCACAACGCCAAGACCAAGCC |
| | | TAGAGAGGAACAGTACAACTCCACCTACA |
| | | GAGTGGTGTCCGTGCTGACCGTGCTGCACC |
| | | AGGATTGGCTGAACGGCAAAGAGTACAAG |
| | | TGCAAGGTGTCCAACAAGGCACTGCCCGC |
| | | TCCTATCGAAAAGACCATCTCCAAGGCTAA |
| | | GGGCCAGCCTCGGGAACCTCAGGTTTACA |
| | | CCCTGCCTCCATCTCGGGAAGAGATGACCA |
| | | AGAACCAGGTGTCCCTGACCTGCCTCGTGA |
| | | AGGGCTTCTACCCTTCCGATATCGCCGTGG |
| | | AATGGGAGTCCAATGGCCAGCCTGAGAAC |
| | | AACTACAAGACAACCCCTCCTGTGCTGGAC |
| | | TCCGACGGCTCATTCTTCCTGTACTCCAAG |
| | | CTGACAGTGGACAAGTCTCGGTGGCAGCA |
| | | GGGCAACGTGTTCTCCTGTTCTGTGATGCA |
| | | CGAGGCCCTGCACAACCACTACACACAGA |
| | | AGTCCCTGTCTCTGTCCCCTGGCAAGTGA |
| 92 | AGX-A07 H2v1 | EVQLVQSGAEVKKPGASVKVSCKASGYTFT |
| | Heavy chain amino acid | NYGVKWVRQAPGQGLEWMGWINTYTGNPI |
| | | YAADFKGRVTMTTDTSTSTAYMELRSLRSD |
| | | DTAVYYCVRFQYGDYRYFDVWGQGTLVTV |
| | | SSASTKGPSVFPLAPSSKSTSGGTAALGCLVK |
| | | DYFPEPVTVSWNSGALTSGVHTFPAVLQSSG |
| | | LYSLSSVVTVPSSSLGTQTYICNVNHKPSNTK |
| | | VDKKVEPKSCDKTHTCPPCPAPEAAGAPSVF |
| | | LFPPKPKDTLMISRTPEVTCVVVDVSHEDPE |
| | | VKFNWYVDGVEVHNAKTKPREEQYNSTYR |
| | | VVSVLTVLHQDWLNGKEYKCKVSNKALPAP |
| | | IEKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 93 | AGX-A07 H2v1 | GAAGTGCAGTTGGTGCAGTCTGGCGCCGA |
| | Heavy chain nucleic acid | AGTGAAGAAACCTGGCGCTTCTGTGAAGG |
| | | TGTCCTGCAAGGCCTCTGGCTACACCTTTA |
| | | CCAACTACGGCGTGAAATGGGTCCGACAG |
| | | GCTCCTGGACAAGGCCTGGAATGGATGGG |
| | | CTGGATCAACACCTACACCGGCAATCCTAT |
| | | CTACGCCGCCGACTTCAAGGGCAGAGTGA |
| | | CCATGACCACCGACACCTCTACCTCCACCG |
| | | CCTACATGGAACTGCGGTCCCTGAGATCTG |
| | | ACGACACCGCCGTGTACTACTGCGTGCGGT |
| | | TTCAGTACGGCGACTACCGGTACTTTGATG |
| | | TGTGGGGCCAGGGCACACTGGTCACCGTTT |
| | | CTTCCGCTTCTACCAAGGGACCCAGCGTGT |
| | | TCCCTCTGGCTCCTTCCTCTAAATCCACCTC |
| | | TGGCGGAACCGCTGCTCTGGGCTGTCTGGT |
| | | CAAGGATTACTTCCCTGAGCCTGTGACCGT |
| | | GTCCTGGAATTCTGGTGCTCTGACATCCGG |
| | | CGTGCACACCTTTCCAGCTGTGCTGCAGTC |
| | | CTCTGGCCTGTACTCTCTGTCCTCTGTCGTG |
| | | ACCGTGCCTTCTAGCTCTCTGGGCACCCAG |
| | | ACCTACATCTGCAACGTGAACCACAAGCCT |
| | | TCCAACACCAAGGTGGACAAGAAGGTGGA |
| | | ACCCAAGTCCTGCGACAAGACCCACACCT |
| | | GTCCTCCATGTCCTGCTCCAGAAGCTGCTG |
| | | GCGCTCCCTCTGTGTTCCTGTTTCCTCCAAA |
| | | GCCTAAGGACACCCTGATGATCTCTCGGAC |
| | | CCCTGAAGTGACCTGCGTGGTGGTGGATGT |
| | | GTCTCACGAGGACCCAGAAGTGAAGTTCA |
| | | ATTGGTACGTGGACGGCGTGGAAGTGCAC |
| | | AACGCCAAGACCAAGCCTAGAGAGGAACA |
| | | GTACAACTCCACCTACAGAGTGGTGTCCGT |
| | | GCTGACCGTGCTGCACCAGGATTGGCTGA |
| | | ACGGCAAAGAGTACAAGTGCAAGGTGTCC |
| | | AACAAGGCACTGCCCGCTCCTATCGAAAA |
| | | GACCATCTCCAAGGCTAAGGGCCAGCCTC |
| | | GGGAACCTCAGGTTTACACCCTGCCTCCAT |
| | | CTCGGGAAGAGATGACCAAGAACCAGGTG |
| | | TCCCTGACCTGCCTCGTGAAGGGCTTCTAC |
| | | CCTTCCGATATCGCCGTGGAATGGGAGTCC |
| | | AATGGCCAGCCTGAGAACAACTACAAGAC |
| | | AACCCCTCCTGTGCTGGACTCCGACGGCTC |
| | | ATTCTTCCTGTACTCCAAGCTGACAGTGGA |
| | | CAAGTCTCGGTGGCAGCAGGGCAACGTGT |
| | | TCTCCTGTTCTGTGATGCACGAGGCCCTGC |
| | | ACAACCACTACACACAGAAGTCCCTGTCTC |
| | | TGTCCCCTGGCAAGTGA |
| 94 | VH-CDR1 | GYTFTNYGVK |
| 95 | VH-CDR2 | WINTYTGNPIYAADFK |
| 96 | VH-CDR3 | FQYGDYRYFDV |
| 97 | AGX-A07 L5 | EIILTQSPATLSLSPGERATLSCRANSGISFIN |
| | Light chain amino acid | WYQQKPGQAPRLLIYGTANLASGIPARFGGS |
| | | GSGRDFTLTISSLEPEDFAVYYCQQWSSNPLT |
| | | FGGGTKVEIKRTVAAPSVFIFPPSDEQLKSGT |
| | | ASVVCLLNNFYPREAKVQWKVDNALQSGNS |
| | | QESVTEQDSKDSTYSLSSTLTLSKADYEKHK |
| | | VYACEVTHQGLSSPVTKSFNRGEC |
| 98 | AGX-A07 L5 | AAGCTTGCCACCATGGAAACCGACACACT |
| | Light chain nucleic acid | GCTGCTGTGGGTGCTGTTGTTGTGGGTGCC |
| | | AGGATCTACCGGAGAGATCATCCTGACAC |
| | | AGAGCCCCGCCACATTGTCTCTGAGTCCTG |
| | | GCGAGAGAGCTACCCTGTCCTGTAGAGCC |
| | | AACTCCGGCATCTCCTTCATCAACTGGTAT |
| | | CAGCAGAAGCCCGGCCAGGCTCCTAGACT |
| | | GCTGATCTATGGCACCGCTAACCTGGCCTC |
| | | TGGCATCCCTGCTAGATTTGGCGGCTCTGG |
| | | CTCTGGCAGAGACTTCACCCTGACCATCTC |
| | | TAGCCTGGAACCTGAGGACTTCGCCGTGTA |
| | | CTACTGCCAGCAGTGGTCTAGCAACCCTCT |
| | | GACCTTTGGCGGAGGCACCAAGGTGGAAA |
| | | TCAAGAGAACCGTGGCCGCTCCTTCCGTGT |
| | | TCATCTTCCCACCATCTGACGAGCAGCTGA |
| | | AGTCTGGCACAGCCTCTGTCGTGTGCCTGC |
| | | TGAACAACTTCTACCCTCGGGAAGCCAAG |
| | | GTGCAGTGGAAGGTGGACAATGCCCTGCA |
| | | GTCCGGCAACTCCCAAGAGTCTGTGACCG |
| | | AGCAGGACTCCAAGGACTCTACCTACAGC |
| | | CTGTCCTCCACACTGACCCTGTCTAAGGCC |
| | | GACTACGAGAAGCACAAGGTGTACGCCTG |
| | | TGAAGTGACCCACCAGGGACTGTCTAGCC |
| | | CCGTGACCAAGTCTTTCAACCGGGGCGAGT |
| | | GCTGA |
| 99 | AGX-A07 L5v1 | EIVLTQSPATLSLSPGERATLSCRANSGISFI |
| | Light chain amino acid | NWYQQKPGQAPRLLIYGTANLASGIPARFSGS |
| | | GSGRDFTLTISSLEPEDFAVYYCQQWSSNPLT |
| | | FGGGTKVEIKRTVAAPSVFIFPPSDEQLKSGT |
| | | ASVVCLLNNFYPREAKVQWKVDNALQSGNS |
| | | QESVTEQDSKDSTYSLSSTLTLSKADYEKHK |
| | | VYACEVTHQGLSSPVTKSFNRGEC |
| 100 | AGX-A07 L5v1 | TCTACAGGCGAGATCGTGCTGACCCAGTCT |
| | Light chain nucleic acid | CCTGCCACATTGTCTCTGAGTCCTGGCGAG |
| | | AGAGCTACCCTGTCCTGTAGAGCCAACTCC |
| | | GGCATCTCCTTCATCAACTGGTATCAGCAG |
| | | AAGCCCGGCCAGGCTCCTAGACTGCTGATC |
| | | TATGGCACCGCTAACCTGGCCTCTGGCATC |
| | | CCTGCTAGATTTTCCGGCTCTGGCTCTGGC |
| | | AGAGACTTCACCCTGACCATCTCTAGCCTG |
| | | GAACCTGAGGACTTCGCCGTGTACTACTGC |
| | | CAGCAGTGGTCTAGCAACCCTCTGACCTTT |
| | | GGCGGAGGCACCAAGGTGGAAATCAAGAG |
| | | AACCGTGGCCGCTCCTTCCGTGTTCATCTT |
| | | CCCACCATCTGACGAGCAGCTGAAGTCTG |
| | | GCACAGCCTCTGTCGTGTGCCTGCTGAACA |
| | | ACTTCTACCCTCGGGAAGCCAAGGTGCAGT |
| | | GGAAGGTGGACAATGCCCTGCAGTCCGGC |
| | | AACTCCCAAGAGTCTGTGACCGAGCAGGA |
| | | CTCCAAGGACTCTACCTACAGCCTGTCCTC |
| | | CACACTGACCCTGTCTAAGGCCGACTACGA |
| | | GAAGCACAAGGTGTACGCCTGTGAAGTGA |
| | | CCCACCAGGGACTGTCTAGCCCCGTGACCA |
| | | AGTCTTTCAACCGGGGCGAGTGCTGA |
| 101 | AGX-A07 L5v2 | EIVLTQSPATLSLSPGERATLSCRAQSGISFI |
| | Light chain amino acid | NWYQQKPGQAPRLLIYGTANLASGIPARFSGS |
| | | GSGRDFTLTISSLEPEDFAVYYCQQWSSNPLT |
| | | FGGGTKVEIKRTVAAPSVFIFPPSDEQLKSGT |
| | | ASVVCLLNNFYPREAKVQWKVDNALQSGNS |
| | | QESVTEQDSKDSTYSLSSTLTLSKADYEKHK |
| | | VYACEVTHQGLSSPVTKSFNRGEC |
| 102 | AGX-A07 L5v2 | TCTACAGGCGAGATCGTGCTGACCCAGTCT |
| | Light chain nucleic acid | CCTGCCACATTGTCTCTGAGTCCTGGCGAG |
| | | AGAGCTACCCTGTCTTGTAGAGCCCAGTCC |
| | | GGCATCTCCTTCATCAACTGGTATCAGCAG |
| | | AAGCCCGGCCAGGCTCCTAGACTGCTGATC |
| | | TATGGCACCGCTAACCTGGCCTCTGGCATC |
| | | CCTGCTAGATTTTCCGGCTCTGGCTCTGGC |
| | | AGAGACTTCACCCTGACCATCTCTAGCCTG |
| | | GAACCTGAGGACTTCGCCGTGTACTACTGC |
| | | CAGCAGTGGTCTAGCAACCCTCTGACCTTT |
| | | GGCGGAGGCACCAAGGTGGAAATCAAGAG |
| | | AACCGTGGCCGCTCCTTCCGTGTTCATCTT |
| | | CCCACCATCTGACGAGCAGCTGAAGTCTG |
| | | GCACAGCCTCTGTCGTGTGCCTGCTGAACA |
| | | ACTTCTACCCTCGGGAAGCCAAGGTGCAGT |
| | | GGAAGGTGGACAATGCCCTGCAGTCTGGC |
| | | AACTCCCAAGAGTCTGTGACCGAGCAGGA |
| | | CTCCAAGGACTCTACCTACAGCCTGTCCTC |
| | | CACACTGACCCTGTCTAAGGCCGACTACGA |
| | | GAAGCACAAGGTGTACGCCTGTGAAGTGA |
| | | CCCACCAGGGACTGTCTAGCCCCGTGACCA |
| | | AGTCTTTCAACCGGGGCGAGTGCTGA |
| 103 | AGX-A07 L5v3 | EIVLTQSPATLSLSPGERATLSCRANSGISFI |
| | Light chain amino acid | NWYQQKPGQAPRLLIYGTANLASGIPARFSGS |
| | | GSGRDFTLTISSLEPEDFAVYYCQQYSSNPLT |
| | | FGGGTKVEIKRTVAAPSVFIFPPSDEQLKSGT |
| | | ASVVCLLNNFYPREAKVQWKVDNALQSGNS |
| | | QESVTEQDSKDSTYSLSSTLTLSKADYEKHK |
| | | VYACEVTHQGLSSPVTKSFNRGEC |
| 104 | AGX-A07 L5v3 | TCTACAGGCGAGATCGTGCTGACCCAGTCT |
| | Light chain nucleic acid | CCTGCCACATTGTCTCTGAGTCCTGGCGAG |
| | | AGAGCTACCCTGTCCTGTAGAGCCAACTCC |
| | | GGCATCTCCTTCATCAACTGGTATCAGCAG |
| | | AAGCCCGGCCAGGCTCCTAGACTGCTGATC |
| | | TATGGCACCGCTAACCTGGCCTCTGGCATC |
| | | CCTGCTAGATTTTCCGGCTCTGGCTCTGGC |
| | | AGAGACTTCACCCTGACCATCTCTAGCCTG |
| | | GAACCTGAGGACTTCGCCGTGTACTACTGC |
| | | CAGCAGTACAGCAGCAACCCTCTGACCTTT |
| | | GGCGGAGGCACCAAGGTGGAAATCAAGAG |
| | | AACCGTGGCCGCTCCTTCCGTGTTCATCTT |
| | | CCCACCATCTGACGAGCAGCTGAAGTCTG |
| | | GCACAGCCTCTGTCGTGTGCCTGCTGAACA |
| | | ACTTCTACCCTCGGGAAGCCAAGGTGCAGT |
| | | GGAAGGTGGACAATGCCCTGCAGTCCGGC |
| | | AACTCCCAAGAGTCTGTGACCGAGCAGGA |
| | | CTCCAAGGACTCTACCTACAGCCTGTCCTC |
| | | CACACTGACCCTGTCTAAGGCCGACTACGA |
| | | GAAGCACAAGGTGTACGCCTGTGAAGTGA |
| | | CCCACCAGGGACTGTCTAGCCCCGTGACCA |
| | | AGTCTTTCAACCGGGGCGAGTGCTGA |
| 105 | AGX-A07 L5v4 | EIVLTQSPATLSLSPGERATLSCRAQSGISFIN |
| | Light chain amino acid | WYQQKPGQAPRLLIYGTANLASGIPARFSGS |
| | | GSGRDFTLTISSLEPEDFAVYYCQQYSSNPLT |
| | | FGGGTKVEIKRTVAAPSVFIFPPSDEQLKSGT |
| | | ASVVCLLNNFYPREAKVQWKVDNALQSGNS |
| | | QESVTEQDSKDSTYSLSSTLTLSKADYEKHK |
| | | VYACEVTHQGLSSPVTKSFNRGEC |
| 106 | AGX-A07 L5v4 | TCTACAGGCGAGATCGTGCTGACCCAGTCT |
| | Light chain nucleic acid | CCTGCCACATTGTCTCTGAGTCCTGGCGAG |
| | | AGAGCTACCCTGTCTTGTAGAGCCCAGTCC |
| | | GGCATCTCCTTCATCAACTGGTATCAGCAG |
| | | AAGCCCGGCCAGGCTCCTAGACTGCTGATC |
| | | TATGGCACCGCTAACCTGGCCTCTGGCATC |
| | | CCTGCTAGATTTTCCGGCTCTGGCTCTGGC |
| | | AGAGACTTCACCCTGACCATCTCTAGCCTG |
| | | GAACCTGAGGACTTCGCCGTGTACTACTGC |
| | | CAGCAGTACAGCAGCAACCCTCTGACCTTT |
| | | GGCGGAGGCACCAAGGTGGAAATCAAGAG |
| | | AACCGTGGCCGCTCCTTCCGTGTTCATCTT |
| | | CCCACCATCTGACGAGCAGCTGAAGTCTG |
| | | GCACAGCCTCTGTCGTGTGCCTGCTGAACA |
| | | ACTTCTACCCTCGGGAAGCCAAGGTGCAGT |
| | | GGAAGGTGGACAATGCCCTGCAGTCTGGC |
| | | AACTCCCAAGAGTCTGTGACCGAGCAGGA |
| | | CTCCAAGGACTCTACCTACAGCCTGTCCTC |
| | | CACACTGACCCTGTCTAAGGCCGACTACGA |
| | | GAAGCACAAGGTGTACGCCTGTGAAGTGA |
| | | CCCACCAGGGACTGTCTAGCCCCGTGACCA |
| | | AGTCTTTCAACCGGGGCGAGTGCTGA |
| 107 | VL-CDR1 (variant 1) | RANSGISFIN |
| 108 | VL-CDR1 (variant 2) | RAQSGISFIN |
| 109 | VL-CDR2 | GTANLAS |
| 110 | VL-CDR3 (variant 1) | QQWSSNPLT |
| 111 | VL-CDR3 (variant 2) | QQYSSNPLT |

Humanized AGX-A01 sequences

| | | |
|---|---|---|
| 112 | AGX-A01 H1 | EVOLVESGGGLVKPGGSLRLSCAASGFTFSS |
| | Heavy chain amino acid | FAMSWVRQAPGKGLEWVSTISSGSIYIYYTD |
| | | GVKGRFTISRDNAKNSLYLQMNSLRAEDTA |
| | | VYYCARRGIYYGYDGYAMDYWGQGTLVTV |
| | | SSASTKGPSVFPLAPSSKSTSGGTAALGCLVK |
| | | DYFPEPVTVSWNSGALTSGVHTFPAVLQSSG |
| | | LYSLSSVVTVPSSSLGTQTYICNVNHKPSNTK |
| | | VDKKVEPKSCDKTHTCPPCPAPEAAGAPSVF |
| | | LFPPKPKDTLMISRTPEVTCVVVDVSHEDPE |
| | | VKFNWYVDGVEVHNAKTKPREEQYNSTYR |
| | | VVSVLTVLHQDWLNGKEYKCKVSNKALPAP |
| | | IEKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 113 | AGX-A01 H1 | GAGGTGCAGCTGGTTGAATCTGGCGGAGG |
| | Heavy chain nucleic acid | ACTTGTGAAGCCTGGCGGCTCTCTGAGACT |
| | | GTCTTGTGCCGCCTCTGGCTTCACCTTCTCC |
| | | AGCTTTGCCATGTCCTGGGTCCGACAGGCT |
| | | CCTGGCAAAGGACTGGAATGGGTGTCCAC |
| | | CATCTCCTCCGGCTCCATCTACATCTACTA |
| | | CACCGACGGCGTGAAGGGCAGATTCACCA |
| | | TCAGCAGAGACAACGCCAAGAACTCCCTG |
| | | TACCTGCAGATGAACAGCCTGAGAGCCGA |
| | | GGACACCGCCGTGTACTATTGTGCCAGACG |
| | | GGGCATCTACTATGGCTACGACGGCTACGC |
| | | TATGGACTATTGGGGACAGGGCACACTGG |
| | | TCACCGTGTCCTCTGCTTCTACCAAGGGAC |
| | | CCAGCGTGTTCCCTCTGGCTCCTTCCTCTA |
| | | AATCCACCTCTGGCGGAACCGCTGCTCTGG |
| | | GCTGTCTGGTCAAGGATTACTTCCCTGAGC |
| | | CTGTGACCGTGTCCTGGAACTCTGGTGCTC |
| | | TGACATCCGGCGTGCACACCTTTCCAGCTG |
| | | TGCTGCAGTCCTCTGGCCTGTACTCTCTGT |
| | | CCTCTGTCGTGACCGTGCCTTCTAGCTCTCT |
| | | GGGCACCCAGACCTACATCTGCAACGTGA |
| | | ACCACAAGCCTTCCAACACCAAGGTGGAC |
| | | AAGAAGGTGGAACCCAAGTCCTGCGACAA |
| | | GACCCACACCTGTCCTCCATGTCCTGCTCC |
| | | AGAAGCTGCTGGCGCTCCCTCTGTGTTCCT |
| | | GTTTCCTCCAAAGCCTAAGGACACCCTGAT |
| | | GATCTCTCGGACCCCTGAAGTGACCTGCGT |
| | | GGTGGTGGATGTGTCTCACGAGGACCCAG |
| | | AAGTGAAGTTCAATTGGTACGTGGACGGC |
| | | GTGGAAGTGCACAACGCCAAGACCAAGCC |
| | | TAGAGAGGAACAGTACAACTCCACCTACA |
| | | GAGTGGTGTCCGTGCTGACCGTGCTGCACC |
| | | AGGATTGGCTGAACGGCAAAGAGTACAAG |
| | | TGCAAGGTGTCCAACAAGGCACTGCCCGC |
| | | TCCTATCGAAAAGACCATCTCCAAGGCTAA |
| | | GGGCCAGCCTCGGGAACCTCAGGTTTACA |
| | | CCCTGCCTCCATCTCGGGAAGAGATGACCA |
| | | AGAACCAGGTGTCCCTGACCTGCCTCGTGA |
| | | AGGGCTTCTACCCTTCCGATATCGCCGTGG |
| | | AATGGGAGTCCAATGGCCAGCCTGAGAAC |
| | | AACTACAAGACAACCCCTCCTGTGCTGGAC |
| | | TCCGACGGCTCATTCTTCCTGTACTCCAAG |
| | | CTGACAGTGGACAAGTCTCGGTGGCAGCA |
| | | GGGCAACGTGTTCTCCTGTTCTGTGATGCA |
| | | CGAGGCCCTGCACAACCACTACACACAGA |
| | | AGTCCCTGTCTCTGTCCCCTGGCAAGTGA |
| 114 | AGX-A01 H1v1 | EVQLVESGGGLVKPGGSLRLSCAASGFTFSS |
| | Heavy chain amino acid | FAMSWVRQAPGKGLEWVSTISSGSIYIYYTD |
| | | SVKGRFTISRDNAKNSLYLQMNSLRAEDTAV |
| | | YYCARRGIYYGYEGYAMDYWGQGTLVTVS |
| | | SASTKGPSVFPLAPSSKSTSGGTAALGCLVK |
| | | DYFPEPVTVSWNSGALTSGVHTFPAVLQSSG |
| | | LYSLSSVVTVPSSSLGTQTYICNVNHKPSNTK |
| | | VDKKVEPKSCDKTHTCPPCPAPEAAGAPSVF |
| | | LFPPKPKDTLMISRTPEVTCVVVDVSHEDPE |
| | | VKFNWYVDGVEVHNAKTKPREEQYNSTYR |
| | | VVSVLTVLHQDWLNGKEYKCKVSNKALPAP |
| | | IEKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 115 | VH-CDR1 | GFTFSSFAMS |
| 116 | VH-CDR2 (variant 1) | TISSGSIYIYYTDGVKG |
| 117 | VH-CDR2 (variant 2) | TISSGSIYIYYTDSVKG |
| 118 | VH-CDR3 (variant 1) | RGIYYGYDGYAMDY |
| 119 | VH-CDR3 (variant 2) | RGIYYGYEGYAMDY |
| 120 | VH-CDR3 (variant 3) | RGIYYGYSGYAMDY |
| 121 | VH-CDR3 (variant 4) | RGIYYGYAGYAMDY |
| 122 | AGX-A01 L10 | AIVLTQSPGTLSLSPGERATLSCRSSQSLVHS |
| | Light chain amino acid | NGNTYLHWYMQKPGQAPRVLIYKVSNRFSG |
| | | IPDRFSGSGSGTDFTLTISRLEPDDFAIYYCSQ |
| | | STHIPLAFGQGTKLEIKRTVAAPSVFIFPPSDE |
| | | QLKSGTASVVCLLNNFYPREAKVQWKVDN |
| | | ALQSGNSQESVTEQDSKDSTYSLSSTLTLSKA |
| | | DYEKHKVYACEVTHQGLSSPVTKSFNRGEC |
| 123 | AGX-A01 L10 | GCCATCGTGTTGACCCAGTCTCCAGGCACA |
| | Light chain nucleic acid | TTGTCTCTGAGCCCTGGCGAGAGAGCTACC |
| | | CTGTCCTGCAGATCTTCTCAGTCCCTGGTG |
| | | CACTCCAACGGCAACACCTACCTGCACTGG |
| | | TACATGCAGAAGCCCGGACAGGCTCCCAG |
| | | AGTGCTGATCTACAAGGTGTCCAACCGGTT |
| | | CTCTGGCATCCCCGACAGATTTTCCGGCTC |
| | | TGGCTCTGGCACCGACTTCACCCTGACCAT |
| | | CTCTAGACTGGAACCCGACGACTTCGCCAT |
| | | CTACTACTGCTCCCAGTCCACACACATCCC |
| | | TCTGGCTTTTGGCCAGGGCACCAAGCTGGA |
| | | AATCAAGAGAACCGTGGCCGCTCCTTCCGT |
| | | GTTCATCTTCCCACCATCTGACGAGCAGCT |
| | | GAAGTCCGGCACAGCTTCTGTCGTGTGCCT |
| | | GCTGAACAACTTCTACCCTCGGGAAGCCA |
| | | AGGTGCAGTGGAAGGTGGACAATGCCCTG |
| | | CAGTCCGGCAACTCCCAAGAGTCTGTGACC |
| | | GAGCAGGACTCCAAGGACTCTACCTACAG |
| | | CCTGTCCTCCACACTGACCCTGTCTAAGGC |
| | | CGACTACGAGAAGCACAAGGTGTACGCCT |
| | | GTGAAGTGACCCACCAGGGCCTGTCTAGC |
| | | CCTGTGACCAAGTCTTTCAACCGGGGCGAG |
| | | TGTTGA |
| 124 | VL-CDR1 (variant 1) | RSSQSLVHSNGNTYLH |
| 125 | VL-CDR1 (variant 2) | RSSQSLVHSSGNTYLH |
| 126 | VL-CDR1 (variant 3) | RSSQSLVHSTGNTYLH |
| 127 | VL-CDR1 (variant 4) | RSSQSLVHSQGNTYLH |
| 128 | VL-CDR2 | KVSNRFS |
| 129 | VL-CDR3 | SQSTHIPLA |

Humanized AGX-A07 H2v1L5v2

| | | |
|---|---|---|
| 130 | AGX-A07 H2v1 | EVQLVQSGAEVKKPGASVKVSCKASGYTFT |
| | Heavy chain variable region | NYGVKWVRQAPGQGLEWMGWINTYTGNPI |
| | amino acid | YAADFKGRVTMTTDTSTSTAYMELRSLRSD |
| | | DTAVYYCVRFQYGDYRYFDVWGQGTLVTV |
| | | SS |
| 131 | AGX-A07 H2v1L5v2 | EIVLTQSPATLSLSPGERATLSCRAQSGISFIN |
| | Light chain variable region | WYQQKPGQAPRLLIYGTANLASGIPARFSGS |
| | amino acid | GSGRDFTLTISSLEPEDFAVYYCQQWSSNPLT |
| | | FGGGTKVEIK |

Humanized AGX-A07 H2L5

| | | |
|---|---|---|
| 132 | AGX-A07 H2 | QVQLVQSGAEVKKPGASVKVSCKASGYTFT |
| | Heavy chain variable region | NYGVKWVRQAPGQDLEWMGWINTYTGNPI |
| | amino acid | YAADFKGRVTMTTDTSTSTAFMELRSLRSD |
| | | DTAVYYCVRFQYGDYRYFDVWGQGTLVTV |
| | | SS |
| 133 | AGX-A07 L5 | EIILTQSPATLSLSPGERATLSCRANSGISFIN |
| | Light chain variable region | WYQQKPGQAPRLLIYGTANLASGIPARFGGS |
| | amino acid | GSGRDFTLTISSLEPEDFAVYYCQQWSSNPLT |
| | | FGGGTKVEIK |

Fc Region Sequences

| | | |
|---|---|---|
| 135 | IgG1 L234A/L235A/G237A | ASTKGPSVFPLAPSSKSTSGGTAALGCLVKD |
| | | YFPEPVTVSWNSGALTSGVHTFPAVLQSSGL |
| | | YSLSSVVTVPSSSLGTQTYICNVNHKPSNTKV |
| | | DKKVEPKSCDKTHTCPPCPAPEAAGAPSVFL |
| | | FPPKPKDTLMISRTPEVTCVVVDVSHEDPEV |
| | | KFNWYVDGVEVHNAKTKPREEQYNSTYRV |
| | | VSVLTVLHQDWLNGKEYKCKVSNKALPAPI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 136 | IgG1 | ASTKGPSVFPLAPSSKSTSGGTAALGCLVKD |
| | L234A/L235A/G237A+N297C | YFPEPVTVSWNSGALTSGVHTFPAVLQSSGL |
| | | YSLSSVVTVPSSSLGTQTYICNVNHKPSNTKV |
| | | DKKVEPKSCDKTHTCPPCPAPEAAGAPSVFL |
| | | FPPKPKDTLMISRTPEVTCVVVDVSHEDPEV |
| | | KFNWYVDGVEVHNAKTKPREEQYCSTYRV |
| | | VSVLTVLHQDWLNGKEYKCKVSNKALPAPI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 137 | IgG1 | ASTKGPSVFPLAPSSKSTSGGTAALGCLVKD |
| | L234A/L235A/G237A + P331G | YFPEPVTVSWNSGALTSGVHTFPAVLQSSGL |
| | | YSLSSVVTVPSSSLGTQTYICNVNHKPSNTKV |
| | | DKKVEPKSCDKTHTCPPCPAPEAAGAPSVFL |
| | | FPPKPKDTLMISRTPEVTCVVVDVSHEDPEV |
| | | KFNWYVDGVEVHNAKTKPREEQYNSTYRV |
| | | VSVLTVLHQDWLNGKEYKCKVSNKALPAGI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 138 | IgG1 | ASTKGPSVFPLAPSSKSTSGGTAALGCLVKD |
| | L234A/L235A/G237A + | YFPEPVTVSWNSGALTSGVHTFPAVLQSSGL |
| | N297C/P331G | YSLSSVVTVPSSSLGTQTYICNVNHKPSNTKV |
| | | DKKVEPKSCDKTHTCPPCPAPEAAGAPSVFL |
| | | FPPKPKDTLMISRTPEVTCVVVDVSHEDPEV |
| | | KFNWYVDGVEVHNAKTKPREEQYCSTYRV |
| | | VSVLTVLHQDWLNGKEYKCKVSNKALPAGI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 139 | IgG1 | ASTKGPSVFPLAPSSKSTSGGTAALGCLVKD |
| | L234A/L235A/G237A + | YFPEPVTVSWNSGALTSGVHTFPAVLQSSGL |
| | K322A/P331G | YSLSSVVTVPSSSLGTQTYICNVNHKPSNTKV |
| | | DKKVEPKSCDKTHTCPPCPAPEAAGAPSVFL |
| | | FPPKPKDTLMISRTPEVTCVVVDVSHEDPEV |
| | | KFNWYVDGVEVHNAKTKPREEQYNSTYRV |
| | | VSVLTVLHQDWLNGKEYKCAVSNKALPAGI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 140 | IgG1 | ASTKGPSVFPLAPSSKSTSGGTAALGCLVKD |
| | L234A/L235A/G237A + | YFPEPVTVSWNSGALTSGVHTFPAVLQSSGL |
| | E233P/P331G | YSLSSVVTVPSSSLGTQTYICNVNHKPSNTKV |
| | | DKKVEPKSCDKTHTCPPCPAPPAAGAPSVFL |
| | | FPPKPKDTLMISRTPEVTCVVVDVSHEDPEV |
| | | KFNWYVDGVEVHNAKTKPREEQYNSTYRV |
| | | VSVLTVLHQDWLNGKEYKCKVSNKALPAGI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 141 | IgG1 | ASTKGPSVFPLAPSSKSTSGGTAALGCLVKD |
| | L234A/L235A/G237A + | YFPEPVTVSWNSGALTSGVHTFPAVLQSSGL |
| | E233P/N297C | YSLSSVVTVPSSSLGTQTYICNVNHKPSNTKV |
| | | DKKVEPKSCDKTHTCPPCPAPEAAGAPSVFL |
| | | FPPKPKDTLMISRTPEVTCVVVDVSHEDPEV |
| | | KFNWYVDGVEVHNAKTKPREEQYCSTYRV |
| | | VSVLTVLHQDWLNGKEYKCKVSNKALPAPI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 142 | IgG1 | ASTKGPSVFPLAPSSKSTSGGTAALGCLVKD |
| | L234A/L235A/G237A + | YFPEPVTVSWNSGALTSGVHTFPAVLQSSGL |
| | N297C/K322A/P331G | YSLSSVVTVPSSSLGTQTYICNVNHKPSNTKV |
| | | DKKVEPKSCDKTHTCPPCPAPEAAGAPSVFL |
| | | FPPKPKDTLMISRTPEVTCVVVDVSHEDPEV |
| | | KFNWYVDGVEVHNAKTKPREEQYCSTYRV |
| | | VSVLTVLHQDWLNGKEYKCAVSNKALPAGI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 143 | IgG1 | ASTKGPSVFPLAPSSKSTSGGTAALGCLVKD |
| | L234A/L235A/G237A + | YFPEPVTVSWNSGALTSGVHTFPAVLQSSGL |
| | E233P/N297C/P331G | YSLSSVVTVPSSSLGTQTYICNVNHKPSNTKV |
| | | DKKVEPKSCDKTHTCPPCPAPEAAGAPSVFL |
| | | FPPKPKDTLMISRTPEVTCVVVDVSHEDPEV |
| | | KFNWYVDGVEVHNAKTKPREEQYCSTYRV |
| | | VSVLTVLHQDWLNGKEYKCKVSNKALPAGI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 144 | IgG1 | ASTKGPSVFPLAPSSKSTSGGTAALGCLVKD |
| | L234A/L235A/G237A + | YFPEPVTVSWNSGALTSGVHTFPAVLQSSGL |
| | E233P/D265A/N297C/K322A/P | YSLSSVVTVPSSSLGTQTYICNVNHKPSNTKV |
| | 331G | DKKVEPKSCDKTHTCPPCPAPEAAGAPSVFL |
| | | FPPKPKDTLMISRTPEVTCVVVAVSHEDPEV |
| | | KFNWYVDGVEVHNAKTKPREEQYCSTYRV |
| | | VSVLTVLHQDWLNGKEYKCAVSNKALPAGI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |
| 145 | IgG1 | ASTKGPSVFPLAPSSKSTSGGTAALGCLVKD |
| | L234A/L235A/G237A + | YFPEPVTVSWNSGALTSGVHTFPAVLQSSGL |
| | E233P/D265A/N297C/K322A/P | YSLSSVVTVPSSSLGTQTYICNVNHKPSNTKV |
| | 331G-PGKKP | DKKVEPKSCDKTHTCPPCPAPEAAGAPSVFL |
| | | FPPKPKDTLMISRTPEVTCVVVDVSHEDPEV |
| | | KFNWYVDGVEVHNAKTKPREEQYCSTYRV |
| | | VSVLTVLHQDWLNGKEYKCAVSNKALPAGI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGKKP |
| 146 | IgG4 | EVQLVQSGAEVKKPGASVKVSCKASGYTFT |
| | S228P (sequence includes AGX- | NYGVKWVRQAPGQGLEWMGWINTYTGNPI |
| | A07 H2v1 heavy chain variable | YAADFKGRVTMTTDTSTSTAYMELRSLRSD |
| | region amino acid) | DTAVYYCVRFQYGDYRYFDVWGQGTLVTV |
| | | SSASTKGPSVFPLAPCSRSTSESTAALGCLVK |
| | | DYFPEPVTVSWNSGALTSGVHTFPAVLQSSG |
| | | LYSLSSVVTVPSSSLGTKTYTCNVDHKPSNT |
| | | KVDKRVESKYGPPCPPCPAPEFLGGPSVFLFP |
| | | PKPKDTLMISRTPEVTCVVVDVSQEDPEVQF |
| | | NWYVDGVEVHNAKTKPREEQFNSTYRVVS |
| | | VLTVLHQDWLNGKEYKCKVSNKGLPSSIEK |
| | | TISKAKGQPREPQVYTLPPSQEEMTKNQVSL |
| | | TCLVKGFYPSDIAVEWESNGQPENNYKTTPP |
| | | VLDSDGSFFLYSRLTVDKSRWQEGNVFSCSV |
| | | MHEALHNHYTQKSLSLSLGK |
| 147 | IgG4 | EVQLVQSGAEVKKPGASVKVSCKASGYTFT |
| | S228P/L235E | NYGVKWVRQAPGQGLEWMGWINTYTGNPI |
| | (sequence includes AGX-A07 | YAADFKGRVTMTTDTSTSTAYMELRSLRSD |
| | H2v1 heavy chain variable | DTAVYYCVRFQYGDYRYFDVWGQGTLVTV |
| | region amino acid) | SSASTKGPSVFPLAPCSRSTSESTAALGCLVK |
| | | DYFPEPVTVSWNSGALTSGVHTFPAVLQSSG |
| | | LYSLSSVVTVPSSSLGTKTYTCNVDHKPSNT |
| | | KVDKRVESKYGPPCPPCPAPEFEGGPSVFLFP |
| | | PKPKDTLMISRTPEVTCVVVDVSQEDPEVQF |
| | | NWYVDGVEVHNAKTKPREEQFNSTYRVVS |
| | | VLTVLHQDWLNGKEYKCKVSNKGLPSSIEK |
| | | TISKAKGQPREPQVYTLPPSQEEMTKNQVSL |
| | | TCLVKGFYPSDIAVEWESNGQPENNYKTTPP |
| | | VLDSDGSFFLYSRLTVDKSRWQEGNVFSCSV |
| | | MHEALHNHYTQKSLSLSLGK |
| 148 | IgG4 | EVQLVQSGAEVKKPGASVKVSCKASGYTFT |
| | S228P/L235E/N297C | NYGVKWVRQAPGQGLEWMGWINTYTGNPI |
| | (sequence includes AGX-A07 | YAADFKGRVTMTTDTSTSTAYMELRSLRSD |
| | H2v1 heavy chain variable | DTAVYYCVRFQYGDYRYFDVWGQGTLVTV |
| | region amino acid) | SSASTKGPSVFPLAPCSRSTSESTAALGCLVK |
| | | DYFPEPVTVSWNSGALTSGVHTFPAVLQSSG |
| | | LYSLSSVVTVPSSSLGTKTYTCNVDHKPSNT |
| | | KVDKRVESKYGPPCPPCPAPEFEGGPSVFLFP |
| | | PKPKDTLMISRTPEVTCVVVDVSQEDPEVQF |
| | | NWYVDGVEVHNAKTKPREEQFCSTYRVVSV |
| | | LTVLHQDWLNGKEYKCKVSNKGLPSSIEKTI |
| | | SKAKGQPREPQVYTLPPSQEEMTKNQVSLTC |
| | | LVKGFYPSDIAVEWESNGQPENNYKTTPPVL |
| | | DSDGSFFLYSRLTVDKSRWQEGNVFSCSVM |
| | | HEALHNHYTQKSLSLSLGK |
| 149 | IgG4 | EVQLVQSGAEVKKPGASVKVSCKASGYTFT |
| | S228P/F234A/L235E/N297C | NYGVKWVRQAPGQGLEWMGWINTYTGNPI |
| | (sequence includes AGX-A07 | YAADFKGRVTMTTDTSTSTAYMELRSLRSD |
| | H2v1 heavy chain variable | DTAVYYCVRFQYGDYRYFDVWGQGTLVTV |
| | region amino acid) | SSASTKGPSVFPLAPCSRSTSESTAALGCLVK |
| | | DYFPEPVTVSWNSGALTSGVHTFPAVLQSSG |
| | | LYSLSSVVTVPSSSLGTKTYTCNVDHKPSNT |
| | | KVDKRVESKYGPPCPPCPAPEAEGGPSVFLF |
| | | PPKPKDTLMISRTPEVTCVVVDVSQEDPEVQ |
| | | FNWYVDGVEVHNAKTKPREEQFCSTYRVVS |
| | | VLTVLHQDWLNGKEYKCKVSNKGLPSSIEK |
| | | TISKAKGQPREPQVYTLPPSQEEMTKNQVSL |
| | | TCLVKGFYPSDIAVEWESNGQPENNYKTTPP |
| | | VLDSDGSFFLYSRLTVDKSRWQEGNVFSCSV |
| | | MHEALHNHYTQKSLSLSLGK |
| 150 | IgG4 | EVQLVQSGAEVKKPGASVKVSCKASGYTFT |
| | S228P/L235E/N297C-LGKKP | NYGVKWVRQAPGQGLEWMGWINTYTGNPI |
| | (sequence includes AGX-A07 | YAADFKGRVTMTTDTSTSTAYMELRSLRSD |
| | H2v1 heavy chain variable | DTAVYYCVRFQYGDYRYFDVWGQGTLVTV |
| | region amino acid) | SSASTKGPSVFPLAPCSRSTSESTAALGCLVK |
| | | DYFPEPVTVSWNSGALTSGVHTFPAVLQSSG |
| | | LYSLSSVVTVPSSSLGTKTYTCNVDHKPSNT |
| | | KVDKRVESKYGPPCPPCPAPEFEGGPSVFLFP |
| | | PKPKDTLMISRTPEVTCVVVDVSQEDPEVQF |
| | | NWYVDGVEVHNAKTKPREEQFCSTYRVVSV |
| | | LTVLHQDWLNGKEYKCKVSNKGLPSSIEKTI |
| | | SKAKGQPREPQVYTLPPSQEEMTKNQVSLTC |
| | | LVKGFYPSDIAVEWESNGQPENNYKTTPPVL |
| | | DSDGSFFLYSRLTVDKSRWQEGNVFSCSVM |
| | | HEALHNHYTQKSLSLSLGKKP |
| 151 | IgG1 | ASTKGPSVFPLAPSSKSTSGGTAALGCLVKD |
| | M252Y/S254T/T256E | YFPEPVTVSWNSGALTSGVHTFPAVLQSSGL |
| | | YSLSSVVTVPSSSLGTQTYICNVNHKPSNTKV |
| | | DKKVEPKSCDKTHTCPPCPAPELLGGPSVFLF |
| | | PPKPKDTLYITREPEVTCVVVDVSHEDPEVKF |
| | | NWYVDGVEVHNAKTKPREEQYNSTYRVVS |
| | | VLTVLHQDWLNGKEYKCKVSNKALPAPIEK |
| | | TISKAKGQPREPQVYTLPPSREEMTKNQVSL |
| | | TCLVKGFYPSDIAVEWESNGQPENNYKTTPP |
| | | VLDSDGSFFLYSKLTVDKSRWQQGNVFSCS |
| | | VMHEALHNHYTQKSLSLSPGK |
| 152 | IgG1 | ASTKGPSVFPLAPSSKSTSGGTAALGCLVKD |
| | T252Q/M428L | YFPEPVTVSWNSGALTSGVHTFPAVLQSSGL |
| | | YSLSSVVTVPSSSLGTQTYICNVNHKPSNTKV |
| | | DKKVEPKSCDKTHTCPPCPAPELLGGPSVFLF |
| | | PPKPKDQLMISRTPEVTCVVVDVSHEDPEVK |
| | | FNWYVDGVEVHNAKTKPREEQYNSTYRVV |
| | | SVLTVLHQDWLNGKEYKCKVSNKALPAPIE |
| | | KTISKAKGQPREPQVYTLPPSREEMTKNQVS |
| | | LTCLVKGFYPSDIAVEWESNGQPENNYKTTP |
| | | PVLDSDGSFFLYSKLTVDKSRWQQGNVFSCS |
| | | VLHEALHNHYTQKSLSLSPGK |
| 153 | IgG1 | ASTKGPSVFPLAPSSKSTSGGTAALGCLVKD |
| | M428L/N434S | YFPEPVTVSWNSGALTSGVHTFPAVLQSSGL |
| | | YSLSSVVTVPSSSLGTQTYICNVNHKPSNTKV |
| | | DKKVEPKSCDKTHTCPPCPAPELLGGPSVFLF |
| | | PPKPKDTLMISRTPEVTCVVVDVSHEDPEVK |
| | | FNWYVDGVEVHNAKTKPREEQYNSTYRVV |
| | | SVLTVLHQDWLNGKEYKCKVSNKALPAPIE |
| | | KTISKAKGQPREPQVYTLPPSREEMTKNQVS |
| | | LTCLVKGFYPSDIAVEWESNGQPENNYKTTP |
| | | PVLDSDGSFFLYSKLTVDKSRWQQGNVFSCS |
| | | VLHEALHSHYTQKSLSLSPGK |
| 154 | IgG4 | EVQLVQSGAEVKKPGASVKVSCKASGYTFT |
| | T250Q/M428L | NYGVKWVRQAPGQGLEWMGWINTYTGNPI |
| | (sequence includes AGX-A07 | YAADFKGRVTMTTDTSTSTAYMELRSLRSD |
| | H2v1 heavy chain variable | DTAVYYCVRFQYGDYRYFDVWGQGTLVTV |
| | region amino acid) | SSASTKGPSVFPLAPCSRSTSESTAALGCLVK |
| | | DYFPEPVTVSWNSGALTSGVHTFPAVLQSSG |
| | | LYSLSSVVTVPSSSLGTKTYTCNVDHKPSNT |
| | | KVDKRVESKYGPPCPSCPAPEFLGGPSVFLFP |
| | | PKPKDQLMISRTPEVTCVVVDVSQEDPEVQF |
| | | NWYVDGVEVHNAKTKPREEQFNSTYRVVS |
| | | VLTVLHQDWLNGKEYKCKVSNKGLPSSIEK |
| | | TISKAKGQPREPQVYTLPPSQEEMTKNQVSL |
| | | TCLVKGFYPSDIAVEWESNGQPENNYKTTPP |
| | | VLDSDGSFFLYSRLTVDKSRWQEGNVFSCSV |
| | | LHEALHNHYTQKSLSLSLGK |
| 155 | IgG4 | EVQLVQSGAEVKKPGASVKVSCKASGYTFT |
| | M428L/N434S | NYGVKWVRQAPGQGLEWMGWINTYTGNPI |
| | (sequence includes AGX-A07 | YAADFKGRVTMTTDTSTSTAYMELRSLRSD |
| | H2v1 heavy chain region amino | DTAVYYCVRFQYGDYRYFDVWGQGTLVTV |
| | acid) | SSASTKGPSVFPLAPCSRSTSESTAALGCLVK |
| | | DYFPEPVTVSWNSGALTSGVHTFPAVLQSSG |
| | | LYSLSSVVTVPSSSLGTKTYTCNVDHKPSNT |
| | | KVDKRVESKYGPPCPSCPAPEFLGGPSVFLFP |
| | | PKPKDTLMISRTPEVTCVVVDVSQEDPEVQF |
| | | NWYVDGVEVHNAKTKPREEQFNSTYRVVS |
| | | VLTVLHQDWLNGKEYKCKVSNKGLPSSIEK |
| | | TISKAKGQPREPQVYTLPPSQEEMTKNQVSL |
| | | TCLVKGFYPSDIAVEWESNGQPENNYKTTPP |
| | | VLDSDGSFFLYSRLTVDKSRWQEGNVFSCSV |
| | | LHEALHSHYTQKSLSLSLGK |
| 156 | IgG1 | EVQLVQSGAEVKKPGASVKVSCKASGYTFT |
| | M252Y/S254T/T256E | NYGVKWVRQAPGQGLEWMGWINTYTGNPI |
| | (sequence includes AGXA07 | YAADFKGRVTMTTDTSTSTAYMELRSLRSD |
| | H2v1 heavy chain variable | DTAVYYCVRFQYGDYRYFDVWGQGTLVTV |
| | region amino acid) | SSASTKGPSVFPLAPSSKSTSGGTAALGCLVK |
| | | DYFPEPVTVSWNSGALTSGVHTFPAVLQSSG |
| | | LYSLSSVVTVPSSSLGTQTYICNVNHKPSNTK |
| | | VDKKVEPKSCDKTHTCPPCPAPELLGGPSVF |
| | | LFPPKPKDTLYITREPEVTCVVVDVSHEDPEV |
| | | KFNWYVDGVEVHNAKTKPREEQYNSTYRV |
| | | VSVLTVLHQDWLNGKEYKCKVSNKALPAPI |
| | | EKTISKAKGQPREPQVYTLPPSREEMTKNQV |
| | | SLTCLVKGFYPSDIAVEWESNGQPENNYKTT |
| | | PPVLDSDGSFFLYSKLTVDKSRWQQGNVFSC |
| | | SVMHEALHNHYTQKSLSLSPGK |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-138. (canceled)

139. An antibody drug conjugate comprising:
(i) an anti-TM4SF1 antibody or an antigen binding fragment thereof,
(ii) a therapeutic molecule; and
(iii) a linker conjugated with said ani-TM4SF1 antibody and said therapeutic molecule;
wherein said linker comprises a first fragment, wherein said first fragment comprises a moiety selected from the group consisting of: 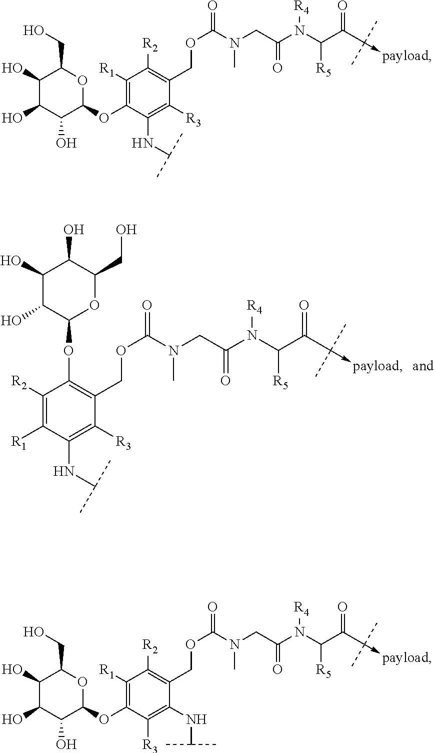
wherein:
payload indicates orientation of said moiety or said linker with respect to conjugation to said therapeutic molecule;
each R₁, R₂, and R₃ is independently H, halide, —CN, or —NO₂; and
each R₄ and R₅ is independently H, C₁-C₆ alkyl, or C₃-C₆ cycloalkyl.

140. The antibody drug conjugate of claim 139, wherein said first fragment comprises a moiety selected from the group consisting of:

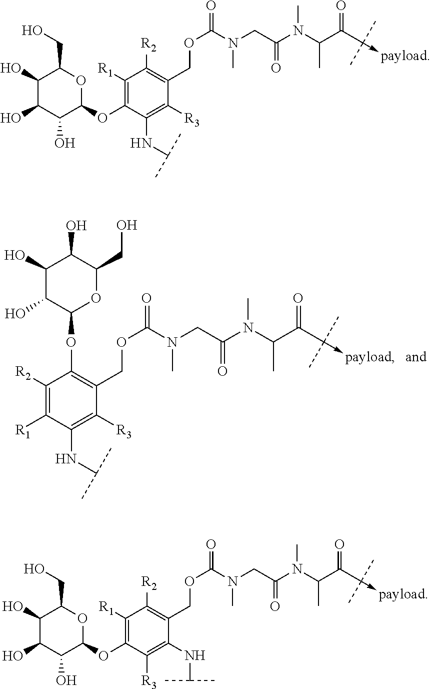

141. The antibody drug conjugate of claim 139, wherein said first fragment comprises a moiety selected from the group consisting of:

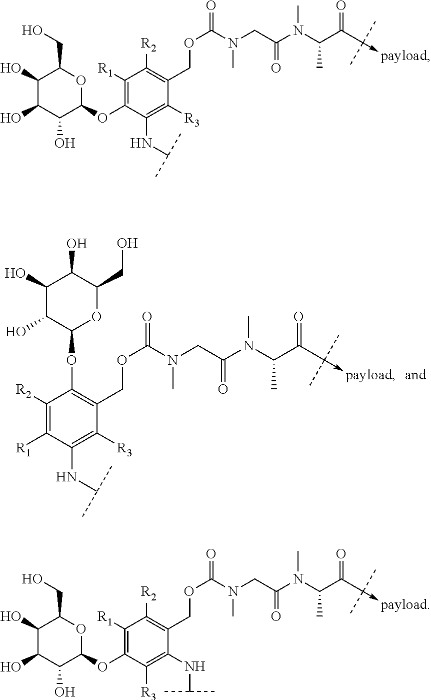

142. The antibody drug conjugate of claim 139, wherein said first fragment comprises a moiety selected from the group consisting of:

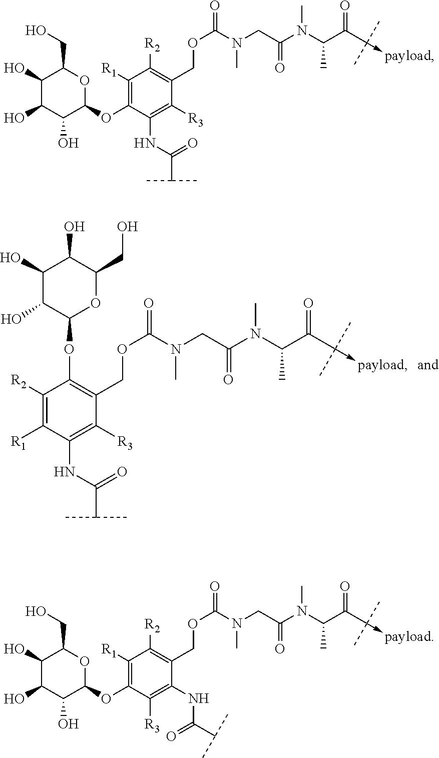

143. The antibody drug conjugate of claim 139, wherein said first fragment comprises a moiety selected from the group consisting of: 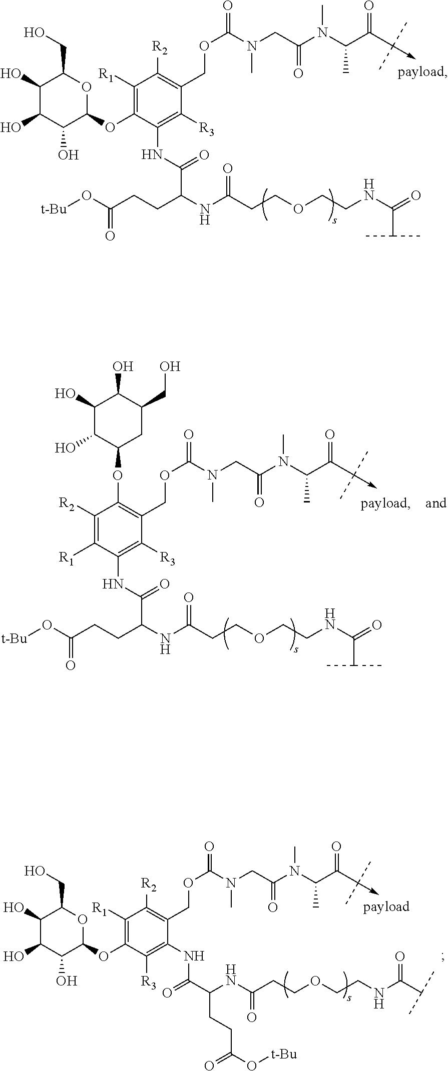
wherein s is 1, 2, 3, 4, 5, 6, 7 or 8

144. The antibody drug conjugate of claim 139, wherein said first fragment comprises a moiety selected from the group consisting of: 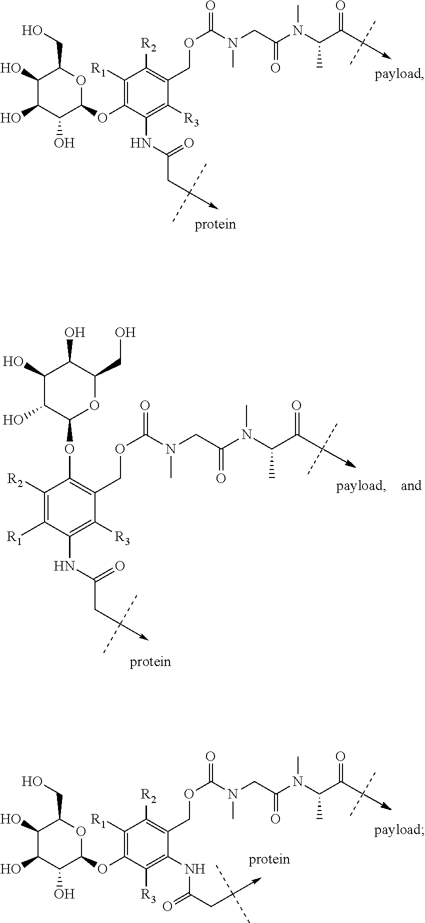
wherein →protein indicates orientation of said moiety or said linker with respect to conjugation to said anti-TM4SF1 antibody or said antigen binding fragment thereof

145. The antibody drug conjugate of claim 139, wherein each R₁, R₂, and R₃ is H.

146. The antibody drug conjugate of claim 139, wherein said first fragment comprises a moiety selected from the group consisting of: 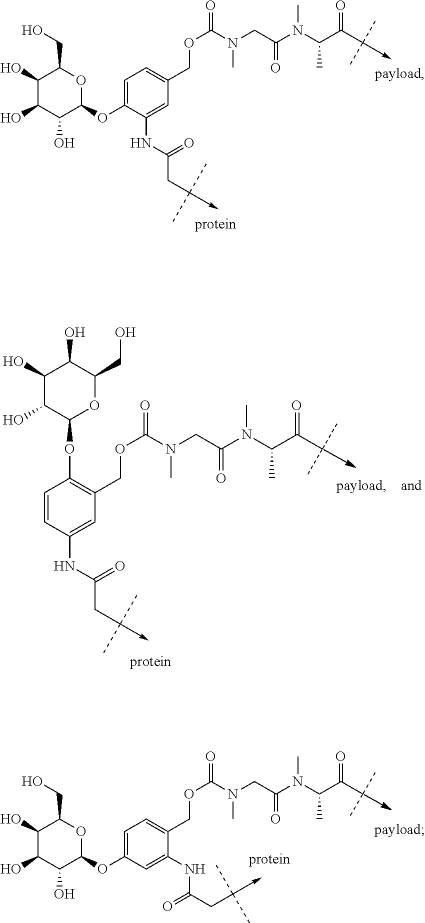
wherein protein is said anti-TM4SF1 antibody or said antigen binding fragment thereof,

147. The antibody drug conjugate of claim 139, wherein the antibody drug conjugate is: 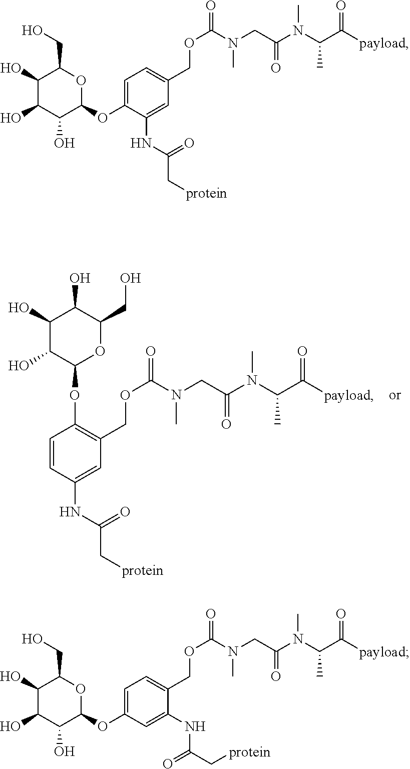
wherein protein is said anti-TM4SF1 antibody or said antigen binding fragment thereof, and wherein payload is said therapeutic molecule.

148. The antibody drug conjugate of claim 139, wherein the antibody drug conjugate is: 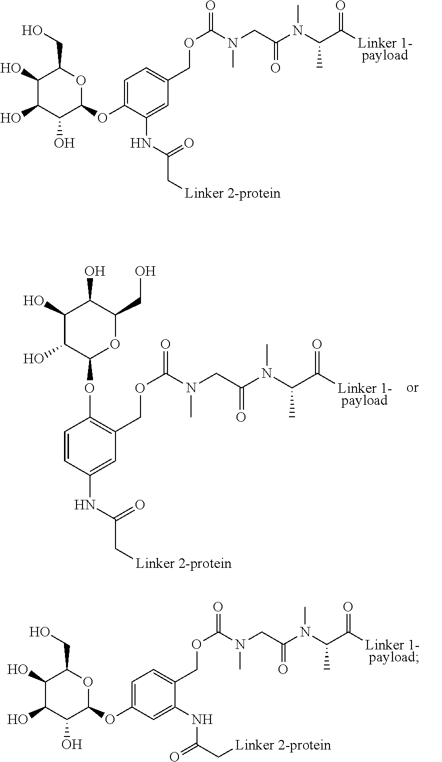
wherein protein is said anti-TM4SF1 antibody or said antigen binding fragment thereof, wherein payload is said therapeutic molecule, wherein each of Linker 1 and Linker 2 is independently alkylene, alkenylene, cycloalkylene with a 3-7 membered ring, alkynylene, arylene, heteroarylene, heterocyclene with a 5-12 membered ring comprising 1-3 atoms of N, O or S, —O—, —NH—, —S—, —N(C₁₋₆ alkyl)-, —C(═O)—, —C(═O)NH—, or combinations thereof, wherein each of said alkylene, alkenylene, cycloalkylene with said 3-7 membered ring, arylene, heteroarylene, and heterocyclene with a 5-12 membered ring comprising 1-3 atoms of N, O or S is independently unsubstituted or independently substituted with halide, amino, —CF₃, C₁-C₃ alkyl, C₃-C₆ cycloalkyl, C₁-C₃ alkoxy, C₁-C₃ alkoxy, or C₁-C₃ alkylthio.

149. The antibody drug conjugate of claim 148, wherein each of said Linker 1 and said Linker 2 is independently: 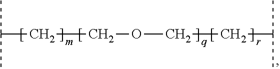
wherein m is 0-3, q is 0-12, and r is 1-3.

150. The antibody drug conjugate of claim 148, wherein each of said Linker 1 and said Linker 2 is independently: 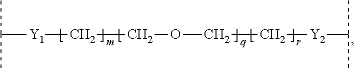
wherein:
each Y₁ and Y₂ is independently a bond, O, S, or NR₆;
R₆ is independently H, deuterium, C₁-C₆ alkyl, C₃-C₆ cycloalkyl, C₁-C₆ alkyl; C₆-C₁₂ aryl, 5-12 membered heteroaryl, C₃-C₁₂ cycloalkyl or 3-12 membered heteroalicyclic, or R₆ together with the nitrogen to which they are bound and another atom of said linker, be combined to form a 3 to 12 membered heteroalicyclic or 5-12 membered heteroaryl group optionally containing 1 to 3 additional heteroatoms selected from the group consisting of N, O, and S; and
m is 0-3, q is 0-12, and r is 1-3.

151. The antibody drug conjugate of claim 139, wherein said therapeutic molecule comprises at least one of: a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder, a DHFR inhibitor, a nucleic acid, a CRISPR enzyme, or any combinations thereof.

152. The antibody-drug conjugate of claim 139, wherein said anti-TM4SF1 antibody or said antigen binding fragment thereof comprises a modified IgG Fc region, wherein said modified IgG Fc region comprises one or more substitutions relative to a wild-type IgG Fc region, wherein said wild-type IgG Fc region is a wild-type IgG1Fc region, and wherein said modified IgG Fc region comprises an IgG1 Fc region comprising:
(a) T250Q and M428L; or
(b) L234A, L235A, and G237A; or
(c) L234A, L235A, G237A, and P331G; or
(d) L234A, L235A, G237A, N297C, and P331G; or
(e) E233P, L234A, L235A, G237A, and P331G; or
(f) E233P, L234A, L235A, G237A, and N297C; or
(g) L234A, L235A, G237A, N297C, K322A, and P331G; or
(h) E233P, L234A, L235A, G237A, D265A, N297C, K322A, and P331G; or
(i) E233P and D265A; or
(j) M252Y, S254T, and T256E; or
(k) M252Y, S254T, T256E, and N297C; or
(l) a combination thereof

153. The antibody-drug conjugate of claim 152, wherein said anti-TM4SF1 antibody or said antigen binding fragment thereof comprises:
(a) a heavy chain comprising a CDR1, CDR2, and CDR3 domain comprising an amino acid sequence of SEQ ID NOs: 6, 7, and 8, respectively; and a light chain comprising a CDR1, CDR2, and CDR3 domain comprising an amino acid sequence of SEQ ID NOs: 12, 13, and 14, respectively;
(b) a heavy chain comprising a CDR1, CDR2, and CDR3 domain comprising an amino acid sequence of SEQ ID NOs: 18, 19, and 20, respectively; and a light chain comprising a CDR1, CDR2, and CDR3 domain comprising an amino acid sequence of SEQ ID NOs: 24, 25, and 26, respectively;
(c) a heavy chain comprising a CDR1, CDR2, and CDR3 domain comprising an amino acid sequence of SEQ ID NOs: 30, 31, and 32, respectively; and a light chain comprising a CDR1, CDR2, and CDR3 domain comprising an amino acid sequence of SEQ ID NOs: 36, 37, and 38, respectively;
(d) a heavy chain comprising a CDR1, CDR2, and CDR3 domain comprising an amino acid sequence of SEQ ID NOs: 42, 43, and 44, respectively; and a light chain comprising a CDR1, CDR2, and CDR3 domain comprising an amino acid sequence of SEQ ID NOs: 48, 49, and 50, respectively;
(e) a heavy chain comprising a CDR1, CDR2, and CDR3 domain comprising an amino acid sequence of SEQ ID NOs: 54, 55, and 56, respectively; and a light chain comprising a CDR1, CDR2, and CDR3 domain comprising an amino acid sequence of SEQ ID NOs: 60, 61, and 62, respectively;
(f) a heavy chain comprising a CDR1, CDR2, and CDR3 domain comprising an amino acid sequence of SEQ ID NOs: 66, 67, and 68, respectively; and a light chain comprising a CDR1, CDR2, and CDR3 domain comprising an amino acid sequence of SEQ ID NOs: 72, 73, and 74, respectively;
(g) a heavy chain comprising a CDR1, CDR2, and CDR3 domain comprising an amino acid sequence of SEQ ID NOs: 78, 79, and 80, respectively; and a light chain comprising a CDR1, CDR2, and CDR3 domain comprising an amino acid sequence of SEQ ID NOs: 84, 85, and 86, respectively;
(h) a heavy chain comprising a CDR1, CDR2, and CDR3 domain comprising an amino acid sequence of SEQ ID NOs: 94, 95, and 96, respectively; and a light chain comprising a CDR1 comprising an amino acid sequence of SEQ ID NOs: 107 or 108, a CDR2 comprising an amino acid sequence of SEQ ID NO: 109, a CDR3 comprising an amino acid sequence of SEQ ID NOs: 110 or 111; or
(i) a heavy chain comprising a CDR1 domain comprising an amino acid sequence of SEQ ID NO: 115, a CDR2 domain comprising an amino acid sequence of SEQ ID NOs: 116 or 117, a CDR3 domain comprising an amino acid sequence of SEQ ID NOs: 118, 119, 120, or 121; and a light chain comprising a CDR1 domain comprising an amino acid sequence of SEQ ID NOs: 124, 125, 126, or 127, a CDR2 domain comprising an amino acid sequence of SEQ ID NO: 128, a CDR3 domain comprising an amino acid sequence of SEQ ID NO: 129.

154. The antibody-drug conjugate of claim 153, wherein said anti-TM4SF1 antibody or said antigen binding fragment thereof comprises:
(A) the heavy chain in (a) comprising an amino acid sequence of SEQ ID NO: 3, and the light chain in (a) comprising an amino acid sequence of SEQ ID NO: 9;
(B) the heavy chain in (b) comprising an amino acid sequence of SEQ ID NO: 15, and the light chain in (b) comprising an amino acid sequence of SEQ ID NO: 21;
(C) the heavy chain in (c) comprising an amino acid sequence of SEQ ID NO: 27, and the light chain in (c) comprising an amino acid sequence of SEQ ID NO: 33;
(D) the heavy chain in (d) comprising an amino acid sequence of SEQ ID NO: 39, and the light chain in (d) comprising an amino acid sequence of SEQ ID NO: 45;
(E) the heavy chain in (e) comprising an amino acid sequence of SEQ ID NO: 51, and the light chain in (e) comprising an amino acid sequence of SEQ ID NO: 57;
(F) the heavy chain in (f) comprising an amino acid sequence of SEQ ID NO: 63, and the light chain in (f) comprising an amino acid sequence of SEQ ID NO: 69;
(G) the heavy chain in (g) comprising an amino acid sequence of SEQ ID NO: 75, and the light chain in (g) comprising an amino acid sequence of SEQ ID NO: 81;
(H) the heavy chain in (h) comprising an amino acid sequence of SEQ ID NO: 90, 92, 130, or 132, and the light chain in (h) comprising an amino acid sequence of SEQ ID NO: 97, 99, 101, 103, 105, 131, or 133; or
(I) the heavy chain in (i) comprising an amino acid sequence of SEQ ID NO: 1, 112, or 114, and the light chain in (i) comprising an amino acid sequence of SEQ ID NO: 2 or 122

155. A pharmaceutical composition comprising (i) an antibody-drug conjugate according to claim 139 and (ii) a pharmaceutically acceptable carrier

156. A method of treating or preventing a disease or disorder in a subject, wherein said disease or disorder is characterized by abnormal endothelial cell (EC)-cell interaction, said method comprising administering to said subject an antibody-drug conjugate according to claim 139, wherein said EC-cell interaction comprises one or more of EC-mesenchymal stem cell, EC-fibroblast, EC-smooth muscle cell, EC-tumor cell, EC-leukocyte, EC-adipose cell, and EC-neuronal cell interactions, and wherein said disease or disorder is an inflammatory disease or a cancer.

157. The method of claim 156, wherein said antibody drug conjugate exhibits longer serum half-life after administration as compared to a control antibody drug conjugate comprising a wild type IgG1 Fc.

158. The method of claim 156, wherein said first fragment cleaves in said lysosomes by a beta-galactosidase enzyme, and wherein at least 50% of said antibody drug conjugate remains intact in extracellular milieu before entering lysosomes.
